SEQUENCE LISTING (22 sequences)

SEQ ID NO: 1 - DNA, 20 bp, Homo sapiens
ctgcacggatctactcgcac

SEQ ID NO: 2 - DNA, 20 bp, Homo sapiens
ctccaagagttggatcctgc

SEQ ID NO: 3 - DNA, 20 bp, Homo sapiens
tcaaataatggaggcggttt

SEQ ID NO: 4 - DNA, 20 bp, Homo sapiens
gagactatccatggagtcac

SEQ ID NO: 5 - DNA, 20 bp, Homo sapiens
gacctgagagcgaacgtccc

SEQ ID NO: 6 - DNA, 20 bp, Homo sapiens
cgcagggcccaataccacgg

SEQ ID NO: 7 - DNA, 20 bp, Homo sapiens
cactacggacagacggttcc

SEQ ID NO: 8 - DNA, 20 bp, Homo sapiens
atgctcggacgccccaccaa

SEQ ID NO: 9 - DNA, 20 bp, Homo sapiens
ccccacgccgatgaagacat

SEQ ID NO: 10 - DNA, 20 bp, Homo sapiens
ggatgcctgggactacgtgc

SEQ ID NO: 11 - DNA, 20 bp, Homo sapiens
aatctagagatccggcccac

SEQ ID NO: 12 - DNA, 20 bp, Homo sapiens
aggatcctcgggtcttacat

SEQ ID NO: 13 - DNA, 20 bp, Homo sapiens
gtgtcaaaccgactatcact

SEQ ID NO: 14 - DNA, 20 bp, Homo sapiens
gacgtacgtttcatctcacc

SEQ ID NO: 15 - DNA, 20 bp, Homo sapiens
gactaaacgggacgtgtacc

SEQ ID NO: 16 - DNA, 20 bp, Homo sapiens
tcggggactattaccacttc

SEQ ID NO: 17 - DNA, 20 bp, Homo sapiens
gcgacccaatctaaagatga

SEQ ID NO: 18 - DNA, 20 bp, Homo sapiens
gcaaggtccaaggcccttac

SEQ ID NO: 19 - DNA, 20 bp, Homo sapiens
tgcgaagcccataacgccaa

SEQ ID NO: 20 - DNA, 20 bp, Homo sapiens
cccgaagccaatgtacctcg

SEQ ID NO: 21 - DNA, 20 bp, Homo sapiens
acggtgtcgtcgacctgaac

SEQ ID NO: 22 - DNA, 20 bp, Homo sapiens
aaggtcctgacacgtcacgt

(19) United States
(12) Patent Application Publication
(10) Pub. No.: US 2023/0158073 A1
(43) Pub. Date: May 25, 2023

(54) MODULATION OF T CELL CYTOTOXICITY AND RELATED THERAPY

(71) Applicant: Cancer Research Technology Limited, London (GB)

(72) Inventors: Sergio Quezada, London (GB); Karl Peggs, London (GB); Charles Swanton, London (GB); Ehsan Ghorani, London (GB); James Reading, London (GB); Felipe Galvez-Cancino, London (GB); Despoina Karagianni, London (GB)

(73) Assignee: Cancer Research Technology Limited, London (GB)

(21) Appl. No.: 17/919,135

(22) PCT Filed: Apr. 16, 2021
(86) PCT No.: PCT/EP2021/059989

§ 371 Date: Oct. 14, 2022

(30) Foreign Application Priority Data
Apr. 17, 2020 (GB) 2005599.2

(51) Int. Cl. A61K 35/17; A61K 38/17; A61P 35/00

(52) U.S. Cl. CPC A61K 35/17; A61K 38/1774; A61P 35/00

(57) ABSTRACT

The present invention provides an engineered T cell for use in a method of treatment of a proliferative disorder, wherein the engineered T cell has modulated expression of one or more genes selected from SIT1, SAMSN1, SIRPG, CD7, CD82, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3. Further provided are activity modulators of one or more proteins encoded by genes selected from SIT1, SAMSN1, SIRPG, CD7, CD82, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3 for use in a method of enhancing immunotherapy in a subject having a proliferative disorder. Also provided are related methods of treatment employing the engineered T cell and/or inhibitor.

### FIELD OF THE INVENTION

The present invention relates to products and methods for modulating, including enhancing, T cell cytotoxicity. In particular, enhancement of T cell cytotoxicity is disclosed for use in the treatment of proliferative disorders, such as cancer.

### BACKGROUND TO THE INVENTION

Tumour neoantigens are a key substrate for T cell-mediated recognition of cancer cells (Schumacher, T. N. & Schreiber, R. D., Science 2015). Neoantigen-specific T cells respond to immune checkpoint-blockade (ICB) and have been detected in the blood and tumours of patients with non-small cell lung (NSCLC) and other cancer types (Rizvi, N. A. et al., Science 2015; McGranahan, N. et al., Science 2016; Gros, A. et al., Nat. Med., 2016). Although tumour mutational burden (TMB) predicts response to checkpoint blockade (Rizvi, N. A. et al., Science 2015; Van Allen, E. M. et al., Science 2015; Snyder, A. et al., N. Engl. J. Med. 2014) clinically evident tumours usually progress without therapy, suggesting functional impairment of anti-tumour T cell responses (Thommen, D. S. & Schumacher, T. N., Cancer Cell 2018; Reading, J. L. et al., Immunol. Rev. 2018).

T cell activation is determined by antigen characteristics including abundance, physiochemical properties, MHC affinity and self-similarity (Zinkernagel, R. M. et al., Immunol. Rev. 1997; Rolland, M. et al., PLoS One 2007; Neefjes, J. & Ovaa, H., Nature Chemical Biology 2013). In acute infection and vaccination, optimal T cell stimulation results in differentiation from progenitor (e.g. naive, central memory) to effector and effector-memory phenotypes concomitant with acquisition of diverse effector functions (Zhu, J., Yamane, H. & Paul, W. E., Annu. Rev. Immunol. 2010; Kaech, S. M. & Wherry, E. J., Immunity 2007). However, persistently high antigen load in cancer and chronic infections drives T cell differentiation into dysfunctional states, mediated by continuous T cell receptor (TCR) stimulation that induces transcription factors, including TOX, that promote gene expression, epigenetic and metabolic changes that progressively limit T cell effector functions (Wherry, E. J. & Kurachi, M. Rev. Immunol., 2015; Philip, M. & Schietinger, A. Curr. Opin. Immunol. 2019; Kallies, A., Zehn, D. & Utzschneider, D. T. Nat. Rev. Immunol. 2019).

The role of antigen exposure on the relative balance and functional characteristics of tumour infiltrating CD4 and CD8 subsets is unknown, and potentially relevant to identify critical targetable pathways restricting anti-tumour T cell function.

Guo et al., Nature Medicine 24, 978-985 (2018) describes a combined single cell expression and T cell antigen receptor based lineage tracking analysis, revealing multiple sub-populations of tumour infiltrating lymphocytes. These included tumour-infiltrating CD8⁺ T cells undergoing exhaustion, as well as cells exhibiting states preceding exhaustion. Lists of specifically expressed in each of the different subpopulations, including exhausted tumour CD8⁺ T cells (90 genes), were identified.

There remains an unmet need for therapeutic agents and methods that enhance immune-mediated cancer therapy. The present invention addresses these and other needs, and provides related advantages as described herein.

### BRIEF DESCRIPTION OF THE INVENTION

Broadly, the present invention relates to the modulation of T cell dysfunction to enhance T cell cytotoxicity and thereby to enhance anti-cancer therapy. In particular, the present disclosure relates to the use of pharmacological agents to enhance an immune response against a tumour and to the use of engineered T cells (including chimeric antigen receptor T cells (CAR-T), T cells engineered to express transgenic T cell receptors and neoantigen reactive T cells (NAR-T)) that exhibit enhanced cytotoxic activity for the treatment of a tumour. As disclosed in detail herein, the present inventors have identified key genes expressed by dysfunctional T cells expanded in tumour-infiltrating lymphocyte populations from tumours with high tumour mutational burden (termed Neo-Dys for neoantigen-associated dysfunctional T cells). They further found that these genes are key factor controlling the restriction of anti-tumour T cell function in dysfunctional T cells, and that targeting these genes potentiates the tumour immune response in cancers with high neoantigen load. In particular, targeting these genes is likely to be particularly useful in the context of tumours that are likely to show some immune escape, for example tumours that are resistant to immunotherapy or that are likely to be resistant to immunotherapy. The inventors have further experimentally validated a subset of these target genes, demonstrating the likely effect of all target genes identified.

Accordingly, in a first aspect of the invention, there is provided an engineered T cell having modulated expression of one or more genes selected from STOM, FURIN, SIT1, CD7, SAMSN1, SIRPG, CD82, FCRL3, IL1RAP, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3, for use in a method of treatment a proliferative disorder. In embodiments, the engineered T cell has reduced expression of one or more genes selected from SIT1, SAMSN1, SIRPG, CD7, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, CD82 and TNIP3, and/or increased expression or activity of one or more genes selected from SIT1, SAMSN1, SIRPG, CD7, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, CD82 and TNIP3. Preferably, the engineered T cell has reduced expression of one or more genes selected from SIT1, SAMSN1, SIRPG, CD7, CD82, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3, and/or increased expression of CD7 and/or SIRPG or increased activity of CD7 and/or SIRPG. In some such embodiments, the one or more genes are selected from AXL, CD7, E2F1, FCRL3, FURIN, IL1RAP, PECAM1, SAMSN1, SIRPG, SIT1, SUV39H1, TNIP3, and STOM. Modulated expression of a gene in the context of the present disclosure encompasses modulation at the transcript level and at the protein product level.

In embodiments, the one or more genes are selected from STOM, FURIN, SIT1, CD7, SAMSN1, SIRPG, CD82, FCRL3, IL1RAP, AXL, E2F1. Modulation of each of these genes has been experimentally demonstrated to have an effect on T cell activation. In particular, the one or more genes may advantageously be selected from the one or more genes are selected from STOM, FURIN, SIT1 and CD7. In some embodiments, the one or more genes are selected from SIT1, SAMSN1, SIRPG, CD7, CD82, FCRL3, IL1RAP, FURIN, STOM, AXL, and E2F1. In embodiments, the one or more genes are selected from STOM, FURIN, SIT1, SIRPG, IL1RAP and CD7. In particular, the one or more genes may advantageously be selected from SIT1, SIRPG and IL1RAP. Preferably, the one or more genes include SIT1.

In some embodiments, the one or more genes are selected from SIT1, CD7, STOM, FURIN, IL1RAP, SIRPG, AXL, E2F1A, CD82, SAMSN1, and FCRL3. Modulation of all of these genes in CD8 T cell was experimentally demonstrated to have an effect on T cell activation. In some such embodiments, the engineered T cell is a CD8⁺ T cell. Preferably, the one or more genes are selected from SIT1, CD7, STOM, FURIN, IL1RAP and SIRPG. In some embodiments, the one or more genes are selected from CD7, CD82, COTL1, DUSP4, FABP5, ITM2A, PARK7, PHLDA1, RAB27A, RBPJ, RGS1, RGS2, SAMSN1, SIT1, SIRPG and TNIP3. In some such embodiments, the engineered T cell is a CD8⁺ T cell. In embodiments, the one or more genes are selected from CD7, CD82, SAMSN1, SIRPG and SIT1. In particular, the one or more genes preferably include SIT1, and/or SIRPG. Preferably, the one or more genes include SIT1.

In some embodiments, the one or more genes are selected from EPHA1, FCRL3, PECAM1, STOM, AXL, FURIN, and IL1RAP. In some embodiments, the one or more genes are selected from EPHA1, FCRL3, PECAM1, STOM, AXL, FURIN, IL1RAP, E2F1, C5ORF30, CLDND1, GFI1, RNASEH2A, SIRPG and SUV39H1. In some embodiments, the one or more genes are selected from EPHA1, FCRL3, PECAM1, STOM, AXL, FURIN, IL1RAP, E2F1, C5ORF30, CLDND1, GFI1, RNASEH2A, and SUV39H1. In some such embodiments, the engineered T cell is a CD4⁺ T cell, such as an effector CD4⁺ T cell. In embodiments, the one or more genes are selected from SIT1, CD7, STOM, FURIN, IL1RAP, SIRPG, AXL, E2F1A, CD82, SAMSN1, and FCRL3. Modulation of all of these genes in CD4 T cell was experimentally demonstrated to have an effect on T cell activation. Preferably, the one or more genes are selected from AXL, FURIN, IL1RAP, STOM, FCRL3, SIRPG and E2F1. In embodiments, the one or more genes are selected from AXL, FURIN, IL1RAP, STOM, FCRL3, and E2F1. In particular, the one or more genes preferably include IL1RAP and/or SIRPG.

In some embodiments the T cell comprises a chimeric antigen receptor T cell (CAR-T), an engineered T cell receptor (TCR) T cell, an engineered T cell derived from PBMCs or a Neoantigen-reactive T cell (NAR-T). Preferably, the T cell comprises a Neoantigen-reactive T cell (NAR-T). In some embodiments, the T cell is engineered to express a transgenic T cell receptor (TCR), such as a cancer-specific TCR (e.g. NY ESO-1). In embodiments, the T cell is an engineered cell as described in Stadtmauer et al. (Science 28 Feb. 2020: vol. 367, Issue 6481, eaba7365), or a cell that has been obtained as described in Stadtmauer et al. In some embodiments, the T cell is engineered to knock-out or downregulate expression of one or more genes encoding the endogenous T cell receptor (e.g. genes encoding the endogenous T cell receptor chains TCRα (TRAC) and TCR (TRBC)). In embodiments, the engineered T cell is a TCR transduced T cell. In embodiments, the one or more genes comprise SIT1 and the engineered T cell comprises an engineered T cell derived from PBMCs. The engineered T cell may be a CAR-T cell or a TCR transduced T cell.

In embodiments, the engineered T cell has been engineered to overexpress CD7 and/or SIRPG. In some such embodiments, the engineered T cell is a tumour-infiltrating lymphocyte engineered to overexpress CD7 or wherein the engineered T cell is not a tumour-infiltrating lymphocyte and the engineered T cell has been engineered to overexpress SIRPG. In embodiments, the engineered T cell has been engineered to have reduced expression of CD7 and/or SIRPG. In some such embodiments, the engineered T cell is a tumour-infiltrating lymphocyte engineered to have reduced expression of SIRPG or wherein the engineered T cell is not a tumour-infiltrating lymphocyte and the engineered T cell has been engineered to have reduced expression of CD7. In some embodiments the T cell is autologous to said subject.

In some embodiments of any aspect of the present disclosure the proliferative disorder comprises a solid tumour. In particular, the solid tumour may be a cancerous tumour including a primary tumour or a metastasised secondary tumour. In some embodiments of any aspect of the present disclosure, the proliferative disorder comprises a tumour predicted to have high neoantigen load. In some embodiments, a tumour is predicted to have high neoantigen load if it has high tumour mutational burden. A tumour may be considered to have a high tumour mutational burden if it has at least 1 somatic mutation per megabase, at least 5 somatic mutations per megabase, or at least somatic mutations per megabase. A tumour may be predicted to have high neoantigen load if the tumour belongs to a cancer type that has high somatic mutation prevalence, for example, a cancer type that has a median numbers of somatic mutations per megabase of at least 1, at least 5 or at least 10. For example, the tumour may be a melanoma or squamous lung cancer. Somatic mutation prevalence for various cancer types have been quantified in Alexandrov et al. (Nature volume 500, pages 415-421(2013)). In some embodiments of any aspect of the present disclosure, the proliferative disorder is selected from melanoma, Lung squamous cell carcinoma, lung adenocarcinoma, bladder cancer, small cell lung cancer, oesophagus cancer, colorectal cancer, cervical cancer, head and neck cancer, stomach cancer, endometrial cancer, and liver cancer.

In some embodiments of any aspect of the present disclosure, the proliferative disorder comprises a tumour predicted to have developed or be at risk of developing immune escape. According to the present disclosure, a tumour predicted to have developed or be at risk of developing immune escape is a tumour that has acquired or is predicted to be likely to acquire or show resistance to immunotherapy. These may include: (i) tumours in patients that have already undergone immunotherapy and have failed to respond, or no longer respond to the immunotherapy, (ii) tumours in patients that are predicted to be unlikely to respond to immunotherapy, where the patients may be (immunotherapy) treatment naïve, (iii) tumours that are determined to have no or low T-cell infiltration, and (iv) tumours that have a high proportion of dysfunctional T cells in the tumour-infiltrating T cell population. In embodiments, a tumour may be considered to have a high proportion of dysfunctional T cells in the tumour-infiltrating T cell population if the expression of one or more markers selected from SIT1, SAMSN1, SIRPG, CD7, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3, is higher than a respective control value, and/or the expression of CD82 is lower than a control value, where the control values may correspond to the respective expression of the one or more markers in a control T cell population. In embodiments, a tumour may be considered to have a high proportion of dysfunctional T cells in the tumour-infiltrating T cell population if the expression of one or more markers selected from SIT1, SAMSN1, SIRPG, CD7, CD82, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3, is higher or lower than a respective control value, where the control values may correspond to the respective expression of the one or more markers in a control T cell population. The control T cell population may be a control tumour-infiltrating T cell population. The control T cell population may be a T cell population that does not show a dysfunctional phenotype. A dysfunctional T cell phenotype may be a T cell exhaustion phenotype or a terminal differentiation phenotype. The control T cell population may be a T cell population that has low PD1 expression, low GZMB expression and/or low Eomes expression. The control values may correspond to the respective expression of the one or more markers in a control T cell population that is able to control tumour proliferation. The control values may correspond to the respective expression of the one or more markers in a control T cell population that expresses IFNγ after stimulation.

In some embodiments of any aspect, the solid tumour comprises a carcinoma. In some embodiments, the carcinoma is selected from non-small cell lung cancer (NSCLC), or a renal cell carcinoma (RCC). Preferably, the carcinoma is non-small cell lung cancer (NSCLC). In embodiments, the solid tumour comprises a melanoma. In some embodiments of any aspect, the proliferative disorder is selected from lung adenocarcinoma, renal clear cell carcinoma, pancreatic adenocarcinoma, renal papillary carcinoma, hepatocellular carcinoma, adrenocortical carcinoma and mesothelioma.

In some embodiments of any aspect, the reduced expression is achieved by knock-down (downregulation) or knock-out of the one or more genes. In some embodiments the knock-out or downregulation is engineered by CRISPR/Cas9-mediated gene editing, transcription activator-like effector nucleases (TALENs) transient downregulation using short hairpin RNA (shRNA), small interfering RNA (siRNA), microRNA (miRNA), or RNA constructs for overexpression. Editing of the selected gene and/or a regulatory element (e.g. promoter) of the same are specifically contemplated.

In some embodiments the engineered T cell is for use in a method of treatment that further comprises simultaneous, sequential or separate administration of an immune checkpoint inhibitor therapy. In some cases immune checkpoint inhibitor therapy may comprise CTLA-4 blockade, PD-1 inhibition, PD-L1 inhibition, Lag-3 (Lymphocyte activating 3; Gene ID: 3902) inhibition, Tim-3 (T cell immunoglobulin and mucin domain 3; Gene ID: 84868) inhibition, TIGIT (T cell immunoreceptor with Ig and ITIM domains; Gene ID: 201633) inhibition and/or BTLA (B and T lymphocyte associated; Gene ID: 151888) inhibition. In particular, the immune checkpoint inhibitor may comprise: ipilimumab, tremelimumab, nivolumab, pembrolizumab, atezolizumab, avelumab or durvalumab.

In a second aspect the present invention provides a method of treatment of a proliferative disorder in a mammalian subject, comprising administering a therapeutically effective amount of an engineered T cell to the subject in need thereof, wherein the T cell has been engineered to have modulated expression of one or more genes selected from STOM, FURIN, SIT1, CD7, SAMSN1, SIRPG, CD82, FCRL3, IL1RAP, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3. In embodiments, the engineered T cell has reduced expression of one or more genes selected from SIT1, SAMSN1, SIRPG, CD7, CD82, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3, and/or increased expression of CD7 and/or SIRPG or increased activity of CD7 and/or SIRPG. In embodiments, the engineered T cell has been engineered to have reduced expression of one or more genes selected from SIT1, SAMSN1, SIRPG, CD7, FCRL3, IL1RAP, FURIN, STOM, AXL, E2F1, C5ORF30, CLDND1, COTL1, DUSP4, EPHA1, FABP5, GFI1, IL1RAP, ITM2A, PARK7, PECAM1, PHLDA1, RAB27A, RBPJ, RGS1, RGS2, RNASEH2A, SUV39H1, and TNIP3, and/or to have increased expression of CD82 or increased activity of CD82.

Preferably, the one or more genes are selected from STOM, FURIN, SIT1, CD7, SAMSN1, SIRPG, CD82, FCRL3, IL1RAP, AXL, E2F1. In embodiments, the one or more genes are selected from STOM, FURIN, SIT1 and CD7. In embodiments, the one or more genes are selected from STOM, FURIN, SIT1, CD7, IL1RAP and SIRPG. In some embodiments, the one or more genes are selected from SIT1, SAMSN1, SIRPG, CD7, CD82, FCRL3, IL1RAP, FURIN, STOM, AXL, and E2F1. In particular, the one or more genes may advantageously be selected from SIT1, SIRPG and IL1RAP. Preferably, the one or more genes include SIT1.

In some embodiments, the one or more genes are selected from SIT1, CD7, STOM, FURIN, IL1RAP, SIRPG, AXL, E2F1A, CD82, SAMSN1, and FCRL3. In some such embodiments, the engineered T cell is a CD8⁺ T cell. In some such embodiments, one or more genes are selected from SIT1, CD7, STOM, FURIN, IL1RAP and SIRPG. In some embodiments, the one or more genes are selected from CD7, CD82, COTL1, DUSP4, FABP5, ITM2A, PARK7, PHLDA1, RAB27A, RBPJ, RGS1, RGS2, SAMSN1, SIT1, SIRPG and TNIP3. In some such embodiments, the engineered T cell is a CD8⁺ T cell. Preferably, the one or more genes are selected from CD7, CD82, SAMSN1, SIRPG and SIT1. In particular, the one or more genes preferably include SIT1, and/or SIRPG. Preferably, the one or more genes include SIT1.

In some embodiments, the one or more genes are selected from EPHA1, FCRL3, PECAM1, STOM, AXL, FURIN, LL1RAP, E2F1, C5ORF30, CLDND1, GFI1, RNASEH2A, SIRPG and SUV39H1. In some such embodiments, the engineered T cell is a CD4⁺ T cell, such as an effector CD4⁺ T cell. In embodiments, the engineered T cell is a CD4⁺ T cell and the one or more genes are selected from SIT1, CD7, STOM, FURIN, IL1RAP, SIRPG, AXL, E2F1A, CD82, SAMSN1, and FCRL3. In some embodiments, the one or more genes are selected from EPHA1, FCRL3, PECAM1, STOM, AXL, FURIN and IL1RAP. Preferably, the one or more genes are selected from AXL, FURIN, IL1RAP, STOM, FCRL3, SIRPG and E2F1. In particular, the one or more genes preferably include SIRPG and/or IL1RAP.

In some embodiments the T cell comprises an engineered T cell derived from PBMCs, a chimeric antigen receptor T cell (CAR-T), an engineered T cell receptor (TCR) T cell or a Neoantigen-reactive T cell (NAR-T). Preferably, the T cell comprises a Neoantigen-reactive T cell (NAR-T). In some embodiments, the T cell is engineered to express a transgenic T cell receptor (TCR), such as a cancer-specific TCR (e.g. NY ESO-1). In embodiments, the one or more genes comprise SIT1 and the engineered T cell comprises an engineered T cell derived from PBMCs. In embodiments, the engineered T cell has been engineered to overexpress CD7 and/or SIRPG. In some such embodiments, the engineered T cell is a tumour-infiltrating lymphocyte engineered to overexpress CD7 or wherein the engineered T cell is not a tumour-infiltrating lymphocyte and the engineered T cell has been engineered to overexpress SIRPG. In embodiments, the engineered T cell has been engineered to have reduced expression of CD7 and/or SIRPG. In some such embodiments, the engineered T cell is a tumour-infiltrating lymphocyte engineered to have reduced expression of SIRPG or wherein the engineered T cell is not a tumour-infiltrating lymphocyte and the engineered T cell has been engineered to have reduced expression of CD7. In some embodiments the T cell is autologous to said subject. In autologous T cell therapy the T cells removed from the subject are typically engineered ex vivo, e.g. to target the T cells to an antigen expressed on the tumour (for example to insert a gene encoding a chimeric antigen receptor). Advantageously in accordance with the present invention the T cells may be additionally engineered during or as part of this ex vivo stage to downregulate expression of the one or more selected genes before the T cells are then returned to the subject.

In some embodiments the T cell is engineered to knock-out or downregulate expression of the one or more selected genes prior to being administered to the subject. For example, the T cell may be engineered to knock-out or downregulate expression of one or more genes encoding the endogenous T cell receptor (e.g. genes encoding the endogenous T cell receptor chains TCRα (TRAC) and TCRβ (TRBC)). In some embodiments the knock-out or downregulation is engineered by CRISPR/Cas9-mediated gene editing, transcription activator-like effector nucleases (TALENs) transient downregulation using short hairpin RNA (shRNA), small interfering RNA (siRNA), microRNA (miRNA), or RNA constructs for overexpression.

In some embodiments the method further comprises simultaneous, sequential or separate administration of an immune checkpoint inhibitor therapy to the subject. Such combination therapy may give rise to synergistic enhancement of anti-tumour effects. In particular, the immune checkpoint inhibitor therapy may comprise CTLA-4 blockade, PD-1 inhibition, Lag-3 (Lymphocyte activating 3; Gene ID: 3902) inhibition, Tim-3 (T cell immunoglobulin and mucin domain 3; Gene ID: 84868) inhibition, TIGIT (T cell immunoreceptor with Ig and ITIM domains; Gene ID: 201633) inhibition, BTLA (B and T lymphocyte associated; Gene ID: 151888) inhibition and/or PD-L1 inhibition. For example, the immune checkpoint inhibitor may be selected from: ipilimumab, tremelimumab, nivolumab, pembrolizumab, atezolizumab, avelumab and durvalumab.

In some embodiments, the method further comprises simultaneous, sequential or separate administration of an activity modulator according to the third aspect.

In a third aspect the present invention provides activity modulators of one or more proteins encoded by genes selected from STOM, FURIN, SIT1, CD7, SAMSN1, SIRPG, CD82, FCRL3, IL1RAP, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3 for use in a method of enhancing immunotherapy in a subject having a proliferative disorder. Preferably, the activity modulators are inhibitors and the one or more genes are selected from: STOM, FURIN, CD7, SIT1, IL1RAP, SAMSN1, SIRPG, CD82, FCRL3, E2F1, and AXL. In embodiments, the activity modulators are inhibitors and the one or more genes are selected from: SIT1, SAMSN1, SIRPG, CD7, CD82, FCRL3, IL1RAP, FURIN, STOM, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3. In embodiments, the activity modulator is an activator of CD82. In embodiments, the activity modulator is an activator of CD7 and/or SIRPG. In particular, the activity modulators are inhibitors and the one or more genes are selected from: SIT1, SIRPG and IL1RAP. Preferably, the one or more genes include SIT1. An activity modulator may be an inhibitor such as a small molecule inhibitor or a blocking antibody. An activity modulator may be an activator, such as an agonist (e.g. an agonist antibody or ligand).

In some embodiments, the activity modulator is a small molecule inhibitor of AXL, CLDND1, E2F1, FABP5, FURIN, IL1RAP, SAMSN1, SUV39H1 or TNIP3. Preferably, the activity modulator is a small molecule inhibitor of AXL, CLDND1, E2F1, FURIN, IL1RAP, SAMSN1, SUV39H1 or TNIP3. Available small molecule inhibitors of AXL include BGB324 (Bemcentinib) and TP-093 (Dubermatinib). Available small molecule inhibitors of E2F1 include HLM006474 (Calbiochem, CAS 353519-63-8). Available small molecule inhibitors of FABP5 include palmitic acid (PubChem Substance ID 24898107).

In some embodiments, the activity modulator is a (poly)peptide, such an antibody or fragment thereof that binds to and inhibits AXL, CD7, FCRL3, EPHA1, IL1RAP, ITM2A, PARK7, PECAM1, TNIP3 or SIRPG. Preferably, the activity modulator is an antibody or fragment thereof that binds to and inhibits AXL, CD7, FCRL3, or SIRPG. Antibodies binding AXL include YW327.652 (Creative Biolabs®), AF154 (R&D Systems®), and h #11B7-T11 (Creative Biolabs®). Antibodies binding CD7 include V55P2F2*B12 (Vertebrates Antibodies Limited), RTF2 (Creative Biolabs®), and CHT₂ (Creative Biolabs®). Antibodies binding EPHA1 include 2G7 (Creative Biolabs®). Antibodies binding FABP5 include HPA051895 and SAB1401130 (Merck®). Antibodies binding IL1RAP include JG38-07 (Creative Biolabs®). Antibodies binding ITM2A include CBACN-303 (Creative Biolabs®). Antibodies binding PARK7 include CBL625 (Creative Biolabs®). Antibodies binding PECAM1 include 2H8 (Thermo Fisher Scientific®), HRCT (Abcam®), 2H8 (Abcam®), 8E3 (Creative Biolabs®), etc. Antibodies binding SIRPG include 3H7 (Creative Biolabs®), and OX-119 (Absolute Antibody). A TNIP3 blocking peptide (NBP1-77365PEP) is available from Novus Biologicals®.

In some embodiments the immunotherapy comprises immune checkpoint inhibition, an anti-tumour vaccine or an autologous T cell therapy. In some embodiments, the immunotherapy comprises administering an engineered T cell according to the first or second aspect. In some embodiments the amount or dose of inhibitor/activator is administered to the subject is sufficient to enhance cytotoxic activity of CD4⁺ and/or CD8⁺ T cells in the subject.

In a fourth aspect the present invention provides activity modulators of one or more proteins encoded by genes selected from SIT1, SAMSN1, SIRPG, CD7, CD82, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3 for use in a method of enhancing the immune response of a subject having a proliferative disorder. The activity modulators may be activators or inhibitors. In embodiments, the activity modulator is an inhibitor, preferably an inhibitor of one or more proteins encoded by genes selected from STOM, FURIN, CD7, SIT1, IL1RAP, SAMSN1, SIRPG, CD7, CD82, FCRL3, IL1RAP, FURIN, STOM, E2F1, and AXL. In embodiments, the activity modulator is an activator, preferably an activator of one or more proteins encoded by genes selected from CD7 and SIRPG. In embodiments, the activity modulators are inhibitors and the one or more genes are selected from: SIT1, SAMSN1, SIRPG, CD7, CD82, FCRL3, IL1RAP, FURIN, STOM, E2F1, C5ORF30, CLDND1, COTL1, DUSP4, EPHA1, FABP5, GFI1, IL1RAP, ITM2A, PARK7, PECAM1, PHLDA1, RAB27A, RBPJ, RGS1, RGS2, RNASEH2A, SUV39H1, and TNIP3. In embodiments, the activity modulator is an activator of CD82. In some embodiments, the activity modulator is an inhibitor of SIT1, SIRPG or IL1RAP. In specific embodiments, the activity modulator is an inhibitor of CD82.

In some embodiments, the activity modulator is a small molecule inhibitor of AXL, CLDND1, E2F1, FURIN, IL1RAP, SAMSN1, SUV39H1 or TNIP3. In specific embodiments, the activity modulator is a small molecule inhibitor of CLDND1, E2F1, FURIN, IL1RAP, SAMSN1, SUV39H1 or TNIP3. In specific embodiments, the activity modulator is a small molecule inhibitor of IL1RAP. In some embodiments, the activity modulator is an antibody or fragment thereof that binds to and inhibits AXL, CD7, FCRL3, or SIRPG. In specific embodiments, the activity modulator is an antibody or fragment thereof that binds to and inhibits CD7, FCRL3, or SIRPG. In specific embodiments, the activity modulator is an antibody or fragment thereof that binds to and inhibits SIRPG.

In some embodiments the method further comprises administration of an immunotherapy. In some such embodiments, the immunotherapy comprises immune checkpoint inhibition, an anti-tumour vaccine or an autologous T cell therapy. In some embodiments, the immunotherapy comprises T cell therapy using an engineered T cell according to the first or second aspect. In some embodiments the amount or dose of inhibitor/activator is administered to the subject is sufficient to enhance cytotoxic activity of CD4⁺ and/or CD8⁺ T cells in the subject.

In a fifth aspect the present invention provides a method of treatment of a proliferative disorder in a mammalian subject, comprising administering a therapeutically effective amount of an activity modulator of one or more proteins encoded by genes selected from STOM, FURIN, CD7, SIT1, IL1RAP, SAMSN1, SIRPG, CD82, FCRL3, AXL, E2F1, C5ORF30, CLDND1, COTL1, DUSP4, EPHA1, FABP5, GFI1, IL1RAP, ITM2A, PARK7, PECAM1, PHLDA1, RAB27A, RBPJ, RGS1, RGS2, RNASEH2A, SUV39H1, and TNIP3 to the subject, wherein the activity modulator enhances cytotoxic activity of one or more T cells in the subject and thereby treats the proliferative disorder.

The activity modulators may be activators or inhibitors. In embodiments, the activity modulator is an inhibitor, preferably an inhibitor of one or more proteins encoded by genes selected from STOM, FURIN, CD7, SIT1, IL1RAP, SAMSN1, SIRPG, CD7, CD82, FCRL3, IL1RAP, FURIN, STOM, E2F1, and AXL. In embodiments, the activity modulator is an activator, preferably an activator of one or more proteins encoded by genes selected from CD7 and SIRPG. In embodiments, the activity modulators are inhibitors and the one or more genes are selected from: SIT1, SAMSN1, SIRPG, CD7, CD82, FCRL3, IL1RAP, FURIN, STOM, E2F1, C5ORF30, CLDND1, COTL1, DUSP4, EPHA1, FABP5, GFI1, IL1RAP, ITM2A, PARK7, PECAM1, PHLDA1, RAB27A, RBPJ, RGS1, RGS2, RNASEH2A, SUV39H1, and TNIP3. In embodiments, the activity modulator is an activator of CD82. In some embodiments, the activity modulator is an inhibitor of SIT1, SIRPG or IL1RAP. In specific embodiments, the activity modulator is an inhibitor of SIT1.

In embodiments, the method of treatment further comprises administering an engineered T cell according to the first or second aspect.

In a sixth aspect the present invention provides a method of treatment of a proliferative disorder in a mammalian subject, comprising administering a therapeutically effective amount of an engineered T cell according to the first or second aspect. In embodiments, the method of treatment further comprises administering an activity modulator according to the third aspect.

According to a seventh aspect, the invention provides a method for producing an engineered T cell, comprising genetically engineering a T cell to enhance expression and/or knock-out or downregulate expression of one or more genes selected from SIT1, SAMSN1, SIRPG, CD7, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, CD82 and TNIP3. In embodiments, the method comprises genetically engineering a T cell to knock-out or downregulate expression of one or more genes selected from SIT1, SAMSN1, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, CD82 and TNIP3. In embodiments, the method comprises genetically engineering a T cell to enhance expression of one or more genes selected from CD7 and SIRPG.

In embodiments, the method further comprises culturing the T cell under conditions suitable for expansion to provide an expanded cell population. In some embodiments, the method is performed in vitro. In embodiments, genetically engineering a T cell is performed by CRISPR/Cas9-mediated gene editing, transcription activator-like effector nucleases (TALENs) transient downregulation using short hairpin RNA (shRNA), small interfering RNA (siRNA), microRNA (miRNA) or RNA constructs for overexpression or by introducing a nucleic acid or vector into the cell.

In some embodiments, the method comprises genetically engineering a T cell to enhance expression of CD82 and/or knock-out or downregulate expression of one or more genes selected from SIT1, SAMSN1, SIRPG, CD7, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3. In some such embodiments, the one or more genes are selected from AXL, CD7, E2F1, FCRL3, FURIN, IL1RAP, PECAM1, SAMSN1, SIRPG, SIT1, SUV39H1, TNIP3, and STOM. In embodiments, the method comprises genetically engineering a T cell to enhance expression of CD7 and/or SIRPG, and/or knock-out or downregulate expression of one or more genes selected STOM, FURIN, SIT1, CD7, SAMSN1, SIRPG, CD82, FCRL3, IL1RAP, AXL, E2F1. In some embodiments, the method comprises genetically engineering a T cell to enhance expression of CD82 and/or knock-out or downregulate expression of one or more genes selected from SIT1, SAMSN1, SIRPG, CD7, FCRL3, IL1RAP, FURIN, STOM, AXL, and E2F1. In particular, the one or more genes may advantageously be selected from SIT1, SIRPG and IL1RAP. Preferably, the one or more genes include SIT1.

In some embodiments, the method comprises genetically engineering a T cell to enhance expression of CD82 and/or knock-out or downregulate expression of one or more genes selected from CD7, COTL1, DUSP4, FABP5, ITM2A, PARK7, PHLDA1, RAB27A, RBPJ, RGS1, RGS2, SAMSN1, SIT1, SIRPG and TNIP3. In some such embodiments, the engineered T cell is a CD8⁺ T cell. Preferably, the method comprises genetically engineering a T cell to enhance expression of CD82 and/or knock-out or downregulate expression of one or more genes selected from CD7, SAMSN1, SIRPG and SIT1. In particular, the one or more genes preferably include SIT1, and/or SIRPG. Preferably, the one or more genes include SIT1.

In some embodiments, the method comprises genetically engineering a T cell to knock-out or downregulate expression of one or more genes selected from EPHA1, FCRL3, PECAM1, STOM, AXL, FURIN, and IL1RAP. In some embodiments, the method comprises genetically engineering a T cell to knock-out or downregulate expression of one or more genes selected from STOM, FURIN, SIT1, SAMSN1, CD82, FCRL3, IL1RAP, AXL, E2F1. In some embodiments, the one or more genes are selected from EPHA1, FCRL3, PECAM1, STOM, AXL, FURIN, IL1RAP, E2F1, C5ORF30, CLDND1, GFI1, RNASEH2A, SIRPG and SUV39H1. In some embodiments, the one or more genes are selected from EPHA1, FCRL3, PECAM1, STOM, AXL, FURIN, IL1RAP, E2F1, C5ORF30, CLDND1, GFI1, RNASEH2A, and SUV39H1. In some such embodiments, the engineered T cell is a CD4⁺ T cell, such as an effector CD4⁺ T cell. Preferably, the one or more genes are selected from AXL, FURIN, IL1RAP, STOM, FCRL3, SIRPG, and E2F1. In embodiments, the one or more genes are selected from AXL, FURIN, IL1RAP, STOM, FCRL3, and E2F1. In particular, the one or more genes preferably include IL1RAP and/or SIRPG. In embodiments, the engineered T cell is a CD8⁺ T cell and the one or more genes are selected from SIT1, CD7, STOM, FURIN, IL1RAP, SIRPG, AXL, E2F1A, CD82, SAMSN1, and FCRL3. In some such embodiments, the one or more genes are selected from SIT1, CD7, STOM, FURIN, IL1RAP and SIRPG. In embodiments, the engineered T cell is a CD4⁺ T cell and the one or more genes are selected from SIT1, CD7, STOM, FURIN, IL1RAP, SIRPG, AXL, E2F1A, CD82, SAMSN1, and FCRL3.

In some embodiments the T cell is a chimeric antigen receptor T cell (CAR-T), an engineered T cell receptor (TCR) T cell or a Neoantigen-reactive T cell (NAR-T). Preferably, the T cell is a Neoantigen-reactive T cell (NAR-T). In embodiments, the T cell is a T cell derived from PBMCs. In some embodiments, the T cell is engineered to express a transgenic T cell receptor (TCR), such as a cancer-specific TCR (e.g. NY ESO-1). In some embodiments, the T cell is engineered to knock-out or downregulate expression of one or more genes encoding the endogenous T cell receptor (e.g. genes encoding the endogenous T cell receptor chains TCRα (TRAC) and TCRβ (TRBC)). In some embodiments, the method comprises genetically engineering a T cell to express the transgenic T cell receptor (TCR), and/or to knock-out or downregulate expression of one or more genes encoding the endogenous T cell receptor. In some embodiments the T cell is autologous to a subject. In some embodiments, the T cell is for use in any of the methods of treatment described herein.

According to a further aspect, the invention provides a method for enhancing the cytotoxicity of an engineered T cell, the method comprising genetically engineering an engineered T cell to enhance expression and/or knock-out or downregulate expression of one or more genes selected from SIT1, SAMSN1, SIRPG, CD7, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, CD82 and TNIP3. In embodiments, the method comprises genetically engineering a T cell to knock-out or downregulate expression of one or more genes selected from SIT1, SAMSN1, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, CD82 and TNIP3. In embodiments, the method comprises genetically engineering a T cell to enhance expression of one or more genes selected from CD7 and SIRPG. In embodiments, the engineered T cell is a chimeric antigen receptor T cell (CAR-T), an engineered T cell receptor (TCR) T cell or a Neoantigen-reactive T cell (NAR-T). In embodiments, the T cell is a T cell derived from PBMCs.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1—Data availability and samples description for Example 1. (A-C) High dimensional flow cytometry, genomic and transcriptomic data from surgically resected early NSCLC specimens obtained from patients in the ‘Tracking Cancer Evolution through Therapy’ (TRACERx) 100 cohort were analysed, along with bulk and single T cell transcriptomic data from independent cohorts (cohorts 1 and 2) (A) Sample data availability and disposition for TRACERx 100 flow cytometry and RNA sequencing cohorts, with details of matched data relevant to key analyses. (B) Patients and regional data availability for flow cytometry cohorts 1 and 2. (C) Demographic details for all TRACERx 100 flow cohort patients.

FIG. 2—Characterisation of NSCLC intratumour CD4 T cell differentiation landscape by flow cytometry (A-I) 19-marker flow cytometry was performed on tumour infiltrating lymphocytes (TILS) from 44 tumour regions of 14 patients in the TRACERx 100 cohort. Unsupervised clustering of combined region data identified 20 CD4 subpopulations that were manually grouped into nine meta-clusters based on marker expression and co-localisation in uniform manifold approximation and projection (UMAP) dimension reduced space. Correlation with TMB was investigated. (A) Heatmap represents min-max scaled marker expression for all 20 clusters identified by unsupervised clustering of flow cytometry data obtained from all samples. Numbers in individual cells represent the median expression level for each marker in a cluster. Bars on the left show the number of events within each cluster. Clusters were combined into metaclusters according to their phenotypic features and co-localisation in the UMAP dimension reduction plot. (B) UMAP dimension reduction of the CD4 differentiation landscape. The position of metaclusters from (A) are numbered. (C) Cluster stability across 1000 iterations of unsupervised clustering. The cluster identity of cell was determined for one representative iteration (labels are on the right of the heatmap). For each cell, the probability of being identified within each cluster (labelled below the plot) across 1000 iterations is represented. (D) Differential abundance of all 20 clusters between tumour vs. NTL tissue. FDR adjusted −log₁₀ p-values and log₂ fold change values shown. (E) Relationship between CD4 population abundance and tumour genomic features. P-values and regression slopes (β coefficients) reflecting the direction and magnitude of relationships tested are from mixed effects regression models. (F) Gating strategy to define Early, Tdys and TDT populations for an exemplary sample. (G) For samples with distinct CD4 staining, the percentage of CD4⁺ cells amongst all manually gated is shown for each subset. (H) Disease free survival (DFS) probability of patients with high vs. low abundance of Early, Tdys and TDT subsets (categorised according to the median value). The number of patients at risk at each time point, log-rank p-value and hazard ratios with 95% confidence intervals are shown. (I) Relationship between CD4 subset abundance and stage. Mixed effects regression model p-values are shown (NS=non-significant).

FIG. 3—CD4 differentiation skewing occurs in association with tumour mutational burden. (A) Iterated clustering of high dimensional flow cytometry data from intratumour CD4 T cells to identify populations that vary with TMB. (B) Heatmap showing populations that were found to stably change in abundance with TMB. Correlation between cluster abundance and TMB is shown on the right (Pearson r-value). (C) Differential cluster abundance in tumour vs. NTL tissue. False discovery rate adjusted p-values and log 2 fold change values are shown. Size of points reflects cluster abundance. (D) The distribution of Early, Tdys and TDT clusters in all tumour regions evaluated. Regional TMB is indicated above the plot. (E) Loss of Early and gain in abundance of dysfunctional subsets with TMB, in independent cohorts drawn from the first 100 TRACERx patients. Independent analysis of manually gated populations (expressed as a percentage of all CD4 cells) within discovery cohort 1, validation cohort 2, (left and middle columns) and a combined analysis (right column) are shown. Each point represents a tumour region, Pearson p- and r-values are shown along with p-values corrected for histology and tumour multiregionality (p_{c}) from mixed effects regression models. (F) Dimension reduction of the CD4 differentiation landscape by UMAP. CD4 differentiation landscape at different levels of TMB and distribution of PD1 and Eomes fluorescence intensities are shown.

FIG. 4—Characterisation of Early, Tdys and TDT subsets. (A) The PD1 vs. CD57 expression profile of manually gated Early, Tdys and TDT subsets. (B) Marker profile of manually gated subsets in validation cohort 2. Ridge plots show marker distribution of individual samples contributing to biaxial plots. (C) Percentage of cells positive for key markers, according to thresholds represented in (A). Wilcoxon rank sum test p-values shown (*p<0.05, **p<0.001, ***p<0.0001, NS=not significant).

FIG. 5—Single cell transcriptomic characterisation of Early, Tdys and TDT subsets reveals distinct regulatory mechanisms. (A) Early, Tdys and TDT subsets were identified by a biaxial gating strategy applied to single cell RNAseq data, based on features identified by flow cytometry. The gating scheme for Tdys and TDT cells is shown (CD3E⁺CD3G⁺CD4⁺CD8⁻ cells were pregated, see Figure S3A). Expression values are represented as normalised, log₁₀ transformed read counts per million (log₁₀ CPM). (B) Change in Tdys and TDT with Early abundance (as a percentage of all CD4⁺ cells). Pearson p- and r-values are shown. (C) Confirmation of subset identity using markers not used in the gating strategy but whose expected expression (log₁₀ CPM) is known based on analysis of flow cytometry data. Each point represents an individual CD4 T cell and Wilcoxon rank sum test p-values are shown (*p<0.05, **p<0.001, ***p<0.0001, NS=not significant). (D) Heatmaps showing differential expression of genes involved in key T cell regulatory pathways. All genes shown are >2-fold differentially expressed between Early vs. Tdys or Early vs. TDT, with FDR adjusted p<0.01. Expression is represented as z-score scaled log₁₀ CPM values. (E, F) Enrichment of CD4 dysfunction signatures amongst genes differentially expressed by Tdys and TDT vs. Early. (G) GSEA of T helper subset signatures enriched in Tdys and TDT vs. Early, using modules from Charoentong et al. 2017. Normalised enrichment scores (NES) and FDR adjusted p-values are shown.

FIG. 6—Single cell transcriptomic characterisation of Early, Tdys and TDT subsets (A) Full gating strategy to identify the CD4 Early subset by single T cell RNA expression. (B) Differential expression of canonical Th1, Th2 and Tfh genes between Early, Tdys and TDT subsets. Wilcoxon rank sum test p-values shown (*p<0.05, **p<0.001, ***p<0.0001, NS=not significant).

FIG. 7—Single cell transcriptomic characterisation of Early, Tdys and TDT subsets Uniquely expressed surface protein encoding (A) and transcription factor encoding genes (B) in Early, Tdys and TDT populations at the single T cell RNA expression level. Each gene has >4-fold differential expression in one subset vs. the others, FDR adjusted p<0.01. Differentially expressed genes encoding adhesion molecules and chemokine receptors (C) and ITIM containing proteins (D); All genes shown are >2-fold differentially expressed between Early vs Tdys or early vs TDT, adjusted p<0.01. (E) GSEA to confirm the T central memory like transcriptional status of Early vs. Tdys/TDT cells. Normalised enrichment scores (NES) and FDR adjusted p-values are shown.

FIG. 8—A validated gene signature of CD4^{ds} predicts lung cancer survival. (A) An overview of gene signature validation. Using regions with both high dimensional flow cytometry and RNAseq, Early, Tdys and TDT subsets were identified within the flow cytometry data and expression signatures measured within the RNAseq data to identify gene signatures that predict the abundance of individual CD4 subsets. (B) Correlation between selected CD4 gene signatures and abundance of Early, Tdys and TDT subsets. Pearson correlation r- and FDR adjusted −log₁₀ p-values are shown. Significantly correlating signatures were further evaluated for their relationship with Tdys (middle panel) and TDT subsets (right panel). (C) The xCell Th2 signature (xCell differentiation skewing; XDS) correlates with TMB in TRACERx RNAseq and TCGA NSCLC cohorts. XDS signature values were z-score scaled, TMB values were log₁₀ transformed. A corrected p-value (p_{c}) is shown for the TRACERx cohort from a mixed effects regression model accounting for tumour multiregionality and histology. Pearson correlation r- and p-values are shown for TCGA analyses. (D) Kaplan-Meier plots representing disease free survival (DFS) in the TRACERx RNAseq cohort and overall survival (OS) in TCGA NSCLC cohorts for patients with high vs. low XDS, categorised according to the upper quartile. Log-rank p-values, hazard ratios and 95% confidence intervals are shown. (E) Multivariable Cox regression analysis for the relationship between XDS as a continuous variable and DFS in TRACERx. (F) Kaplan-Meier plots representing disease free survival (DFS) in six cohorts publicly available from the Cancer Genome Atlas (TCGA) with high vs. low XDS, categorised according to the upper quartile. Log-rank p-values are shown. (G) Multivariable Cox regression analysis for the relationship between XDS as a continuous variable and DFS in TCGA cohorts, corrected for mutational burden, stage and T cell infiltrate.

FIG. 9—A validated gene signature of CD4^{ds} predicts lung cancer survival. (A) Correlation between XDS signature and patient stage in three cohorts. P-values are from mixed effects models accounting for histology and multiregionality. (B) Multivariable Cox regression in the TRACERx RNAseq cohort, corrected for clonal mutational burden. (C) Multivariable Cox regression analysis in TCGA LUAD and LUSC showing the relationship between XDS enrichment and survival.

FIG. 10—Correlation between gene signatures, CD4 subset abundance, TMB and DFS(A) Correlation between CD4 subset abundance and gene signatures of differentiation skewing in the TRACERx RNAseq cohort. Gene signature values have been z-score scaled. The Xue TCF7/LEF1 signature is enriched amongst mouse T cells with double knockout of Tcf7 and Lef1. The XDS.core signature was derived by retaining genes in the original XDS signature that are upregulated by Tcf7/Lef1 knockout cells. The XDS.other signature consists of the remaining XDS genes. The Zheng CD4 exhaustion signature does not correlate with CD4^{ds} measured by flow cytometry and was chosen as a negative control. Pearson p- and r-values are shown on the plots. (B) Signature relationship with TMB. (C) Forest plot showing signature relationship with DFS in multivariable Cox regression models including T cell infiltration, histology, stage and TMB as covariates (each signature was evaluated in a separate model).

FIG. 11—CD4 differentiation skewing is associated with Treg abundance. (A) Manually gated FOXP3⁺ Treg abundance positively correlates with the ratio of TMB:Early abundance in the combined TRACERx flow cytometry cohort. ITH (intratumoural genomic heterogeneity) is defined as [clonal/total mutational burden]. Pearson r- and FDR adjusted −log₁₀ p-values are plotted. (B) Regions were split into high vs. low TMB according to the median. Within each group, regions were further split into high, intermediate and low CD4 Early abundance according to tertiles (left panel). Treg distribution within each of the six defined groups is shown in the right panel (ANOVA p-values shown). (C) Correlation between previously published transcriptional signatures of Treg enrichment and Treg abundance measured by flow cytometry, for TRACERx regions with paired cytometry and RNAseq data. (D) Relationship between Treg and CD4 differentiation signatures in the TracerX RNAseq, and (E) the TCGA LUAD cohorts. Pearson r- and p-values and mixed effects model p-values (p_{c}) corrected for sample multiregionality are shown. (F) Chemokine encoding genes that positively correlated with the Treg infiltration in TCGA LUAD. Genes that also correlate in the TRACERx RNAseq cohort are labelled black, or otherwise greyed out. (G) Log₁₀ CPM expression of genes encoding chemokine receptors corresponding to chemokines in (F), by Early, Tdys, TDT and Treg subsets in the scRNAseq dataset. Signature enrichment values are z-score scaled. (H) A proposed model for the relationship between TMB, changes within the CD4 T cell differentiation landscape and patient outcome. TMB gives rise to antigens that enhance tumour antigenicity, promoting effective immunity in the context of a tumour replete with progenitor-like CD4 T cells. Antigen persistence results in CD4 differentiation skewing. Independent of TMB, Tregs also promote CD4 differentiation skewing. The balance between competing immune promoting and compromising effects of TMB may contribute to determining patient outcome.

FIG. 12—CD4 differentiation skewing is associated with Treg abundance. (A) Relationship between TMB and CD57+, CD57− and total Treg abundance (as a percentage of all CD4 cells) in the TRACERx flow cytometry cohort. (B-D) Relationship between XDS and Treg transcriptional signatures in TRACERx adenocarcinoma (B), squamous cell carcinoma (C) and TCGA LUSC (D). Pearson p- and r-values are shown, along with corrected p-values (pc) from mixed effect regression models accounting for sample multiregionality where appropriate.

FIG. 13—Patient demographic and summary of TRACERx.100 samples used in the study of Example 2. a. CONSORT diagram. b. Clinical characteristics and omics analysis of patient cohort. c. Flow cytometry sample summary according to histological subtype and tissue. LUAD; Lung adenocarcinoma, LUSC; Lung squamous cell carcinoma.

FIG. 14—Neoantigen burden defines the CD8 T cell subset landscape in LUAD. a. Upper panel; Frequency of CD8 T cells in each FlowSOM cluster within LUAD TILs. Clusters are defined by number, coloured headers indicate T cell subset classification. Lower; Heatmap of normalised marker expression for each cluster. x axis denoted cluster description. b. Spearman rank correlation coefficient of the frequency of each CD8 T cell cluster vs load of putative neoantigens in LUAD cases, solid circles and arrows indicate significant correlations. c. Correlation plots of neoantigen load vs cluster frequency in LUAD TILs. d-f. Uniform manifold approximation and projection of CD8 T cells in LUAD tumours showing relative expression of markers (d), all CD8 T cell clusters coloured according to parent subset (e) or clusters associated with neoantigen load (f). g. Correlation plots showing the ratio of clusters (upper) or manually gated CD8 T cell subsets (lower) indicated vs neoantigen load in LUAD. Tumour regions (left) or patients (right). Data in all panels is from n=32 tumour regions of 16 patients. R and pAdj in b,c,g from 2-tailed spearman rank correlation coefficient corrected by BH, FDR 0.05. LUAD; Lung adenocarcinoma, LUSC; Lung squamous cell carcinoma, TEMRA; Terminally differentiated effector memory cell re-expressing CD45RA, TDE: Terminally differentiated effector cell, Tcm: Central-memory like cell, Trm: Tissue resident memory cell, Tdys: T cell with a dysfunctional phenotype, cl: Cluster.

FIG. 15—Unsupervised flow cytometry analysis of CD8 T cells in TRACERx NSCLC samples. a. Gating tree used to export live CD8 T cells for clustering. Histogram shows PD-1 expression in CD8 T cells from concatenated normal tissue and TIL. B. Original FlowSOM clustering heatmap and dendogram, of CD8 T cells in LUAD TILS c. Concatenated FlowSOM heatmap of CD8 T cells in LUAD and LUSC normal tissue and TIL from 50 clustering iterations. D. Lef1, histograms and right flow cytometry plots of indicated clusters in the PD-1^{hi} Trm subset. E. Frequency of CD8 T cells in each cluster in LUAD TILS ordered by descending abundance. f. Frequency of CD8 T cells in each cluster according to tissue type for LUAD and LUSC samples. g. Frequency of CD8 T cells in each subset or cluster indicated in the legend for TIL samples of LUAD tumour regions (denoted as R1-R6) from the patients indicated (CRUK00XX). Tumour regions LUAD n=34 (17 patients), LUSC n=39 (18 patients), NTL LUAD=10, LUSC=12. TIL: Tumour samples, NTL: Non-tumour lung. LUAD; Lung adenocarcinoma, LUSC; Lung squamous cell carcinoma. TEMRA; Terminally differentiated effector memory cell re-expressing CD45RA, TDE: Terminally differentiated effector cell, Tcm: Central-memory like cell, Trm: Tissue resident memory cell, Trm-dys: Tissue resident memory cell with a dysfunctional phenotype, cl: Cluster. *pAdj<0.05 (Two-way ANOVA adjusted by BH correction).

FIG. 16—CD8 T cell subsets in NSCLC identified by flow cytometry. CD8 T cell clusters were identified by iterative unsupervised clustering and classified into the denoted subsets according to supercluster formation on FlowSOM dendograms, location in dimensionally reduced space and manual annotation of function. See “Example 2—results” for references. Clusters within the PD-1^{hi} Trm subset are sub classified in the final 3 rows. TEMRA; Terminally differentiated effector memory cell re-expressing CD45RA, TDE: Terminally differentiated effector cell, Tcm: Central-memory like cell, Trm: Tissue resident memory cell, Trm-dys: Tissue resident memory cell with a dysfunctional phenotype, cl: Cluster, ICB: Immune checkpoint blockade, LN: lymph node.

FIG. 17—Integration of flow cytometry analysis with paired orthogonal data in TRACERx. a. Schematic of sample analysis pipeline for immune-omics correlative analysis using flow cytometry data. b. Sample ID (patient: region) vs omics data availability for tumour regions in flow cytometry cohort, separated according to histology. Path.TIL=pathology TIL estimates of infiltration. c. number of predicted neoantigens in samples with available flow cytometry data in the study. d. Correlation between TMB and neoantigen load, samples from n=32 TLUAD TIL samples used in flow cytometry analysis are shown. LUAD; Lung adenocarcinoma, LUSC; Lung squamous cell carcinoma.

FIG. 18—Unsupervised and manually gated flow cytometric analysis of CD8 T cells in LUAD and LUSC tumours. a. Uniform manifold approximation and projection of CD8 T cells in LUAD tumours showing relative expression of markers indicated and b. individual clusters colored according to parent subset. c. Manual gating strategy used to confirm FlowSOM clusters. d. Correlation matrix comparing the frequency of CD8 T cells identified by clustering and manual gating in LUAD TILS, heat reflect spearman rank correlation coefficient. e. Heat map of spearman correlation between the Tdys:Trm ratio and the number of mutations or neoantigens denoted on the x axis. f. Correlation of indicated CD8 T cell cluster frequency with high affinity clonal neoantigen load in LUAD TIL. g. The frequency of CD8 T cell clusters in LUAD and LUSC tumours. h. Correlation of indicated CD8 T cell cluster frequency with neoantigen load in LUSC TIL. Data in a-d,g from n=33 tumour regions (17 patients), e-f n=32 xy pairs (16 patients). LUSC samples in g-h from n=36 (g) or n=32 xy pairs (h) from 18 patients. *pAdj<0.05 2-tailed spearman rank test (d, e, f, h) or two-way ANOVA (g). <50 nM=threshold of predicted neo affinity TMB=tumour mutational burden, MB=mutational burden, Non-neo=predicted non neoantigen encoding mutations. Neo=neoantigen.

FIG. 19—CD8 T cells associated with neoantigen burden exhibit phenotypic and molecular hallmarks of dysfunction. a. Fluorescence intensity of markers shown were measured on CD8 T cell clusters associated with neoantigen burden. b. Relative MFI of markers indicated on Tdys vs average expression on Trm clusters 2,3 and 5 expressed as log 2-fold change in favour of Tdys. c. Correlation of geometric MFI of indicated markers with neoantigen load in Tdys-gated LUAD CD8 T cells. d. Sort logic of CD8 T cell subsets isolated for RNAseq analysis showing a representative patient. e. Volcano plots showing differential gene expression analysis of RNAseq data of T cell subsets from n=3 NSCLC patients in TRACERx (CRUK0024, CRUK0069, CRUK0017), y axis represents multiple comparison adjusted p values (BH at FDR 0.05). f. GSEA of RNAseq data using gene sets from NSCLC and Melanoma CD8 T cell subsets. g. Enrichment plots from GSEA of 4 out of the 9 gene sets used to analyse RNAseq data. pAdj=BH corrected P value at FDR 0.05.

FIG. 20—Extended analysis of CD8 T cell populations associated with neoantigen burden. a. MFI of markers in specified clusters showing data from 33 tumour regions of 16 LUAD patients. For Trm clusters the average value of cl.2,3,5 is plotted. b. Correlation of MFI of markers indicated on total CD8 T cells vs neoantigen load in n=32 xy pairs from LUAD TILs. c. Histogram of HLA-DR expression in low and hi neoantigen load LUAD TIL regions (split on the median). d. Schematic showing method of analysis for e. e. Correlation matrix showing spearman rank correlation value for each marker vs neoantigen load in the clusters indicated on the x axis. *pAdj<0.05.

FIG. 21—CD8 T cells associated with neoantigen burden exhibit tumour specific dysfunctional reprogramming and clonotypic expansion. a. Frequency of Tdys (cl.1) in ungated or CD45RA⁻CD57⁻PD-1^{hi} CD8 T cells for n=33 tumour regions of 16 LUAD patients, paired T-test. b. Correlation plot of manually gated CD45RA⁻CD57⁻PD-1^{hi} CD8 T cells vs neoantigen load, n=32 xy pairs of tumour regions from 16 LUAD patients. c. Heatmap of genes differentially expressed in RNAseq data of PD-1^{hi}CD57⁻CCR7⁻CD45RA⁻ CD8 T cells (Tdys) or all other CD8 T cells (non-Tdys) sorted from TILs or matched normal tissue from n=3 NSCLC patients in TRACERx (CRUK0024, CRUK0069, CRUK0017). d. GSEA of TILs sorted as above using gene sets from a murine TCR transgenic neoantigen-induced tumour-specific T cell dysfunction model (Schietinger et al Immunity 2016). e. The number of expanded (>2 in 1000) T cell receptor sequences from TCRseq libraries of tumour resections from CRUK0024, CRUK0069, CRUK0017 that were matched in RNAseq data of sorted T cell subsets. Black represents expanded TCR sequences from RNAseq found in TCRseq. f. Overlap or exclusivity amongst expanded TCRs identified in each sorted subset. *pAdj<0.05, **pAdj<0.01.

FIG. 22—Neoepitope-specific CD8 T cells isolated from NSCLC patients display a dysfunctional phenotype. a. Description of neoepitope reactivities examined by MHC multimer analysis in pilot cases from the TRACERx study. b. PD-1 MFI relative to matched PBMC in NTL, TIL and n=3 neoepitope reactivities (Neo-Ag TIL) from L011, L012, L021, error bars represent SEM. c. Expression of denoted markers in populations indicated in patient L011. d. FACS plots and SPICE co-expression profiles of populations indicated in the legend defined by marker expression shown on pie arcs. *pAdj<0.05, **pAdj<0.01.

FIG. 23—Neoepitope specific CD8 T cells express dysfunctional gene programs that associate with mutational burden in multiple cohorts. a. MHC-multimer identification of four neoepitope reactivities in ex-vivo TILS of three NSCLC cases and associated PD-1 expression on multimer positive or negative TILS and matched NTL and PBMC. b. Sort strategy and volcano plot of scRNAseq data from multimer positive (n=33) and negative (n=22) CD8 TILS of patient L011. c. Enrichment plots from GSEA of 4 out of the 9 gene sets used to analyse scRNAseq data. d. GSEA of scRNAseq data using gene sets from NSCLC and Melanoma CD8 T cell subsets. e. Correlation plots showing neoantigen load vs Z-transformed RNAseq score of gene signatures developed from neoepitope specific CD8 T cells and sorted Tdys cells (Neo CD8 T dys) tumour specific dysfunctional CD8 T cells from mice and melanoma patients (Melan.Sv40 CD8 Tdys) or naive CD8 T cells of NSCLC (CD8 T naive). Data from Tx.100 LUAD (upper row, n=68 tumour regions from 35 patients) or TCGA LUAD (n=110). R values and pAdj in correlation plots from spearman rank correlation coefficient. Error bands in e represent 95% confidence intervals. *pAdj<0.05, **pAdj<0.01.

FIG. 24—Generation, validation and application of neoantigen associated dysfunctional CD8 T cell gene scores. a. Genes in the leading edge of GSEA from cl.1 enriched bulk RNAseq (Trm-dys) and neoantigen specific CD8 T cells from L011 (Neo.CD8) neo.CD8 scRNAseq analysis vs ‘Tex’ marker genes from Guo et al in NSCLC (See main text for reference). Red highlights genes enriched in each data set or both which were used as the Neo. dys score. b. Schematic and c. correlation matrix of cluster frequency vs RNAseq score for validation of gene signatures using paired LUAD cases with flow cytometry. d. Schematic illustrating integration of RNAseq scores with WES data and e. Inventory of Tx.100 samples with RNAseq data showing available omics data in LUAD and LUSC patients. f. Pathology TIL estimates in tumour regions of LUAD cases used in RNAseq score analysis split by the median neoantigen load of the sample set. Data shows 24 tumour regions of 12 patients (c) and 74 regions of 35 patients (f). Error bars in F represent the SEM. *pAdj<0.05, 2-tailed spearman test.

FIG. 25—Neoantigen load and MHC pathway disruption co-define CD8 T cell dysfunction. a. Mutations in antigen processing and presentation and HLA LOH in tumour regions from LUAD cases in Tx.100 with available WES plus RNAseq or flow data. b. Frequency of Tdys cells in LUAD tumour regions with or without evidence of antigen presentation defects (n=32 regions). c. RNAseq data from LUAD tumour regions showing the Neo.Tdys z-score value in groups classified by neoantigen load (according to the median value) and antigen presentation disruption. d. Correlation plots displaying neoantigen load vs Neo.Tdys RNAseq score in LUAD tumour regions without (left) or with (right) evidence of defects in antigen presentation. N=74 tumour regions in c-d. *pAdj<0.05. **pAdj<0.01. Analysis via ANOVA (b-c) or 2-tailed spearman rank test (d). Diff in R calculated via R to Z Fisher transformation.

FIG. 26—Association of CD8 T cell subsets with neoantigen-directed immune escape in LUAD. a. Frequency of CD8 T cell FlowSOM clusters in tumours with or without defects in antigen presentation. n=32 tumour regions from 16 LUAD patients. b. Frequency of Tdys cl.1 CD8 T cells in antigen presentation defect-classified tumour regions grouped according to neoantigen load low or high regions (defined by the median). c-d. Tdys:Trm ratio in tumour regions with or without antigen presentation defects analysed by flow cytometry according to c. grouped or d. correlation analysis vs neoantigen load in LUAD tumour regions. e. Neoantigen load amongst neoantigen high tumour regions with RNAseq data, separated according to antigen presentation defect groups. f-g. RNAseq data from LUAD tumour regions showing the Melan.SV40 Tdys z-score value in groups classified by neoantigen load (according to the median value) and antigen presentation defects by f. Correlation plots displaying neoantigen load vs Melan.SV40 Tdys RNAseq score in LUAD tumour regions without (left) or with (right) evidence of immune escape. g. Group analysis. n=74 tumour regions in d-e. h. Model of CD8 T cell differentiation in treatment naive LUAD. *pAdj<0.05. **pAdj<0.01. Analysis via ANOVA or 2-tailed spearman rank test.

FIG. 27—Validation of targets in samples from lung cancer patients from TRACERx. (A-I) Tumour infiltrating lymphocytes obtained from stage IV non-small cell lung cancer were analysed by flow cytometry (data for SIRPG in (A), SIT1 in (D) and FCRL3 in (G)). Target expression was analysed on different subsets of T cells, non up T cells (population 1), PD1-TIM3−CD8 T cells (Non-tumour reactive, population 2), PD1⁺TIM3−CD8 T cells (tumour reactive, non-exhausted, population 3), PD1⁺TIM3⁺CD8 T cells (Exhausted CD8 T cells, population 4) and PD1⁺TIM3⁺CD39+41BB+ CD8 T cells (Neoantigen reactive CD8 T cells, population 5). The expression of each target was analysed in two different patients and its mean fluorescence intensity was graphed (data for SIRPG in (B), SIT1 in (E) and FCRL3 in (H)) and shown in the histogram for each subset of cells (data for SIRPG in (C), SIT1 in (F) and FCRL3 in (I)).

FIG. 28—SIT1 Knock-out T cells show increased production of IFNγ following in vitro restimulation. (A-C) Human peripheral blood mononuclear cells, were stimulated for three days using αCD3 and αCD28 antibodies. On day three, cells were electroporated with the Cas9 protein and with the crRNA targeting SIT1. Cells were kept in culture for 10 days using low doses of interleuquin 2. On day 10 cells were stained with cell trace violet and restimulated for four days with a low dose of dynabeads containing αCD3 and αCD28. On day 14, cells were incubated with brefeldin A for four hours in order to accumulate cytokines. Cells were stained for flow cytometry and acquired in the FACS symphony. (A) Flow cytometry analysis of SIT1 expression on total CD3⁺ T cells after 14 days of culture. (B) Flow cytometry analysis of IFNγ⁺CD4 and CD8 cells that diluted cell trace violet (CTV), unstimulated cells were used as controls. (C) Quantification of IFNγ⁺ T cells, control non-edited versus SIT-1 knock-out. (D) Schematic representation of the protocol used.

FIG. 29—Gene knock-outs of selected proposed targets. Human peripheral blood mononuclear cells, were stimulated for three days using αCD3 and αCD28 antibodies. On day three, cells were electroporated with the Cas9 protein and with the crRNA targeting each of the target genes shown (SIRPg, SIT1, IL1RAP). The figure shows, in the T cell populations (CD8 and CD4 T cells) identified in the plot in the top left corner, the signal (number of events) for each target gene in the FMO (fluorescence minus one) control (top curve in each plot), the unedited control (middle curve in each plot) and the edited cells (bottom curve in each plot), together with the associated frequencies of positive cells indicated as percentages next to the respective curves.

FIG. 30. SIT1 Knock-out tumour infiltrating T cells acquire enhanced proliferative capacity. Tumour infiltrating lymphocytes obtained from NSCLC patients were KO and expanded for 21 days using a rapid expansion protocol (REP). On day 21 cells were stained with CTV and restimulated with a low dose of αCD3/CD28 beads. Four days later, CTV dilution was measured using Flow Cytometry.

FIG. 31. OKT3-expressing tumour cells cocultured with human T cells. (A) PBMC-derived cells: Knock-out of human PBMCs were done using 2 different crRNAs (named AA, AB, AC or AD) per gene followed by electroporation of the Cas9:crRNA complex. 4 days later the edited PBMCs were co-cultured with anti-CD3 expressing lung tumour cells (H228-OXT3). The readouts were measured 24 and 72 hours later using high-dimensional flow cytometry. (B) TILs: Knock-out of NSCLC TILs were done using 2 different crRNAs (named AA, AB, AC or AD) per gene followed by electroporation of the Cas9:crRNA complex. 4 days later the edited TILs were co-cultured with anti-CD3 expressing lung tumour cells (H228-OXT3). The readouts were measured 24 and 72 hours later using high-dimensional flow cytometry.

FIG. 32. Gating strategy to define PD1⁺ populations in CD8 T and CD4 T cells. The plots illustrate the gating strategy applied to define PD1⁻, PD-1^{high} and PD-1^{total} (PD-1^{int}+PD-1^{high}) populations of CD4 (B) and CD8 (A) T cells. Four different conditions are used: unstimulated (top left), stimulated with dynabeads coated with anti-CD3 and anti-CD28 antibodies (top right), cocultured with lung cancer cells (bottom left) and cocultured with lung cancer cells modified to express anti-CD3 (bottom right). The plots show the results for an example sample of modified cells (a single FURIN KO expanded TIL sample).

FIG. 33. OKT3-expressing tumour cells cocultured with human PBMC-derived T cells. The plots show the results of the experimental protocol described on FIG. 31A. (A) In vitro stimulated controls, CD8 T cells that are PD1+LAMP− and PD1+LAMP1+ positive after 72 hours of stimulation. (B) In vitro stimulated controls, CD4 T cells that are PD1+LAMP1− and PD1+LAMP1+ positive after 72 hours of stimulation. (C) Cocultured CD8 T cells that are PD1+LAMP− and PD1+LAMP1+ positive after 72 hours of stimulation. (D) Cocultured CD4 T cells that are PD1+LAMP1− and PD1+LAMP1+ positive after 72 hours of stimulation. (E) Cocultured CD4 T cells that are PD1+TIM3+ positive after 72 hours of stimulation. (F) Cocultured CD8 T cells that are PD1+TIM3+ positive after 72 hours of stimulation. (A,B) unstimulated=unstimulated T cells; dynabeads=in vitro stimulation with αCD3/aDC28 covered beads; PMA/ionomycin=in vitro stimulation with PMA and ionomycin. (C,D) CTRL=unmodified tumour cells (x-axis); αCD3=tumour cells modified to express anti-cd3; 1αCD3 1:10=1 to 10 mixture of tumour cells modified to express anti-cd3 and unmodified tumour cells; CTRL=scrambled crRNA (E,F) H228=unmodified tumour cells, H228-OKT3=tumour cells modified to express anti-cd3; 1/10 H2228-OKT3=1 to 10 mixture of tumour cells modified to express anti-cd3 and unmodified tumour cells; CTRL=scrambled crRNA.

FIG. 34. H2228-OKT3 coculture with NSCLC TILs identifies regulators of PD1 signalling. Knock-out of NSCLC TILs, coculture with lung cancer cells modified to express anti-CD3 and readouts were done as explained in relation to FIG. 31B. The readouts shown on this figure are the % of PD-1^{total} cells (A, C) PD-1^{high} cells (B, D) amongst the CD4 T cell population (A,B) and the CD8 T cell population (C, D). Each condition is a result of two replicates, and shows the average (main bar) and standard deviation around the mean (thin bar). The control is CD4 and CD8 T cells from unmodified NSCLC TILs cocultured with the lung cancer cells modified to express anti-CD3.

FIG. 35. Changes in T cell differentiation and functionality of CD4 and CD8 NSCLC TILs FURIN AB KO after 72 hours of co-culture with H2228-OKT3 tumour cells. Knock-out of NSCLC TILs, co-culture and readouts were performed as described in relation to FIG. 31B. The plots compares the frequencies of the positive populations (for representative markers of T cell differentiation and functionality between non-edited CD4 TILs and FURIN KO CD4 TILs (A) between non-edited CD8 TILs and FURIN KO CD8 TILs (B), quantified by flow cytometry. Each condition is a result of two technical replicates and shows the average and standard deviation around the mean.

FIG. 36. Changes in T cell differentiation and functionality of CD4 and CD8 NSCLC TILs AXL KO after 72 hours of co-culture with H2228-OKT3 tumour cells. Knock-out of NSCLC TILs, co-culture and readouts were performed as described in relation to FIG. 31B. A. Frequencies of the positive populations for representative markers of T cell differentiation and functionality between non-edited CD4 TILs and AXL KO CD4 TILs, quantified by flow cytometry. B. Frequencies of the positive populations for representative markers of T cell differentiation and functionality between non-edited CD8 TILs and AXL KO CD8 TILs, quantified by flow cytometry. For each condition the values for two replicates are shown, together with the average value and standard deviation around the mean for those replicates.

FIG. 37. Changes in T cell differentiation and functionality of CD4 and CD8 NSCLC TILs IL1RAP KO after 72 hours of co-culture with H2228-OKT3 tumour cells. Knock-out of NSCLC TILs, co-culture and readouts were performed as described in relation to FIG. 31B. A. Frequencies of the positive populations for representative markers of T cell differentiation and functionality between non-edited CD4 TILs and IL1RAP KO CD4 TILs, quantified by flow cytometry. B. Frequencies of the positive populations for representative markers of T cell differentiation and functionality between non-edited CD8 TILs and IL1RAP KO CD8 TILs, quantified by flow cytometry. Each condition is a result of two replicates and shows the average and standard deviation around the mean.

FIG. 38. Changes in T cell differentiation and functionality of CD4 and CD8 NSCLC TILs STOM KO after 72 hours of co-culture with H2228-OKT3 tumour cells. Knock-out of NSCLC TILs, co-culture and readouts were performed as described in relation to FIG. 31B. A. Frequencies of the positive populations for representative markers of T cell differentiation and functionality between non-edited CD4 TILs and STOM KO CD4 TILs, quantified by flow cytometry. B. Frequencies of the positive populations for representative markers of T cell differentiation and functionality between non-edited CD8 TILs and STOM KO CD8 TILs, quantified by flow cytometry. Each condition is a result of two replicates and shows the average and standard deviation around the mean.

FIG. 39. Changes in T cell differentiation and functionality of CD4 and CD8 NSCLC TILs E2F1A KO after 72 hours of co-culture with H2228-OKT3 tumour cells. Knock-out of NSCLC TILs, co-culture and readouts were performed as described in relation to FIG. 31B. A. Frequencies of the positive populations (for representative markers of T cell differentiation and functionality between non-edited CD4 TILs and E2F1A KO CD4 TILs, quantified by flow cytometry. B. Frequencies of the positive populations for representative markers of T cell differentiation and functionality between non-edited CD8 TILs and E2F1A KO CD8 TILs, quantified by flow cytometry. Each condition is a result of two replicates and shows the average and standard deviation around the mean.

FIG. 40. Changes in T cell differentiation and functionality of CD4 and CD8 NSCLC TILs SAMSN1 KO after 72 hours of co-culture with H2228-OKT3 tumour cells. Knock-out of NSCLC TILs, co-culture and readouts were performed as described in relation to FIG. 31B. A. Frequencies of the positive populations for representative markers of T cell differentiation and functionality between non-edited CD4 TILs and SAMSN1 KO CD4 TILs, quantified by flow cytometry. B. Frequencies of the positive populations for representative markers of T cell differentiation and functionality between non-edited CD8 TILs and SAMSN1 KO CD8 TILs, quantified by flow cytometry. Each condition is a result of two replicates and shows the average and standard deviation around the mean.

FIG. 41. Changes in T cell differentiation and functionality of CD4 and CD8 NSCLC TILs SIRPg KO after 72 hours of co-culture with H2228-OKT3 tumour cells. Knock-out of NSCLC TILs, co-culture and readouts were performed as described in relation to FIG. 31B. A. Frequencies of the positive populations for representative markers of T cell differentiation and functionality between non-edited CD4 TILs and SIRPg KO CD4 TILs, quantified by flow cytometry. B. Frequencies of the positive populations for representative markers of T cell differentiation and functionality between non-edited CD8 TILs and SIRPg KO CD8 TILs, quantified by flow cytometry. Each condition is a result of two replicates and shows the average and standard deviation around the mean.

FIG. 42. Changes in T cell differentiation and functionality of CD4 and CD8 NSCLC TILs CD7 KO after 72 hours of co-culture with H2228-OKT3 tumour cells. Knock-out of NSCLC TILs, co-culture and readouts were performed as described in relation to FIG. 31B. A. Frequencies of the positive populations for representative markers of T cell differentiation and functionality between non-edited CD4 TILs and CD7 KO CD4 TILs, quantified by flow cytometry. B. Frequencies of the positive populations for representative markers of T cell differentiation and functionality between non-edited CD8 TILs and CD7 KO CD8 TILs, quantified by flow cytometry. Each condition is a result of two replicates and shows the average and standard deviation around the mean.

FIG. 43. Changes in T cell differentiation and functionality of CD4 and CD8 NSCLC TILs CD82 KO after 72 hours of co-culture with H2228-OKT3 tumour cells. Knock-out of NSCLC TILs, co-culture and readouts were performed as described in relation to FIG. 31B. A. Frequencies of the positive populations for representative markers of T cell differentiation and functionality between non-edited CD4 TILs and CD82 KO CD4 TILs, quantified by flow cytometry. B. Frequencies of the positive populations for representative markers of T cell differentiation and functionality between non-edited CD8 TILs and CD82 KO CD8 TILs, quantified by flow cytometry. Each condition is a result of two replicates and shows the average and standard deviation around the mean.

FIG. 44. Changes in T cell differentiation and functionality of CD4 and CD8 NSCLC TILs FCRL3 KO after 72 hours of co-culture with H2228-OKT3 tumour cells. Knock-out of NSCLC TILs, co-culture and readouts were performed as described in relation to FIG. 31B. A. Frequencies of the positive populations for representative markers of T cell differentiation and functionality between non-edited CD4 TILs and FCRL3 KO CD4 TILs, quantified by flow cytometry. B. Frequencies of the positive populations for representative markers of T cell differentiation and functionality between non-edited CD8 TILs and FCRL3 KO CD8 TILs, quantified by flow cytometry. Each condition is a result of two replicates and shows the average and standard deviation around the mean.

Table 1—List of genes targeted by CRISPR-Cas9 knock-out with CRISPR RNA sequences used.

### DETAILED DESCRIPTION OF THE INVENTION

In describing the present invention, the following terms will be employed, and are intended to be defined as indicated below.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

AXL: “AXL” as used herein refers to Tyrosine-protein kinase receptor UFO protein encoded by the gene AXL. The UniProt accession number for the human AXL protein is P30530. The amino acid sequence of human BCL6 is shown at UniProt P30530-1, dated Mar. 28, 2018—v4 (incorporated herein by reference in its entirety). The GeneID for the human AXL gene is 558.

AXL inhibitors: An “AXL inhibitor” as used herein refers to a compound or agent (including an agent interfering with AXL gene expression such as RNAi) that inhibits the function of AXL as a receptor tyrosine kinase. In some embodiments, the AXL inhibitor may be a small molecule or a peptide. In some embodiments the AXL inhibitor may be the small molecule inhibitors BGB324 (Bemcentinib) or TP-093 (Dubermatinib). In another embodiment the AXL inhibitor may be the antibodies YW327.652 (Creative Biolabs®), AF154 (R&D Systems®), or h #11B7-T11 (Creative Biolabs®), or derivatives thereof.

SIT-1: “SIT-1” (Signaling threshold-regulating transmembrane adapter 1, also referred to herein as “SIT1”) is encoded by the gene SIT1. The UniProt accession number for the human SIT-1 is Q9Y3P8. The amino acid sequence of human SIT-1 is shown at Q9Y3P8-1, dated Nov. 1, 1999—v1 (incorporated herein by reference in its entirety). The GeneID for the human SIT-1 gene is 27240.

SAMSN1: “SAMSN1” (SAM domain-containing protein SAMSN-1) is encoded by the gene SAMSN1. The UniProt accession number for the human SAMSN1 is Q9NSI8. The amino acid sequence of human SAMSN1 is shown at Q9NSI8-1, dated Oct. 1, 2000—v1 (incorporated herein by reference in its entirety). The GeneID for the human SAMSN1 gene is 64092.

SIRPG: “SIRPG” (Signal-regulatory protein gamma) is encoded by the gene SIRPG. The UniProt accession number for the human SIRPG is Q9P1W8. The amino acid sequence of human SIRPG is shown at Q9P1W8-1, dated Jan. 23, 2007—v3 (incorporated herein by reference in its entirety). The GeneID for the human SIRPG gene is 55423.

CD7: “CD7” (T-cell antigen CD7) is encoded by the gene CD7. The UniProt accession number for the human CD7 is P09564. The amino acid sequence of human CD7 is shown at P09564-1, dated Jul. 1, 1989—v1 (incorporated herein by reference in its entirety). The GeneID for the human CD7 gene is 924.

CD82: “CD82” (CD82 antigen) is encoded by the gene CD82. The UniProt accession number for the human CD82 is P27701. The amino acid sequence of human CD82 is shown at P27701-1, dated Aug. 1, 1992—v1 (incorporated herein by reference in its entirety). The GeneID for the human CD82 gene is 3732. An association between CD82 activity and immune function was previously suggested (see e.g. Shibagaki et al., Eur J Immunol. 1999 December; 29(12):4081-91 and Eur J Immunol. 1998 April; 28(4):1125-33; Lebel-Binay S et al., J Immunol. 1995 Jul. 1; 155(1):101-10; Laguadriere-Gesbert et al., Eur J Immunol. 1998 December; 28(12):4332-4344). However, the present inventors have for the first time demonstrated that CD82 is abnormally expressed in dysfunctional T cells, and that enhancing CD82 activity (through expression or activation of the protein) could be used to treat proliferative disorders.

FCRL3: “FCRL3” (Fc receptor-like protein 3) is encoded by the gene FCRL3. The UniProt accession number for the human FCRL3 is Q96P31. The amino acid sequence of human FCRL3 is shown at Q96P31-1, dated Dec. 1, 2001—v1 (incorporated herein by reference in its entirety). The GeneID for the human FCRL3 gene is 115352.

IL1RAP: “IL1RAP” (Interleukin-1 receptor accessory protein) is encoded by the gene IL1RAP. The UniProt accession number for the human IL1RAP is Q9NPH3. The amino acid sequence of human IL1RAP is shown at Q9NPH3-1, dated Aug. 22, 2003—v2 (incorporated herein by reference in its entirety). The GeneID for the human IL1RAP gene is 3556.

FURIN: “FURIN” is encoded by the gene FURIN. The UniProt accession number for the human FURIN is P09958. The amino acid sequence of human FURIN is shown at P09958-1, dated Apr. 1, 1990—v2 (incorporated herein by reference in its entirety). The GeneID for the human FURIN gene is 5045.

STOM: “STOM” (Erythrocyte band 7 integral membrane protein) is encoded by the gene STOM. The UniProt accession number for the human STOM is P27105. The amino acid sequence of human STOM is shown at P27105-1, dated Jan. 23, 2007—v3 (incorporated herein by reference in its entirety). The GeneID for the human STOM gene is 2040.

E2F1: “E2F1” (Transcription factor E2F1) is encoded by the gene E2F1. E2Fla refers to the E2F1a transcript of E2F1. Thus, any reference to “E2F1a” herein should be interpreted to refer to E2F1 and any reference to “E2F1” should be interpreted to encompass E2F1a. The UniProt accession number for the human E2F1 is Q01094. The amino acid sequence of human E2F1 is shown at Q01094-1, dated Jul. 1, 1993—v1 (incorporated herein by reference in its entirety). The GeneID for the human E2F1 gene is 1869.

C5orf30: “C5orf30” (UNC119-binding protein C5orf30) is encoded by the gene C5orf30. The UniProt accession number for the human C5orf30 is Q96GV9. The amino acid sequence of human Crorf30 is shown at Q96GV9-1, dated Dec. 1, 2001—v1 (incorporated herein by reference in its entirety). The GeneID for the human C5orf30 gene is 90355.

CLDN1: “CLDN1” (Claudin-1) is encoded by the gene CLDN1. The UniProt accession number for the human CLDN1 is O95832. The amino acid sequence of human CLDN1 is shown at O95832-1, dated May 1, 1999—v1 (incorporated herein by reference in its entirety). The GeneID for the human CLDN1 gene is 9076.

COTL1: “COTL1” (Coactosin-like protein) is encoded by the gene xx. The UniProt accession number for the human COTL1 is Q14019. The amino acid sequence of human COTL1 is shown at Q14019-1, dated Jan. 23, 2007—v3 (incorporated herein by reference in its entirety). The GeneID for the human COTL1 gene is 23406.

DUSP4: “DUSP4” (Dual specificity protein phosphatase 4) is encoded by the gene DUSP4. The UniProt accession number for the human DUSP4 is Q13115. The amino acid sequence of human DUSP4 is shown at Q13115-1, dated Nov. 1, 1996—v1 (incorporated herein by reference in its entirety). The GeneID for the human DUSP4 gene is 1846.

EPHA1: “EPHA1” (Ephrin type-A receptor 1) is encoded by the gene EPHA1. The UniProt accession number for the human EPHA1 is P21709. The amino acid sequence of human EPHA1 is shown at P21709-1, dated Jan. 11, 2011—v4 (incorporated herein by reference in its entirety). The GeneID for the human EPHA1 gene is 2041.

FABP5: “FABP5” (Fatty acid-binding protein 5) is encoded by the gene FABP5. The UniProt accession number for the human FABP5 is Q01469. The amino acid sequence of human FABP5 is shown at Q01469-1, dated Jan. 23, 2007—v3 (incorporated herein by reference in its entirety). The GeneID for the human FABP5 gene is 2171.

GFI1: “GFI1” (Zinc finger protein Gfi-1) is encoded by the gene GFI1. The UniProt accession number for the human GFI1 is Q99684. The amino acid sequence of human GFI1 is shown at Q99684-1, dated Aug. 15, 2003—v2 (incorporated herein by reference in its entirety). The GeneID for the human GFI1 gene is 2672.

ITM2A: “ITM2A” (Integral membrane protein 2A) is encoded by the gene ITM2A. The UniProt accession number for the human ITM2A is 043736. The amino acid sequence of human ITM2A is shown at 043736-1, dated Jul. 15, 1999—v2 (incorporated herein by reference in its entirety). The GeneID for the human ITM2A gene is 9452.

PARK7: “PARK7” (Protein/nucleic acid deglycase DJ-1) is encoded by the gene PARK7. The UniProt accession number for the human PARK7 is Q99497. The amino acid sequence of human PARK7 is shown at Q99497-1, dated Jul. 5, 2004—v2 (incorporated herein by reference in its entirety). The GeneID for the human PARK7 gene is 11315.

PECAM1: “PECAM1” (Platelet endothelial cell adhesion molecule) is encoded by the gene PECAM1. The UniProt accession number for the human PECAM1 is P16284. The amino acid sequence of human PECAM1 is shown at P16284-1, dated Mar. 28, 2018—v2 (incorporated herein by reference in its entirety). The GeneID for the human PECAM1 gene is 5175.

PHLDA1: “PHLDA1” (Pleckstrin homology-like domain family A member 1) is encoded by the gene PHLDA1. The UniProt accession number for the human PHLDA1 is Q8WV24. The amino acid sequence of human PHLDA1 is shown at Q8WV24-1, dated May 5, 2009—v4 (incorporated herein by reference in its entirety). The GeneID for the human PHLDA1 gene is 22822.

RAB27A: “RAB27A” (Ras-related protein Rab-27A) is encoded by the gene RAB27A. The UniProt accession number for the human RAB27A is P51159. The amino acid sequence of human RAB27A is shown at P51159-1, dated Oct. 17, 2006—v3 (incorporated herein by reference in its entirety). The GeneID for the human RAB27A gene is 5873.

RBPJ: “RBPJ” (Recombining binding protein suppressor of hairless, also known as CBF-1, J kappa-recombination signal-binding protein, RBP-J, RBP-JK and Renal carcinoma antigen NY-REN-30) is encoded by the gene RBPJ. The UniProt accession number for the human RBPJ is Q06330 and the Uniprot identifier is SUH_HUMAN. The amino acid sequence of human RBPJ is shown at Q06330-1, dated Jun. 28, 2011—v3 (incorporated herein by reference in its entirety). The GeneID for the human RBPJ gene is 3516.

RGS1: “RGS1” (Regulator of G-protein signaling 1) is encoded by the gene RGS1. The UniProt accession number for the human RGS1 is Q08116. The amino acid sequence of human RGS1 is shown at Q08116-1, dated Mar. 24, 2009—v3 (incorporated herein by reference in its entirety). The GeneID for the human RGS1 gene is 5996.

RGS2: “RGS2” (Regulator of G-protein signaling 2) is encoded by the gene RGS2. The UniProt accession number for the human RGS2 is P41220. The amino acid sequence of human RGS2 is shown at P41220-1, dated Feb. 1, 1995—v1 (incorporated herein by reference in its entirety). The GeneID for the human RGS2 gene is 5997.

RNASEH2A: “RNASEH2A” (Ribonuclease H2 subunit A) is encoded by the gene RNASEH2A. The UniProt accession number for the human RNASEH2A is 075792. The amino acid sequence of human RNASEH2A is shown at 075792-1, dated May 15, 2002—v2 (incorporated herein by reference in its entirety). The GeneID for the human RNASEH2A gene is 10535.

SUV39H1: “SUV39H1” (Histone-lysine N-methyltransferase SUV39H1) is encoded by the gene SUV39H1. The UniProt accession number for the human SUV39H1 is 043463. The amino acid sequence of human SUV39H1 is shown at 043463-1, dated Jun. 1, 1998—v1 (incorporated herein by reference in its entirety). The GeneID for the human SUV39H1 gene is 6839.

TNIP3: “TNIP3” (TNFAIP3-interacting protein 3) is encoded by the gene TNIP3. The UniProt accession number for the human TNIP3 is Q96KP6. The amino acid sequence of human TNIP3 is shown at Q96KP6-1, dated Nov. 24, 2009—v2 (incorporated herein by reference in its entirety). The GeneID for the human TNIP3 gene is 79931.

Chimeric Antigen Receptors

Chimeric Antigen Receptors (CARs) are recombinant receptor molecules which provide both antigen-binding and T cell activating functions. CAR structure and engineering is reviewed, for example, in Dotti et al., Immunol Rev (2014) 257(1), which is hereby incorporated by reference in its entirety.

CARs comprise an antigen-binding domain linked to a transmembrane domain and a signalling domain. An optional hinge domain may provide separation between the antigen-binding domain and transmembrane domain, and may act as a flexible linker.

The antigen-binding domain of a CAR may be based on the antigen-binding region of an antibody which is specific for the antigen to which the CAR is targeted. For example, the antigen-binding domain of a CAR may comprise amino acid sequences for the complementarity-determining regions (CDRs) of an antibody which binds specifically to the target protein. The antigen-binding domain of a CAR may comprise or consist of the light chain and heavy chain variable region amino acid sequences of an antibody which binds specifically to the target protein. The antigen-binding domain may be provided as a single chain variable fragment (scFv) comprising the sequences of the light chain and heavy chain variable region amino acid sequences of an antibody. Antigen-binding domains of CARs may target antigen based on other protein:protein interaction, such as ligand:receptor binding; for example an IL-13Rα2-targeted CAR has been developed using an antigen-binding domain based on IL-13 (see e.g. Kahlon et al. 2004 Cancer Res 64(24): 9160-9166).

The transmembrane domain is provided between the antigen-binding domain and the signalling domain of the CAR. The transmembrane domain provides for anchoring the CAR to the cell membrane of a cell expressing a CAR, with the antigen-binding domain in the extracellular space, and signalling domain inside the cell. Transmembrane domains of CARs may be derived from transmembrane region sequences for CD3-ζ, CD4, CD8 or CD28.

The signalling domain allows for activation of the T cell. The CAR signalling domains may comprise the amino acid sequence of the intracellular domain of CD3-ζ, which provides immunoreceptor tyrosine-based activation motifs (ITAMs) for phosphorylation and activation of the CAR-expressing T cell. Signalling domains comprising sequences of other ITAM-containing proteins have also been employed in CARs, such as domains comprising the ITAM containing region of FcγRI (Haynes et al., 2001 J Immunol 166(1):182-187). CARs comprising a signalling domain derived from the intracellular domain of CD3-ζ are often referred to as first generation CARs.

Signalling domains of CARs may also comprise co-stimulatory sequences derived from the signalling domains of co-stimulatory molecules, to facilitate activation of CAR-expressing T cells upon binding to the target protein. Suitable co-stimulatory molecules include CD28, OX40, 4-1BB, ICOS and CD27. CARs having a signalling domain including additional co-stimulatory sequences are often referred to as second generation CARs.

In some cases CARs are engineered to provide for co-stimulation of different intracellular signalling pathways. For example, signalling associated with CD28 costimulation preferentially activates the phosphatidylinositol 3-kinase (P13K) pathway, whereas the 4-1BB-mediated signalling is through TNF receptor associated factor (TRAF) adaptor proteins. Signalling domains of CARs therefore sometimes contain co-stimulatory sequences derived from signalling domains of more than one co-stimulatory molecule. CARs comprising a signalling domain with multiple co-stimulatory sequences are often referred to as third generation CARs.

An optional hinge region may provide separation between the antigen-binding domain and the transmembrane domain, and may act as a flexible linker. Hinge regions may be flexible domains allowing the binding moiety to orient in different directions. Hinge regions may be derived from IgG1 or the CH₂CH₃ region of immunoglobulin.

Neoantigen Reactive T Cells (NAR-T)

A neoantigen is a newly formed antigen that has not been previously presented to the immune system. The neoantigen is tumour-specific, which arises as a consequence of a mutation within a cancer cell and is therefore not expressed by healthy (i.e. non-tumour) cells.

The neoantigen may be caused by any non-silent mutation which alters a protein expressed by a cancer cell compared to the non-mutated protein expressed by a wild-type, healthy cell. For example, the mutated protein may be a translocation or fusion.

A “mutation” refers to a difference in a nucleotide sequence (e.g. DNA or RNA) in a tumour cell compared to a healthy cell from the same individual. The difference in the nucleotide sequence can result in the expression of a protein which is not expressed by a healthy cell from the same individual. For example, the mutation may be a single nucleotide variant (SNV), multiple nucleotide variants, a deletion mutation, an insertion mutation, a translocation, a missense mutation or a splice site mutation resulting in a change in the amino acid sequence (coding mutation).

The human leukocyte antigen (HLA) system is a gene complex encoding the major histocompatibility complex (MHC) proteins in humans. A neoantigen may be processed to generate distinct peptides which can be recognised by T cells when presented in the context of MHC molecules. A neoantigen presented as such may represent a target for therapeutic or prophylactic intervention in the treatment or prevention of cancer in a subject.

An intervention may comprise an active immunotherapy approach, such as administering an immunogenic composition or vaccine comprising a neoantigen to a subject. Alternatively, a passive immunotherapy approach may be taken, for example adoptive T cell transfer or B cell transfer, wherein a T and/or B cells which recognise a neoantigen are isolated from tumours, or other bodily tissues (including but not limited to lymph node, blood or ascites), expanded ex vivo or in vitro and readministered to a subject.

T cells may be expanded by ex vivo culture in conditions which are known to provide mitogenic stimuli for T cells. By way of example, the T cells may be cultured with cytokines such as IL-2 or with mitogenic antibodies such as anti-CD3 and/or CD28. The T cells may be co-cultured with antigen-presenting cells (APCs), which may have been irradiated. The APCs may be dendritic cells or B cells. The dendritic cells may have been pulsed with peptides containing the identified neoantigen as single stimulants or as pools of stimulating neoantigen peptides. Expansion of T cells may be performed using methods which are known in the art, including for example the use of artificial antigen presenting cells (aAPCs), which provide additional co-stimulatory signals, and autologous PBMCs which present appropriate peptides. Autologous PBMCs may be pulsed with peptides containing neoantigens as single stimulants, or alternatively as pools of stimulating neoantigens.

Engineered T Cell

The present invention provides an engineered T cell in which the expression of genes selected from SIT1, SAMSN1, SIRPG, CD7, CD82, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3, or the expression or activity of the proteins encoded by these genes, has been modulated so as to enhance cytotoxic activity. Indeed, the above mentioned genes were found to be associated with dysfunctional phenotypes in tumour-infiltrating T cells. In particular, upregulated expression of SIT1, SAMSN1, SIRPG, CD7, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3, and downregulation of CD82 was observed in these dysfunctional populations.

The cell may be a eukaryotic cell, e.g. a mammalian cell. The mammal may be a human, or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell may be from, or may have been obtained from, a human subject.

The cell may be a CD4⁺ T cell or a CD8⁺ T cell. In some embodiments, the cell is a target protein-reactive CAR-T cell. In embodiments herein, a “target protein-reactive” CAR-T cell is a cell which displays certain functional properties of a T cell in response to the target protein for which the antigen-binding domain of the CAR is specific, e.g. expressed at the surface of a cell. In some embodiments, the properties are functional properties associated with effector T cells, e.g. cytotoxic T cells.

In some embodiments, the engineered T cell may display one or more of the following properties: cytotoxicity to a cell comprising or expressing the target protein; proliferation, increased IFNγ expression, increased CD107a expression, increased IL-2 expression, increased TNFα expression, increased perforin expression, increased granzyme B expression, increased granulysin expression, and/or increased FAS ligand (FASL) expression in response to the target protein, or a cell comprising or expressing the target protein.

In some embodiments, the engineered T cell expresses an engineered T cell receptor. For example, the engineered T cell may express a cancer-specific T cell receptor, such as the NY-ESO-1 T cell receptor. In embodiments, the engineered T cell does not express an endogenous T cell receptor. In embodiments, the engineered T cell does not express the immune checkpoint molecule programmed cell death protein 1 (PD-1). In embodiments, the engineered T cell has been engineered to remove the endogenous T cell receptor and/or the immune checkpoint molecule programmed cell death protein 1 (PD-1). In embodiments, the engineered T cell is a cell as described in Stadtmauer et al. (Science 28 Feb. 2020: vol. 367, Issue 6481, eaba7365), or a cell that has been obtained as described in Stadtmauer et al.

Gene expression can be measured by a various means known to those skilled in the art, for example by measuring levels of mRNA by quantitative real-time PCR (qRT-PCR), or by reporter-based methods. Similarly, protein expression can be measured by various methods well known in the art, e.g. by antibody-based methods, for example by western blot, immunohistochemistry, immunocytochemistry, flow cytometry, ELISA, ELISPOT, or reporter-based methods.

The present invention also provides a method for producing an engineered T cell according to the present invention, comprising genetically engineering a T cell (e.g. by CRISPR/Cas9-mediated gene editing, transcription activator-like effector nucleases (TALENs) transient downregulation using short hairpin RNA (shRNA), small interfering RNA (siRNA), microRNA (miRNA) or RNA constructs for overexpression or by introducing a nucleic acid or vector into the cell) to enhance expression of CD82 and/or knock-out or downregulate expression of one or more genes selected from SIT1, SAMSN1, SIRPG, CD7, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3. In some embodiments, the methods additionally comprise culturing the T cell under conditions suitable for expansion to provide an expanded cell population. In some embodiments, the methods are performed in vitro.

In some embodiments, the engineered T cell further comprises an introduced T cell receptor (e.g. a chimeric antigen receptor) that specifically recognises an antigen expressed on or in proximity to a tumour (e.g. tumour stroma). The present invention also provides methods of introducing an isolated nucleic acid or vector encoding the T cell receptor into the engineered T cell. In some embodiments the isolated nucleic acid or vector is comprised in a viral vector, or the vector is a viral vector. In some embodiments, the method comprises introducing a nucleic acid or vector according to the invention by electroporation.

Compositions

The present invention also provides compositions comprising a cell according to the invention.

Engineered T cells according to the present invention may be formulated as pharmaceutical compositions for clinical use and may comprise a pharmaceutically acceptable carrier, diluent, excipient or adjuvant.

In accordance with the present invention methods are also provided for the production of pharmaceutically useful compositions, such methods of production may comprise one or more steps selected from: isolating an engineered T cell as described herein; and/or mixing an engineered T cell as described herein with a pharmaceutically acceptable carrier, adjuvant, excipient or diluent.

Uses of and Methods of Using the CARs, Nucleic Acids, Cells and Compositions

The engineered T cells and pharmaceutical compositions according to the present invention find use in therapeutic and prophylactic methods.

The present invention also provides the use of an engineered T cell or pharmaceutical composition according to the present invention in the manufacture of a medicament for treating or preventing a disease or disorder.

The present invention also provides a method of treating or preventing a disease or disorder, comprising administering to a subject a therapeutically or prophylactically effective amount of an engineered T cell or pharmaceutical composition according to the present invention.

Administration

Administration of an activator/inhibitor or engineered T cell or composition according to the invention is preferably in a “therapeutically effective” or “prophylactically effective” amount, this being sufficient to show benefit to the subject. The actual amount administered, and rate and time-course of administration, will depend on the nature and severity of the disease or disorder. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

The activators/inhibitors and engineered T cells, compositions and other therapeutic agents, medicaments and pharmaceutical compositions according to aspects of the present invention may be formulated for administration by a number of routes, including but not limited to, parenteral, intravenous, intra-arterial, intramuscular, subcutaneous, intradermal, intratumoural and oral. The CARs, nucleic acids, vectors, cells, composition and other therapeutic agents and therapeutic agents may be formulated in fluid or solid form. Fluid formulations may be formulated for administration by injection to a selected region of the human or animal body, or by infusion to the blood. Administration may be by injection or infusion to the blood, e.g. intravenous or intra-arterial administration.

Administration may be alone or in combination with other treatments, either simultaneously or sequentially dependent upon the condition to be treated.

In some embodiments, treatment with an activator/inhibitor or engineered T cell or composition of the present invention may be accompanied by other therapeutic or prophylactic intervention, e.g. chemotherapy, immunotherapy, radiotherapy, surgery, vaccination and/or hormone therapy.

Simultaneous administration refers to administration of the activator/inhibitor, engineered T cell or composition and therapeutic agent together, for example as a pharmaceutical composition containing both agents (combined preparation), or immediately after each other and optionally via the same route of administration, e.g. to the same artery, vein or other blood vessel. Sequential administration refers to administration of one therapeutic agent followed after a given time interval by separate administration of the other agent. It is not required that the two agents are administered by the same route, although this is the case in some embodiments. The time interval may be any time interval.

Chemotherapy and radiotherapy respectively refer to treatment of a cancer with a drug or with ionising radiation (e.g. radiotherapy using X-rays or y-rays).

The drug may be a chemical entity, e.g. small molecule pharmaceutical, antibiotic, DNA intercalator, protein inhibitor (e.g. kinase inhibitor), or a biological agent, e.g. antibody, antibody fragment, nucleic acid or peptide aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the cancer. The chemotherapy may be administered by one or more routes of administration, e.g. parenteral, intravenous injection, oral, subcutaneous, intradermal or intratumoural.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment. The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs and biologics may be selected from: alkylating agents such as cisplatin, carboplatin, mechlorethamine, cyclophosphamide, chlorambucil, ifosfamide; purine or pyrimidine anti-metabolites such as azathiopurine or mercaptopurine; alkaloids and terpenoids, such as vinca alkaloids (e.g. vincristine, vinblastine, vinorelbine, vindesine), podophyllotoxin, etoposide, teniposide, taxanes such as paclitaxel (Taxol™), docetaxel; topoisomerase inhibitors such as the type I topoisomerase inhibitors camptothecins irinotecan and topotecan, or the type II topoisomerase inhibitors amsacrine, etoposide, etoposide phosphate, teniposide; antitumour antibiotics (e.g. anthracyline antibiotics) such as dactinomycin, doxorubicin (Adriamycin™), epirubicin, bleomycin, rapamycin; antibody based agents, such as anti-PD-1 antibodies, anti-PD-L1 antibodies, anti-TIM-3 antibodies, anti-CTLA-4, anti 1BB, anti-GITR, anti-CD27, anti-BLTA, anti-OX43, anti-VEGF, anti-TNFα, anti-IL-2, antiGpIIb/IIIa, anti-CD-52, anti-CD20, anti-RSV, anti-HER2/neu(erbB2), anti-TNF receptor, anti-EGFR antibodies, monoclonal antibodies or antibody fragments, examples include: cetuximab, panitumumab, infliximab, basiliximab, bevacizumab (Avastin®), abciximab, daclizumab, gemtuzumab, alemtuzumab, rituximab (Mabthera®), palivizumab, trastuzumab, etanercept, adalimumab, nimotuzumab; EGFR inhibitors such as erlotinib, cetuximab and gefitinib; anti-angiogenic agents such as bevacizumab (Avastin®); cancer vaccines such as Sipuleucel-T (Provenge®).

Further chemotherapeutic drugs may be selected from: 13-cis-Retinoic Acid, 2-Chlorodeoxyadenosine, 5-Azacitidine 5-Fluorouracil, 6-Mercaptopurine, 6-Thioguanine, Abraxane, Accutane®, Actinomycin-D Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Aranesp®, Aredia®, Arimidex®, Aromasin®, Arranon®, Arsenic Trioxide, Asparaginase, ATRA Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, Calcium Leucovorin, Campath®, Camptosar®, Camptothecin-11, Capecitabine, Carac™, Carboplatin, Carmustine, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine Cytosar-U®, Cytoxan®, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxiam, DTIC, DTIC-Dome®, Duralone®, Eligard™, Ellence™, Eloxatin™, Elspar®, Emcyt®, Epirubicin, Epoetin Alfa, Erbitux, Erlotinib, Erwinia L-asparaginase, Estramustine, Ethyol Etopophos®, Etoposide, Etoposide Phosphate, Eulexin®, Everolimus, Evista®, Exemestane, Faslodex®, Femara®, Filgrastim, Floxuridine, Fludara®, Fludarabine, Fluoroplex®, Fluorouracil, Fluoxymesterone, Flutamide, Folinic Acid, FUDR®, Fulvestrant, Gefitinib, Gemcitabine, Gemtuzumab ozogamicin, Gleevec™, Gliadel® Wafer, Goserelin, Granulocyte—Colony Stimulating Factor, Granulocyte Macrophage Colony Stimulating Factor, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant Purinethol®, Raloxifene, Revlimid®, Rheumatrex®, Rituxan®, Rituximab, Roferon-A® (Interferon Alfa-2a), Rubex®, Rubidomycin hydrochloride, Sandostatin® Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®.

Cancer

In some embodiments, the disease or disorder to be treated or prevented in accordance with the present invention is a cancer.

The cancer may be any unwanted cell proliferation (or any disease manifesting itself by unwanted cell proliferation), neoplasm or tumour or increased risk of or predisposition to the unwanted cell proliferation, neoplasm or tumour. The cancer may be benign or malignant and may be primary or secondary (metastatic). A neoplasm or tumour may be any abnormal growth or proliferation of cells and may be located in any tissue. Examples of tissues include the adrenal gland, adrenal medulla, anus, appendix, bladder, blood, bone, bone marrow, bowel, brain, breast, cecum, central nervous system (including or excluding the brain) cerebellum, cervix, colon, duodenum, endometrium, epithelial cells (e.g. renal epithelia), eye, germ cells, gallbladder, oesophagus, glial cells, head and neck, heart, ileum, jejunum, kidney, lacrimal glad, larynx, liver, lung, lymph, lymph node, lymphoblast, maxilla, mediastinum, mesentery, myometrium, mouth, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Without wishing to be bound by theory, it is believed that immune dysfunction may enable the progression of any type of cancer since most cancers exist in the context of the host's immune system. Indeed, most cancers are at least initially recognised and attacked by the immune system, and eventually able to progress through tumour-mediated immunosuppression and tumour evasion mechanisms. Examples of cancer to treat may be selected from bladder cancer, gastric cancer, oesophageal cancer, breast cancer, colorectal cancer, cervical cancer, ovarian cancer, endometrial cancer, kidney cancer (renal cell), lung cancer (small cell, non-small cell and mesothelioma), brain cancer (gliomas, astrocytomas, glioblastomas), melanoma, lymphoma, small bowel cancers (duodenal and jejunal), leukemia, pancreatic cancer, hepatobiliary tumours, germ cell cancers, prostate cancer, head and neck cancers, thyroid cancer and sarcomas. In particular, the present inventors have found that the present invention is likely to be beneficial at least in the context of treatment of lung adenocarcinoma, renal clear cell carcinoma, pancreatic adenocarcinoma, renal papillary carcinoma, hepatocellular carcinoma, adrenocortical carcinoma and mesothelioma. The present invention is likely to be particularly useful in the context of treatment of cancers that are considered immunogenic. These include for example melanoma, Lung squamous cell carcinoma, lung adenocarcinoma, bladder cancer, small cell lung cancer, oesophagus cancer, colorectal cancer, cervical cancer, head and neck cancer, stomach cancer, endometrial cancer, and liver cancer. Indeed, all of these types of cancers have been shown to have high somatic mutation rates (e.g. more than 5 somatic mutations per megabase in Alexandrov et al.).

Further, the present invention is likely to be particularly useful in the context of treatment of cancers that have a high neoantigen load. A cancer may be predicted to have high neoantigen load if it has high tumour mutational burden, which can be quantified by measuring the somatic mutation prevalence (number of somatic mutations per megabase of tumour genome) for a sample or plurality of samples. Somatic mutation prevalence for various cancer types have been quantified in Alexandrov et al. (Nature volume 500, pages 415-421(2013)). Cancer types that have high tumour mutational burden may include those with a median numbers of somatic mutations per megabase of at least 1, at least 5, or at least 10. For example, melanomas and squamous lung cancers are typically considered to have high mutational burden.

The present invention is likely to be particularly useful for the treatment of a tumour that has acquired or is predicted to be likely to acquire or show resistance to immunotherapy. In particular, the present invention may advantageously be used in the treatment of patients with a proliferative disorder (e.g. a cancer or tumour): (i) that have already undergone immunotherapy and have failed to respond, or no longer respond to the immunotherapy, (ii) that are predicted to be unlikely to respond to immunotherapy, where the patients may be (immunotherapy) treatment naïve, (iii) where the patient's tumour has no or low T-cell infiltration, and (iv) where the patient's tumour has a high proportion of dysfunctional T cells in the tumour-infiltrating T cell population. In embodiments, a tumour may be considered to have a high proportion of dysfunctional T cells in the tumour-infiltrating T cell population if the expression of one or more markers selected from SIT1, SAMSN1, SIRPG, CD7, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3, is higher than a respective control value, and/or the expression of CD82 is lower than a control value, where the control values may correspond to the respective expression of the one or more markers in a control tumour-infiltrating T cell population. The control tumour-infiltrating T cell population may be an early differentiated T cell population. Therefore, methods of treatment according to the present disclosure may include determining whether the patient is likely to respond to immunotherapy, whether the patient's tumour has no or low T-cell infiltration, and/or whether the patient's tumour has a high proportion of dysfunctional T cells in the tumour-infiltrating T cell population.

Tumours to be treated may be nervous or non-nervous system tumours. Nervous system tumours may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumours may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer (e.g. small cell), colon cancer, ovarian cancer, pancreatic cancer, thymic carcinoma, NSCLC, haematologic cancer and sarcoma.

T-Cell Dysfunction and Chronic Tumour Antigen Stimulation

In some embodiments, the disease or disorder to be treated or prevented in accordance with the present invention is a cancer that has high tumour mutational burden (TMB).

Tumour neoantigens are a key substrate for T cell-mediated recognition of cancer cells. Although TMB predicts response to immune checkpoint blockade (ICB) (Van Allen, E. M. et al., 2015; Rizvi et al., 2015; Snyder et al., 2014; Goodman et al, 2017), clinically evident tumours usually progress without therapy and eventually acquire resistance to therapy, suggesting functional impairment of anti-tumour T cell responses. In acute infection and vaccination, optimal T cell stimulation results in differentiation from progenitor to effector and effector memory phenotypes. However, persistently high antigen load in cancer and chronic infections drives T cell differentiation into dysfunctional states. Two broad patterns of dysfunction have been described in these settings. Firstly, exhaustion is characterised by high co-inhibitory and co-stimulatory receptor expression, impaired cytokine production and replicative capacity (Crawford, A. et al., 2014). Secondly, terminal differentiation is characterised by features of senescence including shortened telomeres, heightened sensitivity to apoptosis, and expression of markers including CD57, KLRG1 and the T-box transcription factor Eomesodermin (Eomes)(Fletcher, J. M. et al., 2005; Palmer, B. E et al., 2005; Patil, V. S. et al., 2018; Di Mitri D et al., 2007).

In tumours with high mutational burden, the majority of CD8 T cells exhibit a dysfunctional phenotype. However, the genomic determinants of CD8 T cell differentiation in cancer remained poorly defined. Further, whether chronic tumour antigen stimulation impairs the CD4T cell response in human cancer was previously unknown. The present inventors have found that mutational burden in NSCLC correlates with intratumour CD4 T cell differentiation skewing (decline in abundance of early differentiated CD4 T cell population and increase in abundance of dysfunctional and terminally differentiated CD4 T cell populations), have identified distinct regulatory mechanisms in the early differentiated, dysfunctional and terminally differentiated CD4 T cell populations, and have identified a signature of CD4 T cell differentiation skewing that is predictive of survival. From this, genes associated with dysfunctional CD4 T cells were identified, the modulation of which was shown to enhance the immune response to cancer neoantigens. Similarly in the CD8 compartment, the present inventors have found that TMB significantly correlated with skewing of tissue-resident CD8 T cell populations towards a dysfunctional phenotypes. They further demonstrated that unmanipulated, neoantigen-reactive CD8 T cells bear phenotypic and molecular hallmarks of dysfunction, and that signatures of T cell dysfunction correlated with TMB in independent NSCLC cohorts. From this, genes associated with dysfunctional CD8 T cells were identified, the modulation of which was shown to enhance the immune response to cancer neoantigens.

Adoptive Transfer

In embodiments of the present invention, a method of treatment or prophylaxis may comprise adoptive transfer of immune cells, particularly T cells. Adoptive T cell transfer generally refers to a process by which T cells are obtained from a subject, typically by drawing a blood sample from which T cells are isolated. The T cells are then typically treated or altered in some way, optionally expanded, and then administered either to the same subject or to a different subject. The treatment is typically aimed at providing a T cell population with certain desired characteristics to a subject, or increasing the frequency of T cells with such characteristics in that subject. Adoptive transfer of CAR-T cells is described, for example, in Kalos and June 2013, Immunity 39(1): 49-60, which is hereby incorporated by reference in its entirety.

In the present invention, adoptive transfer is performed with the aim of introducing, or increasing the frequency of, target protein-reactive T cells in a subject, in particular target protein-reactive CD8⁺ T cells.

In some embodiments, the subject from which the T cell is isolated is the subject administered with the modified T cell (i.e., adoptive transfer is of autologous T cells). In some embodiments, the subject from which the T cell is isolated is a different subject to the subject to which the modified T cell is administered (i.e., adoptive transfer is of allogenic T cells).

The at least one T cell modified according to the present invention can be modified according to methods well known to the skilled person. The modification may comprise nucleic acid transfer for permanent or transient expression of the transferred nucleic acid.

In some embodiments the method may comprise one or more of the following steps: taking a blood sample from a subject; isolating and/or expanding at least one T cell from the blood sample; culturing the at least one T cell in in vitro or ex vivo cell culture; engineering the at least one T cell to increase expression of CD82 and/or to knock out or downregulate expression of one or more genes selected from SIT1, SAMSN1, SIRPG, CD7, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3; optionally inserting a modified T cell receptor or CAR, or a nucleic acid, or vector encoding the modified T cell receptor or CAR; expanding the at least one engineered T cell, collecting the at least one engineered T cell; mixing the engineered T cell with an adjuvant, diluent, or carrier; administering the engineered T cell to a subject.

In embodiments according to the present invention the subject is preferably a human subject. In some embodiments, the subject to be treated according to a therapeutic or prophylactic method of the invention herein is a subject having, or at risk of developing, a disease or disorder characterised by expression or upregulated expression of the target protein. In some embodiments, the subject to be treated is a subject having, or at risk of developing, a cancer, e.g. a cancer expressing the target protein, or a cancer in which expression of the target protein is upregulated.

In some embodiments, the method additionally comprise therapeutic or prophylactic intervention for the treatment or prevention of a disease or disorder, e.g. chemotherapy, immunotherapy, radiotherapy, surgery, vaccination and/or hormone therapy. In some embodiments, the method additionally comprises therapeutic or prophylactic intervention, for the treatment or prevention of a cancer.

T Cell Therapy

T cell therapy can include adoptive T cell therapy, tumour-infiltrating lymphocyte (TIL) immunotherapy, autologous cell therapy, engineered autologous cell therapy (eACT), and allogeneic T cell transplantation.

The T cells of the immunotherapy can come from any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumours. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in US2013/0287748, which is herein incorporated by references in its entirety.

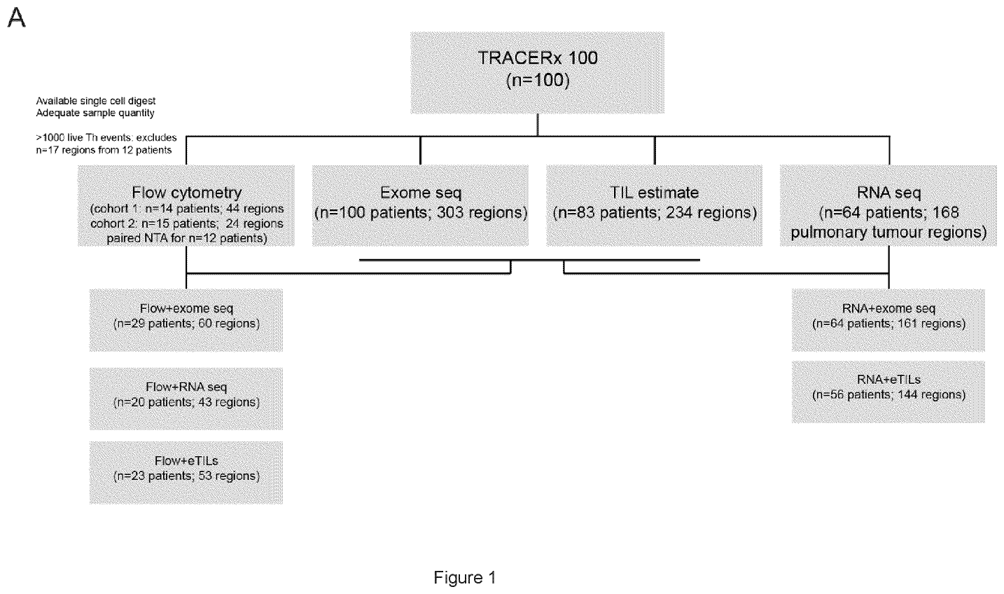

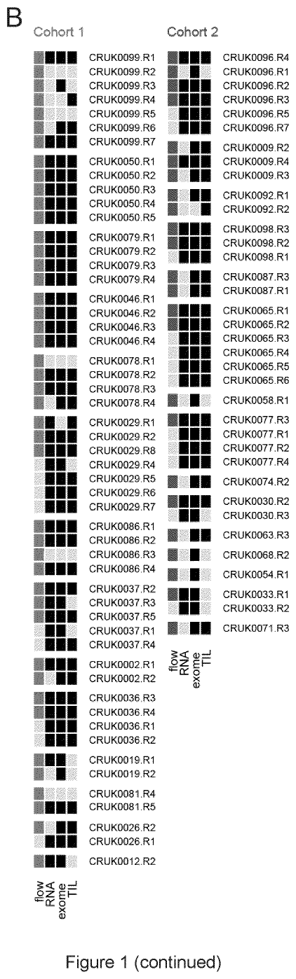

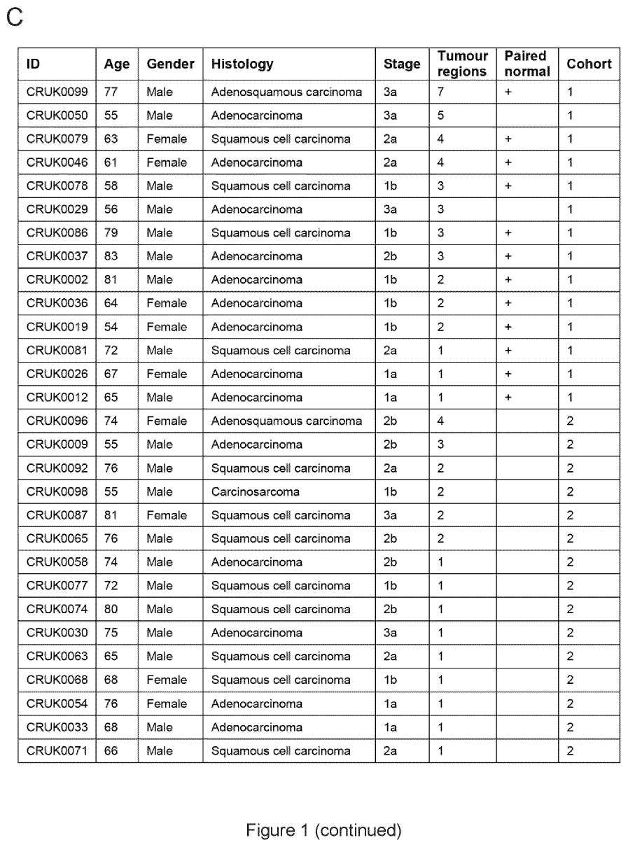

The term “engineered Autologous Cell Therapy,” which can be abbreviated as “eACT™,” also known as adoptive cell transfer, is a process by which a patient's own T cells are collected and subsequently genetically altered to recognize and target one or more antigens expressed on the cell surface of one or more specific tumour cells or malignancies. T cells can be engineered to express, for example, chimeric antigen receptors (CAR) or T cell receptor (TCR). CAR positive (+) T cells are engineered to express an extracellular single chain variable fragment (scFv) with specificity for a particular tumour antigen linked to an intracellular signalling part comprising a costimulatory domain and an activating domain. The costimulatory domain can be derived from, e.g., CD28, and the activating domain can be derived from, e.g., CD3-zeta (FIG. 1). In certain embodiments, the CAR is designed to have two, three, four, or more costimulatory domains. The CAR scFv can be designed to target, for example, CD19, which is a transmembrane protein expressed by cells in the B cell lineage, including all normal B cells and B cell malignances, including but not limited to NHL, CLL, and non-T cell ALL. Example CAR+ T cell therapies and constructs are described in US2013/0287748, US2014/0227237, US2014/0099309, and US2014/0050708, and these references are incorporated by reference in their entirety.

T cells engineered according to the present invention may be engineered at any stage before their use, in particular engineering to overexpress and/or knock-out or decrease expression of one or more genes selected from SIT1, SAMSN1, SIRPG, CD7, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, CD82, and TNIP3 may be carried out prior to or after a step of T cell expansion.

Subjects

The subject to be treated according to the invention may be any animal or human. The subject is preferably mammalian, more preferably human. The subject may be a non-human mammal, but is more preferably human. The subject may be male or female. The subject may be a patient. A subject may have been diagnosed with a disease or condition requiring treatment, may be suspected of having such a disease or condition, or may be at risk from developing such a disease or condition.

The following is presented by way of example and is not to be construed as a limitation to the scope of the claims.

### EXAMPLES

### Example 1—Mutational Burden is Associated with Compartment—Wide Features of Intratumour CD4 T Cell Dysregulation in Lung Cancer

Material and Methods

Patients and samples: All patients within this study were drawn from the first 100 enrolled to the UK multicentre lung TRACERx study as previously described (https://clinicaltrials.gov/ct2/show/NCT01888601, independent Research Ethics Committee approval reference 13/LO/1546). Sample collection and data analysis was carried out with written consent from all participants. Samples for flow cytometry were selected based on availability of single cell digest material of adequate quantity and whole exome sequencing. All tumour samples were verified as such by independent pathology review of H&E slides.

Flow cytometry: Fresh tumour and NTL surgical resection specimens were minced into 1 mm pieces in RPMI-1640 (Sigma) with Liberase TL (Sigma) and DNAase I (Roche) followed by mechanical disaggregation using a gentleMACS dissociator (Miltenyi Biotec) at 37° C. for 1 hour. Single cells were obtained by gently passing the suspension through a 70 μm cell strainer with 5 ml complete RPMI-1640 (PBS containing 2% FBS and 2 mM EDTA) and lymphocytes isolated by density gradient centrifugation (750 g for 10 minutes) on Ficoll Paque Plus (GE Healthcare). The interface was washed twice with complete RPMI-1640, resuspended in 90% FBS with 10% DMSO (Sigma) and cryopreserved prior to staining.

For staining, cell were thawed and washed in FACS buffer (5% FBS). For cohort 1, cells were surface stained with the following antibodies to surface markers; CD45R0 BUV395 (UCLH1), CD8 BUV496 (RPA-T8), CD45RA BUV563 (HI100), CD4 BUV661 (SK3), CD28 BUV737 (28.2), CD3 BUV805 (SK7), PD1 BV421 (EH12.1), CD57 BV605 (NK-1), 41BB BV650 (4B4-1), CD27 BV786 (L128), TIM3 BB515 (7D3), CD25 APC-R700 (2A3), all from BD and ICOS PE-CY7 (C398.4A) from Biolegend, followed by fixation and permeabilisation with FOXP3 Transcription Factor Staining Buffer set (Thermo) and intracellular staining with FOXP3 AF647 (259D/C7), TBET PE (4B10), GZMB PE-CF594 (GB11) and Ki67 BV480 (B56) from BD and EOMES PerCP-e710 (WD1928) from Thermo. For cohort 2, cells were stained with the following antibodies to surface markers; CD8 BUV496 (RPA-T8), CD45RA BUV563 (HI100), HLA-DR BUV661 (G46-6), Fas BUV737 (DX2), CD3 BUV805 (SK7), PD1 BV421 (EH12.1), CD57 BV605 (NK-1), CD127 BB515 (HIL-7R-M21), CD28 APC-R700 (28.2) all from BD and CD4 biotin (OKT4), CD27 BV510 (O323), CCR7 BV650 (G043H7), CD103 BV711 (Ber-ACT8), ICOS BV786 (C398.4A), CTLA4 PE-CY7 (L3D10) from Biolegend. Streptavidin BUV395 was purchased from BD. Intracellular staining was done with antibodies to EOMES PerCP-e710 (WD1928) and FOXP3 PE (PCH101) from Thermo, CTLA4 PE-CY7 (L3D10) and NKG2D AF647 (1D11) from Biolegend and GZMB PE-CF594 (GB11) from BD. In both cohorts, eBioscience Fixable Viability Dye eFluor 780 (Thermo) was used to exclude non-viable cells. Data were acquired on a BD Symphony flow cytometer and cells gated for size, singlets, viability and CD3⁺CD8− T cells in FlowJo v10 (Treestar) for further analysis.

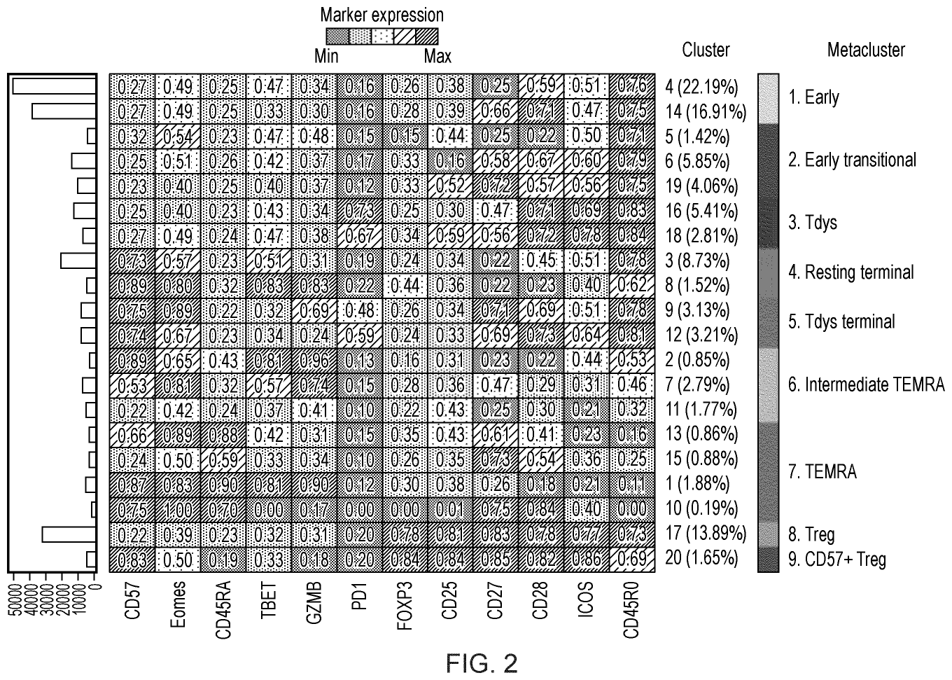

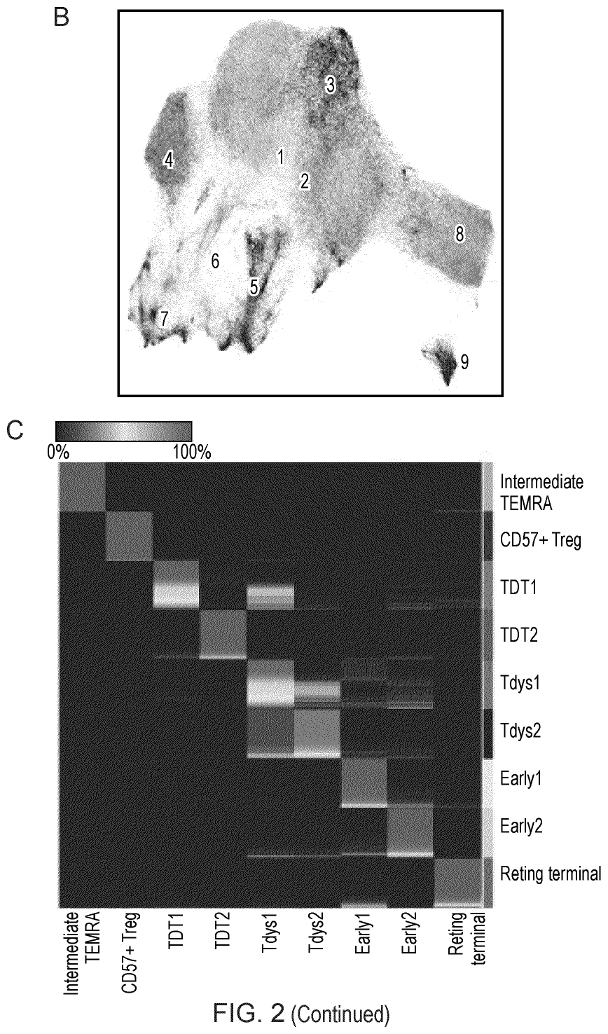

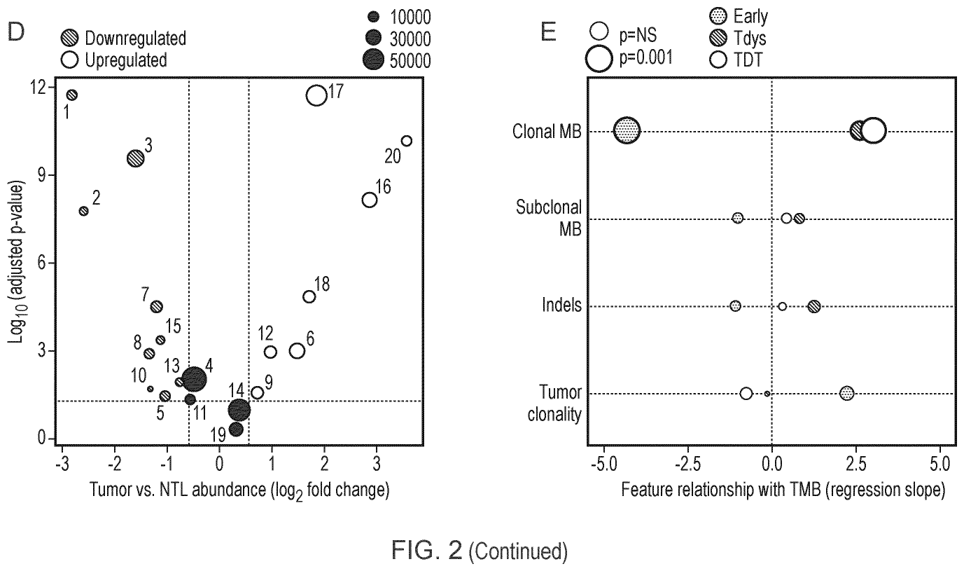

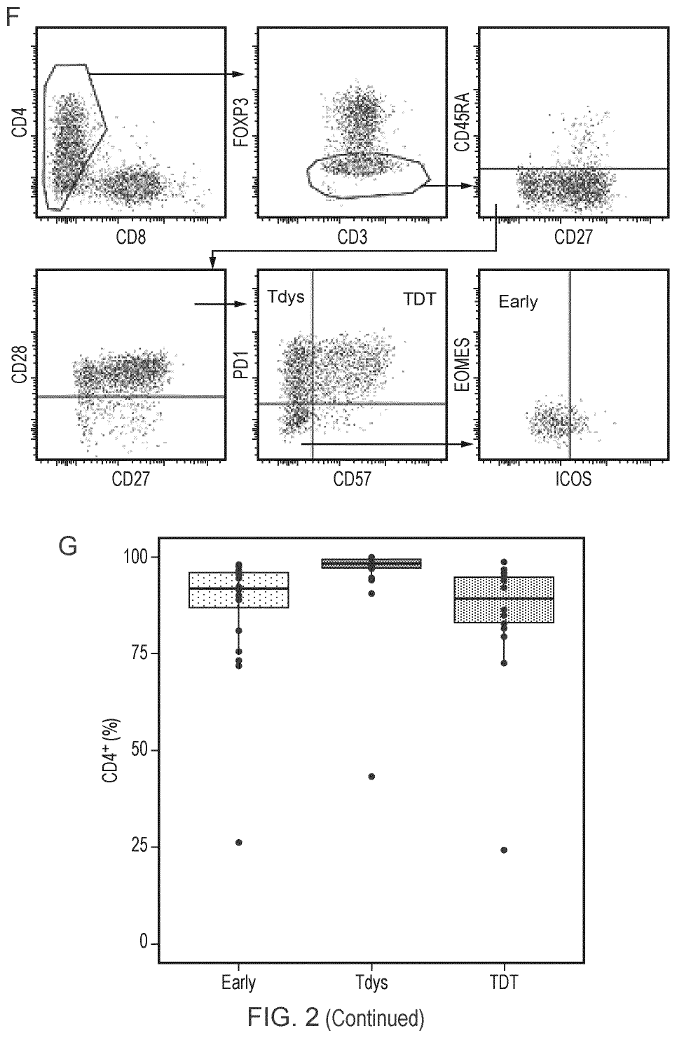

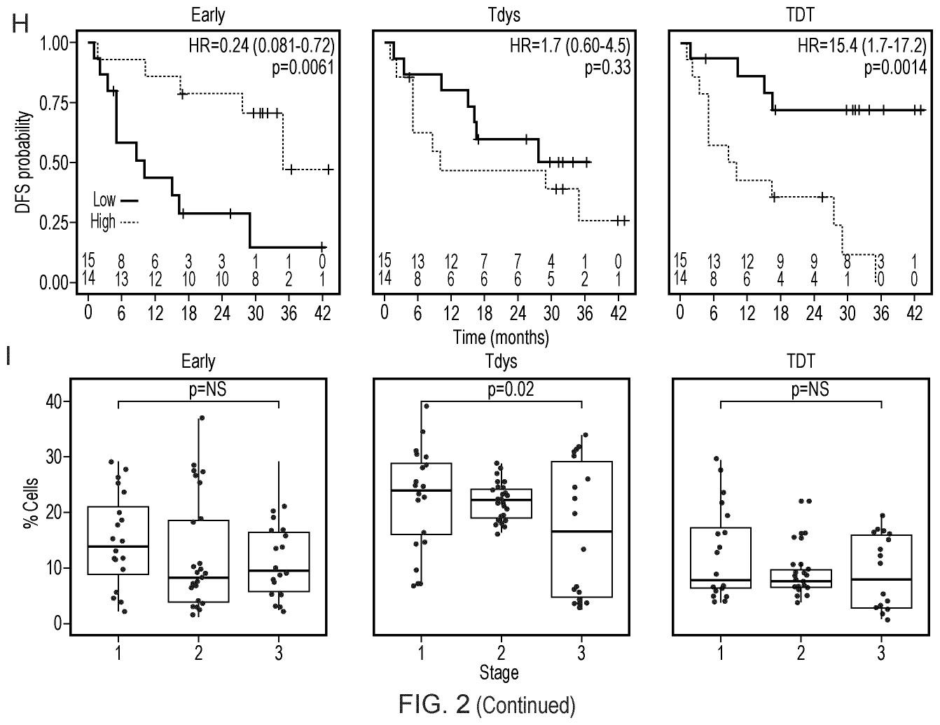

CD4 T cell identification: Liberase treatment has previously been described to cleave the CD4 antigen resulting in variable detection of this marker37. We therefore gated CD3⁺CD8− to ensure complete capture of the T helper population. We confirmed the CD4 status of Early, Tdys and TDT populations gated from amongst CD3+CD8− cells using regions with a clear CD4+ population (n=20/61 across both cohorts). Evaluation of the percentage of CD4+ cells amongst these three subsets revealed over 85% CD4 expression (mean CD4+86.8, 95.2 and 85.7% in early, Tdys and TDT subsets respectively; FIG. 2G).

Sequencing: Multi-regional whole exome sequencing, mutation calling and clonality estimation were carried out as described before (Jamal-Hanjani, M. et al., 2017). Briefly, raw paired end whole exome sequencing reads from tumour and matched germline samples were aligned to the hg19 genomic assembly. Non-synonymous mutations were identified and classified as clonal or subclonal using a modified version of PyClone (Roth, A. et al., 2014), considering variant allele frequency, copy number and tumour purity. Synonymous and non-synonymous mutations from each tumour region were identified by comparing germline and tumour DNA. RNA was extracted using a modification of the AllPrep kit (Qiagen) and ribosome depleted prior to library preparation of samples with an RNA integrity score of >=5, measured by TapeStation (Agilent Technologies). Second-strand cDNA synthesis incorporated dUTP. The cDNA was end-repaired, A-tailed and adaptor-ligated. Before amplification, samples underwent uridine digestion. The prepared libraries were size-selected, multiplexed and underwent quality control before paired-end sequencing. 75 bp paired end sequencing with an average of 50 million reads per sample was carried out. FASTQ data underwent quality control and were aligned to the hg19 genome using STAR (Dobin, A. et al., 2013). Transcript quantification was performed using RSEM with default parameters (Li, B. & Dewey, C. N., 2011).

TIL evaluation: TIL estimation was carried out according to International Immuno-Oncology Biomarker Working Group guidelines (Hendry, S. et al., 2017) that have been shown to be reproducible amongst trained pathologists (Denkert, C. et al., 2016). Using region level H&E slides, the relative proportion of stromal to tumour area was determined and percentage TILs reported for the stromal compartment by considering the area of stroma occupied by mononuclear inflammatory cells divided by total stromal area. In an intra-personal concordance test, high reproducibility was demonstrated. The International Immuno-Oncology Biomarker Working Group has developed a freely available training tool to train pathologists for optimal TIL-assessment on H&E slides (www.tilsincancer.org).

TCGA data: Pancancer TCGA data were downloaded from the GDC website (https://gdc.cancer.gov/about-data/publications/panimmune)(Thorsson, V. et al., 2018). This included upper quartile normalized gene transcript count estimates, clinical and mutational burden data. Clinical data were used as previously published (Liu et al., 2018). To test the relationship between the XDS signature and TMB in TCGA lung cancer cohorts, non-synonymous mutational burden as an absolute count was calculated using data generated by the MC3 project (Ellrott et al., 2018) for comparison with TRACERx data. For survival and linear regression analyses, z-score scaled non-silent mutations per Mb were used and found to give very similar results to mutational burden estimated from the MC3 project data.

Statistical analysis: All calculations were carried out in the R statistical programming environment version 3.4.3. Individual regions were treated as independent data points in exploratory analyses. Correlation analysis was carried out according to the Pearson method and the two-tailed Wilcoxon rank-sum test was utilised to evaluate whether two samples were derived from the same population. Hypothesis tests using mixed effects linear regression models to account for dependencies within the data due to tumour multiregionality and effects of histology (resulting in within patient and within group similarities respectively) were additionally conducted. Mixed effects modelling was implemented using the package nlme. Where appropriate, p-values were adjusted by the Benjamini-Hochberg method, to control the type 1 error rate in the context of multiple testing. Survival analysis was carried out with Cox regression models implemented in the survival package. Kaplan-Meier plots and log-rank p-values were generated using the package survminer.

Unsupervised Analysis of Flow Cytometry Data

Clustering: Clustering was carried out using a pipeline modified from Nowicka et al., on all samples with over 1000 live CD3⁺CD8⁻ events. FCS files were read in and logicle transform applied using the estimateLogicle function of the flowCore package (Hahne et al., 2009). Markers with low contribution to intercellular phenotypic variance were removed prior to clustering analysis based on low expression above background and calculation of the PCA based non-redundancy score, as previously defined (Nowicka et al., 2017; Levine et al., 2015), resulting in exclusion of the markers TIM3, Ki67 and 41BB. Data were clustered onto a 7×7 node square self-organising map (SOM) implemented in the FlowSOM package (Van Gassen et al, 2015), followed by hierarchical consensus clustering of nodes using the ConsensusClusterPlus package (Wilkerson & Hayes 2010) as previously described. Based on inspection of consensus matrix and tracking plots, the data were overclustered into 20 populations. To understand cluster relationships, we applied the UMAP algorithm (Becht, E. et al., 2018) for dimension reduction of all events, in view of its superior preservation of input space topological properties compared to t-SNE. UMAP was carried out using the package uwot and similar clusters were manually grouped into metaclusters based on UMAP co-localisation and marker expression.

Differential abundance analysis: To determine differential abundance of clusters between tumour and NTL tissue accounting for sample multiregionality and pairing, we applied negative binomial generalised linear models using the package edgeR (Robinson et al., 2010), as recently described for cytometry data (Lun et al, 2017).

Discovery of populations differentially abundant with TMB: As initial node weights are randomly initialised prior to SOM training, there is inherent stochasticity in the process. To address this, we iterated the clustering procedure ×1000 with random seeds and at each recursion carried out Pearson correlation analysis to test the relationship between abundance of each FlowSOM cluster and sample TMB. Positive and negatively correlating clusters with a Benjamini-Hochberg false discovery rate (FDR) of <0.1 were retained at each iteration. Similar clusters found across multiple iterations were manually combined based on their UMAP proximity and marker profile to identify populations that stably change with TMB. The most abundant populations (composed of individual clusters observed over 200 times across 1000 iterations; n=9) were retained for further analysis. To evaluate clustering stability, we first labelled the population identity of each cell in a representative clustering iteration. Then for each cell, we calculated a probability of being identified within each of the nine populations above by dividing its frequency of identification within a given population by the total frequency of identification across the nine populations in 1000 iterations to generate the Figure S2C heatmap.

Tumour clonal diversity: Tumour clonal diversity was estimated by calculating the Shannon entropy for each region, based on the number and prevalence of each clone, implemented using the entropy package. A region composed of a single subclone was assigned a value of 0.

Single Cell RNA-Sequencing Analysis

Data processing and imputation: Count and metadata from the study of Guo et al. were downloaded from the Gene Expression Omnibus website (accession number GSE99254). Cells with library size or number of genes with count >0 below three median absolute deviations (MADs) from the median of all cells were excluded, as were genes with an average count of <1 or those expressed in fewer than 10 cells. The package scImpute was used to identify and perform imputation on dropout expression values (Li et al, 2018).

Gating: Both flow cytometry and scRNAseq provide continuous measurements of individual markers expressed at a single cell level. For samples with matched cytometry and scRNAseq data, good between-technique concordance in identification of populations has been reported, supporting flow cytometry-like gating approaches to scRNAseq data (Oetjen et al, 2018). Counts per million (CPM) expression data were normalised by the trimmed mean of M-values (TMM) procedure to account for compositionality, followed by login transformation for manual gating of populations on biaxial plots.

Differential gene expression analysis: Genes differentially expressed between the three subsets were identified using the edgeR edgeRQLFDetRate procedure recently described as a top-ranking approach to differential expression analysis in single-cell RNA-seq data⁸⁷. The analyses were conducted with patient as a co-factor. Differential analysis was carried out on genes with >1 CPM in over 25% of cells. In the Soneson et al. study, this approach resulted in a type I error control rate of slightly above the imposed level of p=0.05 (Soneson et al, 2018). To apply a strict control to this, genes identified by edgeR as differentially expressed between groups with fold change >2 and FDR<0.05 were retained for further analysis if they were additionally identified as differentially expressed (p<0.05) between subsets using a Wilcoxon rank-sum test. Heatmaps were generated using log₁₀ CPM expression values with the ComplexHeatmap package (Gu et al, 2016).

GSEA: The package fgea (Sergushichev, 2016) was used for preranked GSEA⁹⁰ with 10 000 permutations. Genes were ranked according to their log₂ fold change (log FC) between groups using edgeR::glmFit with prior.count=5. GSEA was carried out using signatures of CD4 dysfunction previously described in mouse studies of chronic viral infection (Crawford et al., 2014), lupus nephritis (Tilstra et al, 2018) and autoimmune colitis (Shin et al., 2018). We constructed these signatures by selecting the top 100 differentially expressed genes in each study. Human orthologues were identified using Ensembl and NCBI HomoloGene databases. To confirm enrichment of T cell progenitor-like signatures amongst the Early subset, we carried out GSEA on C7 gene-sets from MSigDB (Subramanian et al., 2005), filtered to include effector T central memory signatures only (n=18) and represent the top four pathways in Figure S4E, from the following publications; GSE11057 (Abbas et al, 2009), GSE26928 (Chevalier et al, 2011), GSE3982 (Jeffrey et al, 2006).

Bulk RNA-Sequencing Data Analysis

CD4 subset gene signatures: Gene signature enrichment was evaluated using upper quartile normalised TCGA and TRACERx RNA sequencing RSEM count data. Effector CD4 subset T cell gene signatures were tested for correlation with flow cytometry data. For patients with matched RNA sequencing and pathologist evaluated TILs (n=56 patients, 144 regions), we found the Danaher T cell transcriptional signature to closely correlate and therefore used this to estimate TIL density (Danaher et al., 2017). For each signature, expression of constituent genes was log₁₀ transformed, z-score scaled and the mean value per sample used to represent enrichment. Non-protein coding genes and those not represented in both TCGA and TRACERx data were excluded. For Treg signatures, enrichment values were corrected for TIL infiltrate by deducting the corresponding Danaher T cell signature values for each region.

TCGA xCell signatures were used as previously calculated (Aran et al., 2017). For TRACERx RNAseq data, xCell signature values were generated using the published package (https://github.com/dviraran/xCell) and z-score scaled across all samples for which RNA sequencing was available.

TCF7/LEF1 signature: Xing et al. have previously published RNA sequencing data on genes differentially expressed by mouse Tcf7/Lef1 knockout vs. wildtype CD8 thymocytes (Xing et al, 2016). Genes upregulated in knockout cells characterise later differentiated T cells, whilst genes downregulated characterise progenitor-like T cells. We selected 141 upregulated and 68 downregulated genes (fold change >4) to generate late differentiation and sternness gene sets respectively. As CD4^{ds} involves a loss of early differentiated cells and a gain of later differentiated subsets, a signature of CD4^{ds} was defined as the value of the stemness minus late differentiation gene sets.

### Example 1.1—Mutational Burden Correlates with Intratumour CD4 T Cell Differentiation Skewing

We characterised the NSCLC intratumour CD4 T cell differentiation landscape with 19-marker flow cytometry on tumour infiltrating lymphocytes (TILS) from 44 tumour regions of 14 patients in the TRACERx 100 cohort (FIG. 1A). Samples were selected for adequate single cell digest material and paired exome sequencing (n=37 regions). Matched non-tumour lung (NTL) regions were available for 12 patients.

Due to previously reported CD4 staining attenuation following enzymatic tumour dissociation (Ahmadzadeh et al., 2019), CD3+CD8− cells were analysed to ensure complete capture of T helper cells (FIG. 2F; see Methods for validation details). Unsupervised clustering of combined region data identified 20 CD4 subpopulations (see Methods) that were manually grouped into nine meta-clusters based on marker expression and co-localisation in uniform manifold approximation and projection (UMAP, Becht et al., 2018) dimension reduced space (FIG. 2A, B). These populations included an antigen experienced subset with low activation marker expression (CD45R0+CD28+PD1−ICOSlowCD57−) and a similar population with intermediate ICOS expression (CD45R0+CD28⁺PD1-ICOSintCD57−), labelled early differentiated (Early) and early transitional respectively. A population with high co-inhibitory and co-stimulatory receptor expression (CD45R0+PD1+ICOShighCD57−) was labelled T dysfunctional (Tdys) (Day et al., 2006; Crawford et al., 2014). Four populations had features of CD4 terminal differentiation including Eomes and CD57 expression; 1. An activated population with high PD1 and intermediate ICOS expression reminiscent of Tdys, termed Tdys/terminal effector (TDT; CD45R0+PD1+ICOSintEomes+CD57+); 2. A non-activated subset with low PD1 and ICOS expression (CD45R0+PD1−ICOSlowEomes+CD57+) termed terminally differentiated resting; 3. T effector memory cells re-expressing CD45RA (TEMRA) and 4. A CD45R0/CD45RA intermediate population termed intermediate TEMRA. Although TDT cells expressed co-stimulatory receptors CD27 and CD28 usually associated with early differentiation (Mahnke et al., 2013), their expression can also mark T cell activation (Warrington et al., 2003; Salazar-Fontana et al, 2001). We additionally identified two FOXP3+CD25+T regulatory (Treg) populations distinguishable by CD57 expression.

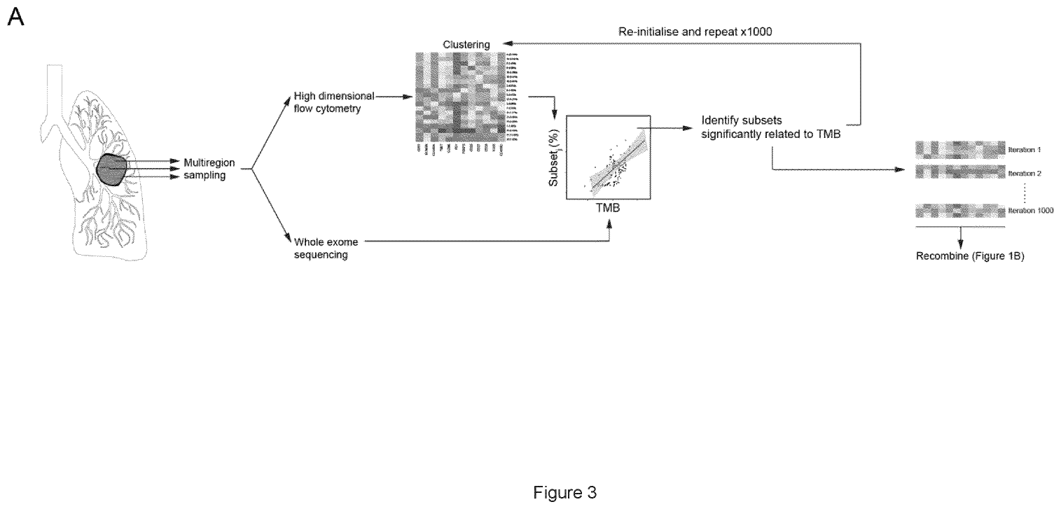

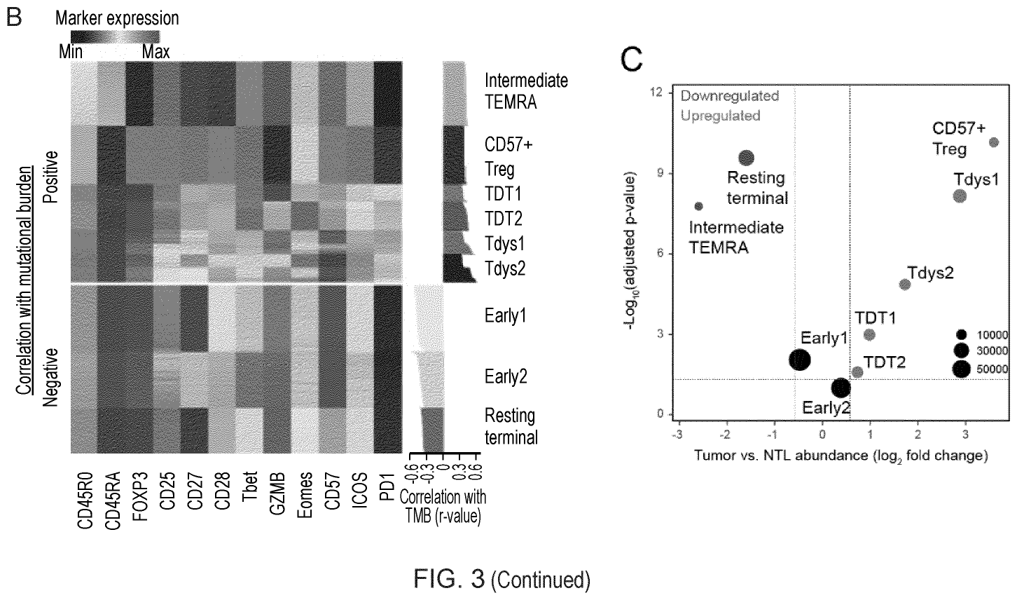

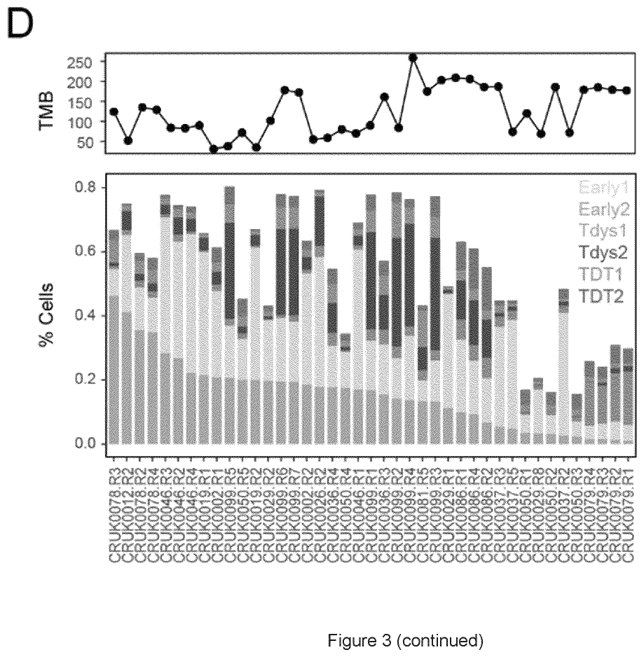

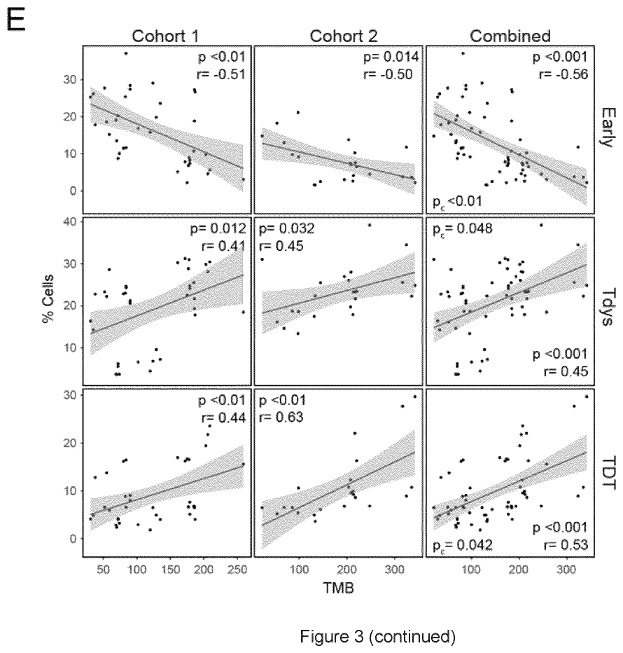

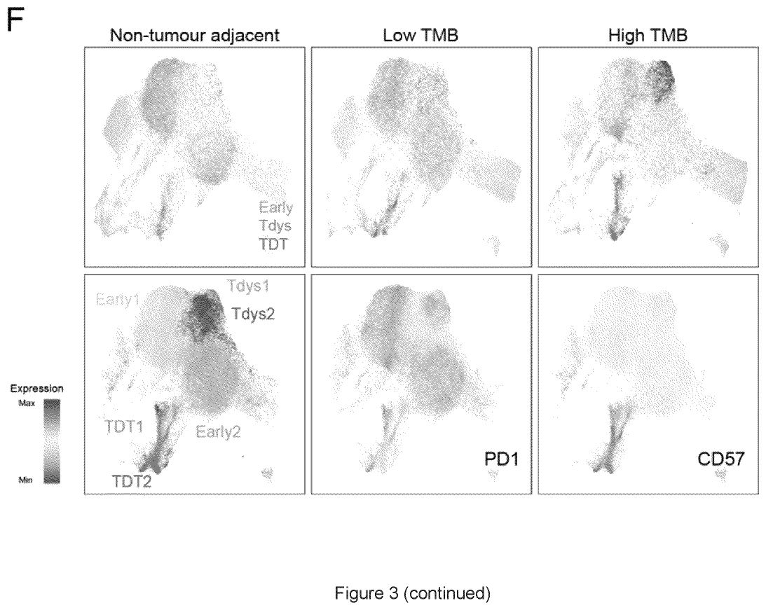

We identified CD4 subsets whose abundance varies with exonic, non-synonymous tumour mutational burden (TMB) using samples with paired flow cytometry and exome sequencing data (n=14 patients; 37 regions; FIG. 1A). To account for stochasticity in population identification, unsupervised clustering was repeated 1000 times. At each iteration, the relationship between cluster abundance and TMB was evaluated to identify populations that consistently vary in abundance with mutational burden (FIG. 3A). This approach identified eight effector subsets including two early differentiated populations that declined, whilst two Tdys and two TDT populations increased in abundance with TMB (FIG. 3B, D). Intermediate TEMRA and resting TD populations additionally correlated with TMB, but their quiescence expression profile and greater abundance in NTL vs. tumour regions suggested a reduced likelihood of anti-tumour engagement and we excluded them from further analyses (FIG. 3C, 2D).

To confirm the results of unsupervised analysis, we selected a validation cohort of TRACERx patients with the same criteria as before (n=15 patients, 24 regions) for TIL flow cytometry with an overlapping marker panel. The subsets of interest were manually gated for further analysis. In both cohorts, manually gated Early abundance inversely associated with TMB, whilst Tdys and TDT abundance was positively associated (FIG. 3E). In a combined analysis, these findings remained significant, independent of tumour histological type and multiregionality. We use the term CD4 differentiation skewing (CD4^{ds}) to describe this pattern of Early abundance decline and dysfunctional subset gain.

We considered the role of mutation clonality and found the burden of non-synonymous clonal but not subclonal mutations to correlate with CD4^{ds} (FIG. 2E). Neither insertion-deletion mutation burden nor tumour clonal diversity as measured by the Shannon index correlated with CD4^{ds}.

In chronic viral infection, loss of early differentiated (Okoye et al., 2007) and gain in dysfunctional CD4 subsets associate with impaired immunity (Day et al., 2006; Kaufmann et al., 2007). In the combined cohort, low Early and high frequency of TDT cells (grouped according to the median) correlated with worse disease free survival (DFS), suggesting CD4^{ds} may mark impaired anti-tumour immunity (FIG. 2H). There was no relationship between CD4^{ds} and tumour stage (FIG. 2I).

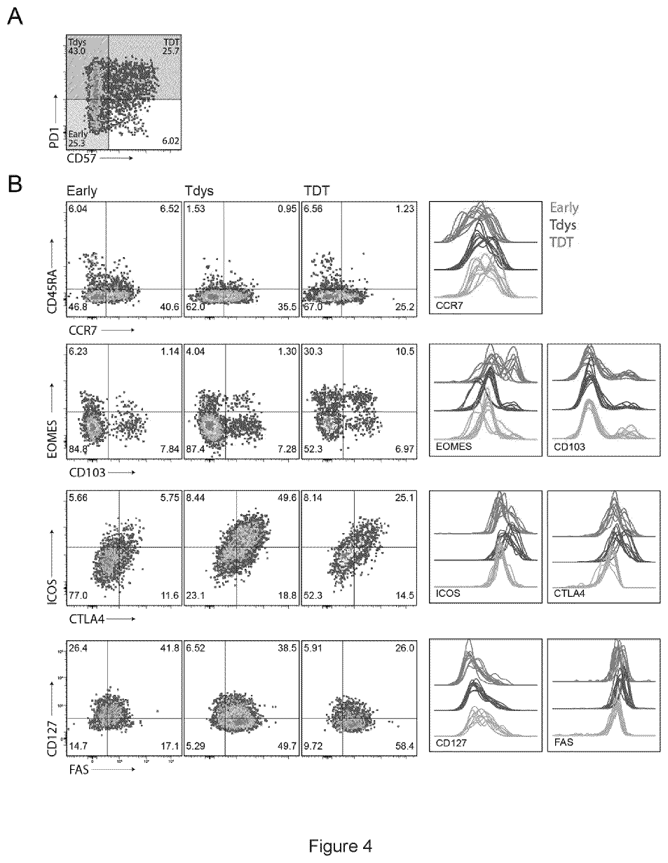

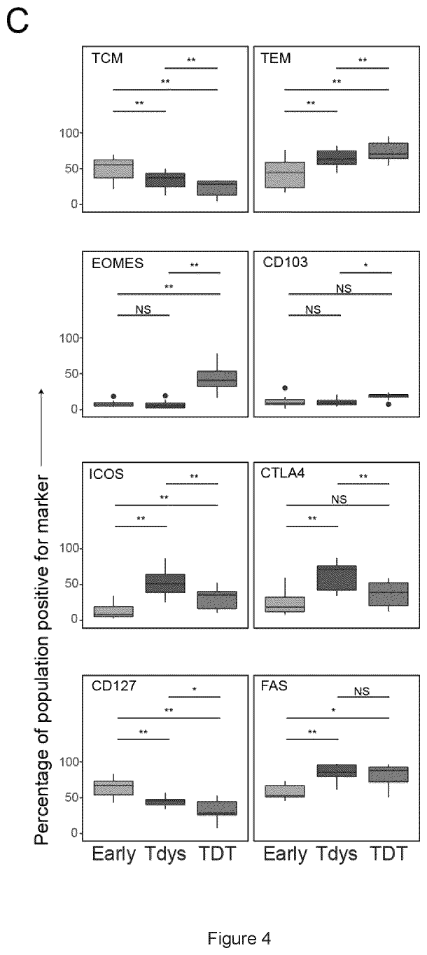

CD4 subset identity was confirmed in the validation cohort. The PD1 vs. CD57 profiles of manually gated populations are shown in FIG. 2A. CCR7 expression confirmed Early population T central memory (Tcm) enrichment, whilst Tdys and TDT were predominantly CD45R0+CCR7− effector memory cells (FIG. 4B, C). Consistent with dysfunction, Tdys highly expressed ICOS and the co-inhibitory receptor CTLA4, whilst TDT had high Eomes and low IL-7 receptor (CD127) expression (Patil et al., 2018). TDT had the highest CD103+ tissue resident memory (Trm) cell frequency. Both Tdys and TDT highly expressed the late differentiation marker CD95 (Fas) (Malinke et al., 2013).

### Example 1.2—Single Cell Transcriptional Characterisation of Early, Tdys and TDT Subsets Unveils Distinct Developmental and Regulatory Programmes

We characterised the transcriptional features of these populations using a recently reported NSCLC TIL single cell RNA sequencing (scRNAseq) dataset (Guo et al., 2018).

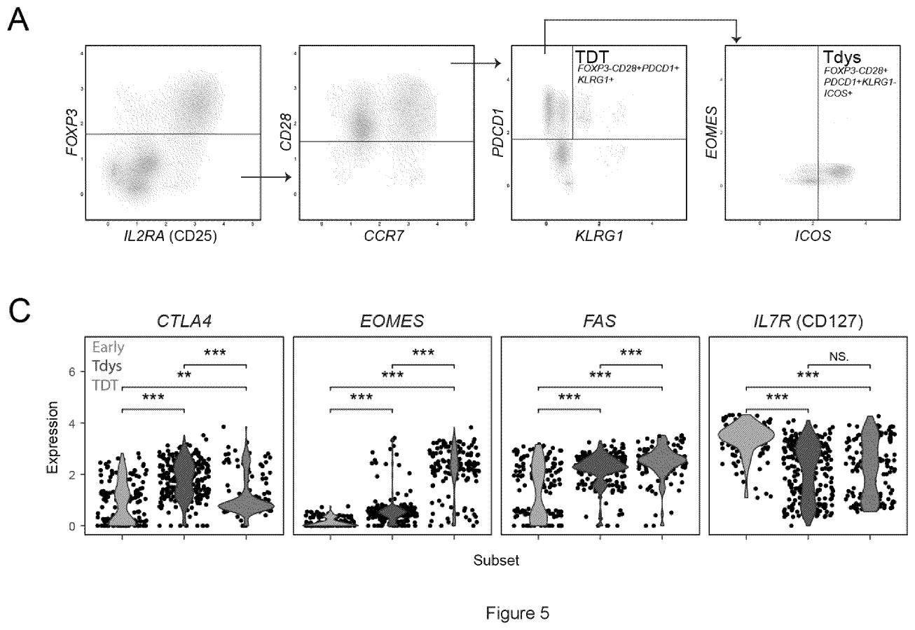

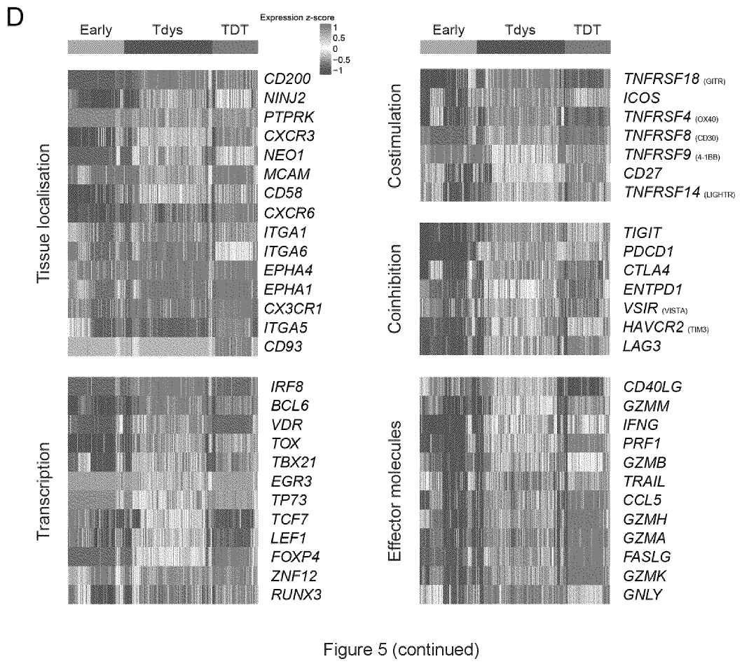

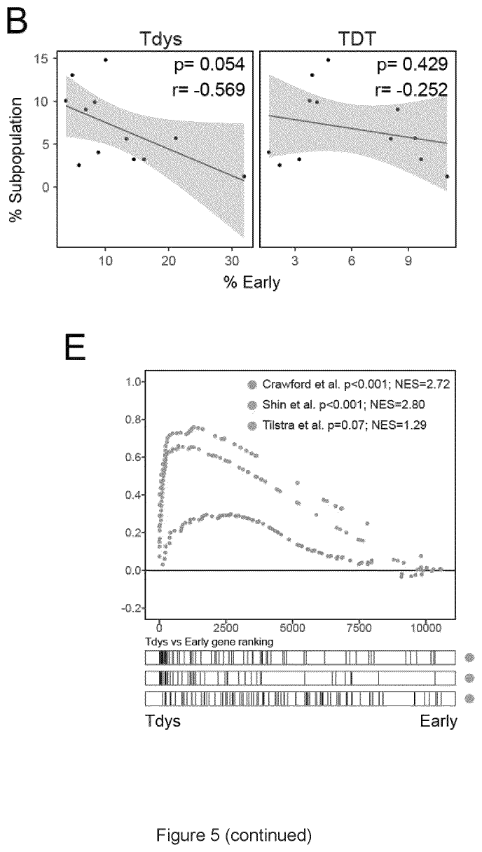

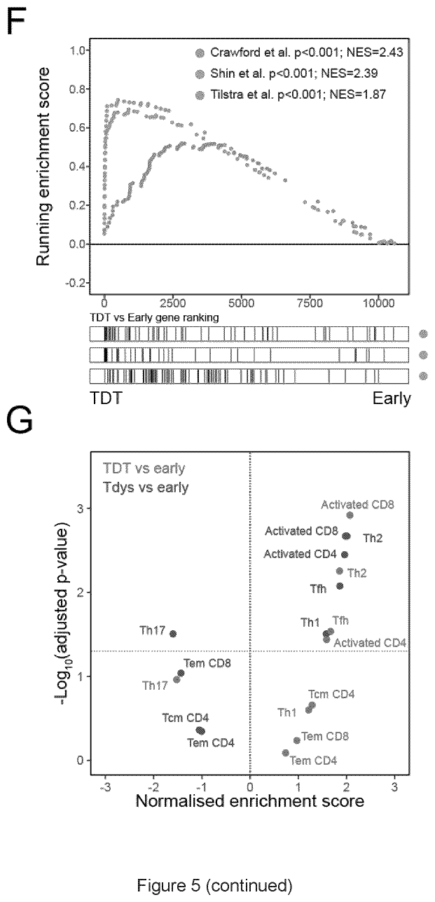

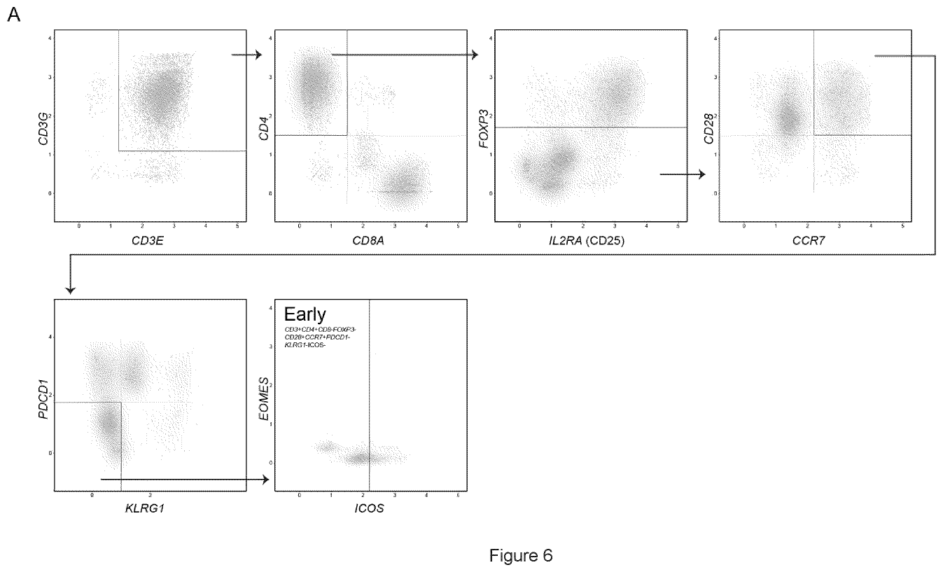

Subsets were identified by a manual gating strategy based on our flow cytometry analysis (FIGS. 5A and 6A). Of 2469 CD4 T cells from 14 patients, we identified 175 Early (FOXP3−CD28+CCR7+PDCD1−KLRG1−ICOSlow), 272 Tdys (FOXP3−CD28+PDCD1+KLRG1−ICOShigh) and 143 TDT (FOXP3−CD28+PDCD1+KLRG1+) cells. B3GAT1 that generates the CD57 antigen had a high dropout rate (80.3% of CD4+ cells). As KLRG1 and CD57 are highly coexpressed upon terminally differentiated T cells (Di Mitri et al., 2011), we used the former to identify TDT cells.

Concordance between scRNAseq and flow cytometry identified populations was confirmed by evaluating expression of genes characterised by flow cytometry and not used for scRNAseq gating (CTLA4, EOMES, FAS and IL7R; FIG. 5C). In keeping with their cytometry profile, scRNAseq Tdys and TDT populations had high CTLA4 and EOMES expression respectively and elevated FAS compared to Early. Early population identity was confirmed by high expression of IL7R, encoding CD127.

TRACERx flow cytometry measured abundances of Early and Tdys/TDT populations were inversely related. Similar relationships between scRNAseq identified populations provided evidence of CD4^{ds} in this cohort (FIG. 5B).

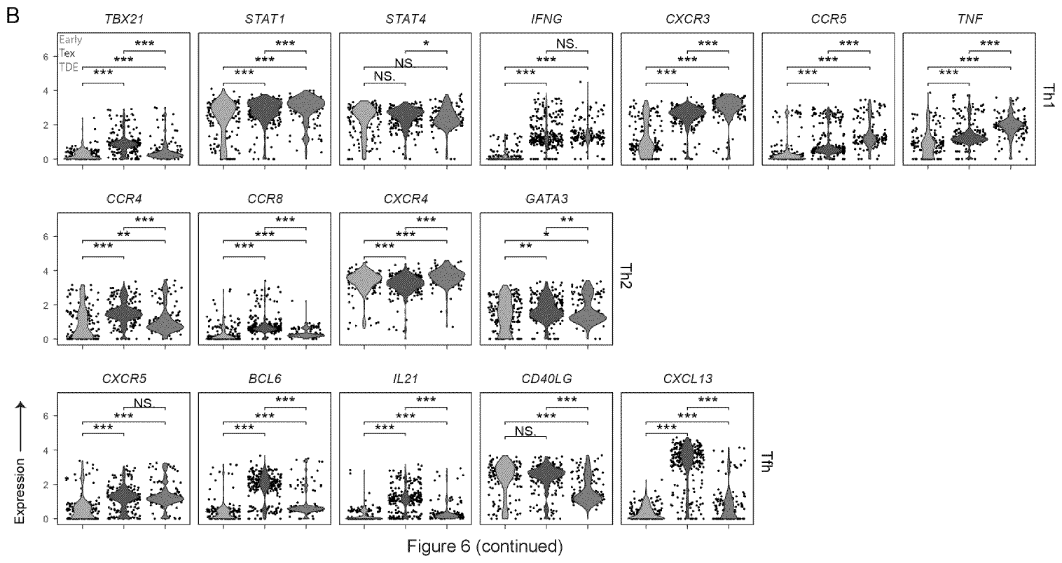

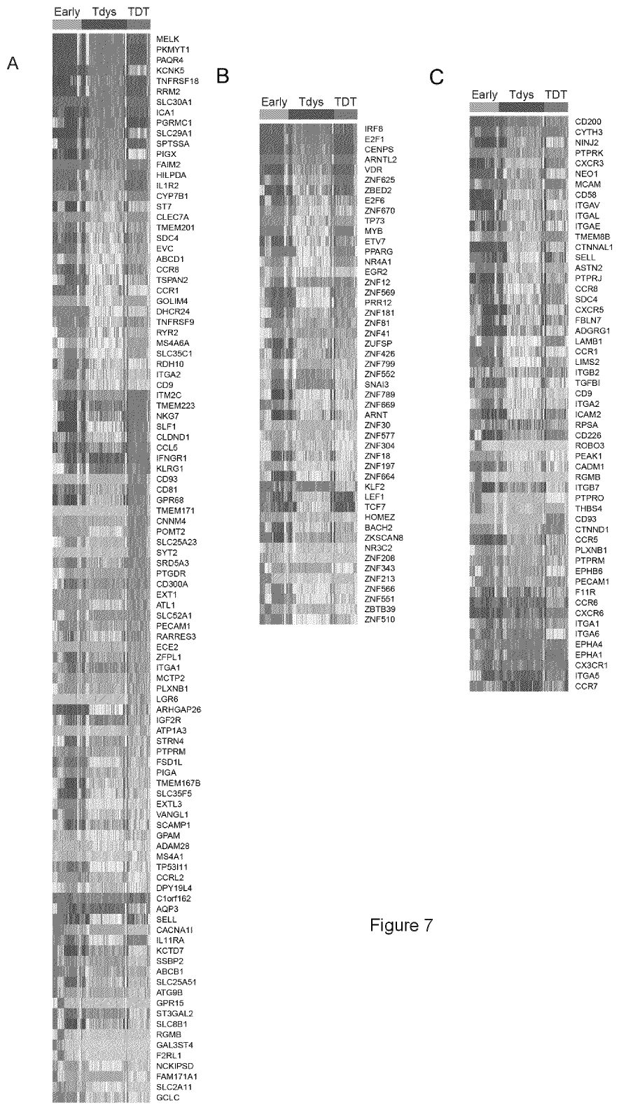

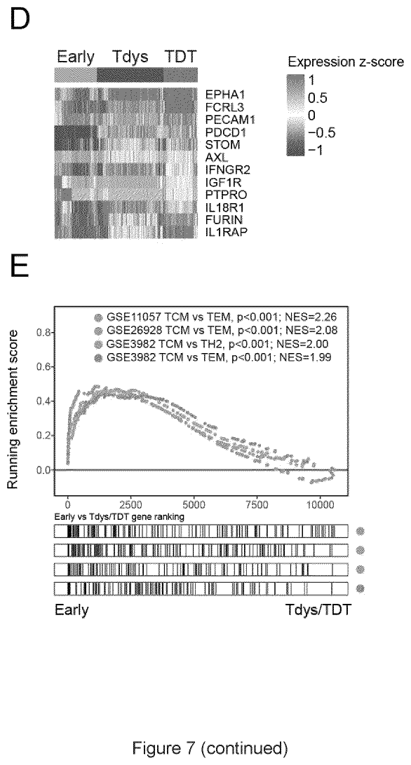

To further characterise the scRNAseq populations, we carried out gene set enrichment analysis (GSEA) with signatures of progenitor-like and dysfunctional CD4 T cell differentiation in the context of infection (Crawford et al., 2014) and autoimmunity (Shin et al, 2018, Tilstra et al., 2018). Tcm signature genes were significantly upregulated by Early cells (FIG. 7E), whilst Tdys and TDT subsets had transcriptional profiles of CD4 dysfunction related to persistent antigen exposure (FIG. 5E, F).

Differential gene expression analysis revealed significant transcriptional differences between the scRNAseq identified subsets (FIG. 5D, 7A-D). To explore potential mediators of Tdys and TDT tissue accumulation, we analysed genes encoding adhesion molecules and chemokine receptors. Amongst genes implicated in tissue residency, both subsets expressed CXCR3, involved in CD4 tissue surveillance in autoimmunity (Nankin et al., 2002), whereas TDT cells specifically expressed ITGA1 that identifies epithelial CD8 Trm cells (Cheuk et al., 2017) (FIG. 5D, 7C).

Effector gene analysis revealed both Early and Tdys cell expression of CD40LG, suggesting antigen engagement and helper function (Quezada et al., 2004). TDT cells expressed genes characteristic of CD8 cytotoxicity, including those encoding perforin, granzyme molecules and Fas ligand, as previously described for CD4 terminal differentiation (Hirschhorn-Cymerman et al., 2012).

As effector gene expression suggested Tdys and TDT subsets may retain functional potential that can be therapeutically enhanced, we explored their expression of co-stimulatory and co-inhibitory receptor encoding genes and found discordant expression patterns suggesting their differential regulation by actionable immunotherapy targets (FIG. 5D). Whilst expression of genes encoding GITR and OX40 expression was highest amongst Tdys cells, TDT cells preferentially expressed CD27 in keeping with our flow cytometry data (FIG. 3B), in addition to TNFRSF14 (encoding LIGHTR). The Tdys subset expressed high levels of multiple co-inhibitory receptor encoding genes, whereas TDT cells were distinguished by LAG3 expression.

We found characteristic transcription factor expression profiles including Early expression of TCF7/LEF1 that maintain T cell sternness (Gattinoni et al., 2009), and specific Tdys expression of negative regulators including IRF850 and NRF151. Both Tdys and TDT expressed the dysfunction related gene TOX52 (FIG. 5D, 7B).

As shown on FIG. 7D, Tdys and TDT populations were also found to differentially express multiple genes encoding ITIM (immune-receptor tyrosine-based inhibition motif) domain proteins, compared to the Early population. These are potentially inhibitory molecules that could represent novel candidates for therapy. Indeed, after ITIM-possessing inhibitory receptors interact with their ligand, their ITIM motif becomes phosphorylated by enzymes of the Src kinase family. This enables them to recruit phosphatases such as SHP1 and SHP2 that dephosphorylate the T cell receptor complex, decreasing T cell activation. Therefore, targeting these molecules that appear to be abnormally active in Tdys and TDT populations could lead to enhanced T cell activation in these populations due to lack of dephosphorylation of the T cell receptor complex, which should in turn improve the antitumor immune response. A subset of these (in particular: EPHA1, FCRL3, PECAM1, AXL, FURIN, IL1RAP, STOM, SIRPG) is particularly promising and some of these (FCRL3, AXL, FURIN, IL1RAP, STOM, SIRPG) were selected for validation. Validation data for IL1RAP and SIRPG is shown in Example 3.

CD4 T cells can develop features of specific lineage commitment, characterised by marker expression and functional attributes. We explored this amongst scRNAseq identified subsets by GSEA using previously published signatures (Charoentong et al., 2017) and expression profiling of key lineage specific genes (FIG. 6B). In comparison to Early, both Tdys and TDT populations upregulated genes related to Th2 and T follicular helper (Tfh) differentiation (FIG. 5G). Whilst Tdys cells had similar Th1 and Th2 enrichment, TDT had non-significant Th1 enrichment and an activated CD8 signature, in keeping with expression of cytotoxicity related effector genes. Finally, we found an enrichment of Th17 signature genes in the Early population. These results suggest differential and heterogeneous acquisition of CD4 function amongst the subsets, as previously observed amongst dysfunctional CD4 cells in murine chronic LCMV25.

### Example 1.3—A Transcriptional Signature of Intratumour CD4^{ds} Predicts Survival in Independent Cohorts

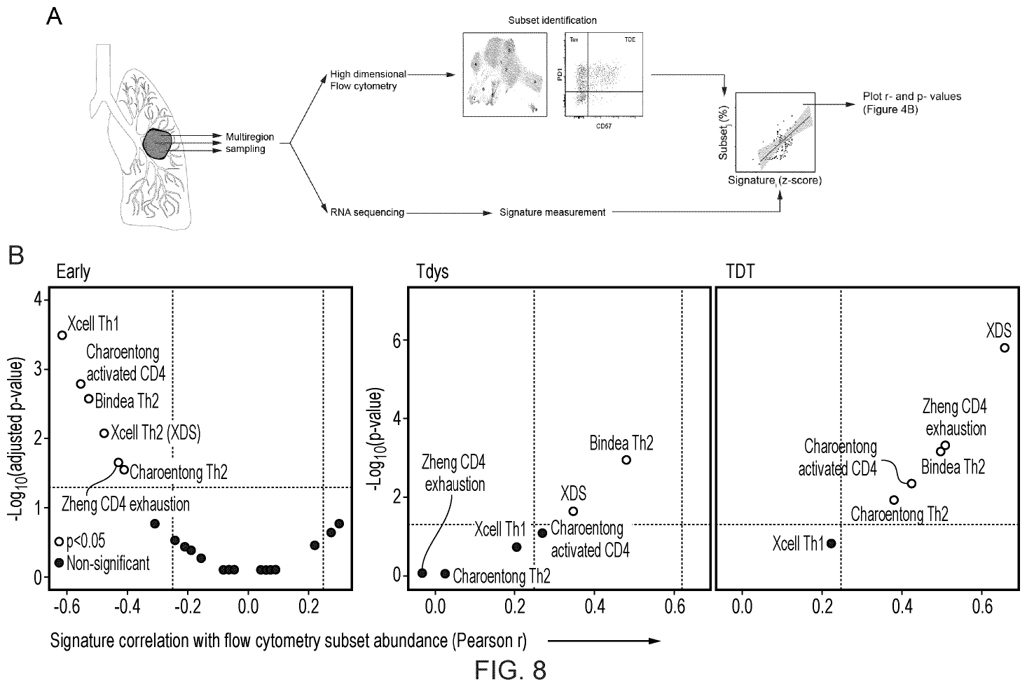

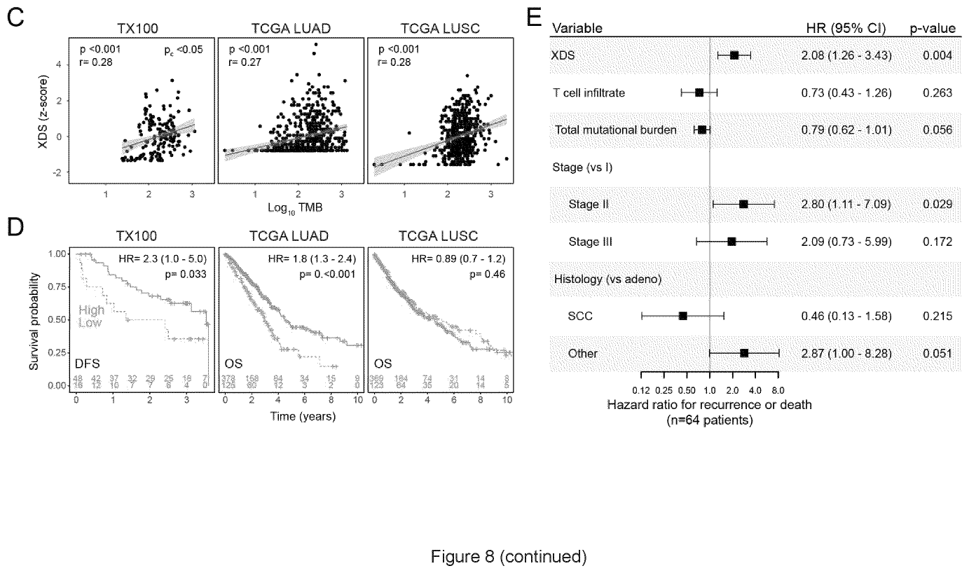

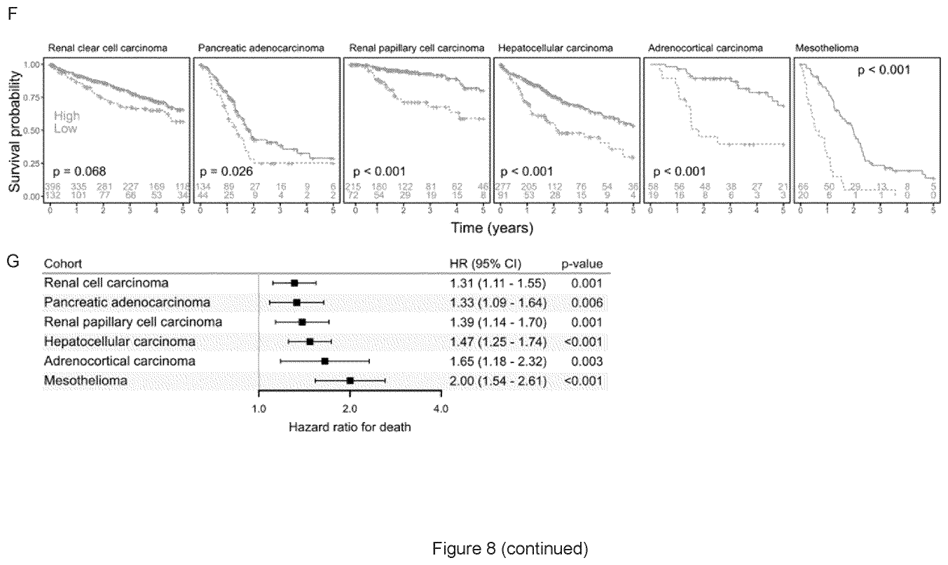

We next validated a gene signature of CD4^{ds} amongst TRACERx samples with paired flow cytometry and RNA sequencing (n=20 patients, 43 regions). Firstly, we identified signatures of CD4 differentiation and found 6/25 to significantly inversely correlate with cytometry measured Early abundance, after correction for multiple testing (FIG. 8A, 8B left panel). Three of these were Th2 signatures, reflecting the scRNAseq profiles of Tdys and TDT populations (FIG. 5G).

Secondly, we tested whether these six signatures positively correlate with Tdys and TDT abundance as expected. The xCell Th253 and Bindea Th254 signatures correlated with both subsets (FIG. 8B right panels) and we continued analysis with the xCell signature (hereafter termed xCell CD4 differentiation skewing; XDS).

Finally, we confirmed the XDS signature correlated with TMB in TRACERx samples with paired RNA and exome sequencing (n=64 patients, 161 regions), and independent NSCLC TCGA cohorts (FIG. 8C; lung adenocarcinoma [LUAD], n=507; lung squamous cell carcinoma [LUSC], n=479). The XDS signature therefore predicts loss of Early and gain in abundance of Tdys and TDT populations measured by flow cytometry and correlates with TMB, hence serving as a transcriptional indicator of CD4^{ds}.

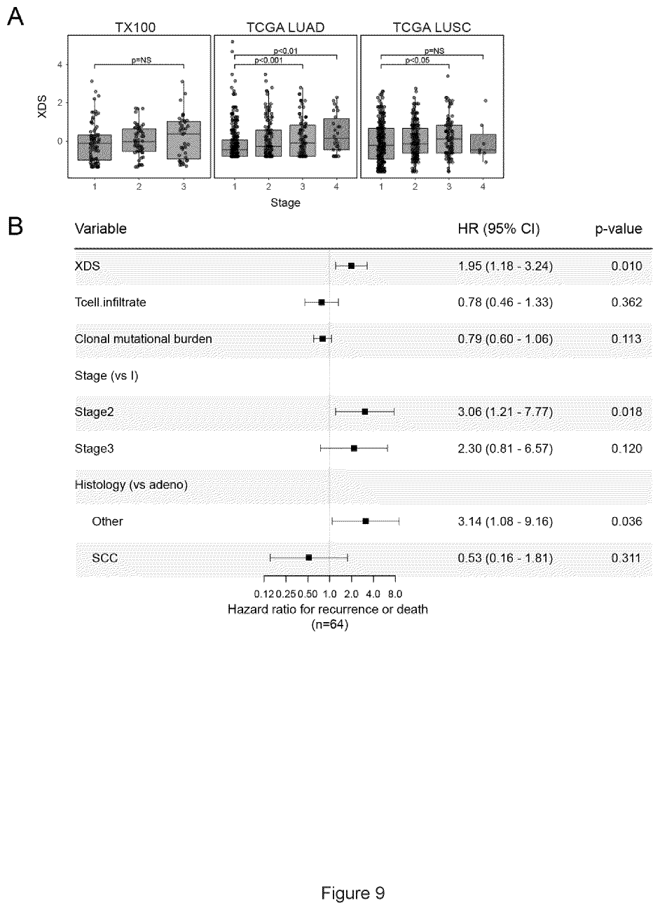

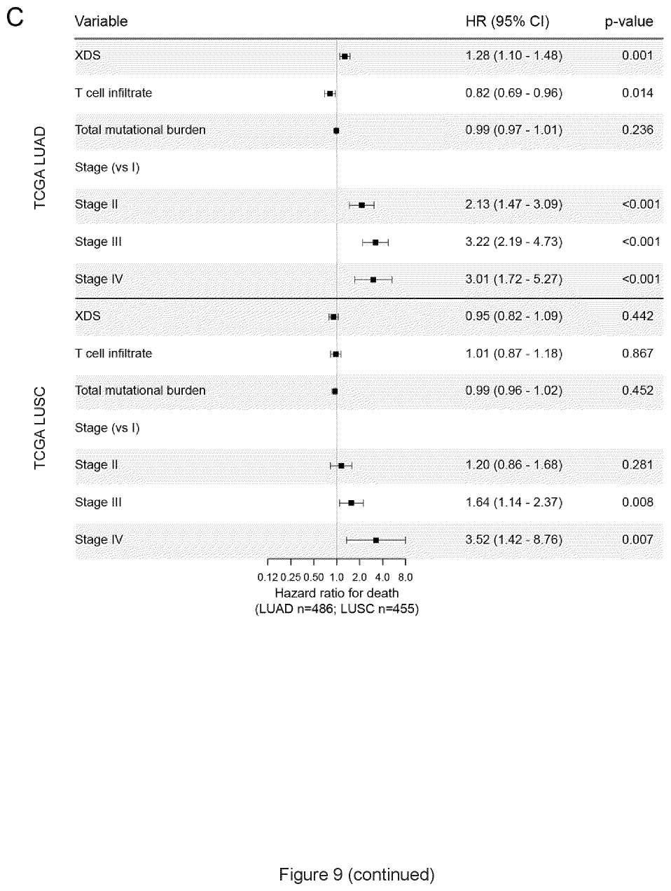

CD4^{ds} predicted survival in the TRACERx flow cytometry cohort (FIG. 2H), and we tested whether the XDS signature performs similarly in the larger TRACERx RNAseq and TCGA NSCLC cohorts. In a univariate analysis, TRACERx patients with high (above upper quartile) XDS enrichment had worse outcomes (p=0.039, hazard ratio [HR] 2.29). This relationship was confirmed in TCGA LUAD (p<0.001, HR 1.79) but not TCGA LUSC cohorts. As a continuous variable in a multivariable analysis adjusting for stage (FIG. 9A), histological subtype, TIL infiltration and mutational burden, the XDS signature remained a negative predictor of survival in TRACERx (adjusted for TMB in FIG. 8E, p=0.003, HR 2.11; adjusted for clonal mutational burden in Figure S5B, p=0.007, HR 1.99) and TCGA LUAD (Figure S5C, p=0.001, HR 1.27).

We also tested whether the XDS signature relates to outcomes in other TCGA cohorts in a pan-cancer analysis (n=5290 patients across 23 cohorts previously described to have adequate data for survival analysis (Miller et al., 2019)). In addition to LUAD, we found six tumour types (renal clear cell carcinoma, pancreatic adenocarcinoma, renal papillary carcinoma, hepatocellular carcinoma, adrenocortical carcinoma and mesothelioma) where the XDS signature negatively correlated with overall survival as a continuous variable in a multivariable analysis accounting for TIL infiltration and TMB (FIG. 8F-G). XDS did not associate with better outcome in any of the other 23 cohorts tested. The XDS signature therefore can serve as a transcriptional indicator of CD4^{ds}, and genes associated with CD4 dysfunction identified herein may represent promising therapeutic targets at least for LUAD, renal clear cell carcinoma, pancreatic adenocarcinoma, renal papillary carcinoma, hepatocellular carcinoma, adrenocortical carcinoma and mesothelioma.

A further set of genes was identified that correlates with the XDS signature in these cohorts, and that could be associated with loss of effector function based on what is currently known about their function (data not shown). The strong correlation of the expression of these genes with the T cell dysfunction signature indicates that they are likely to be deregulated in the dysfunctional populations, and the inventor postulated that some of these may have functions such that “correcting” this deregulation could enhance T cell activity. Amongst these, potential negative regulators of T-cell function that represent promising targets for therapy were identified (in particular: E2F1, C5ORF30, CLDND1, GFI1, RNASEH2A, and SUV39H1) and one of these (E2F1) was selected for validation.

### Example 1.4—a Transcriptional Signature of TCF7/LEF1 Loss Predicts CD4^{ds}

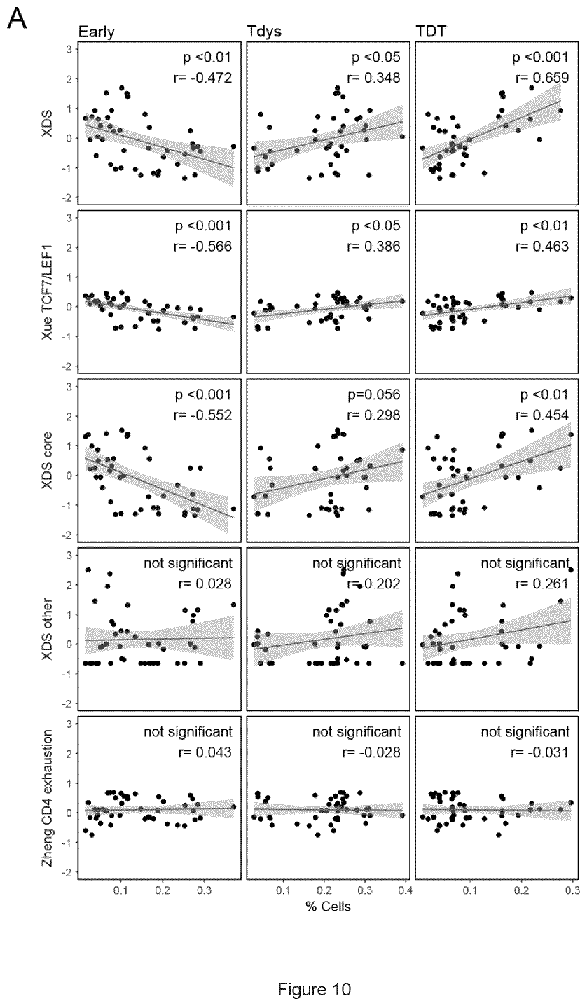

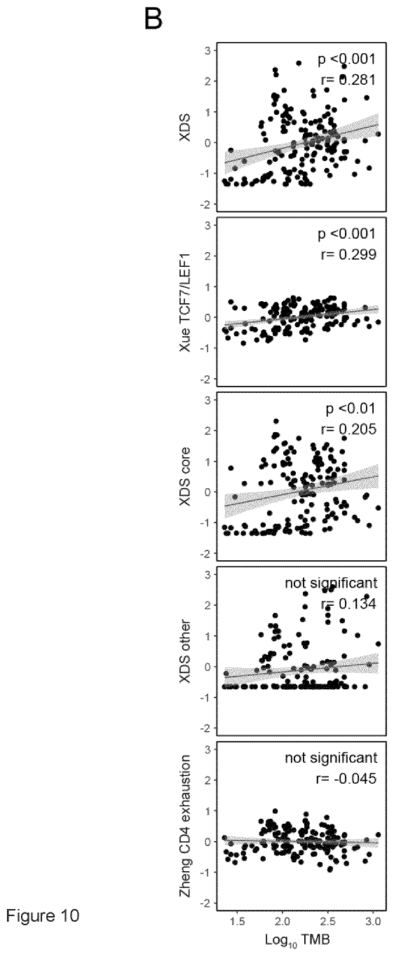

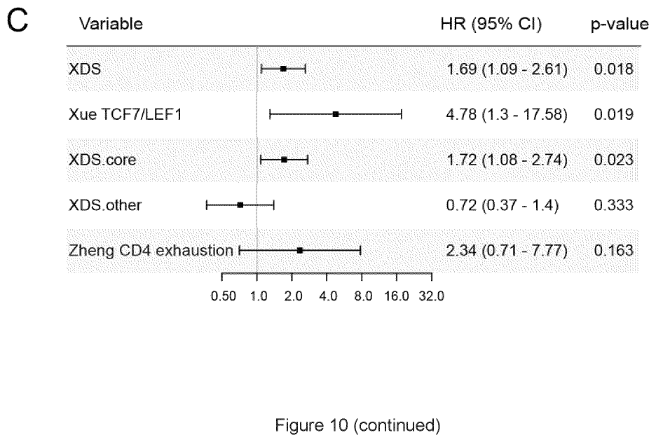

Although three Th2 gene signatures correlated with Tdys and TDT abundance, these populations were not characterised by Th2 gene expression in the scRNAseq dataset (FIG. 6B), suggesting the signatures could reflect non-Th2 specific differentiation features. The Th2 signatures used were generated from T cells differentiated by in vitro IL4 exposure, a treatment known to represses expression of the stemness maintaining transcription factors TCF7 and LEF155. The XDS signature may therefore correlate with CD4^{ds} by reflecting a transcriptional programme of T cell maturation. To test this, we generated a signature of Tcf7/Lef1 deficiency using RNAseq from mouse T cells lacking these genes (Xing et al., 2016). Within the TRACERx cohort, this signature highly correlated with CD4^{ds}, mutational burden and survival (FIGS. 10A, B and C), supporting the hypothesis that mutational burden may accelerate intratumour CD4 loss of progenitor-like potential and negatively impact survival.

We found the XDS signature contained 5/22 genes upregulated upon Tcf7/Lef1 knockout (CEP55, RRM2, NPHP4, MAD2L1, NUP37). Reducing the signature to only these genes preserved correlations with CD4^{ds}, whilst their removal completely abrogated predictive power (FIG. 10). These results suggest the XDS signature captures features of T cell maturation and loss of stemness that occur with CD4ds by virtue of including genes upregulated by TCF7/LEF1 loss.

### Example 1.5—Regulatory T Cell Abundance Associates with CD4^{ds}

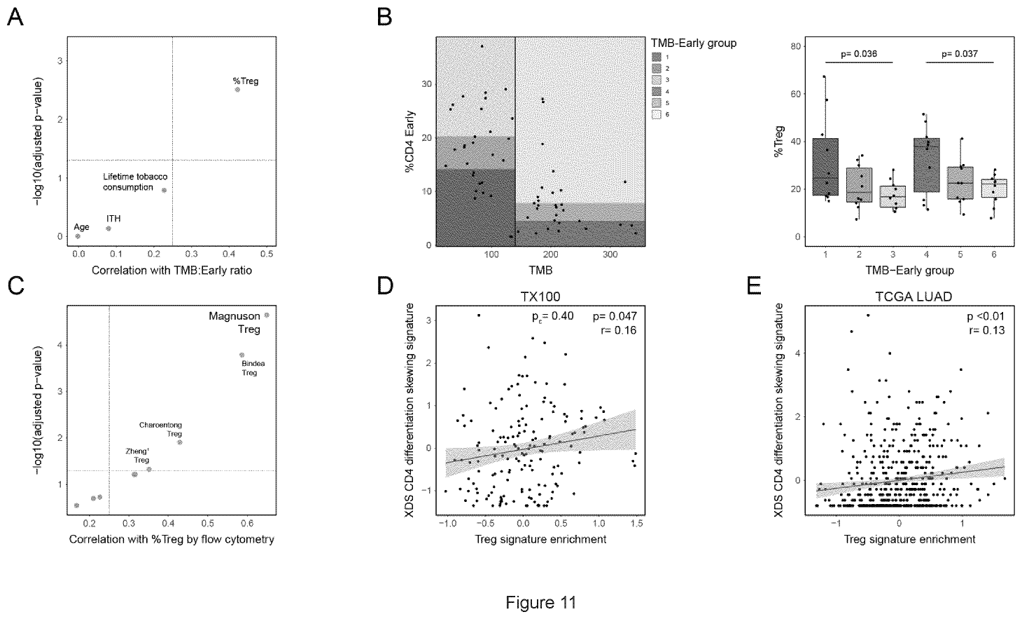

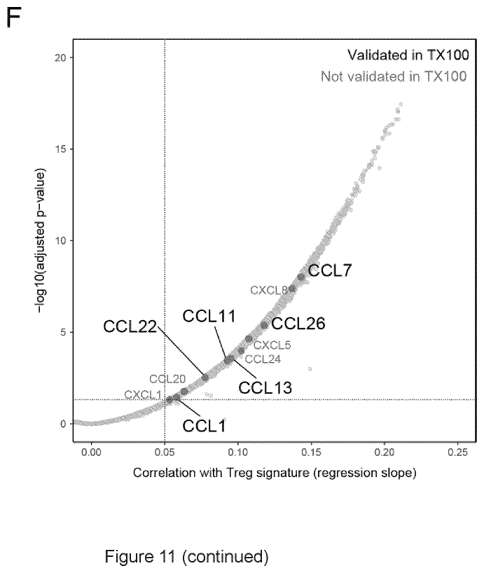

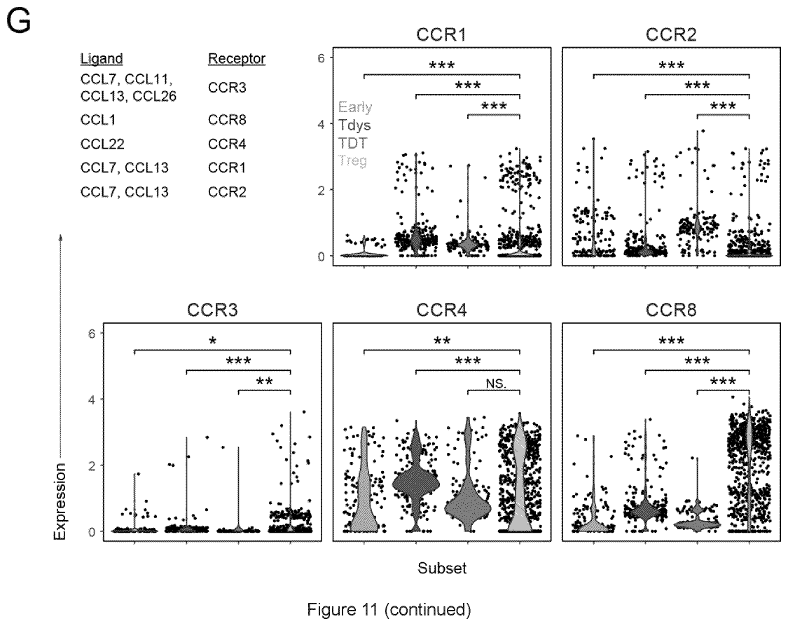

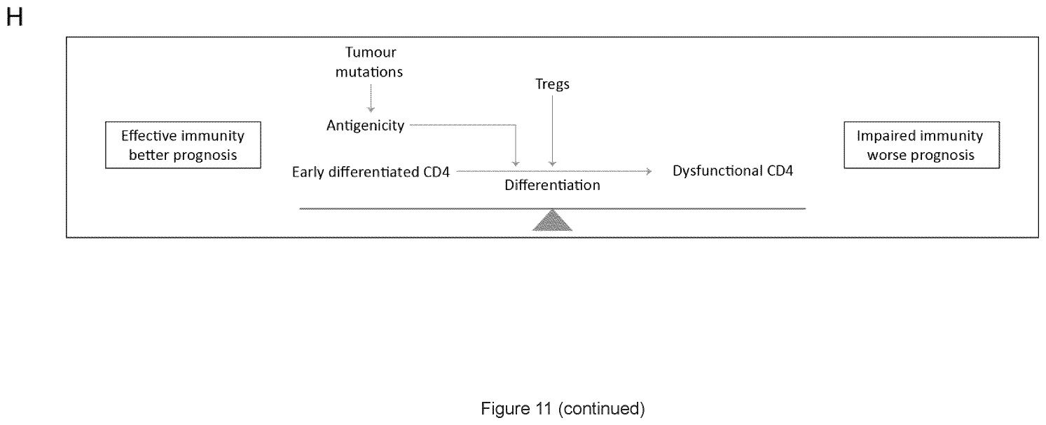

Whilst TMB associated with CD4^{ds}, which independently correlated with worse outcomes, multivariable analysis of TRACERx patients suggests mutational burden itself predicts good survival (FIG. 8E). This suggests factors other than TMB contribute to differences in CD4^{ds}, illustrated by regions with Early abundance and TMB that are concordantly high or low. We therefore sought other factors that shape the TMB-CD4^{ds} relationship and found total Treg abundance to correlate with the ratio between TMB and Early abundance, whilst other parameters (age, smoking and tumour mutation clonal distribution) did not (FIG. 11A).

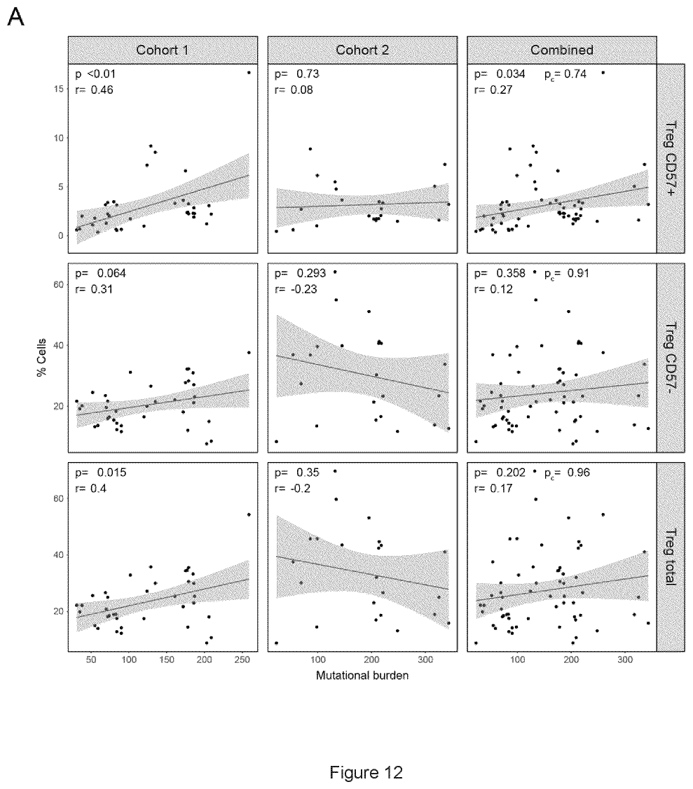

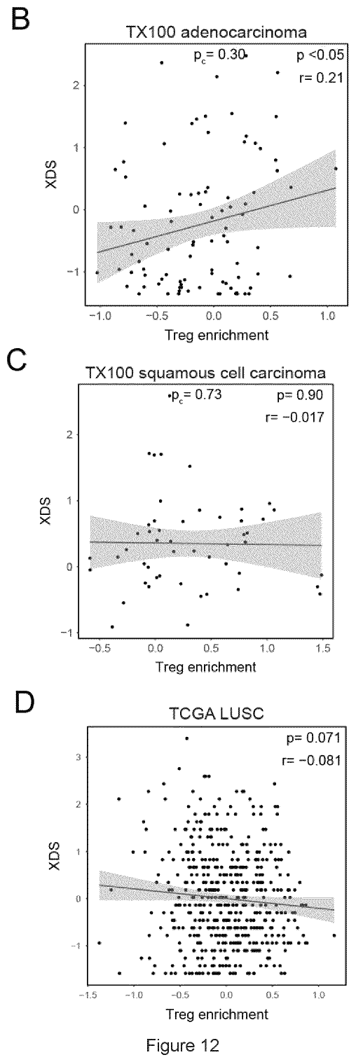

As TMB and CD57⁺ Treg abundance were positively associated in the unsupervised analysis of cohort 1 (FIG. 3A), the relationship between Treg abundance and TMB:Early ratio may reflect a correlation between Tregs and TMB. However, neither manually gated total, CD57⁺ nor CD57⁻ Tregs significantly correlated with TMB in the combined flow cytometry cohort (FIG. 12A) suggesting Treg abundance is not associated with TMB.

We next tested whether Treg and Early subset abundance are related independently of TMB. We divided TRACERx flow cytometry regions into high vs. low TMB based on the median, and within each category further divided regions into high, intermediate and low Early abundance groups according to tertiles, thus generating six subcategories (FIG. 11B). We found a significant inverse correlation between Treg and Early abundance within both TMB high and low groups, suggesting Treg infiltration may contribute to reduced Early abundance independent of mutational burden.

To evaluate this relationship in TRACERx RNAseq and NSCLC TCGA cohorts, we first benchmarked Treg signatures and found the Magnuson et al. signature to best correlate with cytometry measured Treg abundance amongst those tested in TRACERx samples with paired RNAseq data (FIG. 11C). In agreement with the flow cytometry data, CD4^{ds} and Treg signatures were positively correlated amongst TRACERx RNAseq samples, although this did not reach significance in a mixed effects model correcting for histology and tumour multiregionality (FIG. 11D). Amongst TCGA cohorts, CD4^{ds} and Treg signatures were significantly correlated in LUAD (FIG. 11E) but not LUSC patients (FIG. 12D). We therefore analysed TRACERx adenocarcinoma and squamous cell carcinoma patients separately and found a significant relationship between CD4^{ds} and Treg signatures restricted to the former histological group, in agreement with analysis of TCGA (FIG. 12B, C).

Finally, we sought transcriptional differences to explain variation in Treg abundance by carrying out linear regression to test the relationship between expression of individual genes and Treg signature enrichment in TCGA LUAD. Reasoning that Treg abundance may associate with TME chemokine expression, we focused on chemokine encoding genes and discovered 11 candidates that positively correlated with Treg abundance. Of these, CCL1, CCL22, CCL11, CCL13, CCL26 and CCL7 also positively correlated with predicted Treg abundance in the TRACERx RNAseq cohort (FIG. 11G). These chemokines are recognised by five chemokine receptor encoding genes (CCR1, CCR2, CCR3, CCR4 and CCR8), amongst which CCR1, CCR3, CCR4 and CCR8 were highly expressed upon manually identified FOXP3⁺ Tregs in the scRNAseq dataset. These results suggest chemokine receptor expression may contribute to NSCLC intratumour Treg abundance.

### Example 1—Discussion

In this example we combined high dimensional flow cytometry, genomic, bulk and single cell transcriptional data to characterise the NSCLC intratumour CD4 T cell compartment. We present evidence of global CD4 dysregulation in association with TMB and Treg infiltration, suggesting the process may be neoantigen driven and sensitive to microenvironmental factors.

As a negative predictor of outcome, CD4^{ds} could indicate impaired CD4 T cell anti-tumour efficacy arising from loss of CD4 progenitors and/or gain of dysfunctional subsets. Progenitor loss could be critical to intratumour CD4 T cell failure. These cells are known to sustain anti-viral (Okoye et al., 2007; Wu et al., 2016) and autoimmune responses (Paroni et al., 2017; Orban et al., 2014; Shi et al., 2018), with emerging evidence suggesting the importance of CD8 progenitors in anti-tumour control and response to checkpoint immunotherapy. Analysis of scRNAseq data revealed Early subset expression of the transcription factor encoding genes TCF7 and LEF1 that maintain T cell stemness, and a transcriptional signature of deficiency of these genes correlated with CD4^{ds} and worse survival (FIG. 10), further supporting early differentiated CD4 loss as a key feature of anti-tumour immune failure. The decline in Early subset abundance may result from activation induced depletion. Additionally, as the Tdys and TDT populations differentially express chemokine receptors and adhesion molecules at protein (CD103; FIG. 4B) and transcriptional levels (FIG. 5D), their accumulation within the spatially constrained TME may be favoured at the expense of CCR7⁺ early differentiated cells that preferentially recirculate between lymphoid organs.

Whilst the CD4 Tdys and TDT subsets have phenotypic and transcriptional features of impaired function, they may retain anti-tumour potential. Both subsets express the CD4 effector gene IFNG, whilst the Tdys population additionally expresses CD40LG that is a key mediator of CD4 helper function. Additionally, TDT expression of a CD8-like transcriptional profile of effector genes is suggestive of cytotoxic capability. These indicators of functional potential are consistent with recent studies showing dysfunctional intratumour CD8 T cells retain proliferative capacity (Simoni et al., 2018). However, the observation that dysfunctional CD4 T cells co-exist with progressing tumours and their abundance inversely correlates with patient outcomes suggests an overall impaired status. Together, these findings support the hypothesis that chronically stimulated T cell function is tuned down, possibly to protect against off-target tissue autoimmunity, but not eliminated altogether.

Recent studies suggest mutational burden is positively associated with cancer outcomes, particularly amongst immunotherapy treated patients. Conversely, studies have shown differentiation skewing and T cell dysfunction to occur with persistent antigen exposure. Our finding in the TRACERx cohort that TMB positively correlates with outcome, whilst CD4^{ds} has a negative relationship supports the notion that there may be opposing effects of mutations on immune function, depending on the context of antigen encounter (FIG. 8E). Opposing effects of TMB may occur if mutations generate antigenic targets for recognition and control by early differentiated T cells that are driven to dysfunctional states by chronic target exposure or deprived of niche within the TME as later differentiated cells accumulate (FIG. 11H). Independent of TMB, Treg abundance correlated with measures of CD4^{ds}, suggesting their presence may alter the extent of antigen driven CD4 dysregulation. Treg promotion of naïve and effector CD4 dysfunctional through the induction of senescence and co-inhibitory receptor expression (Liu et al, 2018; Sawant et al., 2019) may underlie this relationship. As TMB most strongly predicts survival amongst immunotherapy treated patients, checkpoint inhibition may also modify the balance between antigen driven T cell anti-tumour efficacy vs. CD4^{ds} arising from chronic exposure.

The relationship between CD4^{ds} and clonal but not subclonal mutations suggests the importance of antigen abundance (FIG. 2E). As the majority of NSCLCs do not express MHC II (He et al., 2017) required for CD4 recognition, class II bearing antigen presenting cells are likely key mediators of CD4 anti-tumour immune responses. Clonal mutations may preferentially drive CD4^{ds} by generating neoantigen levels above minimum thresholds for immune activation, compared to subclonal mutations (Zingernagel et al., 1997). However, the low range of subclonal mutations in our cohort may limit accurate evaluation of a relationship with CD4^{ds} and further work is warranted to explore this.

Our study suggests multiple potential therapeutic targets and a guide for rational immunotherapy selection. Single cell RNAseq analysis revealed divergent and previously undescribed features of the co-stimulatory and co-inhibitory receptor landscape of Tdys and TDT subsets and we identify actionable subset specific (e.g. GITR, ICOS and OX40 upon Tdys, CD27 and LIGHTR upon TDT) and shared targets (e.g. TIGIT and TIM3). Amongst these, as mentioned above, some genes were selected as particularly promising actionable targets (EPHA1, FCRL3, PECAM1, STOM, AXL, FURIN, LL1RAP, E2F1, C5ORF30, CLDND1, GFI1, RNASEH2A, SIRPG and SUV39H1), and a subset of these were selected for experimental validation (AXL, FURIN, IL1RAP, STOM, FCRL3, SIRPG and E2F1). Data for IL1RAP and SIRPG is shown in Example 3.

Taken together, we show profound changes within the intratumoural CD4 differentiation landscape that occur in association with TMB and Treg abundance, reminiscent of alterations in the peripheral T cell compartment of mice and humans during persistent antigen exposure. CD4^{ds} predicts worse outcomes in multiple cohorts and combining data from single cell and bulk RNA sequencing reveals biological insights into the process with potential therapeutic value.

### Example 2—Neoantigen-Associated CD8 T Cell Dysfunction is Coupled to Tumour Immune Escape in Non-Small Cell Lung Cancer

Material and Methods

Patients and samples: as described in Example 1 except that samples from the TRACERx lung pilot study (UCLHRTB 10/H1306/42) were additionally included (specified with an LO prefix).

Lymphocyte isolation for immune assays: Tissue samples were collected and transported in RPMI-1640 (Sigma, cat #R0883-500ML). Single-cell suspensions were produced by enzymatic digestion using a Liberase TL (Roche, cat #05401127001) and DNase I (Roche, cat #11284932001) with subsequent cellular disaggregation using a Miltenyi gentleMACS OctoDissociator. Lymphocytes were isolated from single-cell suspensions by gradient centrifugation on Ficoll Paque Plus (GE Healthcare, cat #17-1440-03), cryopreserved in fetal bovine serum (Gibco, cat #10270-106) containing 10% DMSO (Sigma, cat #D2650-100ML) and stored in liquid nitrogen. Blood samples were collected in BD Vacutainer EDTA blood collection tubes (BD cat #367525), PBMC's were then isolated by gradient centrifugation on Ficoll Paque (GE Healthcare, cat #17-1440-03) and stored in liquid nitrogen.

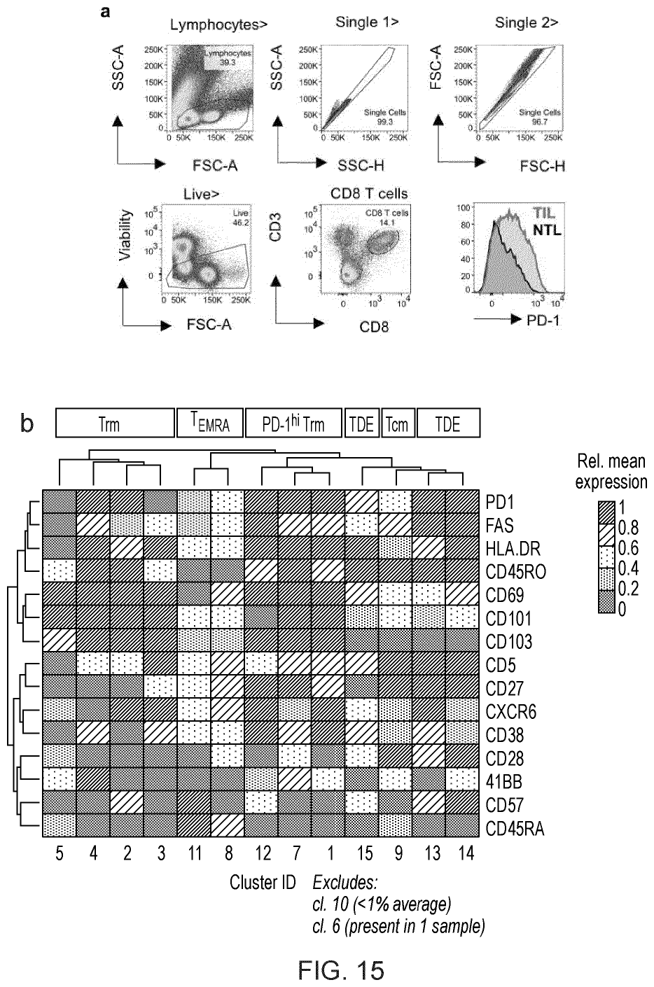

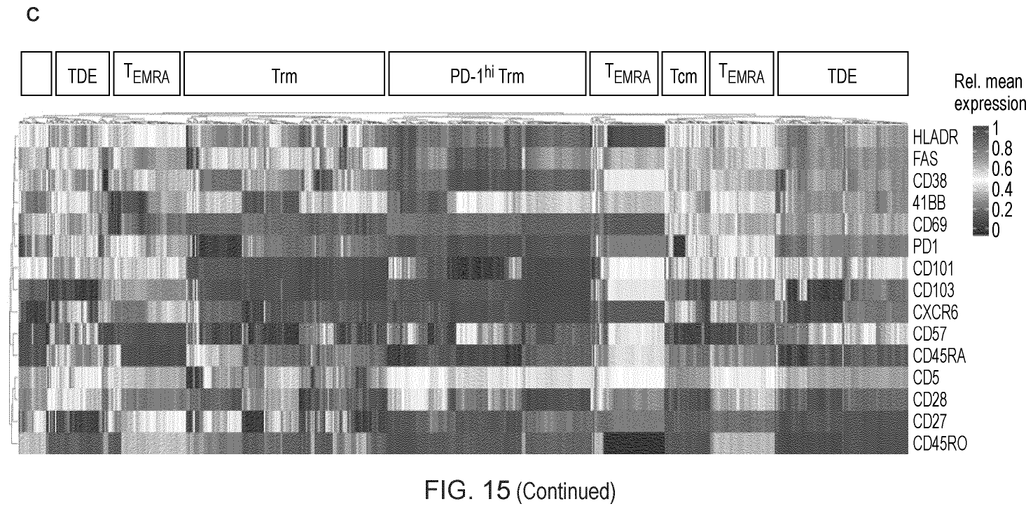

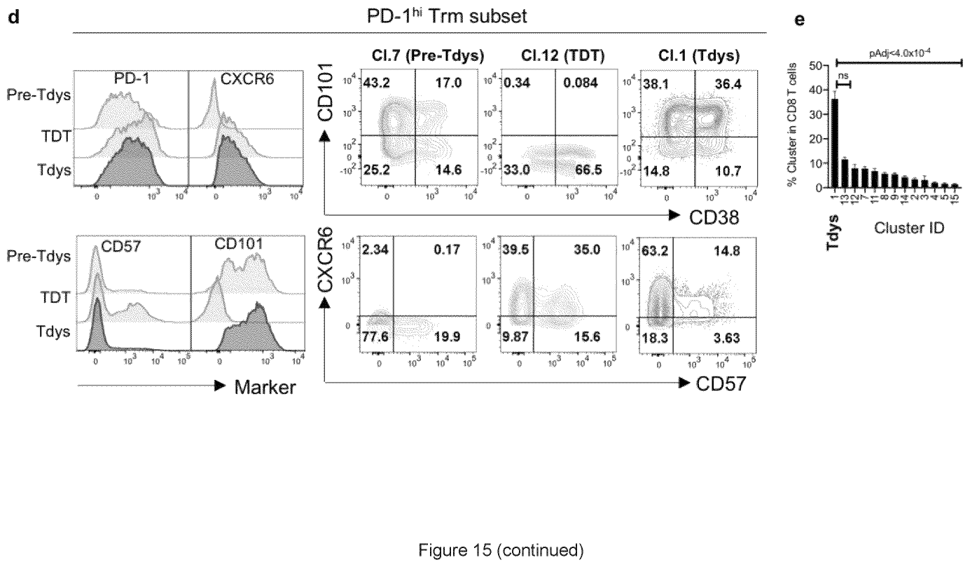

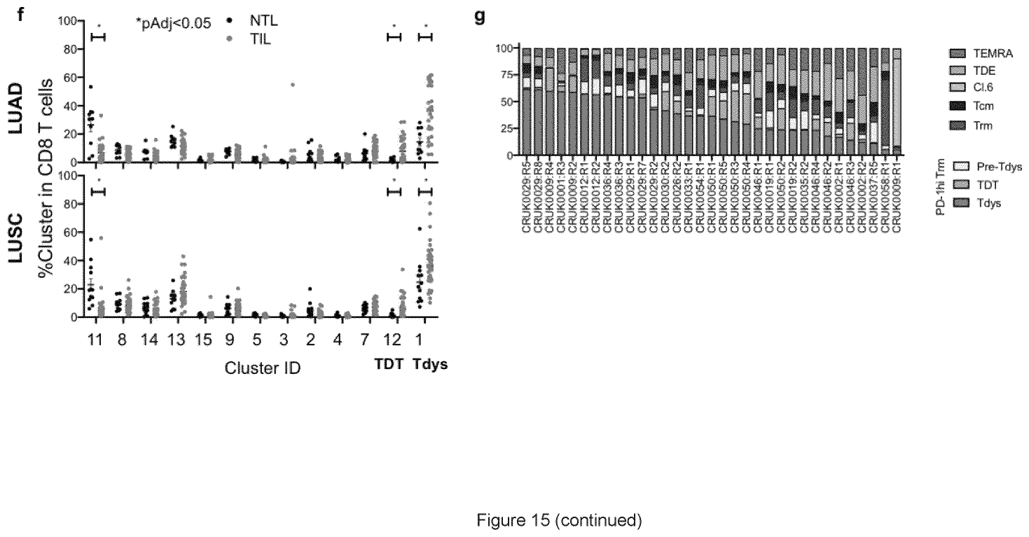

Flow cytometry: FC receptors were blocked with Human Fc Receptor Binding Inhibitor (Thermo, cat #572 14-9161-73) before staining. Non-viable cells were stained using the eBioscience Fixable Viability Dye eFluor 780 (Thermo, cat #65-0865-14). Cells were stained in BD Brilliant stain buffer (BD cat #563794), the following monoclonal antibodies were used were BUV395 conjugated antibody to human CD45R0 (clone UCLH1; BD cat #576 564291); BUV496 conjugated antibody to human CD8 (clone RPA-T8; BD cat #564804); BUV563 conjugated antibody to human CD45RA (clone HI 100; BD cat #565702); BUV661 conjugated antibody to human CD4 (clone SK3; BD cat #566003); BUV737 conjugated antibody to human CD28 (clone 28.2; BD cat #564438); BUV805 conjugated antibody to human CD3 (clone SK7; BD cat #565511); BV421 conjugated antibody to human PD-1 (clone EH12; BD cat #562516); BV605 conjugated antibody to human CD57 (clone NK-1; BD cat #563896); BV711 conjugated antibody to human CD69 (clone FN50; BD cat #563836); BV786 conjugated antibody to human CD27 (clone L128; BD cat #563327). BV480 conjugated antibody to human CD5 (clone UCHT2; BD cat #566122); BV650 conjugated antibody to human CD38 (clone HIT2; BD cat #740574); BB515 conjugated antibody to human CD103 (clone Ber-ACT8; BD cat #564578); PerCP-Cy5.5 conjugated antibody to human CXCR6 (clone K041E5; Biolegend cat #356010); PE conjugated antibody to human CCR5 (clone 2D7/CCR5; BD cat #555993); PE/Dazzle 594 conjugated antibody to human 4-1BB (clone 4B4-1; Biolegend cat #309826); PE-Cy7 conjugated antibody to human FAS (clone DX2; Biolegend cat #305622); APC conjugated antibody to human CD101 (clone BB27; Biolegend cat #331010) and APC-R700 conjugated antibody to human HLA-DR (clone G46-6; BD cat #565127). Data was acquired on a BD Symphony flow cytometer and analyzed in FlowJo v10.5.3 (Treestar). Cells were gated for size, single 594 cells, live cells, CD3⁺CD8⁺ T cells as seen in FIG. 15a.

Unsupervised flow cytometry analysis: Raw FCS files were processed using a custom made pipeline, ‘Cytofpipe’, developed for the automated analysis of flow- and mass cytometry data, based on cytofkit (Chen et al., 2016), SCAFFoLD (Spitzer et al., 2015) and CITRUS (Bruggner et al., 2014) R packages. Specifically, marker expression values were transformed using the autoLgcl transformation from cytofkit, and a fixed number of 2000 cells were then randomly sampled without replacement from each file and merged for analysis. Unsupervised analysis was performed using FLowSOM (Van Gassen et al., 2015) as implemented in the pipeline. Clustering was based on expression of markers exhibiting intercellular phenotypic variance; CD38, CD45R0, CD69, CXCR6, FAS, PD1, CD103, HLA-DR, CD27, CD57, CD45RA, CD5, CD28 and CD101 (excluding CCR5 and 4-1BB which yielded low signal). FlowSOM was performed on NTL and TIL samples using k=15 predefined numbers of clusters informed by prior Phenograph (Weber et al., 2016) analysis. FlowSOM clustering was repeated 50 times with a different random seed across recursions to ensure subset stability. Clusters were filtered to remove those with <1° average frequency and those only present in rare samples (<10% samples run). Median intensity values per cluster were used to generate heatmaps. Each cluster was inspected using an N×N series of biaxial plots for all channels. CCR5 was excluded from downstream analysis due to low intercellular variance. Subsets were assigned based on grouping of superclusters on heatmap dendrograms, common expression profiles of key markers, topology of populations in dimensionally reduced space together and manual annotation from the literature. Clusters and subsets pertinent to the study (significant correlations with mutational landscape) were validated using manual gating strategies. UMAP (Becht et al., 2019) dimension reduction was used to visualize cell populations given its superiority in the topological preservation of cluster similarity and improved data continua relative to alternative dimension reduction techniques (Becht et al., 2019). UMAPs were projected with relative marker expression or over-plotting of FlowSOM clusters or subsets and leveraged to infer subset and inter-cluster relationships as detailed in the main text.

Multi-region whole exome sequencing: Whole exome sequencing (WES) of multi-region tumour specimens and matched germline samples derived from whole blood was performed as previously described (Jamal-Hanjani et al, 2017). Synonymous and non-synonymous mutations from each tumour region were identified by comparing germline and tumour DNA.

Clonal and subclonal mutation calling: The clonality of each non-synonymous mutation was determined using a modified version of PyClone (Roth et al., 2014; McGranahan et al., 2016) taking gene copy number, tumour purity and variant allele frequency (VAF) into account. The sequencing data is deposited in the European Genome-Phenome Archive under the accession number EGAS00001002247. Briefly, for each mutation, two values were calculated, obsCCF and phyloCCF. obsCCF corresponds to the observed cancer cell fraction (CCF) of each mutation. Conversely, phyloCCF corresponds to the phylogenetic CCF of a mutation. To clarify the difference between these two values, consider a mutation present in every cancer cell within a tumour. A subclonal copy number event in one tumour region may lead to loss of this mutation in a subset of cancer cells. While, the obsCCF of this mutation is therefore below 1, from a phylogenetic perspective the mutation can be considered ‘clonal’ as it occurred on the trunk of the tumour's phylogenetic tree, and, as such, the phyloCCF may be 1. To calculate the obsCCF of each mutation, local copy number (obtained from ASCAT), tumour purity (also obtained from ASCAT), and variant allele frequency were integrated. In brief, for a given mutation we first calculated the observed mutation copy number, nₘᵤₜ, describing the fraction of tumour cells carrying a given mutation multiplied by the number of chromosomal copies at that locus using the following formula:

nₘᵤₜ=VAF¹ₚ[pCNₜ+CNₙ(1−p)]

where VAF corresponds to the variant allele frequency at the mutated base, and p, CNₜ, CNn are respectively the tumour purity, the tumour locus specific copy number, and the normal locus specific copy number (CNₙ was assumed to be 2 for autosomal chromosomes). We then calculated the expected mutation copy number, n_{chr}, using the VAF and assigning a mutation to one of the possible local copy numbers states using maximum likelihood. In this case only the integer copy numbers were considered. All mutations were then clustered using the PyClone Dirichlet process clustering (Roth et al., 2014). For each mutation, the observed variant count was used and reference count was set such that the VAF was equal to half the pre-clustering CCF. Given that copy number and purity had already been corrected, we set the major allele copy numbers to 2 and minor allele copy numbers to 0 and purity to 0.5; allowing clustering to simply group clonal and subclonal mutations based on their pre-clustering CCF estimates. We ran PyClone with 10,000 iterations and a burn-in of 1000, and default parameters, with the exception of --var_prior set to ‘BB’ and -ref_prior set to ‘normal’. To determine the phyloCCF of each mutation, a similar procedure to that described above was implemented, with the exception that mutations were corrected for subclonal copy number events. Specifically, if the observed variant allele frequency was significantly different from that expected (P<0.01, using prop.test in R) given a clonal mutation, we determined whether a subclonal copy number event could result in a non-significant (P>0.01) difference between observed and expected VAFs. The pre-clustering CCF for each mutation was then calculated by dividing nₘᵤₜ by n_{chr}. Subclonal copy number events were estimated using the raw values from ASCAT output. Finally, to ensure potentially unreliable VAFs of indels did not lead to separate mutation clusters, each estimated indel CCF was multiplied by a region-specific correction factor. Assuming the majority of ubiquitous mutations, present in all regions, are clonal, the region-specific correction factor was calculated by dividing the median mutation CCF of ubiquitous mutations by the median indel CCF of ubiquitous indels.

Neoantigen binders: Novel 9-11mer peptides that could arise from identified non-silent mutations present in the sample were determined. The predicted IC50 binding affinities and rank percentage scores, representing the rank of the predicted affinity compared to a set of 400,000 random natural peptides, were calculated for all peptides binding to each of the patient's HLA alleles using netMHCpan-2.8 and netMHC-4.0. Predicted binders were considered those peptides that had a predicted binding affinity <500 nM or rank percentage score <2° by either tool. Strong predicted binders were those peptides that had a predicted binding affinity <50 nM or rank percentage score <0.5%.

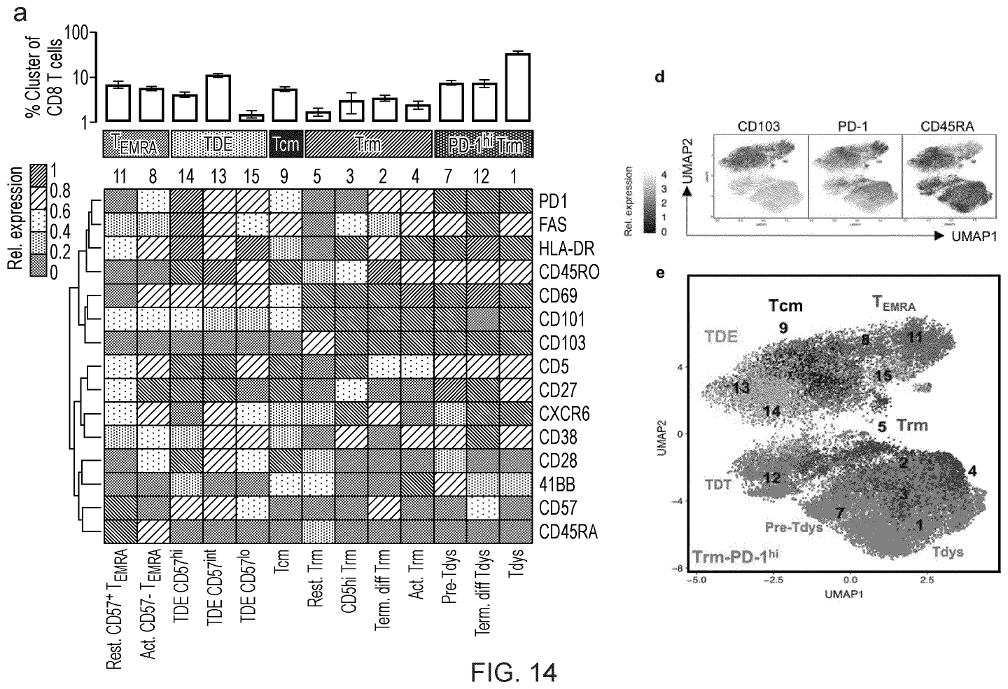

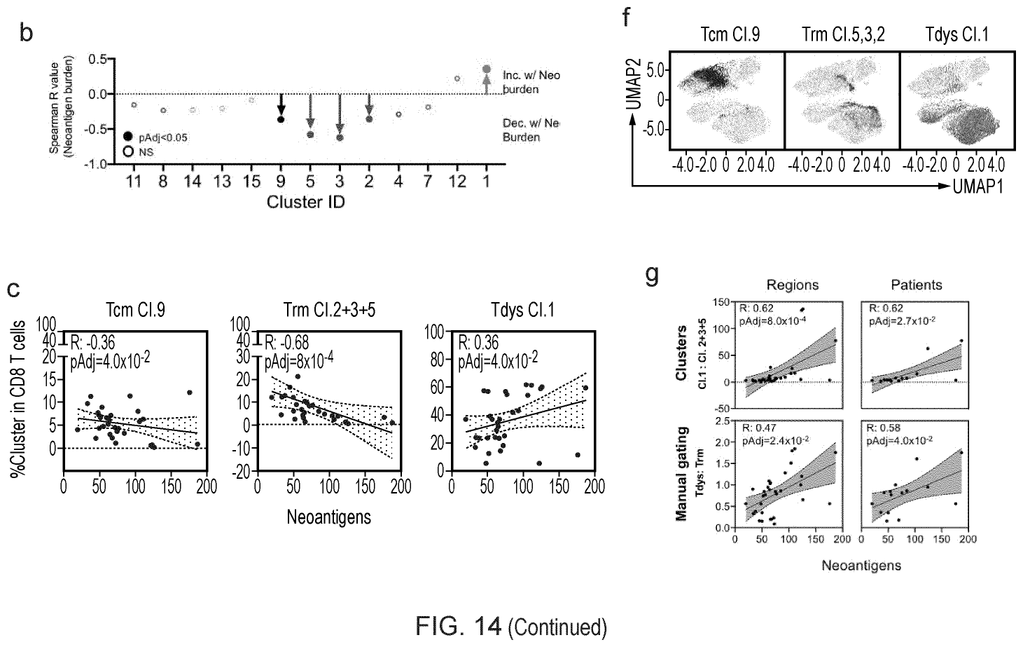

Correlative analysis of flow cytometry data: The k=15 CD8 T cell clusters identified in the first of the 50 FlowSOM iterations described above were used as representative clusters for initial study. Clusters were filtered to remove those with average frequency <1% (cluster 10) and those present in <10% of samples (cluster 6, detected only in CRUK009:R1). The frequency of the 13 remaining clusters was initially analysed in the context of paired WES data by 2-tailed spearman rank correlation vs the number of i) Total neoantigens and ii)<50 nM affinity (‘strong’), clonal neoantigens on the basis of previously identified reactivities in NSCLC (McGranahan et al., 2016). P values were corrected for multiple adjustment to control for type I errors using the original Benjamini-Hochberg (BH) FDR procedure at FDR 0.05, correcting p values from both sets of analysis. Trm clusters sharing a negative correlation with neoantigen load (cl.2,3,5) were combined for downstream analysis based on similarity in phenotype, clustering in dimensionally reduced space via UMAP and their shared negative correlation with neoantigen load. The Tdys:Trm ratio was used as a single measure to confirm relationships with additional genomic features including total neoantigens, TMB, clonal- or subclonal mutations, clonal- or subclonal neoantigens, mutations predicted to not give rise to neoantigens (non-binders) and ‘strong’ total- or clonal neoantigens (with <50 nM affinity for cognate HLA). The Tdys:Trm ratio remained significantly correlated with neoantigen load when manually gating populations to confirm unsupervised analyses and when initial observations using individual tumour regions as discrete data points were re-analyzed using the average of multi-region samples per patient (FIG. 14e).

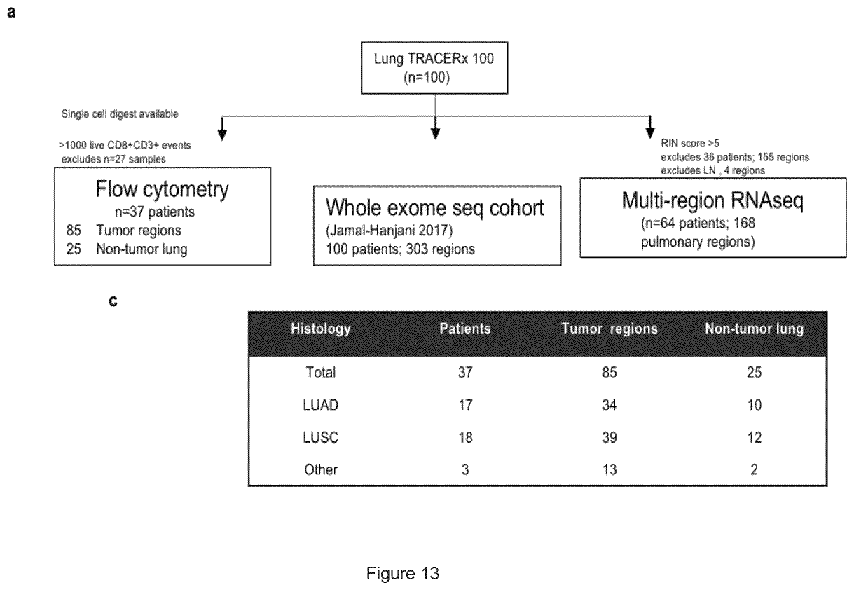

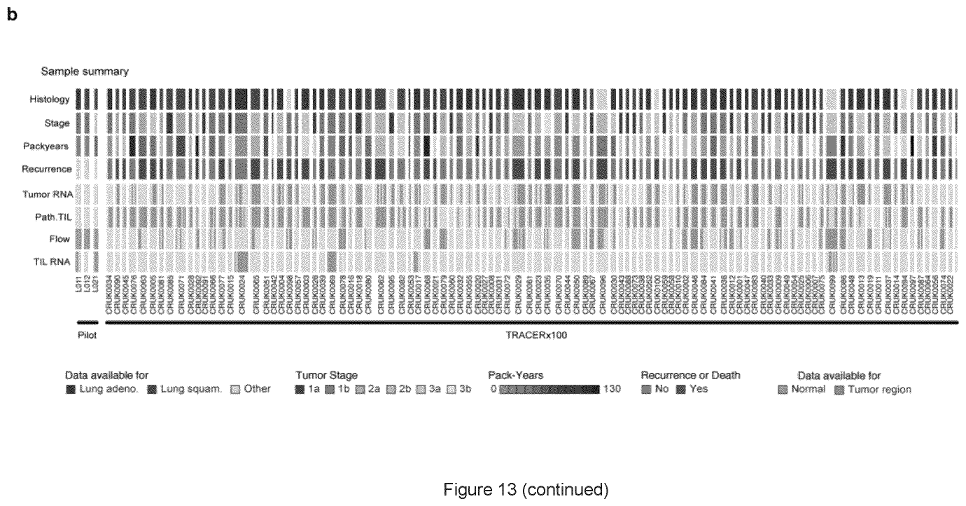

Multimer analysis of neoantigen reactive T cells: Neoantigen-specific CD8 T cells were identified using high throughput MHC multimer screening (Hadrup et al., 2009) of candidate mutant peptides generated from patient-specific neoantigens of predicted <500 nM affinity for cognate HLA as previously described (McGranahan et al., 2016). 288 and 354 candidate mutant peptides (with predicted HLA binding affinity <500 nM, including multiple potential peptide variations from the same missense mutation) were synthesized and used to screen expanded L011 and L012 TILS respectively. In patient L011, TILS were found to recognize the HLA-B*3501 restricted, MTFR2D326Y-derived mutated sequence FAFQEYDSF (SEQ ID NO: 23)(netMHC binding score: 22), but not the wild type sequence FAFQEDDSF (SEQ ID NO: 24)(netMHC binding score: 10). No responses were found against overlapping peptides AFQEYDSFEK (SEQ ID NO: 25) and KFAFQEYDSF (SEQ ID NO: 26). In patient L012, TILS were found to recognize the HLA-A*1101 restricted, CHTF18L769V-derived mutated sequence LLLDIVAPK (SEQ ID NO: 27) (netMHC binding score: 37) but not the wild type sequence: LLLDILAPK (SEQ ID NO: 28) (netMHC binding score: 41). No responses were found against overlapping peptides CLLLDIVAPK (SEQ ID NO: 29) and IVAPKLRPV (SEQ ID NO: 30). Finally, in patient L012, TILs were found to recognize the HLA-B*0702 restricted, MYADMR30W-derived mutated sequence SPMIVGSPW (SEQ ID NO: 31) (netMHC binding score: 15) as well as the wild type sequence SPMIVGSPR (netMHC binding score: 1329). No responses were found against overlapping peptides SPMIVGSPWA (SEQ ID NO: 32), SPMIVGSPWAL (SEQ ID NO: 33), SPWALTQPLGL (SEQ ID NO: 34) and SPWALTQPL (SEQ ID NO: 35). Patient L021 was a 72 year old male smoker (50 pack years) with stage IIIA LUSC (poorly differentiated, right upper lobe, 51 mm, lymph node 2/6 hilar), as displayed in FIG. 13b. For patient L021, 235 peptides were screened from a library of predicted clonal neoantigens.

Simultaneously, TIL responses to HLA matched viral peptides were assessed. TILS were found to recognize the HLA-A*3002 restricted, ZNF704 L301F-derived mutated sequence YFVHTDAY (SEQ ID NO: 36) (netMHC binding score: 61) as well as the wild type sequence YLVHTDHAY (SEQ ID NO: 37) (netMHC binding score: 27). No response to overlapping peptides TLYFVHTDH (SEQ ID NO: 38), TLYFVHTDHAY (SEQ ID NO: 39), LYFVHTDHAY (SEQ ID NO: 40) and APTTLYFVH (SEQ ID NO: 41) were detected. Neoantigen-specific CD8⁺ T cells were tracked with peptide-MHC multimers conjugated with either streptavidin PE (Biolegend, cat #405203), APC (Biolegend, cat #405207) BV650 (Biolegend, cat #405231) or PE-Cy-7 (Biolegend, cat #405206) and gated as double (L011, L021) or single (L012) positive cells among live, single CD8⁺ cells. Phenotypic characterization of neoantigen-specific CD8 T cells in L011 and L012 was performed as previously described 22. In addition, MHC multimers for neoantigens in L021 and L011 were stained in TILS, PBMC and NTL with flow cytometry Neo panel 1: anti-CD3 conjugated to BV711 (Biolegend, cat #344838, clone SK7); anti-CD4 conjugated to BV785 (Biolegend cat #344642, clone SK3); anti-CD8 conjugated to BV510 (Biolegend, cat #301048, clone RPA-T8); anti-CD45RA conjugated to PE/Cy7 (Biolegend, cat #304126, clone HI100); anti-CCR7 conjugated to BV605 (Biolegend cat #353224, clone G043H7); anti-PD-1 conjugated to BV650 (Biolegend, cat #329950, clone EH12.2H7); MHC-Multimer-PE, MHC-Multimer-APC, or flow cytometry Neo panel 2: anti-CCR7 conjugated to FITC (Biolegend, cat #353216, clone G043H7); anti-CXCR6 PerCP/Cy-5.5 (Biolegend, cat #356010 clone K041E5); anti-CD8 conjugated to BV510 (Biolegend, cat #301048, clone RPA-T8); anti-PD-1 conjugated to BV605 (Biolegend, cat #329924 clone EH12.2H7); anti-CD4 conjugated to BV650 (Biolegend, cat #300536 clone RPA-T4); anti-CD45RA conjugated to BV711 (Biolegend, cat #304138 clone HI100); anti-CD69 conjugated to BV785 (Biolegend, cat #310932, clone FN50); anti-4-1BB conjugated to PE-Dazzle-CF594 (Biolegend, cat #309826, clone 4B4-1); anti-ICOS conjugated to PE-Cy7 (Biolegend, cat #313520, clone C398.4A); anti-CD3 conjugated 763 to BUV395 (BD, cat #564001, clone SK7); anti-CD28 conjugated to Alexa-Fluor700 (Biolegend, cat #302920, clone CD28.2).

Single-Cell RNA sequencing of Neoantigen Reactive T cells (Neo.CD8): We have previously identified CD8⁺ neoantigen reactive T cells (NARTs) targeted against a clonal neoantigen (arising from the mutated MTFR2 gene) in NSCLC tumour regions derived from patient L01122. We repeated the staining of neoantigen reactive T cells based on dual fluorescent multimer labelling using Neo.Panel 1 described above and a freshly thawed vial of cryopreserved TILS from the same patient. Multimer-positive and negative single CD8+ T cells from NSCLC specimens were sorted directly into the C1 Integrated Fluidic Circuit (IFC; Fluidigm). Cell lysing, reverse transcription, and cDNA amplification were performed as specified by the manufacturer. Briefly, 1000 single, multimer positive or negative CD8 T cells were flow sorted directly into a 10- to 17-μm-diameter C1 Integrated Fluidic Circuit (IFC; Fluidigm). Ahead of sorting, the cell inlet well was preloaded with 3.5 μl of PBS 0.5% BSA. Post-sorting the total well volume was measured and brought to 5 ul with PBS 0.5% BSA. 1 μl of C1 Cell Suspension Reagent (Fluidigm) was added and the final solution was mixed by pipetting. Each C1 IFC capture site was carefully examined under an EVOS FL Auto Imaging System (Thermo Fisher Scientific) in bright field, for empty wells and cell doublets. An automated scan of all capture sites was also obtained for reference. Cell lysing, reverse transcription, and cDNA amplification were performed on the C1 Single-Cell Auto Prep IFC, as specified by the manufacturer. The SMARTer v4 Ultra Low RNA Kit (Takara Clontech) was used for cDNA synthesis from the single cells. cDNA was quantified with Qubit dsDNA HS (Molecular Probes) and checked on an Agilent Bioanalyser high sensitivity DNA chip. Illumina NGS libraries were constructed with Nextera XT DNA Sample Preparation kit (Illumina), according to the Fluidigm Single-Cell cDNA Libraries for mRNA sequencing protocol. Sequencing was performed on IlluminaR NextSeq 500 using 150 bp paired end kits. All sequencing data was assessed to detect sequencing failures using FASTQC and lower quality reads were filtered or trimmed using TrimGalore. Outlier samples containing low sequencing coverage or high duplication rates were discarded. Analyses using the RNAseq data were performed in the R statistical computing framework, version 3.5 using packages from BioConductor version 3.7. The single cell RNAseq samples were mapped to the GRCh38 reference human genome, as included in Ensembl version 84, using the STAR algorithm and transcript and gene abundance were estimated using the RSEM algorithm. After quantification, the scater package was used to set filtering thresholds, based on using spike ins and mitochondrial genes to filter out bad quality cells, filtering by total number of genes and filtering by total number of sequenced reads. The remaining cells were used after normalizing using size-factors estimated by the SCRAN package. Downstream analyses used log 2 transformed normalized count data. All count data, metadata and intermediate results were kept within a SummarisedExperiment/SingleCellExperiment R object. The data was processed using the edgeR BioConductor package that was used for outlier detection and differential gene expression analyses. Differentially expressed genes were assessed based on their protein coding status. For all differential gene expression comparisons, informative heatmaps with the top differentially expressed genes were generated using the pheatmap package (Kolde, R., 2012, version rHAA4DHf). Unsupervised clustering for single cells as implemented in the M3Drop package was used and significant marker genes for each cluster were produced for downstream analyses.

RNA Sequencing and analysis of bulk cell preparations: The BD FACSAria II flow cytometer was used to sort CD8+ tumour-infiltrating lymphocytes from NSCLC samples. 1000-50'000 CD8+ TILs were sorted into two populations described in the main text, with 1.5-fold maximum difference in cell number for each population across tissues within a patient. Cells were sorted directly into 800 μl Trizol reagent (Invitrogen) and snap frozen in dry ice (long term storage at −80C). At the time of extraction, the samples were thawed at RT and 160 μl of chloroform was added to each. Following a centrifugation step the RNA was isolated from the aqueous phase and precipitated through the addition of equal volumes of isopropanol supplemented with 20 μg linear polyacrylamide. Samples were washed twice in 80% ethanol (first wash over night at 4° C., second wash 5 minutes at RT). RNA pellets were resuspended in 3-15 μl of diethylpyrocarbonate treated water (DEPC). RNA was then quantified by loading of 0.5-1 μl on an Agilent Bioanalyser RNA 6,000 pico chip. Where possible equivalent amounts of total RNA (100 μg) from all samples were used for first strand synthesis with the SmartERv3 kit (Takara Clontech) followed by 15-18 cycles of amplification (according to manufacturers' instruction). cDNA was purified on Agencourt AMPureXP magnetic beads, washed twice with fresh 80% ethanol and eluted in 17 μl elution buffer. 1 μl cDNA was quantified with Qubit dsDNA HS (Molecular Probes) and checked on an Agilent Bioanalyser high sensitivity DNA chip. Sequencing libraries were produced from 150 μg input cDNA using Illumina Nextera XT library preparation kit. A 1:4 miniaturized version of the protocol was adopted (see “Fluidigm Single-Cell cDNA Libraries for mRNA sequencing”, PN_100-7168_L1). Tagmentation time was 5 mins, 837 followed by 12 cycles of amplification using Illumina XT 24 or 96 index primer kit. Libraries were then pooled (1-2 μl per sample depending on the total number of samples) and purified with equal volumes (1:1) of Agencourt AMPureXP magnetic beads. Final elution was in 66-144 μl of resuspension buffer (depending on the total number of pooled samples). Libraries were checked on an Agilent Bioanalyser high sensitivity DNA chip (size range 150-2000 bp) and quantified by Qubit dsDNA HS (Molecular Probes). Libraries were sequenced on IlluminaR NextSeq 500 using 150 bp paired end kits as per manufacturer's instructions. Raw counts for 60675 genes were input using the tximport v1.9.8 package in R. A gene whose mean count across all samples was <15 was removed, leaving 14648 genes. The remaining genes' raw counts (as a tximport object) were then converted to a DESeq2 v1.21.10 object for normalization with betaPrior set to FALSE. Differential expression analyses were then conducted on the negative binomial-distributed normalized counts with FDR set at 5%). Following differential expression, log (base 2) fold changes (log 2FC) were shrunk via the lfcshrink function of DESeq2. For downstream analyses, the negative binomial distributed normalized counts were converted to regularized log (rlog) counts via the rlog function of DESeq2 in R, with blind set to FALSE. The distribution of dispersion of normalized counts was checked by plotting the maximum-likelihood estimate of dispersion (overlaid with the final dispersion estimates) versus the mean of normalized counts. The distribution of rlog counts across samples was additionally checked via a box-and-whisker plot via the boxplot function. Principal components analysis using rlog counts was performed using the prcomp function of the stats base package, with bi-plots subsequently generated to compare eigenvectors, i.e., principal components (PCs), 1 to 3. Supervised clustering was performed by filtering in genes from each differential expression analysis at Benjamini-Hochberg Q≤0.05 and absolute log 2FC≥1. Regularized log counts for these statistically significantly differentially expressed genes were converted to the Z scale and then clustered via 1 minus Pearson correlation distance and Ward's linkage using the Heatmap function of the ComplexHeatmap package. Violin plots of Z-scores per sample were added to the heatmap bottom in order to show distributions across these statistically significantly differentially expressed genes. Colour bars indicating the different sample groups were added at the heatmap top. Partitioning around medoids (PAM) clustering with preselected values of k was performed to identify clusters of genes, with the gene-to-cluster assignment then being used to split the heatmap and gene dendrograms into separate entities.

TCR retrieval from bulk RNAseq: TCRs were identified by TCRseq of RNA from tumour regions performed according to a recently published protocol (Oakes et al., 2017) detail below (see ‘TCR sequencing’ below). RNA from sorted populations of CD8+ TILS were mined for the presence of specific TCRs identified in TCRseq using a bespoke script in R. Briefly, a 20 base pair sequence selected from the CDR3 region of each of the 100 most abundant TCRs in a tumour region was aligned against the bulk RNAseq transcripts. The number of exact matches was compared to the number of matches obtained using a constant (alpha or beta) region sequence of the same length. Typically, a few hundred TCR constant regions could be identified using this approach, per RNAseq library (1-10 million reads).

Multi-region RNA sequencing from tumour tissue: Paired-end RNA sequencing was performed on whole RNA (ribosome depleted) from each tumour specimen within the TRACERx 100 cohort. Reads were 75 base pairs in length, with an average of 50 million reads (25 million each end). In depth analysis of the RNAseq data obtained from the TRACERx 100 cohort. The RNA sequencing data will be deposited in the European Genome-Phenome Archive following publication.

Copy number: Copy number neoantigen depletion was identified by first dividing tumours into immune classifications. All non-synonymous mutations were annotated as either in a region of subclonal copy number loss or not. Then a test for enrichment was performed to determine if non-synonymous mutations that were neoantigens were more likely to be in regions of subclonal copy number loss as compared to the non-synonymous mutations which were not predicted to be neoantigens.

Identifying tumour regions with HLA LOH: Tumour regions harbouring an HLA LOH event were identified using the LOHHLA method, described in (McGranahan, 2017).

Immune evasion alterations: Antigen presentation pathway genes were compiled from Arrieta et al (2018) and affected the HLA enhanceosome, peptide generation, chaperones, or the MHC complex itself. They included disruptive events (non-synonymous mutations or copy number loss defined relative to ploidy, (Jamal-Hanjani et al, 2017)) of the following genes: IRF1, PSME1, PSME2, PSME3, ERAP1, ERAP2, HSPA, HSPC, TAP1, TAP2, TAPBP, CALR, CNX, PDIA3, B2M.

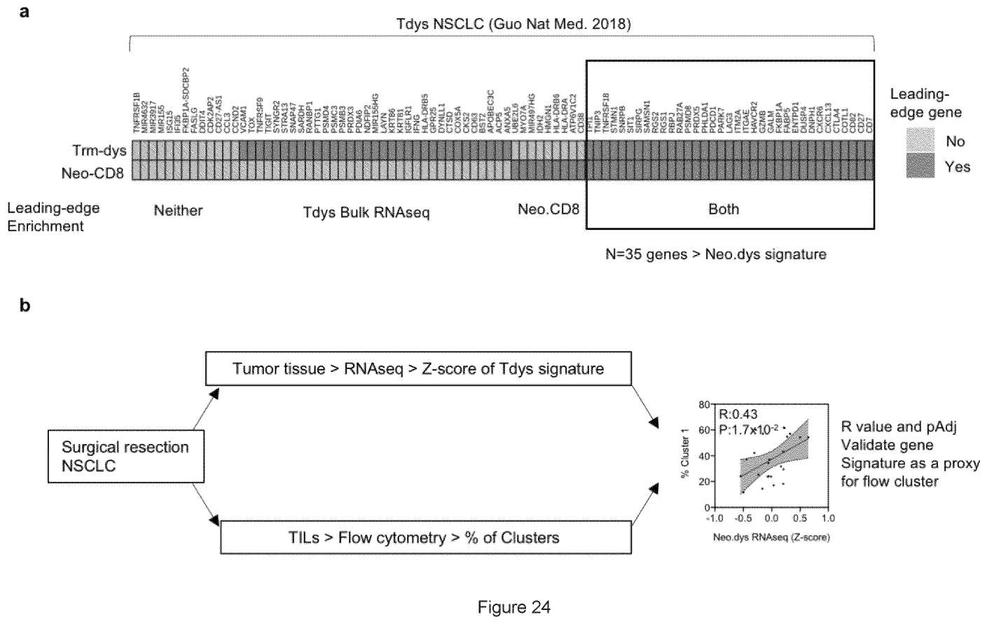

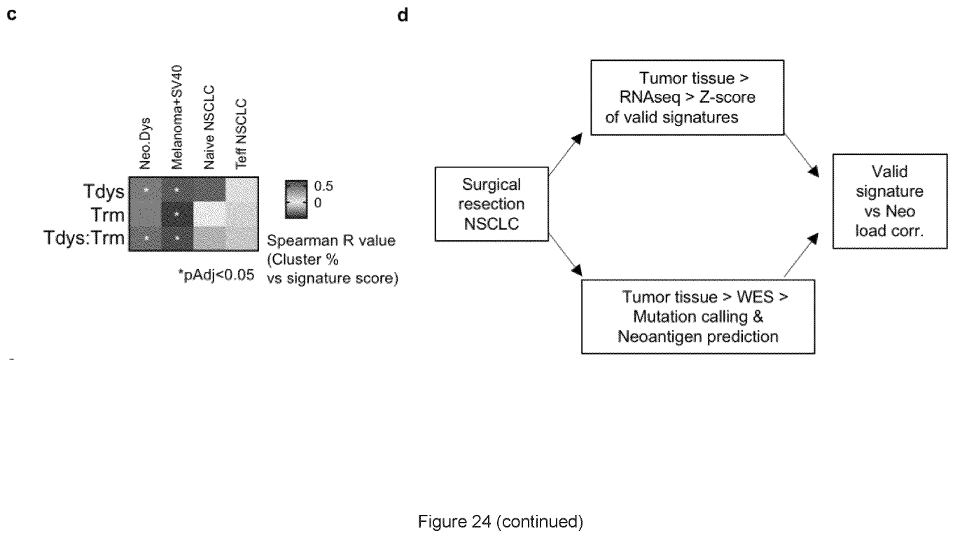

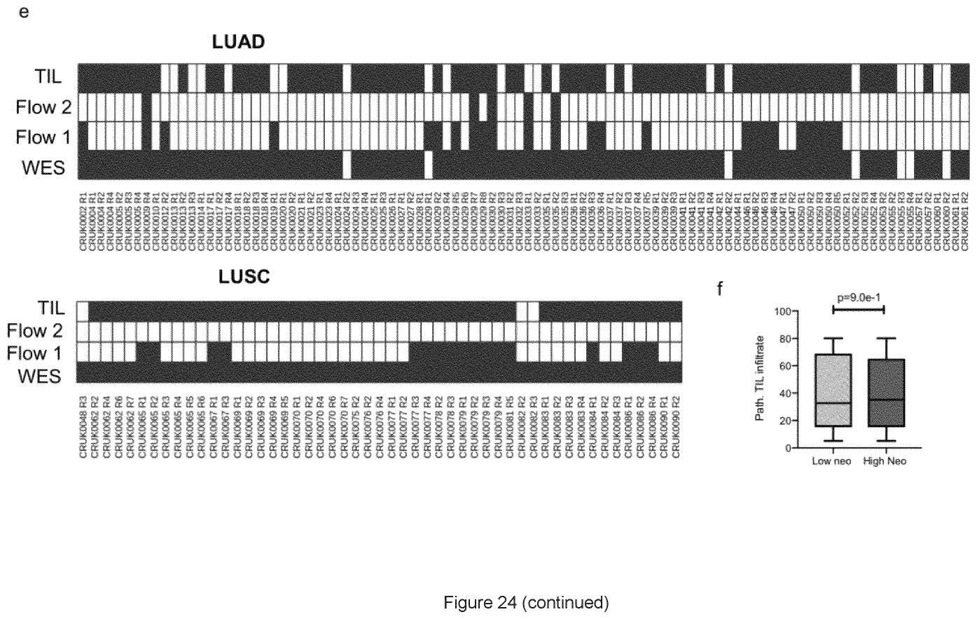

Gene signatures: Gene signature enrichment was evaluated using upper quartile normalized TCGA and TRACERx RNA sequencing count data, estimated by expectation maximization (RSEM). TCGA RNA sequencing data (Thorsson et al, 2018) were downloaded from the GDC website (https://gdc.cancer.gov/about-data/publications/panimmune). For NeoTdys score (curated as per FIG. 24a) or Melan.SV40.Tdys genes signatures (retrieved from Schietinger et al 2016, specifically from Fig S4C of this publication) log₁₀+1 transformed, z-score standardized and the mean value per sample used to represent enrichment. Non-protein coding genes and those not represented in both TCGA and TRACERx data were excluded. All other gene signatures used were generated using this approach. TCGA-LUAD cohort selection was selected to include samples with neoantigen load defined in our previous study (Van Allen et al, 2015).

TCR sequencing: TCR alpha and beta sequencing was performed utilizing whole RNA extracted from NSCLC tumour samples and non-tumour lung tissue or from cryopreserved PBMC samples, using a quantitative experimental and computational TCR sequencing pipeline (Oakes et al., 2017). An important feature of this protocol is the incorporation of a unique molecular identifier (UMI) attached to each cDNA TCR molecule that enables correction for PCR and sequencing errors. The suite of tools used for TCR identification, error correction and CDR3 extraction are freely available at https://github.com/innate2adaptive/Decombinator. The raw DNA fastq files and the processed TCR sequences will be available on the NCBI Short Read Archive and Github respectively, following publication. The number of alpha and beta transcripts is highly correlated. We consistently detect more beta chains than alpha chains, most likely due to the higher number of beta TCR transcripts. In order to validate the sequencing efficiency, we correlated the number of alpha and beta TCR transcripts with matched bulk RNA sequencing data for the tumour regions studied, quantifying T cell infiltration either by the expression of CDR3 gamma, delta and epsilon chains, or with by RNAseq expression of a T cell gene signature. We note that on average, each unique TCR:UMI combination is seen more than 10 times in the raw uncorrected data, making it unlikely that these singletons arise from sequencing errors.

Gene set enrichment analysis (GSEA): A ranked list of genes from the Tdys-enriched (bulk RNAseq) and Neo.CD8 (scRNAseq) datasets was generated using, as metric score, the sign of the fold change multiplied by the inverse of the p-values obtained from the differential gene expression analysis described above. The PreRanked Module of GSEA v3.0 was then used to test for enrichment of previously published CD8 T-cell gene sets in the ranked lists of genes. CD8 T cell gene sets were derived from recent publications; Guo et al (‘TDYS NSCLC’=90 gene T cell exhaustion signature, ‘PRE-TDYS NSCLC’=GZMK transitional, ‘Naive NSCLC’=LEF1, ‘TEFF NSCLC’=CX3CR1, ‘TCM NSCLC’=CD28, ‘TRM-NSCLC’=ZNF683), Thommen et al (‘PD1HI Tdys NSCLC’=merged C1,C3,C5,C7, ‘PD1LO INT NSCLC’=merged C2,C4,C6,C8), Li et al (‘MELANOMA TDYS’=Dysfunctional CD8 T cell gene signature) as originally described (Subramanian et al, 2005; Houstis et al., 2003). A custom R script was used to create dot plots to visualize the GSEA results. Genes that contributed the most to the enrichment signal of the NSCLC Tdys gene set in each Tdys-enriched bulk RNAseq and Neo.CD8 scRNAseq datasets (consensus leading-edge genes) were identified, resulting in a list of 35 genes referred to as Neo.dys core.

Pathology TIL estimation: TIL estimation was carried out according to International Immuno-Oncology Biomarker Working Group guidelines that have been shown to be reproducible amongst trained pathologists (Hendry et al., 2017). Using region level H&E slides, the relative proportion of stromal to tumour area was determined and percentage TILS reported for the stromal compartment by considering the area of stroma occupied by mononuclear inflammatory cells divided by total stromal area. In an intra-personal concordance test, high reproducibility was demonstrated. The International Immuno-Oncology Biomarker Working Group has developed a freely available training tool to train pathologists for optimal TIL-assessment on H&E slides (www.tilsincancer.org).

Statistical analyses: Data were analysed according to the statistical tests indicated in the figure legends using Prism version 8.0.0 or R V 3.5.3 with the packages stated.

### Example 2—Results

To dissect the CD8 T cell compartment in NSCLC at single-cell resolution we developed a custom, high parameter flow cytometry panel including markers that could be used to define lineage (CD3, CD8), antigen engagement (4-1BB, HLA-DR, CD38), dysfunction (PD-1, CD101, FAS)(Thommen & Schumacher, 2018; Philip et al., 2017; Schietinger & Greenberg, 2014), tissue-residency (CD69, CD103, CXCR6)(Hombrink et al., 2016; MacKay et al., 2013) and early (CD28, CD27, CD5) or terminal (CD45RA, CD57) differentiation (Appay et al., 2008) (methods). Flow cytometry was performed on 110 surgically resected specimens from 37 patients with early stage, treatment-naive NSCLC from the lung TRACERx (TRAcking non-small-cell lung Cancer Evolution through therapy (Rx)) study first 100 cohort (Jamal-Hanjani et al., 2017) (FIG. 13a-b). Samples included tumour regions (ranging from one to six per patient) and matched non-tumour lung (n=25) from adenocarcinoma (LUAD), squamous cell carcinoma (LUSC) and other histological subtypes (Other, FIG. 13c).

To characterise the diversity of CD8 T cell subsets in NSCLC we performed unsupervised analysis of CD8 T cells in samples from all tumour regions and non-tumour lung using FlowSOM (Van Gassen et al., 2015). FlowSOM analysis generated 15 clusters (cl) (FIG. 14a, FIG. 15a-b) that were classified into five CD8 T cell subsets based on grouping of clusters on dendograms, topology in dimensionally reduced space and subsequent manual annotation (FIG. 14a, FIG. 15a-b). CD8 T cell subsets comprised three well described CD103⁻ (migratory) populations, including terminally differentiated effector memory cells re-expressing CD45RA (TERMA; CD45RA⁺CD103⁻; cl.8, 11), terminally-differentiated effector cells (TDE; CD45RA⁻ CD103⁻CD57⁺FILA-DR⁺; cl.13,14,15) and central memory-like cells (Tcm; CD45RA⁻CD103⁻CD57⁻CD28^{hi}CD5^{hi}; cl.9). In the CD103⁺ pool (characterised as non-recirculating, tissue-resident populations which stably reside in non-lymphoid tissues (Mueller & Mackay, 2016)) we identified two subsets representing classical tissue-resident memory cells (Trm; CD45RA⁻CD103⁺CD69⁺PD−1^{lo-int}CD27^{lo}FAS^{lo-int}; cl.2, 3, 4, 5) and PD-1^{hi} Trm cells that contained heterogeneous populations with features of T cell dysfunction (6) (PD-1^{hi}-Trm; CD45RA⁻CD103⁺PD-1^{hi}CD27^{hi}FAS^{hi}; cl.1,7,12; FIG. 14a, FIG. 15b). Two clusters were excluded due to abundance (cl.10; <1% average frequency across samples) or distribution (cl.6; present in only CRUK009:R1). Each of the five major CD8 T cell subsets were observed in clustering analyses using multiple iterations of FlowSOM to account for stochasticity in cluster generation (FIG. 15c).

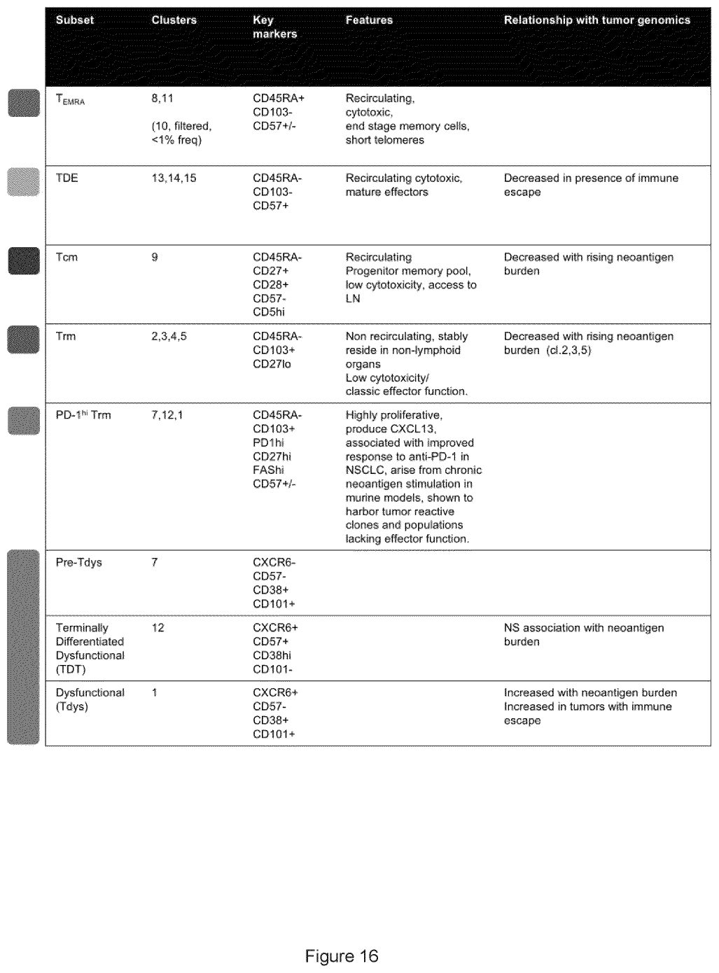

Within each subset, cluster identity was refined further according to activation, migration or maturation status (FIG. 14a lower annotation). Amongst the PD-1^{hi}-Trm subset cl.7 was labelled as pre-dysfunctional (pre-Tdys) due to lower levels of CD38 and CXCR6 which are expressed on dysfunctional CD8 T cells in NSCLC (Thommen et al., 2018; Guo et al., 2018). cl.12 was devoid of the inhibitory molecule CD10119 but expressed the terminal differentiation marker CD57 (‘terminally-differentiated dysfunctional; TDT) and cl.1 expressed CXCR6 and co-expressed high levels of CD38 and CD101 associated with a lack of effector function in solid tumours (Philip et al., 2017) (dysfunctional ‘Tdys’), FIG. 15d, FIG. 16. Tdys (cl.1) was the most abundant population in NSCLC tumours (LUAD 36.2%+/−Std.dev 18.1, LUSC 35.9%+/−15.6; pAdj<5.0×10⁻⁴ vs. all other clusters) though this was non-significant compared to TDE cl.13 (pAdj=4.0×10⁻¹), FIG. 15e. Notably, Tdys (cl.1) and TDT (cl.12) were both enriched in the tumour relative to normal tissue (Cl.1: LUAD, pAdj<1.0×10⁻⁵, LUSC pAdj<1.0×10⁻⁵, Cl.12: LUAD pAdj=4.1×10⁻², LUSC pAdj=4.9×10⁻²; FIG. 15f), yet their frequency was highly variable across TIL samples (e.g. range of Tdys cl.1 in LUAD TILs 5.6% to 61.8%; FIG. 15f-g). These data suggest that, relative to adjacent lung tissue, NSCLC tumours harbour CD8 T cells with a dysfunctional phenotype that vary in abundance between tumour region and patient.

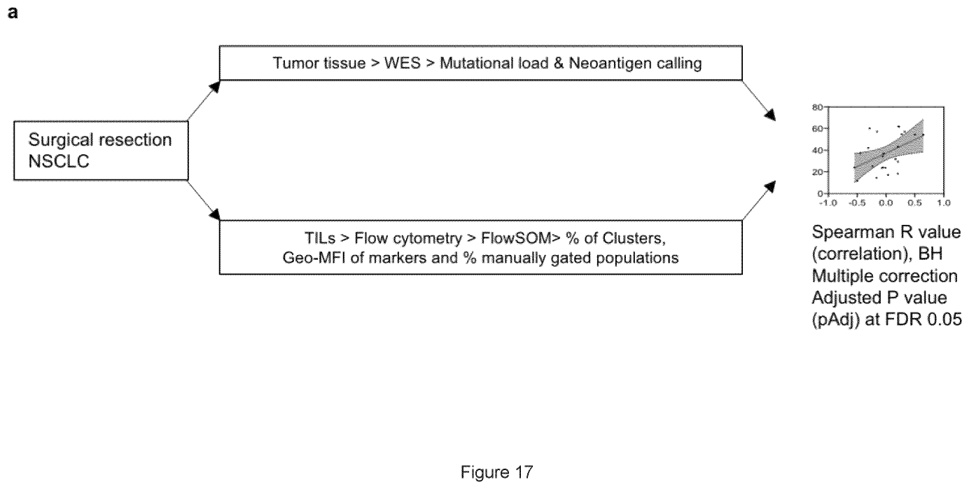

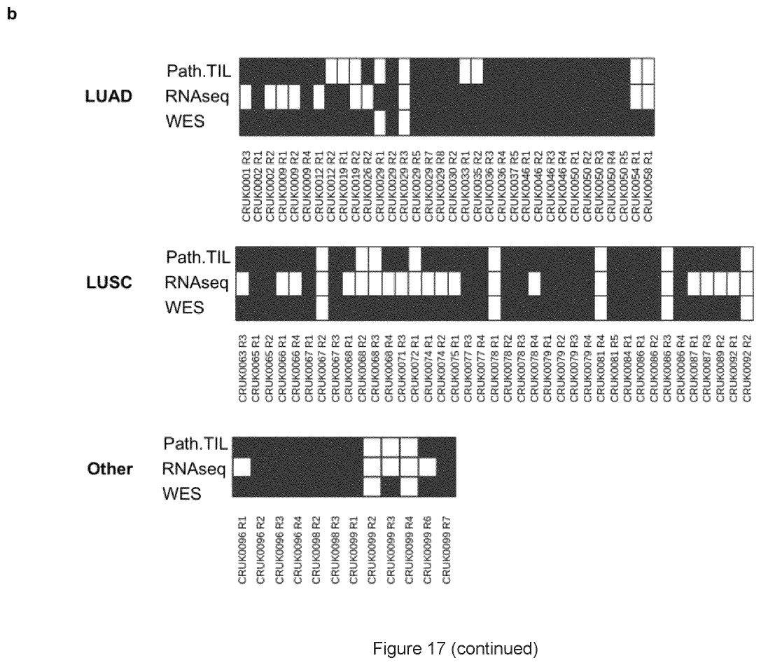

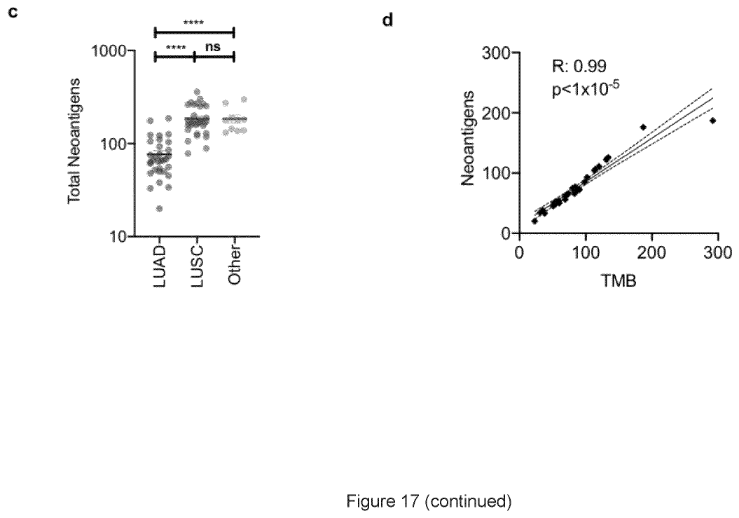

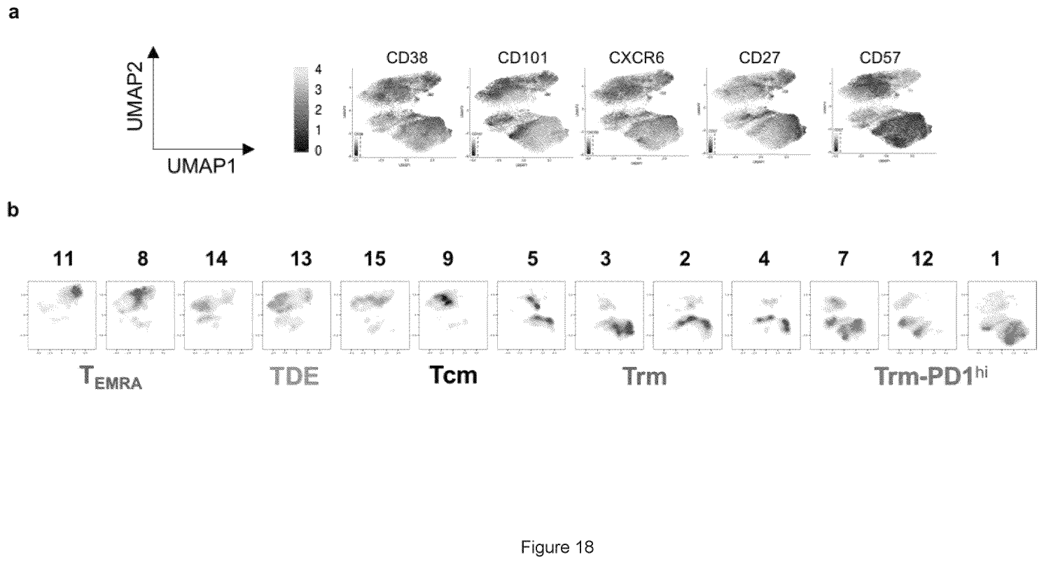

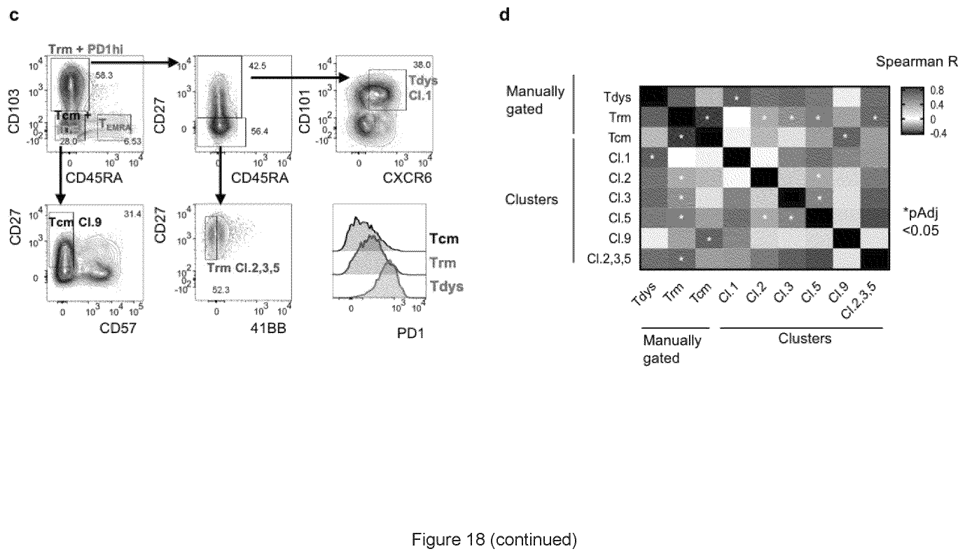

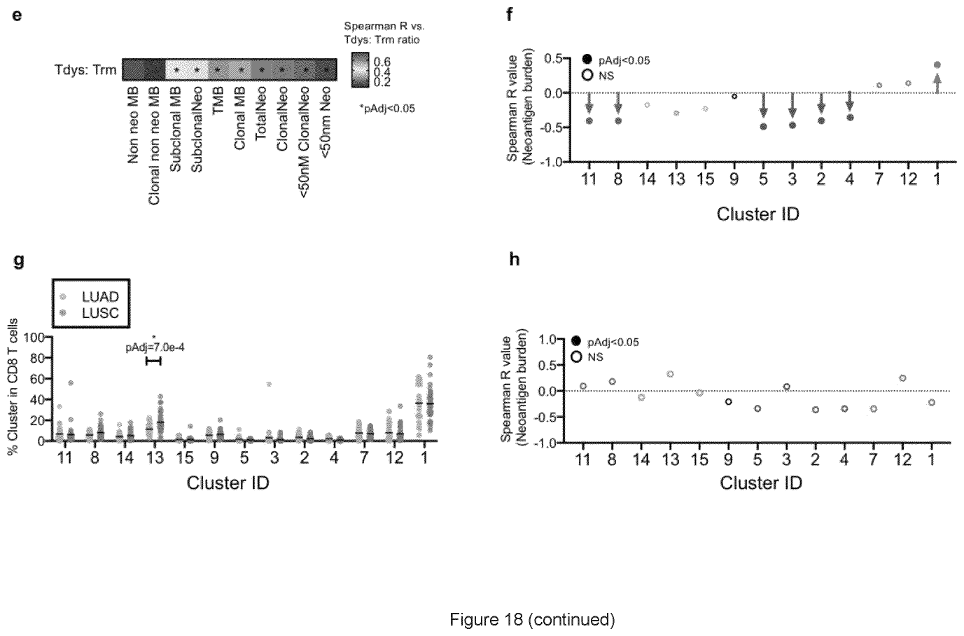

To determine whether the extent of intra-tumoural CD8 T cell differentiation was related to tumour neoantigen burden we examined the correlation between predicted neoantigen load (derived from WES, see methods), and abundance of all CD8 T cell clusters in each tumour region (FIG. 17a-d). In LUAD, Tcm-like cells (2-tailed spearman rank correlation co-efficient R=−0.36, pAdj=4.0×10⁻²) and Trm clusters cl.2 (R=−0.36 pAdj=2.7×10⁻²) cl.3 (R=−0.62, pAdj=8×10⁻⁴) and cl.5 (R=−0.58, pAdj=1.9×10⁻³) were negatively correlated with neoantigen burden. In contrast, the abundance of Tdys cells (cl.1, within the PD-1^{hi}-Trm subset) positively correlated with neoantigen load (R=0.36, pAdj=4.0×10⁻², FIG. 14b-c). Visualization of flow cytometry data in dimensionally reduced space using the Uniform Manifold approximation and Projection algorithm (UMAP) (Becht et al., 2019) exhibited a clear segregation between CD103⁻ (TEMRA, Tcm, TDE) and CD103⁺ subsets (Trm and PD-1^{hi}-Trm containing Tdys cells). The close relationship between Tdys (positively correlated with neoantigen load) and Trm clusters 2,3,5 (negatively correlated with neoantigen load) in UMAP is consistent with the documented TCR overlap between these subsets (Guo et al., 2018), collectively supporting a notion of neoantigen-induced Trm to Tdys differentiation. Tcm-like cells (also negatively correlated with neoantigen load) were within a separate CD103⁻ branch (FIG. 14d-e, FIG. 18a-b).

We next focused on the anti-correlated Tdys and Trm subsets within the tissue-resident pool, given the tumour reactivity of CD103⁺CD8 T cells in NSCLC (Ganesan et al., 2017). To capture the intra-tumoural balance between Tdys and Trm cells we expressed their relative abundance as a ratio (Tdys: Trm) which positively correlated with neoantigen load, both when quantifying FlowSOM clusters (R=0.62, pAdj=8×10⁻⁴) or identifying populations by manual gating (FIG. 14g, see FIG. 18c-d for gating strategy). This correlation was observed irrespective of whether each tumour region was treated independently or if the average of multi-region samples for a given patient was used (FIG. 14g).

We next applied the Tdys: Trm ratio to test associations with alternative genomic measures of mutational load. The Tdys: Trm ratio significantly correlated with TMB as expected (FIG. 18e). Interestingly, we observed a trend towards stronger correlations with predicted high affinity (<50 nM for cognate HLA) vs non-binding neoantigens (R=0.7.54 vs 0.146) and clonal neoantigens previously shown to elicit reactivity in NSCLC (McGranahan et al., 2016) vs sub clonal (R=0.65 vs 0.44) (FIG. 18e-f), however, these comparisons require validation in larger cohorts. Despite having a similar CD8 T cell subset composition (FIG. 18g), LUSC tumours did not show a significant association between cluster frequency and neoantigen load (FIG. 18h), which may relate to a higher TMB (FIG. 17c) or microenvironmental differences associated with smoking history (Jamal-Hanjani et al., 2017). This data support a model of tumour neoantigen-driven differentiation of CD8⁺CD103⁺ tissue resident populations in LUAD, in which Tdys populations accumulate at the expense of non-dysfunctional Trm cells, consistent with tumour reactivity in the CD103⁺ pool (Ganesan et al., 2017; Djeinidi et al., 2015), and specifically amongst PD-1^{high} (Thommen et al., 2018) tissue resident CD8 T cells.

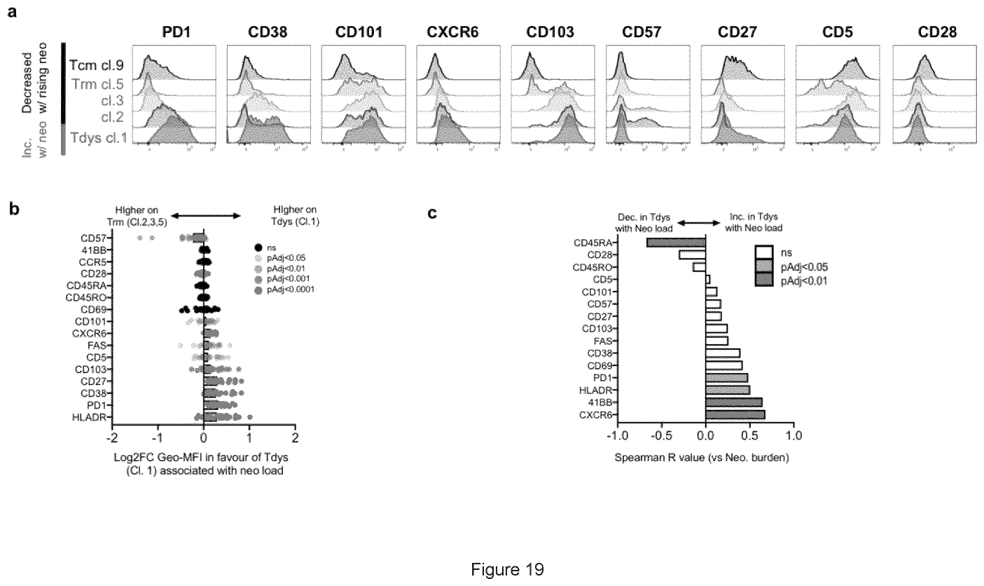

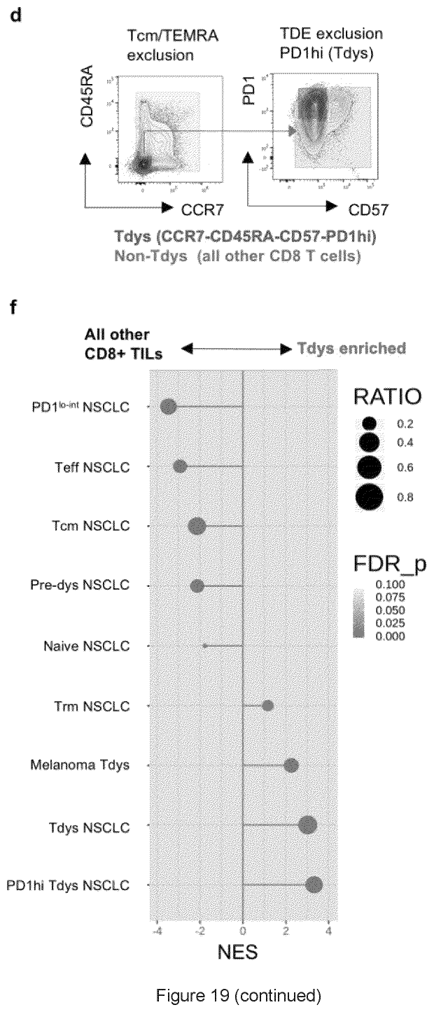

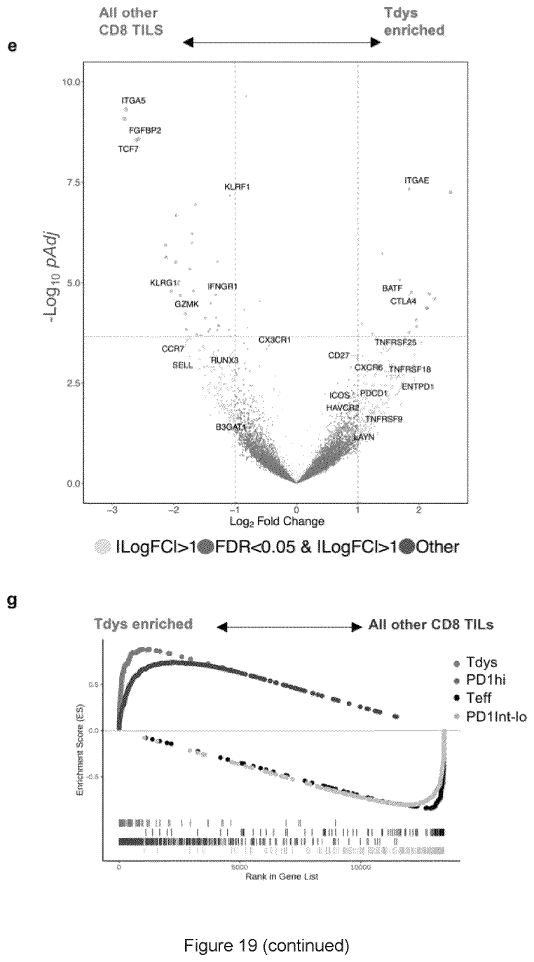

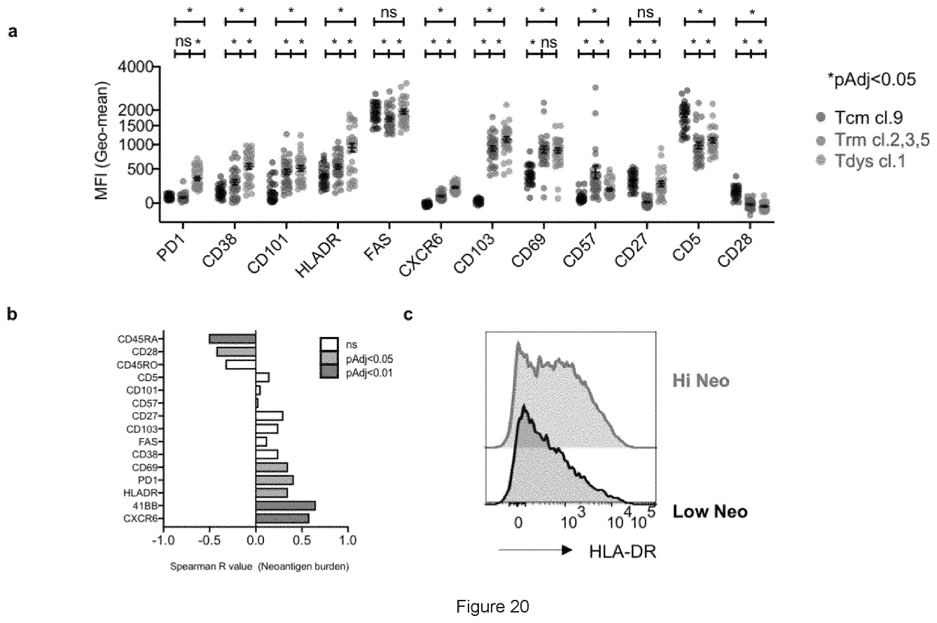

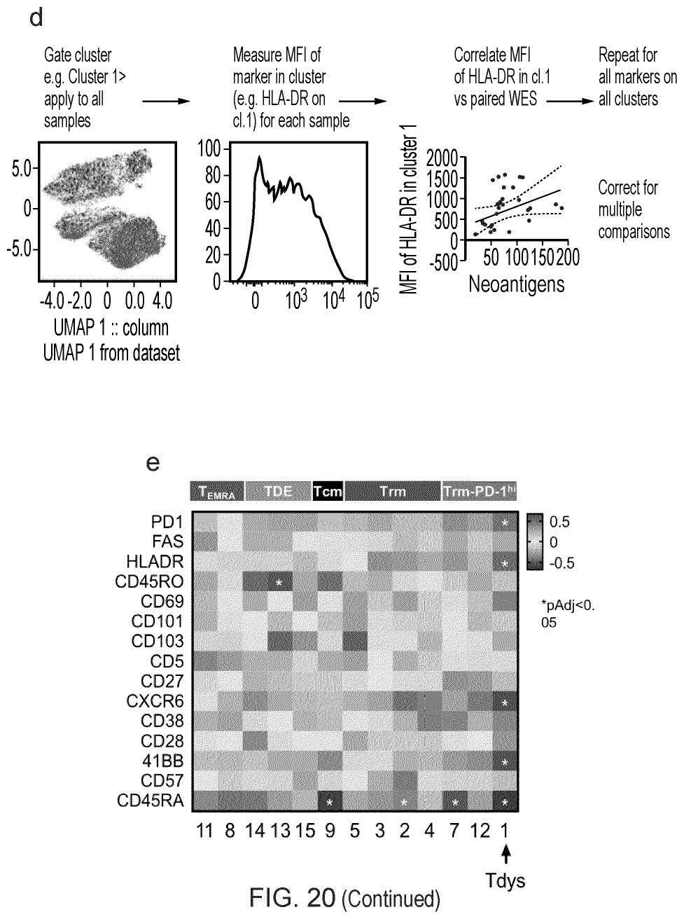

To gain deeper insight in to how changes in TMB may affect the CD8 T cell landscape we explored the phenotype and molecular profile of CD8 Tdys cells relative to other CD8 T cell subsets. Compared to Trm (cl2,3,5 which decrease with neoantigen burden), Tdys displayed evidence of increased antigenic stimulation (marked by high HLA-DR, CD38, PD-1 and CD27 levels), enhanced sensitivity to apoptosis (FAS), decreased effector maturation (CD57) and increased inhibitory receptor expression (CD101)(Philip et al., 2017) (FIG. 14a, FIG. 19a-b, FIG. 20a). Furthermore, within the PD-1^{hi} Trm subset (Cl 7,12,1) the Tdys population (cl1) displayed a unique combination of markers associated with T cell responses in the lung (CXCR6) (Lee et al., 2010), intrinsic inhibition (CD101), sustained TCR ligation (CD38) and a lack of terminal differentiation (CD57⁻). Thus, compared to other tissue resident populations neoantigen-associated Tdys cells exhibited pronounced features of chronic stimulation (see FIG. 14a and FIG. 15c).

We subsequently examined whether the expression of individual markers varied with neoantigen load. In the total CD8 T cell compartment the expression level of molecules related to TCR engagement (PD1, HLA-DR, 41BB) and antigen-specific responses in the lung (CXCR6)(Lee et al., 2010) correlated with neoantigen burden, consistent with the current hypothesis in the field that TMB is related to intra-tumoural T cell activation (FIG. 20b-c). We next repeated this analysis for all CD8 T cell clusters. Changes in Tdys cells reflected those of the total CD8 T cell compartment (FIG. 19c), whilst a similar, non-significant trend was seen in Trm (e.g. Cl.2) and PD-1^{hi} Trm (cl.7, 12) clusters (FIG. 20d-e). These data further support the hypothesis that the Trm pool is activated in high TMB tumours and suggest that CD8 Tdys cells are specifically responsive to neoantigen dose.

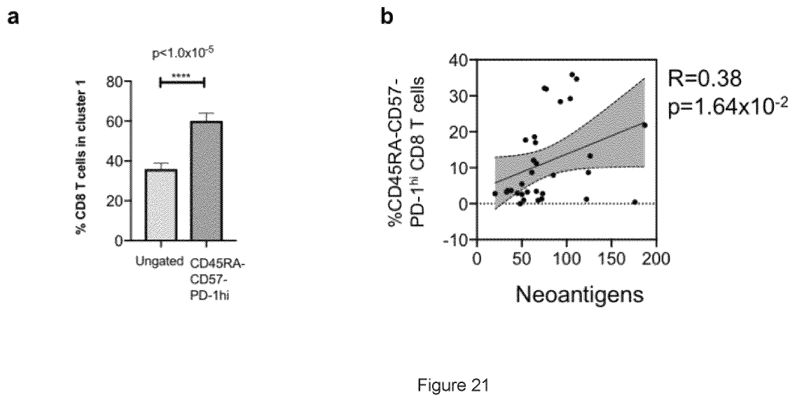

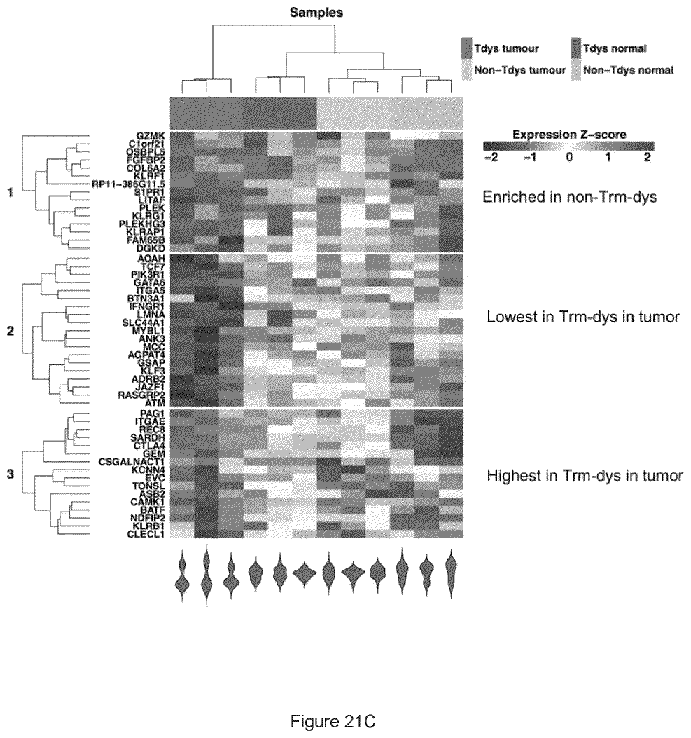

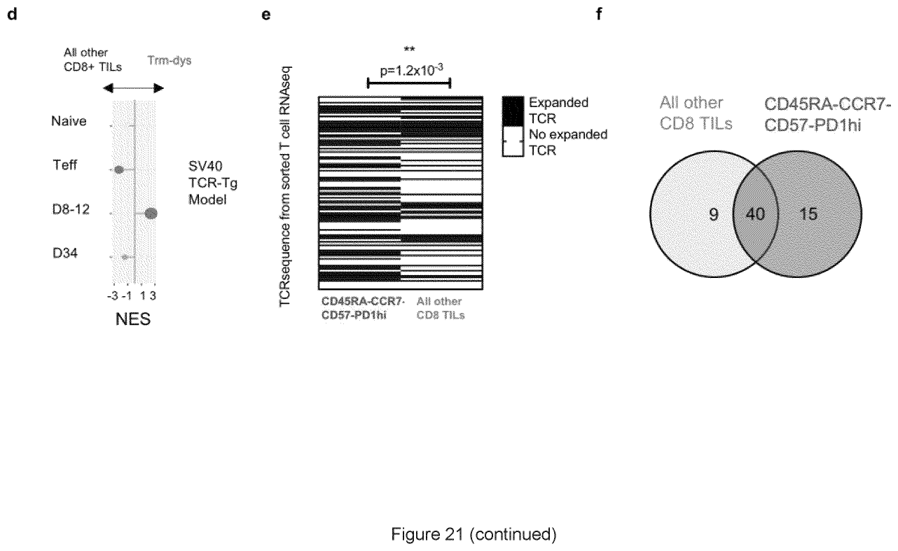

RNAseq analysis was performed on NSCLC CD8 TILs sorted from 3 patients in TRACERx based on a gating strategy (CD45RA⁻CD57⁻PD-1^{hi}) that used highly expressed markers to enrich for Tdys (FIG. 19d, FIG. 21a) and yielded a population that correlated with neoantigen load (FIG. 21b). Relative to the remainder of the CD8 T cell compartment, Tdys-enriched CD8 T cells exhibited significantly lower levels of genes linked to cytotoxicity (FGFBP2, GZMK, KLRG1, KLRF1), stem potential (TCF7) and lymphocyte trafficking/lymph node-homing (ITGA5, SIPR1, CCR7, SELL; FIG. 19e, FIG. 21c). Conversely, genes involved in tissue residency (ITGAE encoding CD103) co-inhibition (CTLA4) and transcriptional regulation of effector function (BATF) were up-regulated (FIG. 19e), and more highly expressed in TILs compared to normal lung tissue (FIG. 21c). Several additional dysfunctional genes shared this expression pattern but were non-significant after adjustment for multiple testing (e.g. PDCD1, ENTPD1 encoding CD39, CXCR6, CD27, ICOS, HAVCR2 encoding TIM-3, TNFRSF18 encoding GITR, LAYN). These data supported that T cell populations that accumulate with neoantigen load exhibit a molecular state of dysfunction.

To formally test this, we performed GSEA using gene sets of defined CD8 T cell subsets from tumour infiltrating lymphocytes from Melanoma (Rizvi et al., 2016) and NSCLC (Guo et al., 2018; Thommen et al., 2018) samples. The sorted CD45RA⁻CD57⁻PD-1^{hi} CD8 T cells in our cohort were strongly enriched for dysfunctional CD8 T cells signatures from melanoma (Normalized enrichment score, NES 2.25, pAadj<1.0×10⁻⁵) and NSCLC(NES 3.015 to 3.3318, pAdj<1.0×10⁻⁵) whilst the remaining fraction of CD8 T cells were enriched for effector, central memory, and transitional/pre-dysfunctional signatures consistent with the diversity of non-Tdys subsets identified by flow cytometry (FIG. 19f-g). Notably, when running a similar analysis using gene sets from TCR transgenic, neoantigen-specific CD8 T cells purified from a mouse tumour model (Schietinger et al., 2016), we observed a significant enrichment of genes induced in early, reversible stages of dysfunction (Day 8-12) but not late (D34), irreversibly fixed states (FIG. 21d). This is in keeping with ongoing activation, rather than terminal dysfunctional in human Tdys cells.

TCRs from RNAseq of sorted Tdys-enriched CD8 T cells were mapped to quantitative, multi-region TCRseq libraries (Oakes et al., 2017) of matched patients. Analysis of TCRs showed increased clonal expansion in Tdys enriched cells relative to non-Tdys, but also clonotype sharing between these populations (FIG. 21e-f), suggesting that Tdys cells undergo antigen-driven expansion and differentiate from a progenitor population in the non-Tdys subset. This finding is consistent with the progressive differentiation of CD8 T cell subsets described in mouse models of cancer (Philip et al., 2017; Schietinger et al., 2016; Miller et al, 2019; Boldajipour et al., 2016), a potential progenitor role for Tcm or Trm cells, and the predicted trajectory of Trm cells to a dysfunctional fate documented in scRNAseq-based pseudotemporal models (Guo et al., 2018). Collectively, these data support a model in which tumour neoantigens activate intra-tumoural CD8 T cells yet may also drive differentiation into a phenotypic and molecular state of dysfunction.

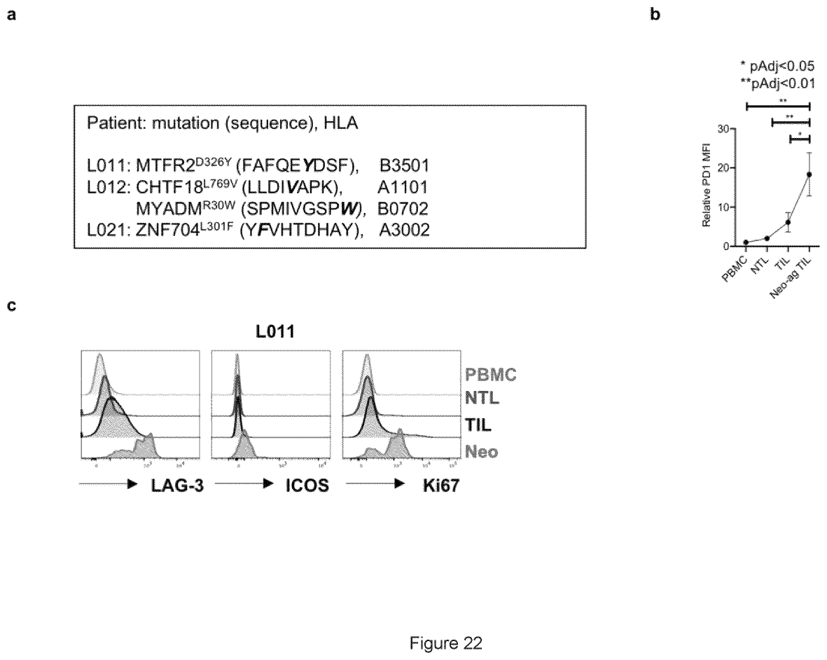

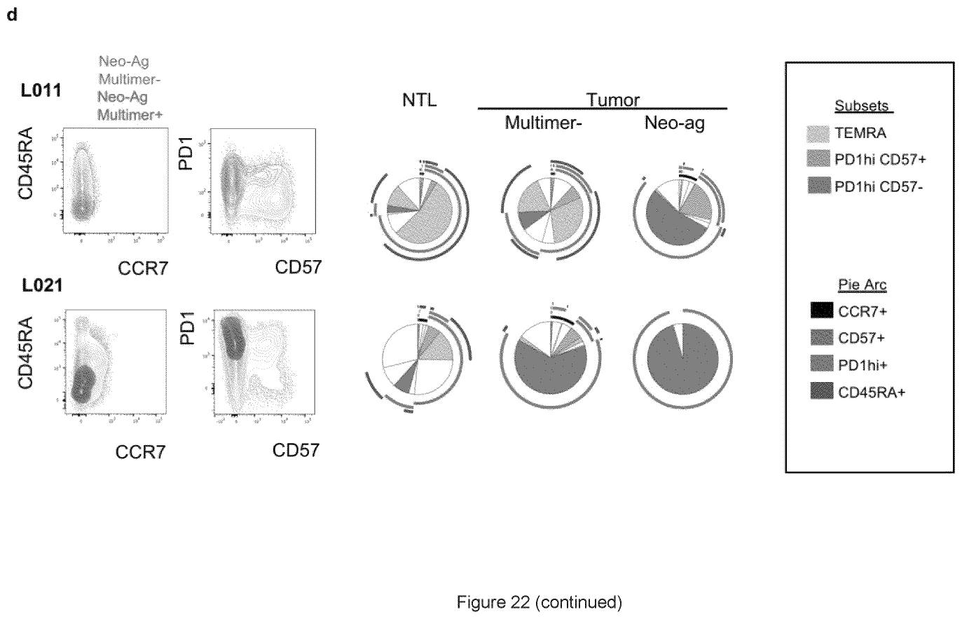

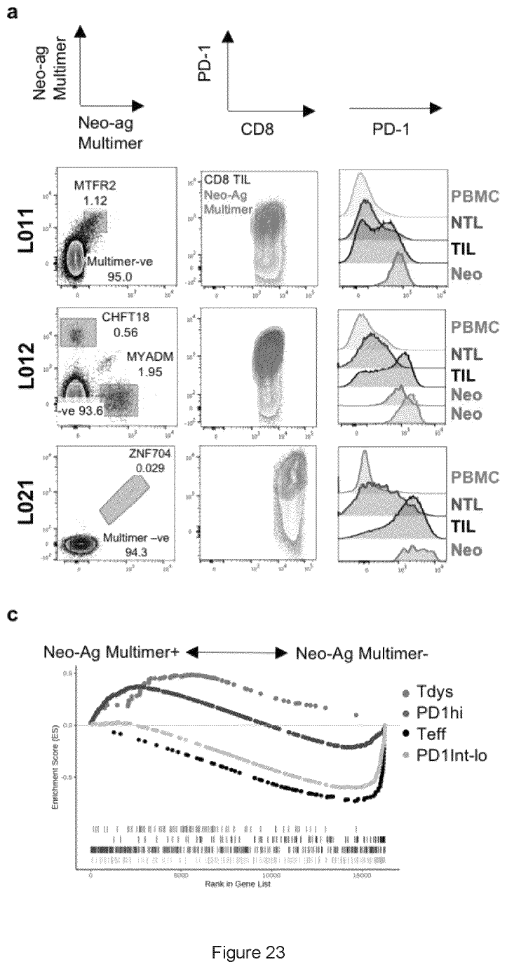

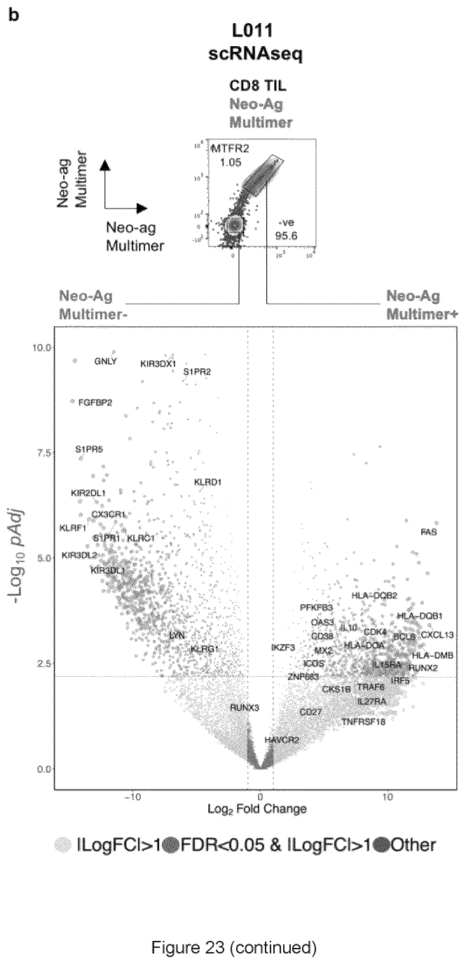

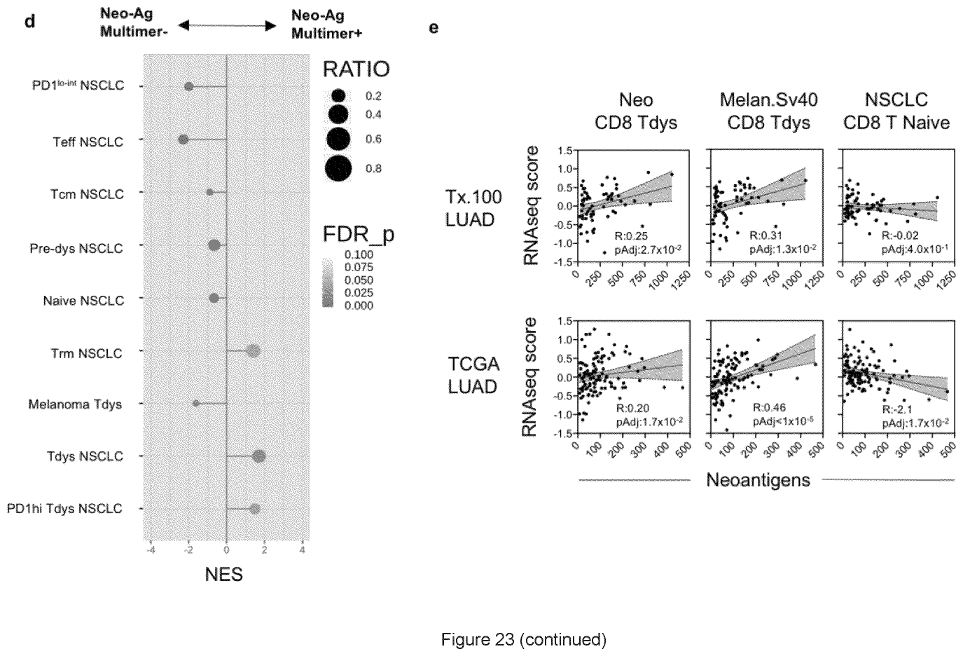

To validate that T cell dysfunction occurred in CD8 T cells specific to neoantigens, we next analysed T cells reactive to four tumour neoepitopes in ex-vivo, unmanipulated TILS from three treatment-naïve NSCLC patients. MHC multimers specific for neoepitopes identified previously (McGranahan et al., 2016) or de novo (FIG. 22a) were used to stain TILS for flow cytometry analysis. MHC-multimer positive CD8 T cells (Neo.CD8) expressed levels of PD-1 that were significantly higher than those in matched PBMC (18.31+/−9.59 fold, pAdj=6.5×10⁻³), NTL (8.89+/−3.41 fold, pAdj=6.5×10⁻³) and multimer negative CD8 TILS (3.23+/−0.52 fold, pAdj=1.6×10⁻²), FIG. 23a, FIG. 22b. Levels of PD-1 on CD8 T cells that exceed those of autologous PBMC have recently been shown to coincide with a lack of TNFa and IFNg production in NSCLC (Thommen et al., 2018). Similar results were seen for Neo.CD8 when measuring other markers that characterise dysfunctional CD8 T cells in NSCLC (ICOS, LAG-3 and Ki67; FIG. 22c)(Guo et al., 2018; Thommen et al., 2018).

In addition, TILS from two patients with available material were stained for markers of differentiation, revealing that Neo.CD8 lack expression of CCR7 and CD45RA and showed low expression of CD57 (FIG. 22d), consistent with the phenotype of Tdys cells in our cohort. scRNAseq of Neo.CD8 (and matched multimer negative CD8 TILS) from ex-vivo TILS of patient L011 revealed 864 genes significantly up-regulated in Neo.CD8 and 1441 that were higher in multimer negative cells (FIG. 23b). Genes preferentially expressed in multimer-negative CD8 TILS included those encoding killer like receptor sub family members (KLRG1, KLRC1, KLRD1, KLRF1), Killer cell immunoglobulin like receptors (KIR2DL1, KIR3DL1, KIR3DL2, KIR3DX1), and other cytotoxicity-associated proteins (GNLY, FGFBP2), molecules involved in potentiation of T cell activation (e.g. LYN) and receptors that coordinate T cell recirculation (S1PR1, S1PR2, S1PR5, CXC3R1), FIG. 23b.

These data were concordant with bulk RNAseq and flow cytometry analysis of Tdys, collectively supporting that neoantigen reactive T cells lack molecular features of effector, central memory or cytotoxic T cells. Up-regulated genes in Neo.cd8 included those involved in MyD88-signaling (IRF5, TRAF6) and the type I IFN response (MX2, OAS3) which contributes to T cell exhaustion in viral infection (Wilson et al., 2013). In addition IL27RA was expressed in neo.CD8 consistent with an increased susceptibility to IL-27 mediated T cell dysfunction (Chihara et al., 2018). Furthermore, neo.CD8 expressed several transcription factors including those that regulate memory cell persistence during chronic infection (RUNX2)(Olesin et al., 2018), suppress IL-2 production (IKZF3) Quintana et al., 2012) and demarcate chronically stimulated memory CD8 T cells subsets that remain sensitive to anti-PD-1 in vivo (BCL-6) (Im et al., 2016). In addition, Neo.CD8 expressed genes related to cell-cycle (e.g. CDK4, CKS1B), components of the MHCII complex (HLA-DOA, HLA-DQB1, HLA-DMB, HLA-DQB2) and markers of activation (CD38, FAS, ICOS), indicative of ongoing TCR signalling and proliferation, in keeping with the phenotype of neo.CD8, Tdys and dysfunctional CD8 T cells in solid tumours (Li et al., 2019; Thommen et al., 2018). Dysfunction-associated cytokines (IL-10) and chemokines (CXCL13) produced by CD8 T cells in NSCLC (Thommen et al., 2018) were also preferentially expressed in neo.CD8 vs multimer negative cells, together with receptors for homeostatic cytokines that sustain dysfunctional CD8 T cells in vivo (Boldajipour et al., 2016) (IL-15RA) and genes that identify Trm cells in NSCLC (PFKFB3, ZNF683)(Guo et al., 2018). Several genes encoding other immune co-receptors and inhibitory molecules were associated with Neo.CD8 but non-significant after adjustment for multiple testing (TNFRSF18, CD27, HAVCR2, ENTPD1), FIG. 23b. These data suggest that Neo.CD8 express tissue-resident associated genes, engage antigen, and proliferate, yet are suppressed by and/or sensitive to multiple pathways of T cell extrinsic and intrinsic regulation. In accordance, GSEA revealed that Neo.CD8 were strongly enriched for Trm and dysfunctional gene sets of CD8 T cells from NSCLC (Guo et al., 2018; Thommen et al., 2018) and melanoma4 cohorts FIG. 23c-d. These data verify that neoantigen specific CD8 T cells express features of tissue residency and dysfunction.

We subsequently tested if the commonly enriched dysfunctional genes from GSEA of our bulk (FIG. 19e-f) and scRNAseq (FIG. 23b-d) analyses could serve as a gene signature of neoantigen-associated CD8 T cell dysfunction (hereafter Neo.Tdys score; method for gene signature generation shown in FIG. 24a—briefly: genes were selected to form part of the signature if they were (i) in the leading edge of GSEA from cl.1 enriched bulk RNAseq (Trm-dys), (ii) in the neoantigen specific CD8 T cells from L011 scRNAseq analysis, and (iii) in the ‘Tdys’ marker genes list from Guo et al. in NSCLC). Of the genes in the Neo.Tdys signature, some were selected as particularly promising actionable targets (CD7, CD82, COTL1, DUSP4, FABP5, ITM2A, PARK7, PHLDA1, RAB27A, RBPJ, RGS1, RGS2, SAMSN1, SIT1, SIRPG and TNIP3) based on their level of expression in dysfunctional CD8 T cells, and a subset of these were selected for experimental validation (CD7, CD82, SAMSN1, SIRPG and SIT1). Data for SIRPG and SIT1 is shown in Example 3.

In parallel, we evaluated a second, unrelated signature derived from Mart1/MelanA-specific CD8 T cells from late stage human tumours and murine early dysfunctional neoantigen specific CD8 T cells developed by Schietinger et al (2016) (hereafter Melan.SV40Tdys). Neo.Tdys and Melan.SV40Tdys scores were validated using RNAseq samples from TRACERx, alongside paired flow cytometry data, which confirmed that both could be used as a proxy for the frequency of Tdys cells and the Tdys:Trm ratio (FIG. 24b-c). We subsequently examined the levels of dysfunctional gene scores in RNAseq data sets from the TRACERx 100 LUAD cohort (regions used in validation of signatures above were excluded) and TCGA-LUAD. Neo.Tdys and Melan.SV40.Tdys scores (but not control naïve CD8 T cell gene scores (Guo et al., 2018)) significantly correlated with neoantigen burden in LUAD cases from TRACERx 100 (n=68 regions from 35 patients; Neo.Tdys R=0.25, pAdj=2.7×10⁻², Melan.SV40.Tdys R=0.31, pAdj=1.3×10⁻²) and LUAD TCGA (n=110 patients with available neoantigen counts, Neo.Tdys R=0.2, pAdj=1.7×10⁻², Melan.SV40.Tdys R=0.46, pAdj<1.0×1⁻⁵), FIG. 23e. Pathology estimated TIL infiltration was not related to neoantigen load, suggesting these results were not confounded by an undue influence of infiltration (FIG. 24f). Taken together, these data suggest that molecular features of neoantigen-specific T cell dysfunction are linked with mutational burden in independent TRACERx and TCGA cohorts, suggesting that neoepitope availability may be a co-factor for CD8 T cell dysfunction in specific LUAD tumours.

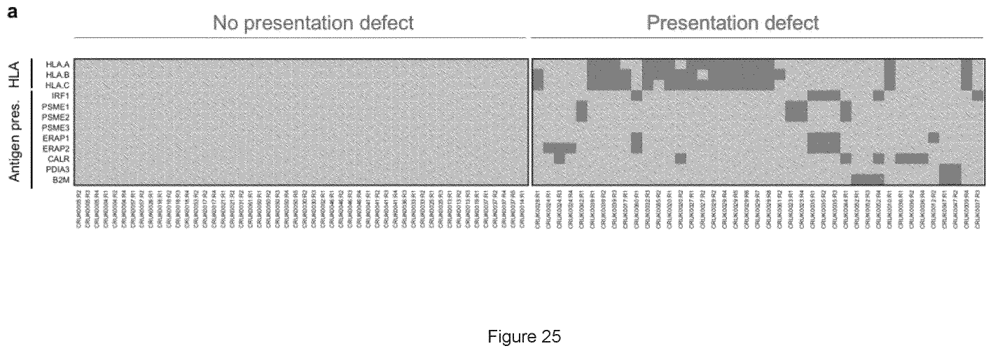

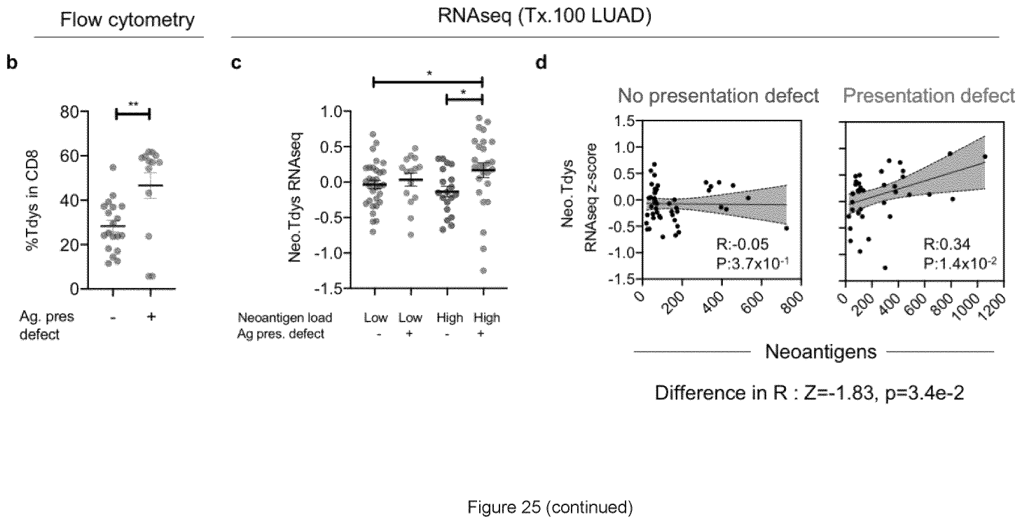

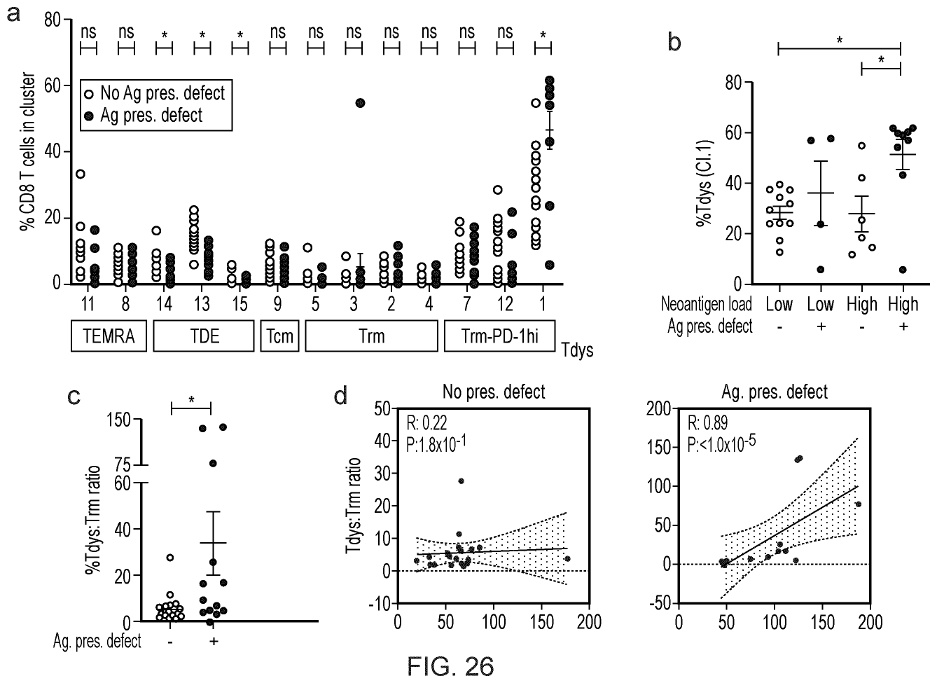

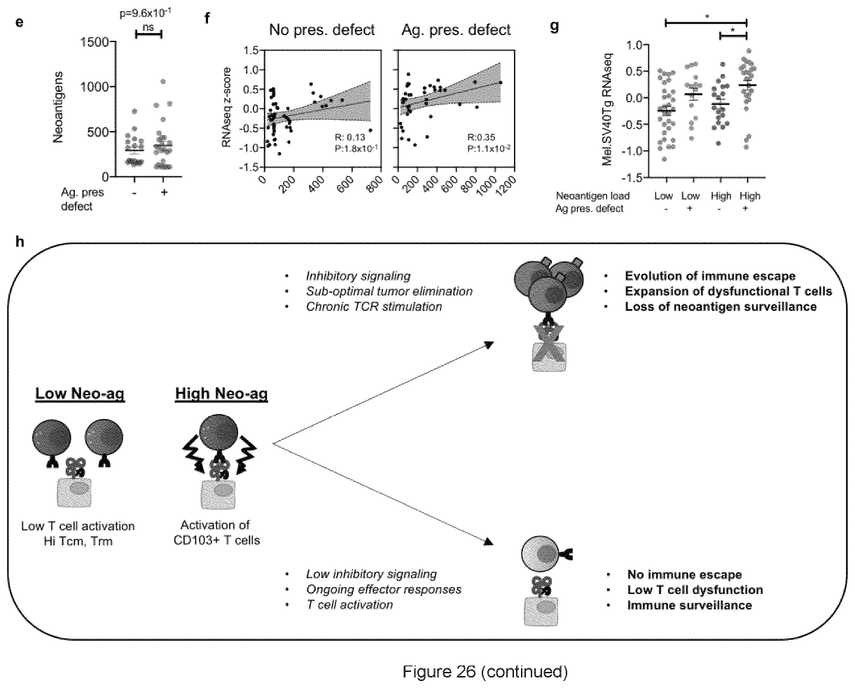

Tumour immune escape mechanisms are pervasive in NSCLC and manifest in regions of active T cell surveillance, indicative of tumour genomic evolution in response to immune selection pressure (34-36). We therefore examined whether the CD8 T cell landscape differed in regions with evidence of immune escape. HLA LOH and antigen presentation defects in the Tx.100 cohort were characterised as previously described (Rosenthal et al., 2019; McGranahan et al., 2017) (methods). Individual LUAD tumour regions were classified as those with evidence of defective antigen presentation (LOH in HLA A, B, C or any non-synonymous mutations or deleterious copy number events in IRF1, PSME1, PSME2, PSME3, ERAP1, ERAP2, CALR, PDIA3, B2M, as recently reviewed in Arrieta et al., 2018) or not (no HLA LOH or mutations/copy number antigen presentation machinery), FIG. 25a. Of all FlowSOM CD8 T cell clusters, only Tdys was increased in tumours featuring defects in antigen presentation (FIG. 25b, FIG. 26a). This was consistent amongst neoantigen high regions (defined according to the median value of samples tested, FIG. 26b), and was reflected by an increased Tdys:Trm ratio (FIG. 26c). In addition, the Tdys:Trm ratio correlated with neoantigen load in tumour regions with immune escape but not those without (FIG. 26d).

To validate these data we used the Neo.Tdys score in the Tx.100 LUAD RNAseq cohort. Consistent with flow cytometry data, we found the Neo.Tdys score to be increased in tumours that exhibited immune escape (FIG. 25c). This increase was only observed in regions with both a high neoantigen load and the presence of immune escape mechanisms (FIG. 25c) and was not due to a difference in neoantigen load between high mutational burden tumours with or without immune escape (FIG. 26e). Finally, we validated that a correlation between neoantigen burden and Neo.Tdys score was exclusively observed in tumour regions with evidence of immune escape (FIG. 25d). Similar results were seen using the SV40.Tdys gene score (FIG. 26f-g). These data suggest that neoantigen-driven CD8 T cell dysfunction preferentially occurs in regions under high immune selection pressure.

The current hypothesis in the immune-oncology field is that TMB corresponds to the breadth and magnitude of tumour specific T cell responses, potentiating more favourable clinical outcome during checkpoint inhibition. However, whilst the relationship between tumour evolution and T cell surveillance has become increasingly apparent (Marty et al., 2017; Luksza et al., 2017; Havel et al., 2019), the genomic determinants of intra-tumoural CD8 T cell activation and dysfunction have not been systematically studied. Our work supports that TMB is proportional to tumour immunogenicity, reflected by a correlation with HLA-DR, CD38, 4-1BB on total CD8 T cells. In addition, our data is consistent with a process of neoantigen-directed CD8 T cell differentiation, resulting in expansion of Tdys at the expense of Tcm and Trm subsets.

Most crucially, our results suggest that the relationship between TMB and the CD8 T cell landscape is dependent upon the evolutionary context. Collectively these data support a model whereby tumour-reactive T cells elicit initial cytotoxic responses, yet are subject to chronic neoantigen stimulation, leading to T cell dysfunction, sub-optimal tumour elimination and consequent evolution of immune escape (FIG. 26i). In contrast, high TMB tumours lacking defects in antigen presentation and exhibiting low T cell dysfunction may represent an earlier phase of the response and/or a microenvironment conducive to neoantigen surveillance (FIG. 26i). This notion is consistent with the improved outcome of NSCLC patients that bear a high TMB and low immune evasion capacity (Rosenthal et al., 2019) or a low Tdys:Trm ratio (Guo et al., 2018). Notably, Tdys may continue to incur chronic stimulation in regions with MHC I pathway dysregulation, since bi-allelic loss of antigen presentation pathway genes15 or homozygous deletion of HLA (McGranahan et al., 2017) have not been detected in the TRACER×100 cohort. Taken collectively, the data suggest that the Trm pool in untreated NSCLC is subject to dynamic neoantigen stimulation which progresses to T cell dysfunction in parallel with evolution of immune escape.

### Example 3—Validation of Actionable Targets Associated with T Cell Dysfunction in Cancer

Material and Methods

Flow cytometry. For tumour samples, FC receptors were blocked with Human Fc Receptor Binding Inhibitor (Cat #14-9161-73, Invitrogen) 15 minutes before staining. Non-viable cells were stained using the eBioscience Fixable Viability Dye eFluor 780 (Thermo, cat #65-0865-14). Cell were washed with PBS and incubated with a mixture of flow cytometry monoclonal antibodies per 20 minutes. In order to detect intracellular epitopes cells were fixed and permeabilized using the eBioscience Foxp3/Transcription Factor Staining Buffer Set (eBioscience, Cat: 00-5523-00) following the manufacturer protocol. Cells were resuspended in PBS and acquired using the BD FACSymphony machine. Antibodies for flow cytometry were purchased from Biolegend, BD and ThermoFisher Scientific (eBioscience): KI67 (Cat #564071 BD Biosciences, Clone: B56), CD8 (Cat #564804 BD Biosciences, Clone: RPA-T8), CD45RA (Cat #565702 BD Biosciences, Clone: HI100), CD38 (Cat #565069 BD Biosciences, Clone: HIT2), CD39 (Cat #564726 BD Biosciences, Clone: TU66), CD3 (Cat #565511 BD Biosciences, Clone: SK7), PD1 (Cat #562516 BD Biosciences, Clone: EH12.1), SIRPγ (Cat #747683 BD Biosciences, Clone: OX-119), CCR7 (Cat #353224 Biolegend, Clone G043H7), CD25 (Cat #302634 Biolegend, Clone: BC96), TIM3 (Cat #565566 BD Biosciences, Clone: 7D3), CD4 (Cat #317442 Biolegend, Clone: OKT4), CD28 (Cat #11-0289-42 eBiosicence, Clone: CD28.2), LAG3 (Cat #369312 Biolegend, Clone: 11C3C65), TCF1 (Cat #655208 BD Biosciences, Clone: 7F11A10), 41BB (Cat #309826 Biolegend, Clone: 4B4-1), FCRL3 (Cat #374409 Biolegend, Clone: H5/FcrL3), SIT1 (Cat #367804 Biolegend, Clone: SIT-01), GZMB (Cat #367804 Biolegend, Clone: QA16A02). Data was acquired on a BD Symphony flow cytometer and analyzed in FlowJo v10.5.3 (Treestar).

Target validation in samples of lung cancer patients from TRACER×. Tissue samples were collected and transported in RPMI-1640 (Cat #R0883-500ML, Sigma). Single-cell suspensions were produced by enzymatic digestion using a Liberase TL (Cat #05401127001, Roche) and DNase I (Cat #11284932001, Roche) with subsequent cellular disaggregation using a Miltenyi gentleMACS OctoDissociator. Lymphocytes were isolated from single-cell suspensions by gradient centrifugation on Ficoll Paque Plus (Cat #17-1440-03, GE Healthcare), cryopreserved in fetal bovine serum (Cat #10270-106, Gibco) containing 10% DMSO (Cat #D2650-100ML, Sigma) and stored in liquid nitrogen. Tumour infiltrating lymphocytes obtained from stage IV lung cancer patients were stained as explained before using monoclonal antibodies for each one of the proposed targets. The expression of the proposed targets was assessed in the different populations of CD4 and CD8 T cells. Dysfunctional tumour reactive CD4 and CD8 T cells were identified by the expression of PD1, TIM3 (Dysfunctional) and CD39 and 41BB (Neoantigen reactive). The expression of the different markers was used to define populations of exhausted and non-exhausted T cells expecting to find an increasing expression of the proposed targets on each population, from non-neoantigen reactive towards the exhausted tumour reactive population.

Gene-editing of human PBMC-derived T cells using CRISPR/Cas9 technology. Frozen peripheral blood mononuclear cells are thawed and incubated on overnight in as 12-well plate (Cat. No 353043, Falcon) containing 2 mL of growth media (RPMI 1640 (Cat. No 51536C, Sigma-Aldrich) supplemented with 5 mL of Penicillin-Streptomycin (Cat. No P4333, Sigma-Aldrich) and 50 mL of Human Serum (10% final conc) (Cat: G7513-100 mL, Sigma-Aldrich)) supplemented with 50 IU/mL of IL2 (Proleukin, Novartis). The following day, cells are transferred into a 12-well plate coated with 10 μg/mL of αCD3 antibody (Cat No. BE0001-2, BioXcell, Clone: OKT3) and cultured in 2 mL of growth media containing 100 IU/mL of IL2 and 10 μg/mL of αCD28 antibody (Cat. No BE0248, BioXcell, Clone: 9.3) for 72 hours. The sgRNA is prepared by mixing the Alt-R tracrRNA (Cat. No 1072534 IDT) and the Alt-R crRNA 200 μM (custom-made, IDT) in a 0.6 mL tube and incubated 5 minutes at 95 C. The sgRNA is mixed with the Nuclease-Free Duplex Buffer (Cat. No 11050112, IDT) and then mixed with the Cas9 protein (Cat. No 1081059 IDT) to form the ribonucleoprotein complex. Cells are electroporated with the ribonucleoprotein complex (For 4D-Nucleofector X Kit Small (V4XP-3032 Lonza) following the manufacturer protocol and left on growth media containing 100 IU/mL for three days. Finally, cells are stained for flow cytometry and the knock-out of the target gene is measured by quantifying the number of cells positive for the target relative to the number of CD4 or CD8 T cells. The sequences of the cRNAs used are shown in Table 1.

TABLE 1
List of targets knocked-out by CRISPR/Cas9
and cRNA sequences used for each target.

| Target | |
|---|---|
| gene | cRNAs used for sgRNAs |
| SIT1 | 1. CTGCACGGATCTACTCGCAC (SEQ ID NO: 1) |
| | 2. CTCCAAGAGTTGGATCCTGC (SEQ ID NO: 2) |
| SAMSN1 | 1. TCAAATAATGGAGGCGGTTT (SEQ ID NO: 3) |
| | 2. GAGACTATCCATGGAGTCAC (SEQ ID NO: 4) |
| SIRPG | 1. GACCTGAGAGCGAACGTCCC (SEQ ID NO: 5) |
| | 2. CGCAGGGCCCAATACCACGG (SEQ ID NO: 6) |
| CD7 | 1. CACTACGGACAGACGGTTCC (SEQ ID NO: 7) |
| | 2. ATGCTCGGACGCCCCACCAA (SEQ ID NO: 8) |
| CD82 | 1. CCCCACGCCGATGAAGACAT (SEQ ID NO: 9) |
| | 2. GGATGCCTGGGACTACGTGC (SEQ ID NO: 10) |
| FCRL3 | 1. AATCTAGAGATCCGGCCCAC (SEQ ID NO: 11) |
| | 2. AGGATCCTCGGGTCTTACAT (SEQ ID NO: 12) |
| IL1RAP | 1. GTGTCAAACCGACTATCACT (SEQ ID NO: 13) |
| | 2. GACGTACGTTTCATCTCACC (SEQ ID NO: 14) |
| FURIN | 1. GACTAAACGGGACGTGTACC (SEQ ID NO: 15) |
| | 2. TCGGGGACTATTACCACTTC (SEQ ID NO: 16) |
| STOM | 1. GCGACCCAATCTAAAGATGA (SEQ ID NO: 17) |
| | 2. GCAAGGTCCAAGGCCCTTAC (SEQ ID NO: 18) |
| AXL | 1. TGCGAAGCCCATAACGCCAA (SEQ ID NO: 19) |
| | 2. CCCGAAGCCAATGTACCTCG (SEQ ID NO: 20) |
| E2F1 | 1. ACGGTGTCGTCGACCTGAAC (SEQ ID NO: 21) |
| | 2. AAGGTCCTGACACGTCACGT (SEQ ID NO: 22) |

In vitro reactivation of gene-edited T cells using low dose of αCD3 and αCD28. Following gene-editing 1×106 human T cells were kept in growth media containing 50 UI/mL of IL2 (Proleukin, Novartis) during 10 days and in a 96 well plate (Cat. No 163320, Thermo Scientific). Media was changed every two-three days by removing 100 μL of old media and adding 100 μL of fresh media. On day 10 1×106 cells were counted, stained with cell trace violet (Cat. No 34557, Thermofisher Scientific) and reactivated using a 1/800 dilution of αCD3/αCD28 dynabeads (Cat. No 32-14000, Gibco) for four days. On day four cells were incubated with 1 μL/mL of GolgiPlug (Cat. No 555029, BD Biosciences) and then stained for flow cytometry. Flow cytometry staining was performed using the BD CytoFix/Cytoperm kit (Cat. No 554714 BD Biosciences) following the manufacturer protocol. The antibodies used are the following: CD8 (Cat. No 564804 BD Biosciences, Clone: RPA-T8), CD3 (Cat. No 565511 BD Biosciences, Clone: SK7), CD4 (Cat. No 563877 BD Biosciences, Clone SK3), TNFα (Cat. No 17-7349082 Invitrogen, Mab11), IFNγ (Cat. No 12-7319-42, Clone 4S.B3), IL2 (Cat. No 17-7029-82 eBioscience, Clone: MQ1-17H2).

Generation of NY-ESO-1 transduced T cells. The NY-ESO-1 T cell receptor was cloned into a retroviral vector fused to the RQR8 gene using an E2A self-cleaving peptide. Virus were produced using HEK 393 T cells and supernatants were used to transduce the T cells. Peripheral blood mononuclear cells were activated using plate bound αCD3 (Cat No. BE0001-2, BioXcell, Clone: OKT3) and αCD28 (Cat. No BE0248, BioXcell, Clone: 9.3) for three days. On day three cells are incubated with the supernatants containing viral particles and left on culture for another 3-4 days. Transduced cells were then purified using the Miltenyi CD34+ isolation kit (Cat. No 130-046-702) and grown on IL2 in order to expand them. This approach was previously disclosed in Stadtmauer et al. (2020). The resulting T cells express a T cell receptor recognising a known antigen, which is expressed by available tumour cell lines. As such, the approach enables the redirection of T cells towards the cancer-specific antigen, for the purpose of testing the effect of targeting specific genes on T cell function in cancer.

In vitro evaluation of inhibitors. CD4 and CD8⁺ T cells can be stimulated in vitro with low dose of plate bound anti CD3 antibodies and in presence or absence of increasing concentrations of each inhibitor to evaluate proliferation, activation phenotype and upregulation of granzymes over time by high dimensional flow cytometry. Experiments may be performed in triplicated with mouse and human T cells isolated from spleen and peripheral blood respectively.

In vivo evaluation of inhibitors. Mice can be challenged in the flank and intradermally with MCA205 sarcomas as their tumour immune microenvironment is well characterised in SQ laboratory. 6, 9 and 10 days after tumour implantation mice may be treated with vehicle, each inhibitor and anti-CTLA4 as positive control for the upregulation of granzyme B by CD4 TILS. Fifteen days post tumour challenge mice are sacrificed, and lymph nodes and tumours are harvested for high dimensional analysis of immune cell differentiation and acquisition of cytotoxic activity. Experiments may be performed in triplicated with n−=5 mice per group.

OKT3-expressing tumour cells cocultured with gene edited PBMC-derived human T cells. The protocol used for these assays is illustrated on FIG. 31A. The following control conditions were used: (i) unstimulated cells: T cells kept in media without stimulation (negative control); (ii) cells cocultured with tumour cells that do not express anti-CD3 (CTRL (H2228), negative control); (iii) PMA/Ionomycin incubation which activates T cells and promote cytokine production following 4 hours of in vitro stimulation (positive control); (iv) dynabeads coated with anti-CD3/CD28 antibodies (positive control). T cells derived from PBMCs that were electroporated with a scrambled non-targeting crRNA were used as a control for the effect of the KO (CTRL). Only SIT1, SIRPG and CD7 were tested with this assay.

Small Arrayed CRISPR screen on NSCLC TILs. The protocol used for these assays is illustrated on FIG. 31B. Knock-out of NSCLC TILs were done using 2 different crRNAs (named AA, AB, AC or AD) per gene followed by electroporation of the Cas9:crRNA complex (see detailed protocol for gene editing below). We refer to single-guide RNA as the duplex of crRNA and tracrRNA (crRNA:tracrRNA). 4 days later the edited TILs were co-cultured with anti-CD3 expressing lung tumour cells (see detailed protocol for coculture below). A first readout (Readout 1, upregulation of PD1 and LAMP1) was measured after 24 hours using high-dimensional flow cytometry. A second readout (Readout 2, cytokine production) was measured after 72 hours using high-dimensional flow cytometry. See detailed protocol for flow cytometry below. Each condition is a result of two replicates. The following markers were measured in each of the CD4+ and CD8+ T cell populations: proportion of PD-1⁻ cells, proportion of PD-1^{high} cells, proportion of PD-1^{total} cells (PD-1^{high} cells+PD-1^{int} cells), proportion of TIM3+ cells, proportion of LAG3+ cells, proportion of LAMP1+ cells, proportion of IFNg+ cells, proportion of IL-2 cells, proportion of GZMB+ cells. PD1 and TIM3 are negative regulators of T cell activation. In NSCLC tumours, PD-1^{high} CD8 TILs display a dysfunctional state and their presence has been correlated with hampered response to PD-1 blockade after polyclonal stimulation of the T cells. Thommen, Daniela S et al, Nat. Med. 2018. We expect that since PD-1 is an activation marker in both T cell compartments, PD-1^{high} CD4 TILs will also display a dysfunctional state like in the CD8 TILs. LAMP1 is a marker of degranulation, a process used by several immune cells to release cytotoxic molecules (e.g. perforin and granzyme, by cytotoxic T cells) from secretory vesicles. The following control conditions were used: (i) unstimulated cells: T cells kept in media without stimulation (negative control); (ii) cells cocultured with tumour cells that do not express anti-CD3 (CTRL (H2228), negative control); (iii) PMA/Ionomycin incubation which activates T cells and promote cytokine production following 4 hours of in vitro stimulation (positive control); (iv) dynabeads coated with anti-CD3/CD28 antibodies (positive control). T cells that were electroporated with a scrambled non-targeting crRNA were used as a control for the effect of the KO (CTRL).

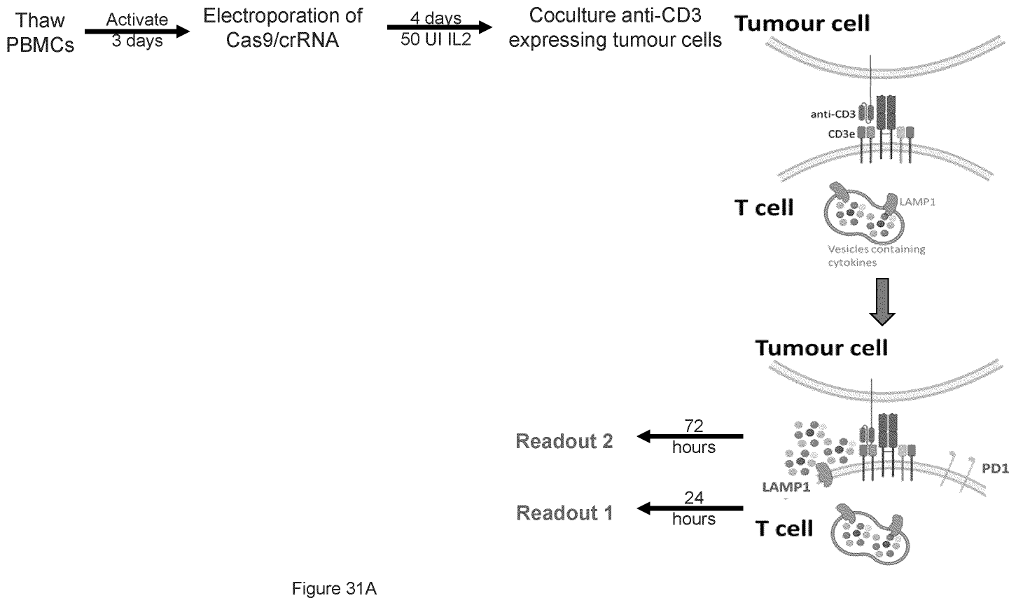

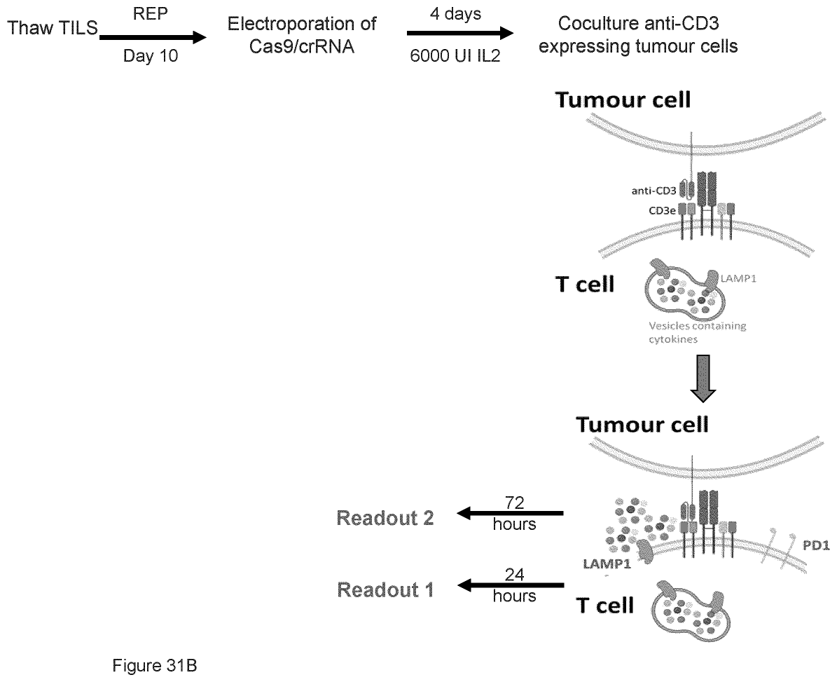

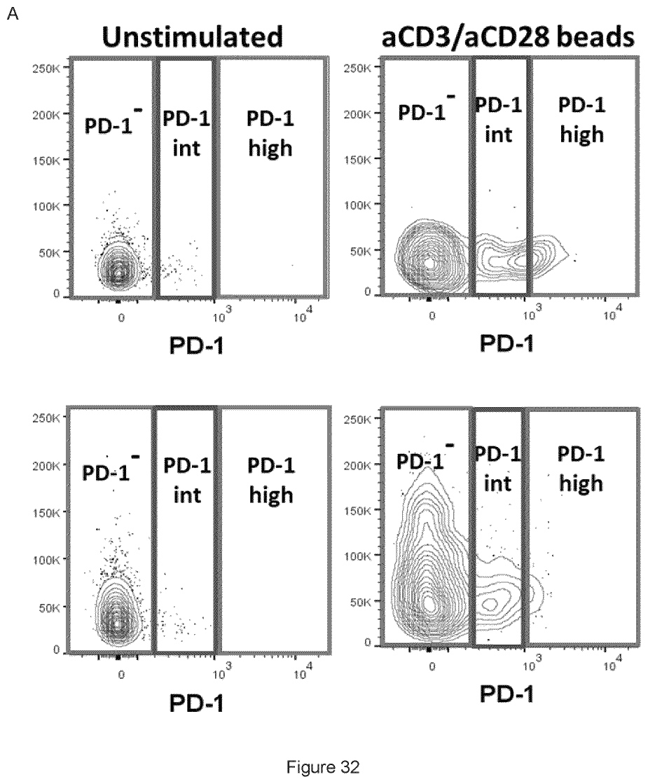

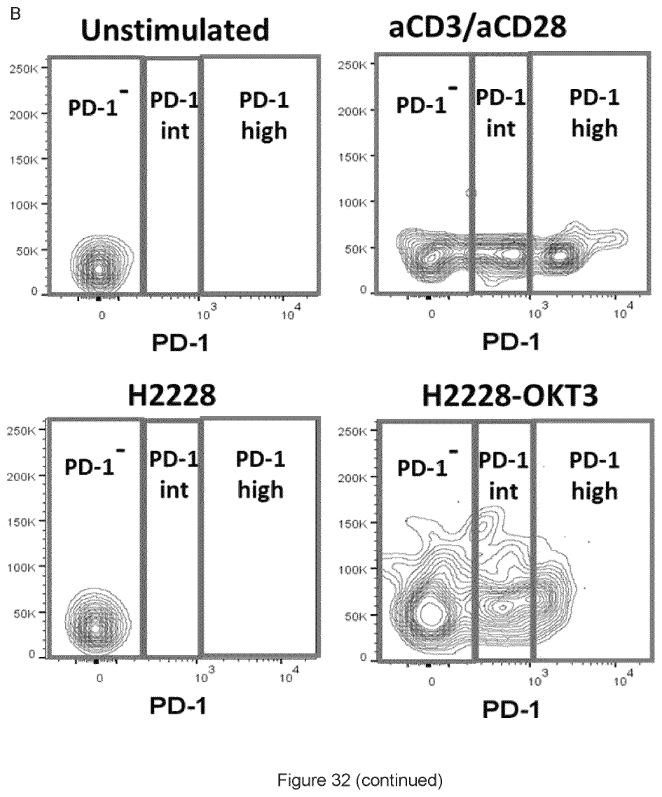

The gating strategy used to define the different PD1 populations in CD4 and CD8 T cells is illustrated on FIG. 32. FIG. 32A show the results for an exemplary KO (FURIN) for CD8 T cells. The plots show that in the unstimulated and H2228 conditions (negative controls), the PD1^{total} population is low (respectively 4.14% and 3.49%). By contrast, in the positive control condition (αCD3/acd28 beads) and the test condition with the aCD3 expressing tumour cells (H228-OKT3) the PD1^{total} population is higher (respectively 35.6% and 22.5%) indicating successful activation of the T CD8+ T cells in the sample as expected. Similar results are shown on FIG. 32B for the CD4+ T cells, where the unstimulated condition was associated with 7.59% PD1^{total} cells, the H2228 negative control condition was associated with 7.62% PD1^{total} cells, the aCD3/aCD28 condition was associated with 71.4% PD1^{total} cells, and the H2228-OXT3 condition was associated with 53.4% PD1^{total} cells. Thus, the data on FIG. 32 confirms both that the approach used and the gating strategy applied are appropriate to detect activation of PD-1 signalling, which is indicative of T cell activation.

Flow cytometry (Examples 3.5-3.7): Cells were washed with PBS and incubated with a mixture of flow cytometry monoclonal surface antibodies per 20 minutes at 4C protect from light. Non-viable cells were stained using the eBioscience Fixable Viability Dye eFluor 780 (Thermo, cat #65-0865-14). To detect intracellular epitopes cells were fixed and permeabilized using the Fixation/Permeabilization Solution Kit (Cat. No 555028, BD Biosciences) following the manufacturer protocol. In the case of staining for transcription factors, the eBioscience Foxp3/Transcription Factor Staining Buffer Set (eBioscience, Cat: 00-5523-00) was used following manufacturer protocol. Once stained, cells were resuspended in PBS and acquired using the BD FACS Symphony machine. Antibodies for flow cytometry were purchased from Biolegend, BD and ThermoFisher Scientific (eBioscience): KI67 (Cat. No 564071 BD Biosciences, Clone: B56), CD69 (Cat. No 564364 BD Biosciences, Clone: FN50), CD8 (Cat. No 564804 BD Biosciences, Clone: RPA-T8), CD45RA (Cat. No 565702 BD Biosciences, Clone: HI100), CD38 (Cat. No 565069 BD Biosciences, Clone: HIT2), CD39 (Cat. No 564726 BD Biosciences, Clone: TU66), SIRPγ (Cat. No 747683 BD Biosciences, Clone: OX-119), CD3 (Cat. No 565511 BD Biosciences, Clone: SK7), Ki-67 (Cat. No 350505 Biolegend, Clone: Ki-67), CD25 (Cat. No 356119 Biolegend, Clone: M-A251), CCR7 (Cat. No 353224 Biolegend, Clone G043H7), IL-2 (Cat. No 564166, BD Biosciences, Clone: MQ1-17H12), LAMP-1 (Cat. No 328640 Biolegend, Clone: H4A3), CD4 (Cat. No 317442 Biolegend, Clone: OKT4), CD4 (Cat. No 563877 BD Biosciences, Clone: SK3), TIM3 (Cat. No 565566 BD Biosciences, Clone: 7D3), GMCSF (Cat. No 502312, Biolegend, Clone: BVD2-21C11), LAG3 (Cat. No 61-2239-42, eBioscience, Clone: 3DS223H), PD-1 (Cat. No 329618, Biolegend, Clone: EH12.2H7), CD28 (Cat. No 11-0289-42 eBiosicence, Clone: CD28.2), LAG3 (Cat. No 369312 Biolegend, Clone: 11C3C65), TCF1 (Cat. No 655208 BD Biosciences, Clone: 7F11A10), 41BB (Cat. No 309826 Biolegend, Clone: 4B4-1), FCRL3 (Cat. No 374409 Biolegend, Clone: H5/FcrL3), FURIN (Cat. No IC1503R-100UG, R&D systems-Biotechne), SIT1 (Cat. No 367804 Biolegend, Clone: SIT-01), GZMB (Cat. No 367804 Biolegend, Clone: QA16A02), TNFα (Cat. No 17-7349082 Invitrogen, Mab11), IFNγ (Cat. No 12-7319-42, Clone 4S.B3), IL2 (Cat. No 17-7029-82 eBioscience, Clone: MQ1-17H2). Data was acquired on a BD Symphony flow cytometer and analyzed in FlowJo v10.5.3 (Treestar).

Target validation in samples from lung cancer patients. Tissue samples were collected and transported in RPMI-1640 (Cat. No R0883-500ML, Sigma). Single-cell suspensions were produced by enzymatic digestion using a Liberase TL (Cat. No 05401127001, Roche) and DNase I (Cat #11284932001, Roche) with subsequent cellular disaggregation using a Miltenyi gentleMACS OctoDissociator. Lymphocytes were isolated from single-cell suspensions by gradient centrifugation on Ficoll Paque Plus (Cat. No 17-1440-03, GE Healthcare), cryopreserved in fetal bovine serum (Cat. No 10270-106, Gibco) containing 10% DMSO (Cat. No D2650-100ML, Sigma) and stored in liquid nitrogen. Tumour infiltrating lymphocytes obtained from stage IV lung cancer patients were stained as explained before using monoclonal antibodies for each one of the proposed targets. The expression of the proposed targets was assessed in the different populations of CD4 and CD8 T cells. The activation and proliferation patterns of the cells were identified by the expression of Ki67, CD25, CD69, PD-1, TIM3 and other markers of T cell differentiation. Their functionality was determined by the expression of LAMP-1, GM-CSF, GZMB and the cytokines IFNγ and IL-2.

Rapid Expansion Protocol. Tumour Infiltrating Lymphocytes (TILs) were expanded to reach large numbers using OKT3 stimulation and a high dose of interleukin 2 (IL-2) in the presence of feeder cells as established by Dudley et al. 20031. To prepare the feeder cells, peripheral blood mononuclear cells were isolated from blood of healthy donors by gradient centrifugation using Ficoll-paque Plus (Cat. No 17-1440-03, GE Healthcare). The PBMC obtained by the different donors were pooled together and irradiated with 50 Gy. The irradiated stock was resuspended in Fetal Bovine Serum with 10% Dimethyl sulfoxide (DMSO) and cryopreserved at −80° C. freezers. 1×106 TIL and irradiated feeder cells were thawed, mixed in 1:200 ratio and resuspended in a 175 cm2 tissue culture flask (Cat. No 83.3912.002, Starstedt)) which contained 75 mL complete growth media (RPMI 1640 (Cat. No 51536C, Sigma-Aldrich) supplemented with 5 mL of Penicillin-Streptomycin (Cat. No P4333, Sigma-Aldrich) and 50 mL of Human Serum (10% final conc) (Cat. No G7513-100 mL, Sigma-Aldrich)), 75 mL of serum-free AIM-V medium (Cat. No 12055091, Gibco) and 30 ng/ml OKT3 antibody (Cat. No BE0001-2, BioXcell). The flask was incubated upright at 37° C. in 5% CO2. On day two, 6000 IU/mL of IL-2 (Proleukin, Novartis) were added to the culture. From day five, the media was changed by removing 120 mL of the media and replacing it with fresh mixture of 1:1 GM/AIM-V and 6000 IU/mL IL-2. The cell concentration was determined every two days and the cultures were split as needed to maintain the cell density approximately at 1×106/mL.

Gene-editing of human T cells using CRISPR/Cas9 technology. Frozen peripheral blood mononuclear cells (PBMCs) were thawed and incubated overnight in a 12-well plate (Cat. No 353043, Falcon) containing 2 mL of growth media (RPMI 1640 (Cat. No 51536C, Sigma-Aldrich) supplemented with 5 mL of Penicillin-Streptomycin (Cat. No P4333, Sigma-Aldrich) and 50 mL of Human Serum (10% final conc) (Cat: G7513-100 mL, Sigma-Aldrich)) supplemented with 50 IU/mL of IL2 (Proleukin, Novartis). The following day, cells were transferred into a 12-well plate coated with 10 μg/mL of αCD3 antibody (Cat No. BE0001-2, BioXcell, Clone: OKT3) and cultured in 2 mL of growth media containing 100 IU/mL of IL2 and 10 μg/mL of αCD28 antibody (Cat. No BE0248, BioXcell, Clone: 9.3) for 72 hours. In the case of tumour infiltrating lymphocytes (TILs), at day ten of the REP, an aliquot of the cell suspension was retrieved and kept on ice. For both, PBMCs and TILs the sgRNA was prepared by mixing the Alt-R tracrRNA 200 μM (Cat. No 1072534 IDT) and the Alt-R crRNA 200 μM (custom-made, IDT) in a 0.6 mL tube and incubated 5 minutes at 95° C. The sgRNA was diluted to 61 μM with the Nuclease-Free Duplex Buffer (Cat. No 11050112, IDT) and then mixed with the Cas9 protein 61 μM (Cat. No 1081059 IDT) to form the ribonucleoprotein complex. For each knock-out condition 1×106 T cells were resuspended in the Lonza P3 Nucleofection Solution and were electroporated with the ribonucleoprotein complex (4D-Nucleofector X Kit Small, Cat. No V4XP-3032, Lonza) following the manufacturer protocol. Post-electroporation, PBMCs and TILs were rested in growth media containing 100 and 6000 IU/mL of IL-2 respectively.

In vitro reactivation of gene-edited T cells using low dose of αCD3 and αCD28 beads. Following gene-editing, 1×106 human T cells were kept in growth media containing 50 UI/mL of IL2 (Proleukin, Novartis) for 10 days and in a 96 well plate (Cat. No 163320, Thermo Scientific). Media was changed every two-three days by removing 100 μL of old media and adding 100 μL of fresh media. On day 10 1×106 cells were counted, stained with cell trace violet (Cat. No 34557, Thermofisher Scientific) and reactivated using a 1/800 dilution of αCD3/αCD28 dynabeads (Cat. No 32-14000, Gibco) for four days. On day four, cells were incubated with 1 μL/mL of GolgiPlug (Cat. No 555029, BD Biosciences) and then stained for flow cytometry. Flow cytometry staining was performed using the BD CytoFix/Cytoperm kit (Cat. No 554714 BD Biosciences) following the manufacturer protocol.

Co-culture of gene-edited T cells with H2228 tumour cells expressing OKT3. Following gene-editing TILs were rested and at day four cocultured with H2228-OKT3 or control cells. 2.5×104 H2228, H2228-OKT3 or 10:1 mixture (H2228/H2228-OKT3) were seeded in 96-well flat-bottom plates (Cat. No ThermoFischer). 5×104 gene-edited T cells were added on top of the cancer cells (1:2 ratio). 5×104 edited T cells were seeded and activated using αCD3/αCD28 dynabeads (Cat. No 32-14000, Gibco) following the manufacturer protocol. 5×104 edited T cells were also activated with PMA (Cat. No P1585, Sigma-Aldrich)/Ionomycin (Cat. No I0634-1MG, Sigma-Aldrich) for 4 hours prior to staining. Unstimulated T cells were also kept as control. The cultures were incubated for 72 hours at 37° C., 5% CO2. The day of the readout, 1 μg/mL of GolgiPlug (Cat. No 555029, BD Biosciences) was added to the cells for 4 hours. Following incubation, the cells were stained for flow cytometry. Readouts were performed at 24, 48 and 72 hours.

### Example 3.1—Targets are Expressed in Tumour Infiltrating Lymphocytes in a Population—Specific Manner

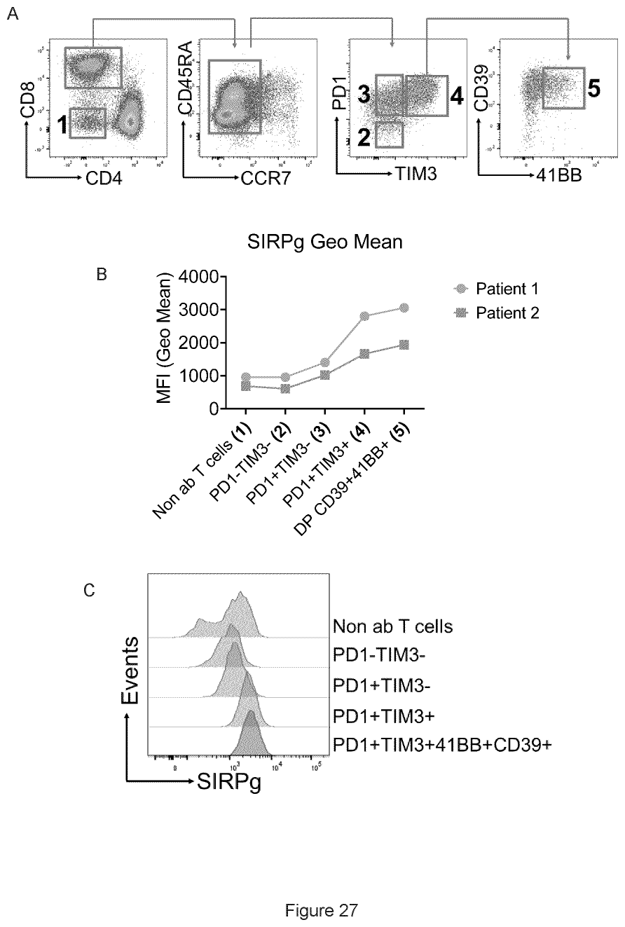

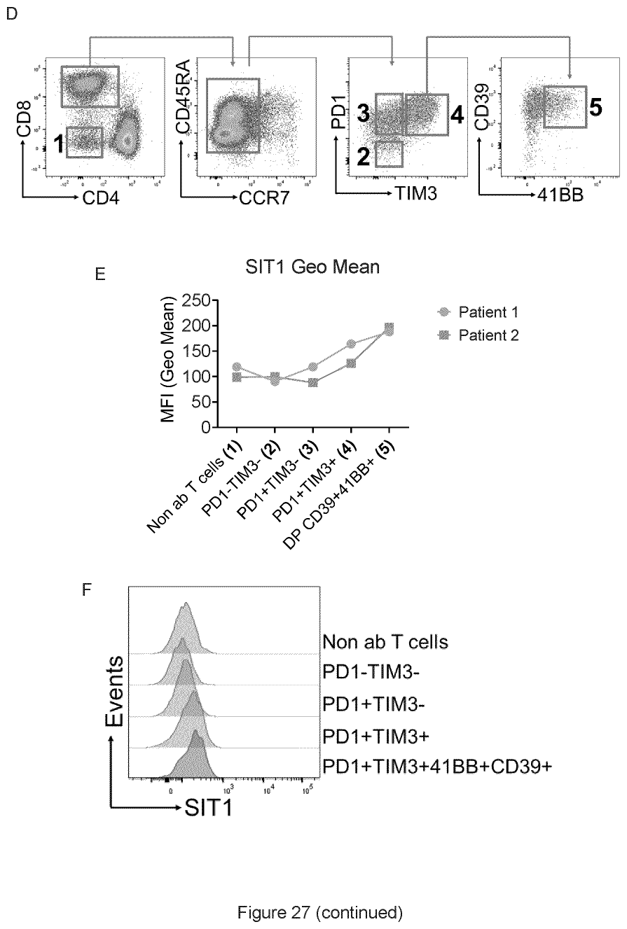

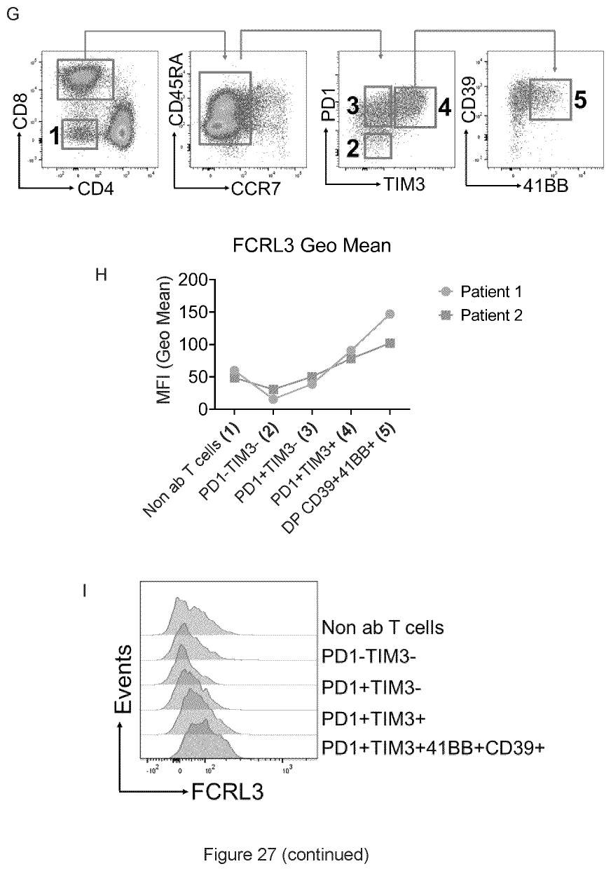

Tumour infiltrating lymphocytes obtained from stage IV non-small cell lung cancer were analysed by flow cytometry. SIRPG, SIT1 and FCRL3 expression was analysed on different subsets of T cells, non αβ T cells (population 1), PD1−TIM3−CD8 T cells (Non-tumour reactive, population 2), PD1+TIM3−CD8 T cells (tumour reactive, non-exhausted, population 3), PD1+TIM3+CD8 T cells (Exhausted CD8 T cells, population 4) and PD1+TIM3+CD39+41BB+CD8 T cells (Neoantigen reactive CD8 T cells, population 5). The expression of each protein was analysed in two different patients and its mean fluorescence intensity was graphed and shown in the graphs on FIGS. 27c,f and i for each subset of cells.

### Example 3.2—Gene Knock—Outs are Obtained for Selected Targets

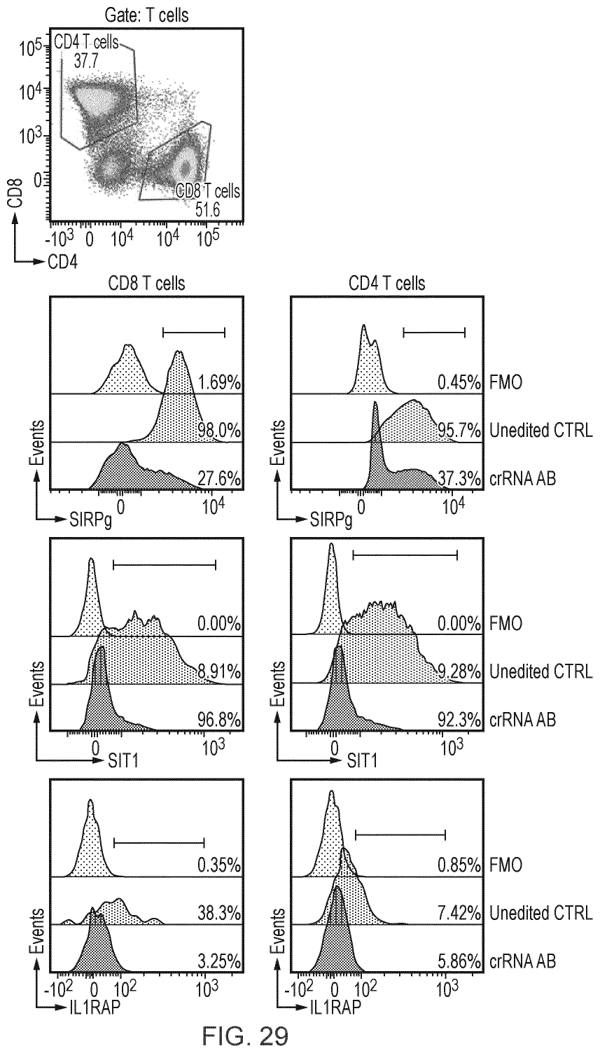

Human peripheral blood mononuclear cells, were stimulated for three days using αCD3 and αCD28 antibodies. On day three, cells were electroporated with the Cas9 protein and with the crRNA targeting each of the target genes shown (SIRPg, SIT1, IL1RAP). FIG. 29 shows, in the T cell populations (CD8 and CD4 T cells) identified in the plot in the top left corner, the signal (number of events) for each target gene in the FMO (fluorescence minus one) control (top curve in each plot), the unedited control (middle curve in each plot) and the edited cells (bottom curve in each plot), together with the associated frequencies of positive cells indicated as percentages next to the respective curves. This data shows that the knock-out strategy applied in this example effectively enables the modulation of expression of the targets in CD4 and CD8 cells.

### Example 3.3—Knock-Out T Cells Show Increased Production of IFNγ Following In Vitro Restimulation

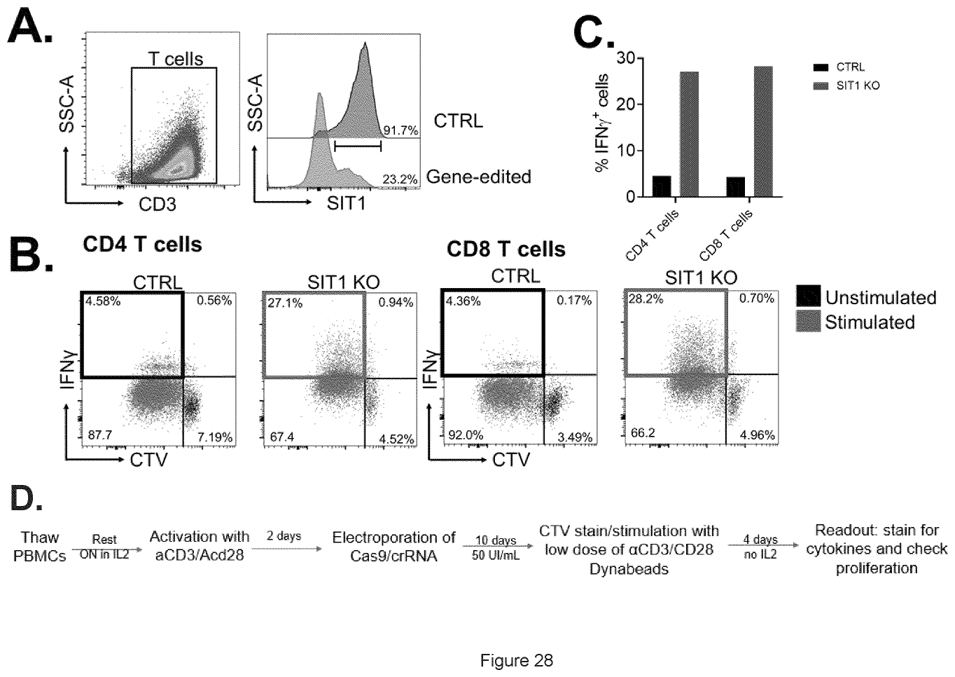

As illustrated on FIG. 28D, human peripheral blood mononuclear cells, were stimulated for three days using αCD3 and αCD28 antibodies. On day three, cells were electroporated with the Cas9 protein and with the crRNA targeting SIT1. Cells were kept in culture for 10 days using low doses of interleuquin 2. On day 10 cells were stained with cell trace violet and restimulated for four days with a low dose of dynabeads containing αCD3 and αCD28. On day 14, cells were incubated with brefeldin A for four hours in order to accumulate cytokines. Cells were stained for flow cytometry and acquired in the FACS symphony. FIG. 28A shows the expression of SIT1 Knock-out on total CD3⁺ T cells after 14 days of culture. FIG. 28B show IFNγ⁺CD4 and CD8 cells that diluted cell trace violet (CTV), unstimulated cells were used as controls. FIG. 29C shows the quantification of IFNγ⁺ T cells, control non-edited versus SIT-1 knock-out. IFNγ production is a commonly accepted readout of T cell cytotoxicity, and is indicative of their ability to kill tumour cells (see Kaplan et al., 1998; Gao et al., 2016; Zaretsky et al., 2016). The data on FIG. 28 shows that reducing expression of SIT1 in T cells increases their cytotoxicity compared to control, after in vitro restimulation. This indicates that downregulation of SIT1 expression in T cells renders such T cells better able to kill cancer cells and/or limit or reduce tumour growth in a subject having a proliferative disorder.

### Example 3.4—SIT1 KO NSCLC TILs Acquire a Higher Proliferative Capacity Following Restimulation with CD3/CD28 Beads

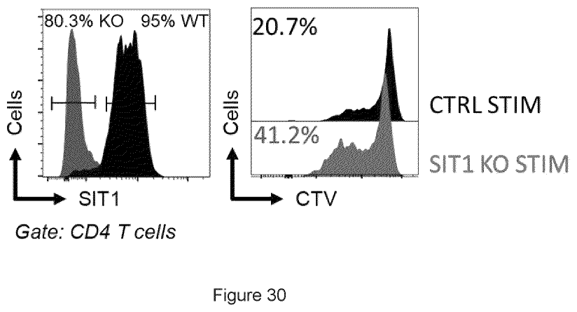

Tumour infiltrating lymphocytes obtained from NSCLC patients were KO and expanded for 21 days using a rapid expansion protocol (REP). On day 21 cells were stained with CTV and restimulated with a low dose of αCD3/CD28 beads. Four days later, CTV dilution was measured using Flow Cytometry. The results on FIG. 30 show that SIT1 Knock-out tumour infiltrating T cells acquire enhanced proliferative capacity. This is indicate of a potential effect of SIT modulation in potentiating an immune response as higher amounts of TILs (i.e. higher TIL proliferative capacity) should be associated with a stronger potential response.

### Example 3.5—H2228-OKT3 Coculture with PBMC-Derived Gene Edited Human T Cells Identifies Regulators of T Cell Activation

As illustrated on FIG. 31A, human PBMCs were modified by knocking out targets of interest (in particular, CD7, SIRPG and SIT1), then co-cultured with anti-CD3 expressing tumour cells (either 100% of cells, marked as “αCD3” or “H228-OXT3”, or 10% of cells, marked as “αCD31:10” or “ 1/10H228-OXT3”) or the same tumour cells not expressing anti-CD3 (marked as “CTRL” or “H2228”). The use of 10% of anti-CD3 expressing tumour cells is believed to represent a more physiological condition than the use of a population of tumour cells that all express anti-CD3. The proportion of CD4 and CD8 cells that are: PD1+LAMP1−, PD1+LAMP1+, and PD1+TIM3+ was measured after 72 hours of coculture. The cells were also cultured in a negative control condition (unstimulated), and in two positive control conditions (stimulation with anti-cd3 anti-cd28 coated dynabeads, and stimulation with PMA and ionomycin which stimulates cytokine production but is not expected to upregulate PD1 or LAMP1). In each condition, a control cell population was used in which the cells were electroporated with a control non-targeting crRNA.

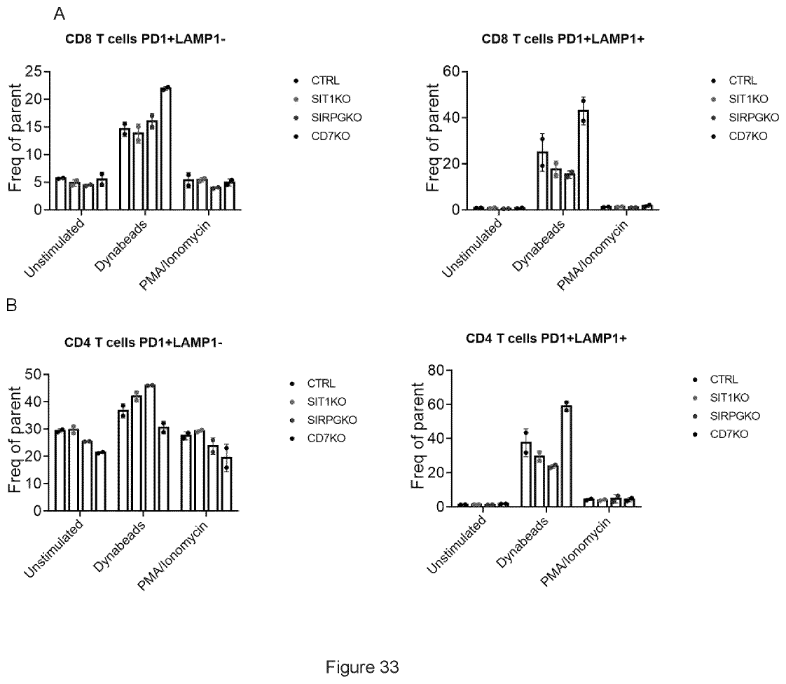

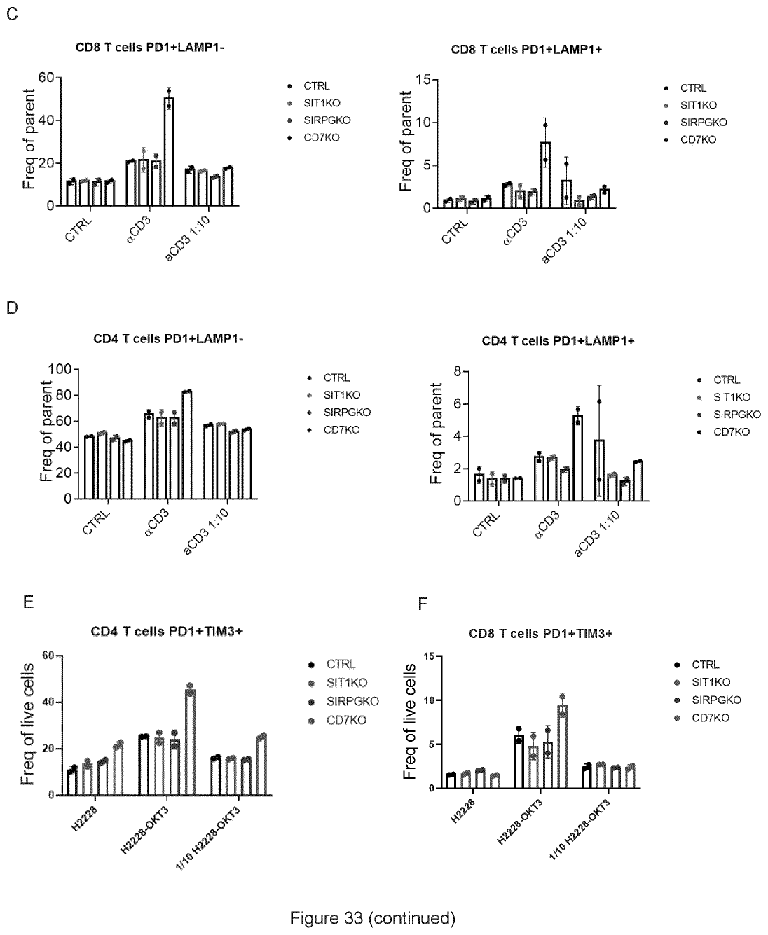

The results of these are shown on FIG. 33, where FIGS. 33A and B shows the results for the control conditions (anti-CD3, anti-CD28) and FIGS. 33C-F show the results for the cells incubated with the tumour cells. FIG. 33A shows that the CD7 and SIRPG KOs had an effect on PD1+LAMP1+CD8 cells when stimulated with the anti-CD3/anti-CD28 coated beads. In particular, the CD7 KO was associated with an increase in PD1+LAMP+CD8 T cells compared to the control crRNA. The SIRPGKO was associated with a decrease in PD1+LAMP+CD8 cells compared to the scrambled crRNA. FIG. 33B shows a similar picture in CD4 cells. FIG. 33C shows that coculture between tumour cells expressing αCD3 and PBMCs led to an upregulation of both PD1 and LAMP1 on CD8 T cells when CD7 was knocked-out. FIG. 33C also shows that the SIRPGKO had an effect on PD1+LAMP1+CD8 T cells at least in the αCD3 condition. FIG. 33D shows that coculture between tumour cells expressing αCD3 and PBMCs led to an upregulation of both PD1 and LAMP1 on CD4 T cells when CD7 was knocked-out. FIG. 33D also shows that the SIRPG KO also had an effect on PD1+LAMP1+CD4 cells. FIG. 33E shows that coculture between tumour cells expressing αCD3 and PBMCs led to an upregulation of PD1 and TIM3 on CD4 T cells when CD7 was knocked-out. FIG. 33F shows that a similar picture is present in CD8 T cells.

Thus, this data shows that CD7 knock out leads to upregulation of PD1, TIM3 and increased cytokine production in PBMCs following coculture with anti-CD3 expression tumour cells. PD1 and TIM3 are negative regulators of T cell activation, that are upregulated when T cells are activated, to avoid dying by apoptosis (Activation Induced Cell Death). This suggests that CD7 may also act as an activation break that when is deleted T cells need to press their alternative breaks (PD1 and TIM3) to avoid going into apoptosis as a result of overactivation. This data therefore indicates that modulation of CD7, in particular negative modulation, is likely to enhance an immune response in a therapeutic context. The data also suggests that SIRPG may have a role in T cell co-stimulation as the T cells appear to be less activated when this gene is knocked out. Thus, the assay suggests that activation/upregulation of SIRPG may be a promising therapeutic strategy for enhancing an immune response. In this assay, no effect was observed for SIT1. However, an effect was observed in other assays, as explained in Examples 3.3 and 3.4. The assay used here differs from the assay used in Examples 3.3 and 3.4 in multiple ways. In particular, the present assay uses a single stimulation with anti-CD3 expressed on tumour cells which are complex systems that may produce a variety of other signals. By contrast, the assay used in Examples 3.3 and 3.4 used a double stimulation with anti-cd3 and anti-cd28 on beads, which replicates both the signal that occurs upon T cell receptor-MHC-peptide interaction (ending on CD3 signalling) and the co-stimulation involving CD28 signalling but provides no further signal as the beads themselves are inert. Thus, it seems likely that in the present system the tumour cells themselves produce additional signals that inhibit T cell activation, such as e.g. PD-L1 signalling, thereby masking the effect of the SIT1 KO. Further, the dynabeads control here is also not directly comparable to the stimulation in Example 3.3. Indeed, the resting time of the cells was shorter here (4 days) than in the assay in Example 3.3 (10 days), and the concentration of beads was higher.

### Example 3.6—H2228-OKT3 Coculture with NSCLC TILs Identifies Regulators of PD1 Signalling

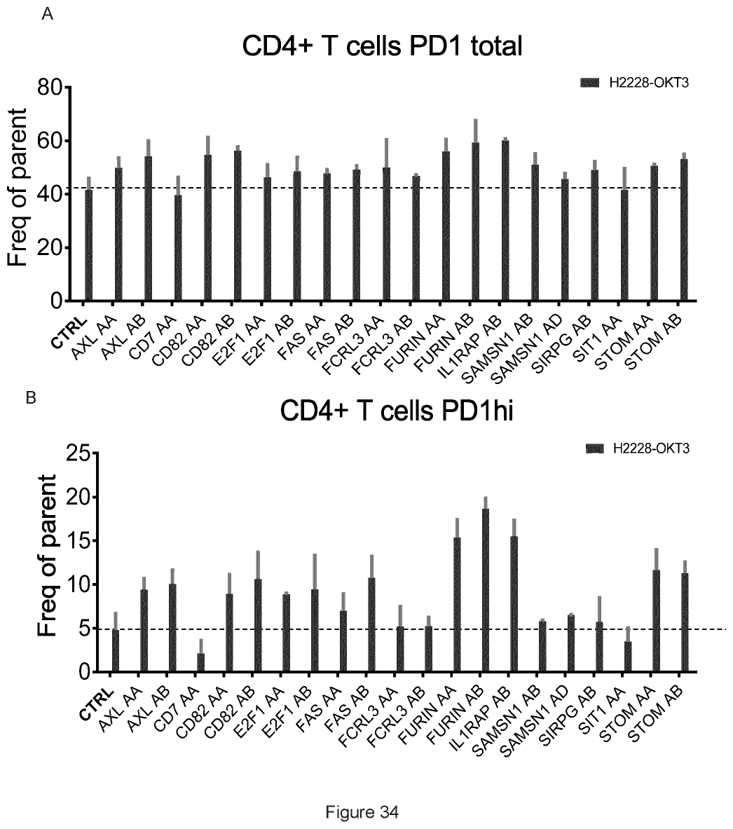

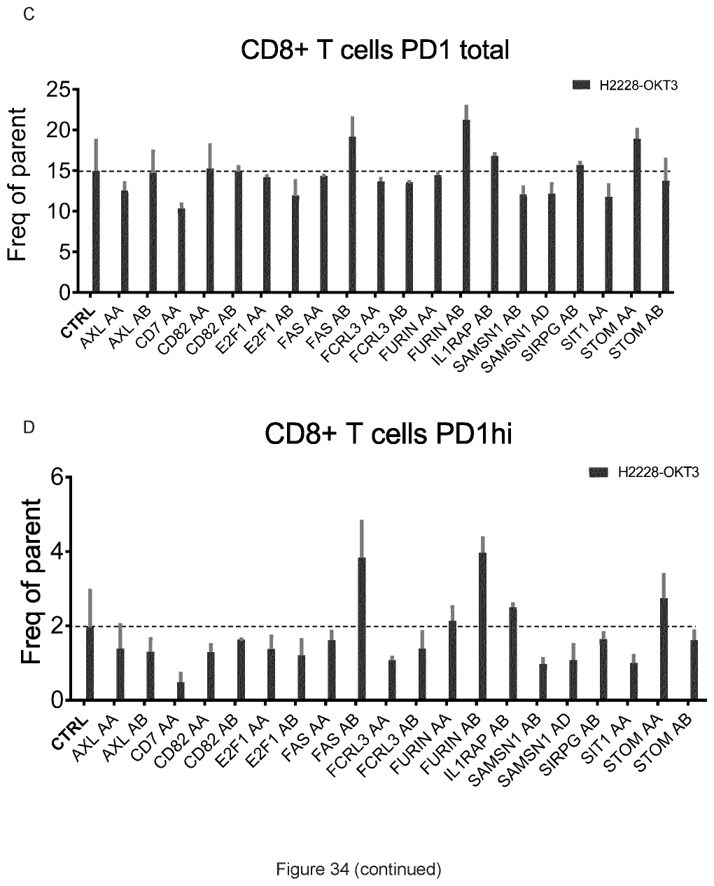

Human NSCLC TILs were modified by knocking out targets of interest, then the modified TILs were co-cultured with anti-CD3 expressing lung tumour cells. The proportion of PD1+ cells was measured after 72 hours of coculture using flow cytometry. The results of this are shown on FIG. 34. The total amount of PD1+ cells showed no major difference in CD4 (FIG. 34A) or CD8 T cells (FIG. 34C). The PD-1^{high} population showed an increase on CD4 cells (FIG. 34B) for at least FURIN, STOM, IL1RAP, AXL, CD82 and E2F1A. The PD-1^{high} population showed an increase in CD8 T cells (FIG. 34D) for at least FURIN, IL1RAP and STOM. Thus, the data indicates a higher level of activation following the knock-out of all of these genes in TILs. This data therefore indicates that modulation of these genes, in particular negative modulation, is likely to enhance an immune response in a therapeutic context.

Note that when only a single construct is illustrated (as is the case for CD7, IL1RAP and SIT1), that is because the construct was functionally validated. In all other cases two constructs were used because the constructs were only validated in silico. Thus, a lack of effect in this assay (whether in one or both constructs) may simply reflect a poor knock-out performance of the construct. Further, we note that these assays did not show an effect associated with CD7 knock-out, whereas an effect was observed in PBMC-derived cells in Example 3.5. PBMC cells and TILs are different cell populations that may be regulated differently (Scott et al., 2019; Brummelman et al., 2019). In particular, TILs are differentiated/exhausted cells. Therefore, the data indicates that modulation of CD7 in TILs may be a less promising strategy than its modulation in other cells, for example in the context of engineered T cells such as CART and TCR transduced T cells (see e.g. D'Angelo et al., 2018) or any modulation that does not rely exclusively on TILs (such as for example through the use of small/large molecule inhibitor). In other words, just because a gene modulation is not able to activate TILs that are exhausted cells does not mean that it will not be able to activate cells that have not yet reached this state. However, strategies that have a demonstrated effect in the context of TILs are likely to be useful in any context not limited to TILs. This is because even other types of cells that may not be differentiated/exhausted to start with will become exhausted. Thus, a gene modulation this is effective in TILs may eventually prevent terminal differentiation or exhaustion in all types of adoptive T cell therapies.

### Example 3.7—Changes in T Cell Differentiation and Functionality of CD4 and CD8 NSCLC TILs KO after 72 Hours of Co-Culture with H2228-OKT3 Tumour Cells

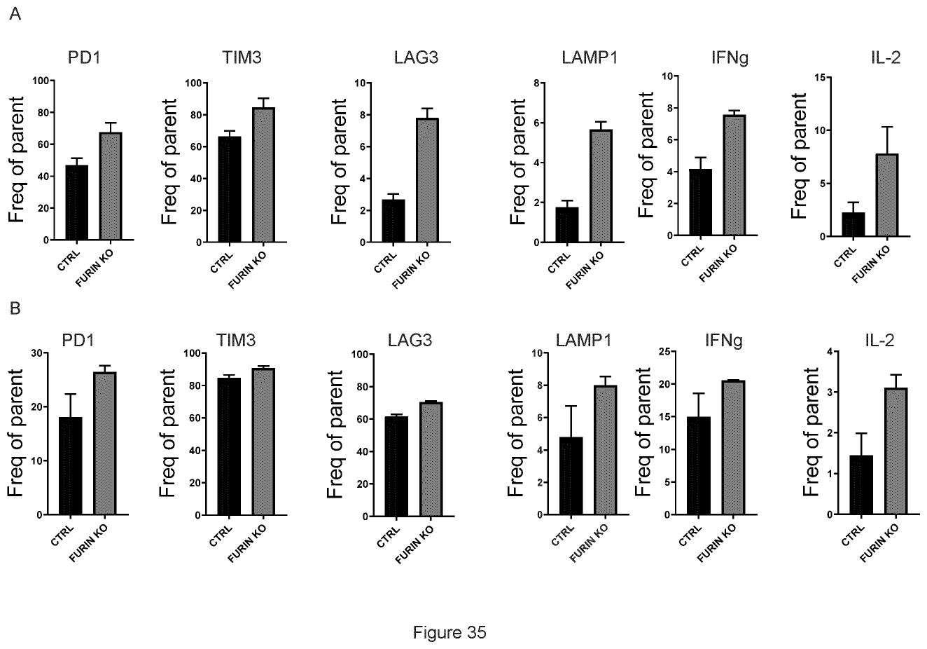

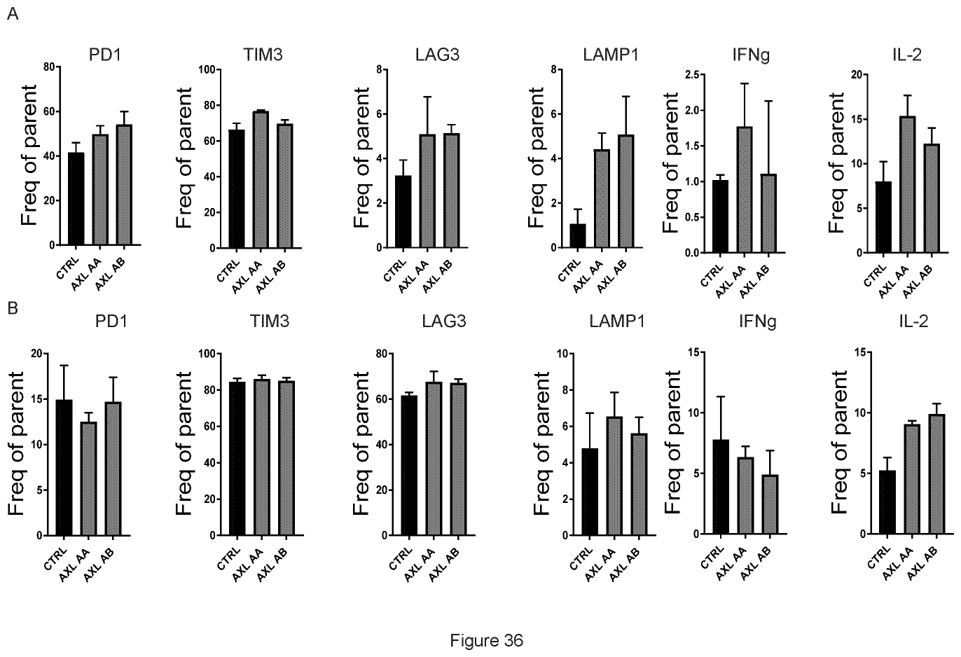

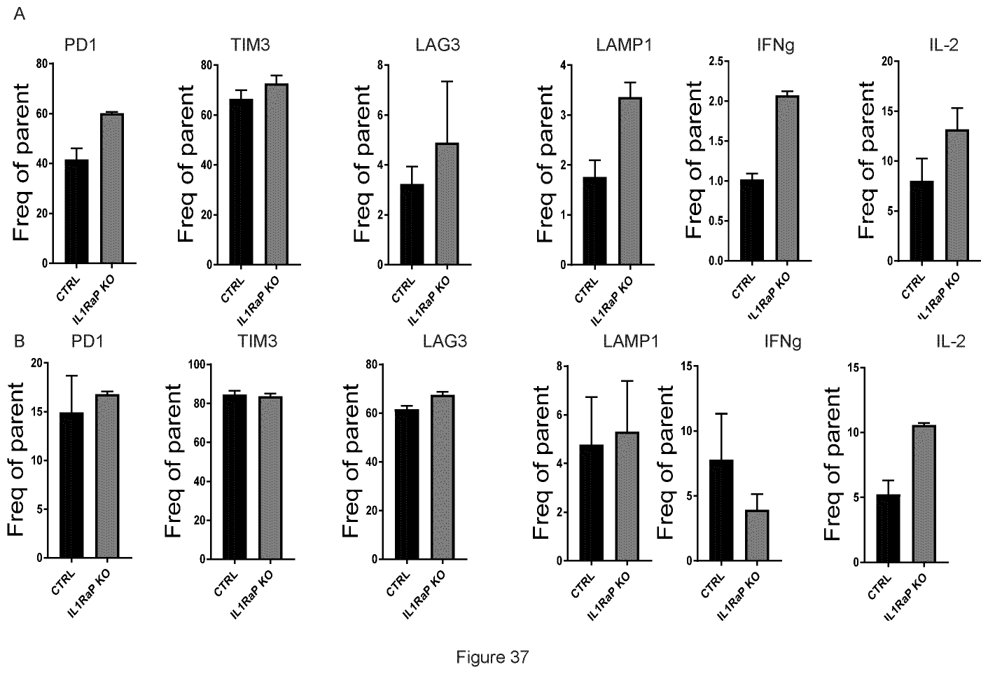

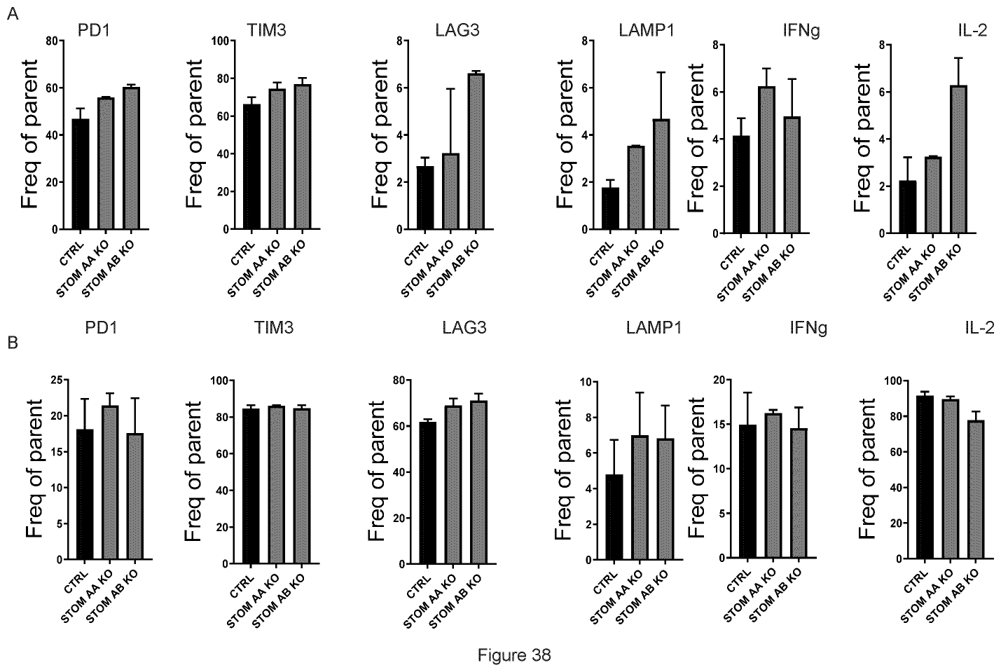

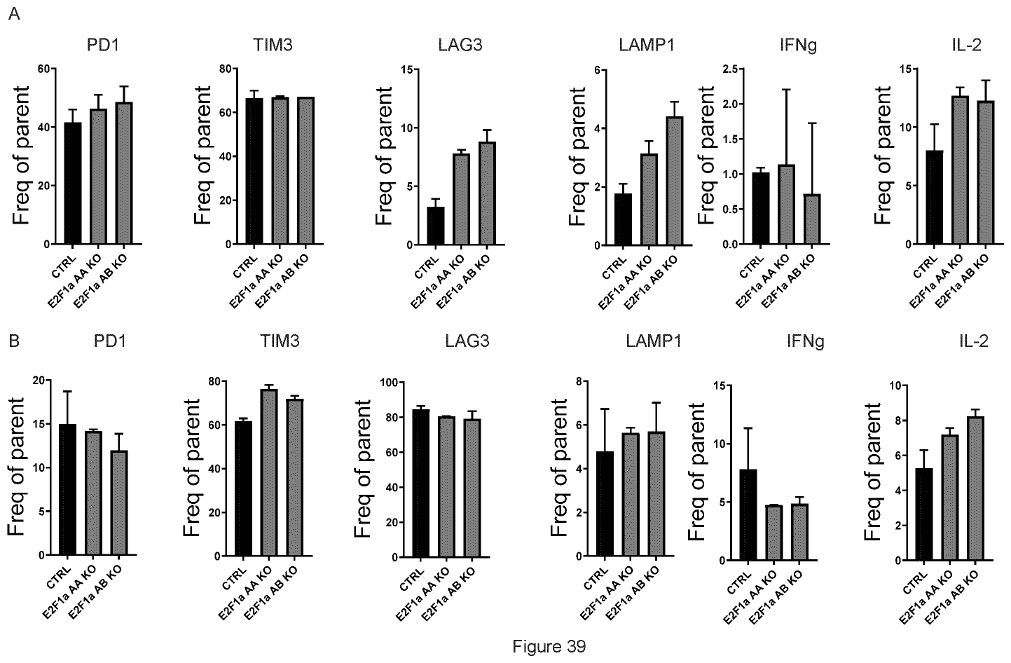

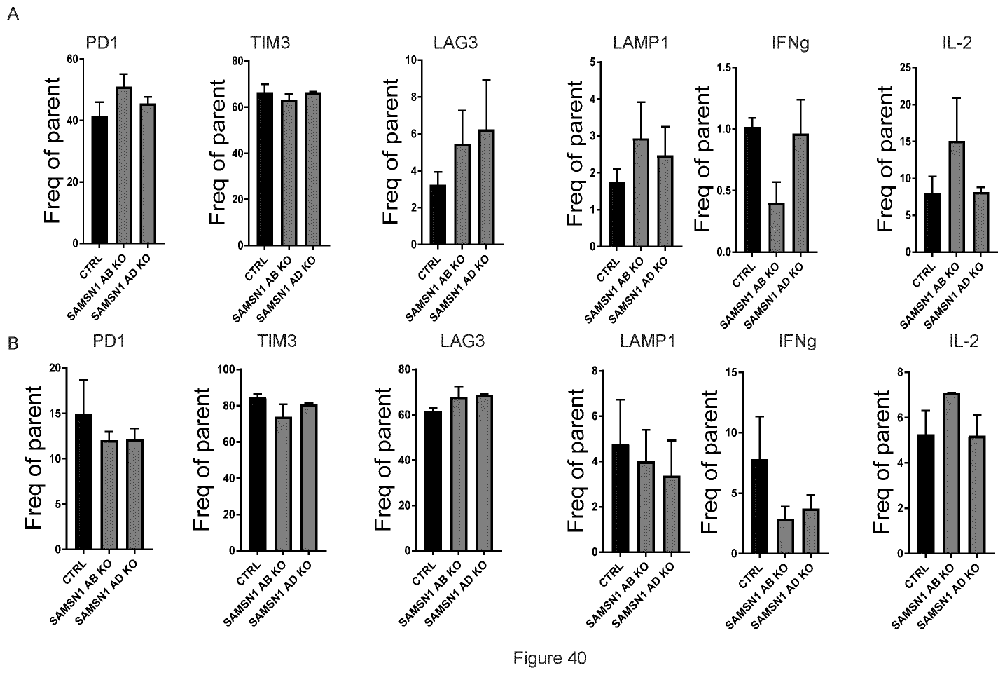

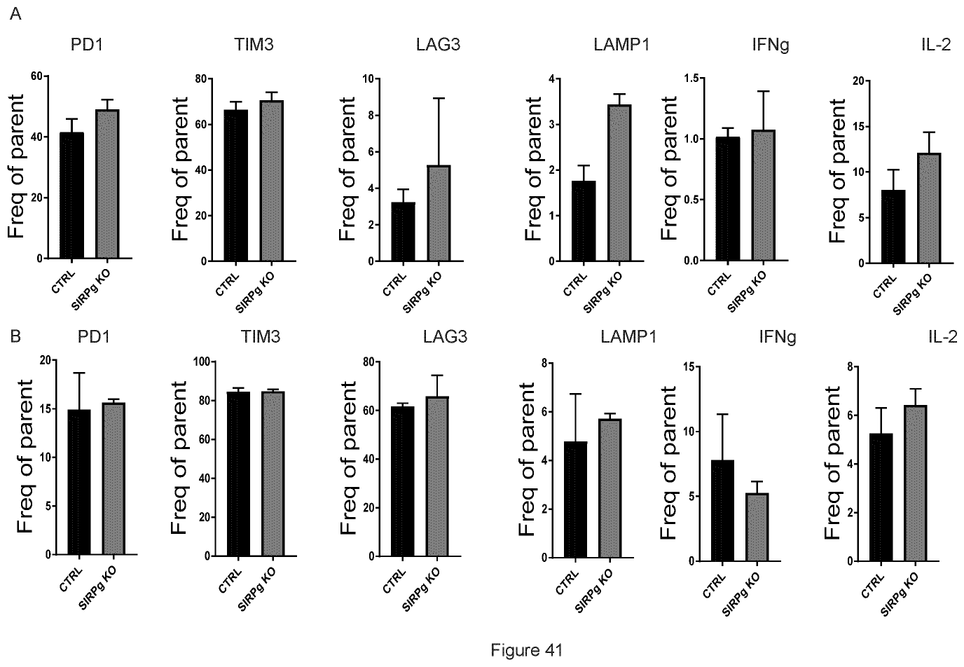

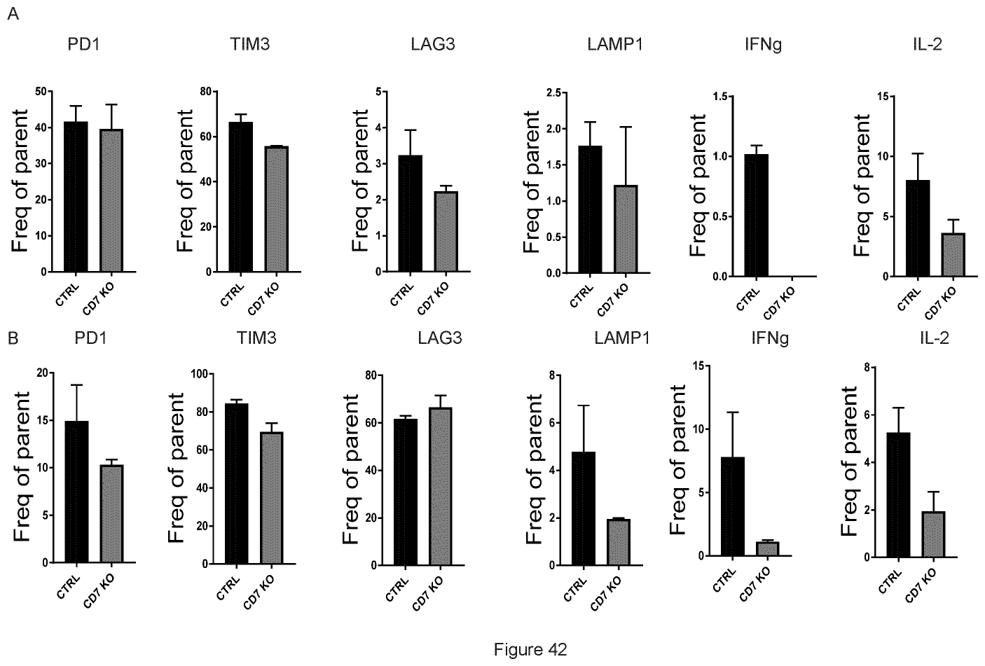

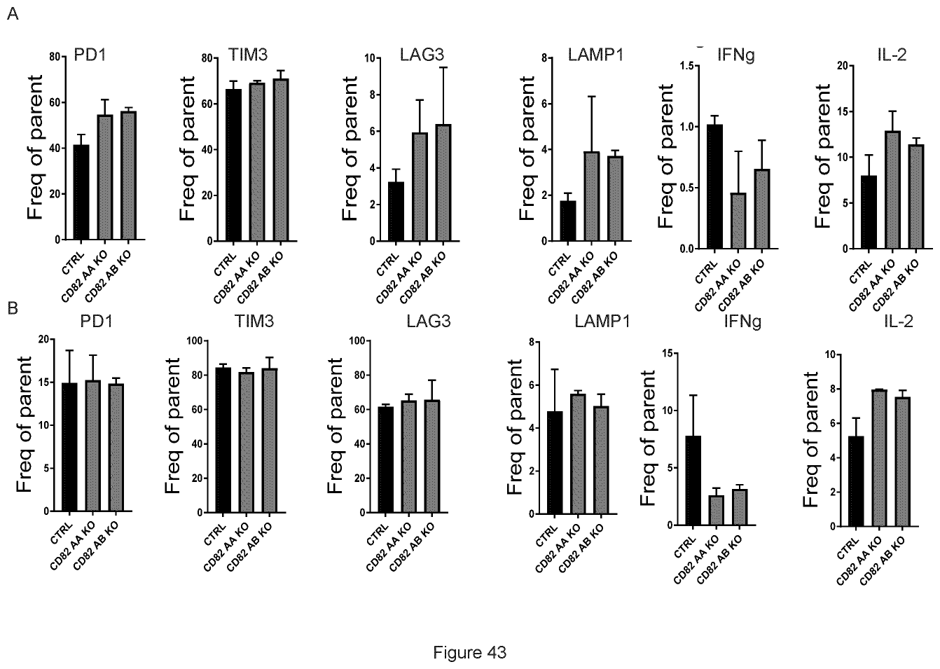

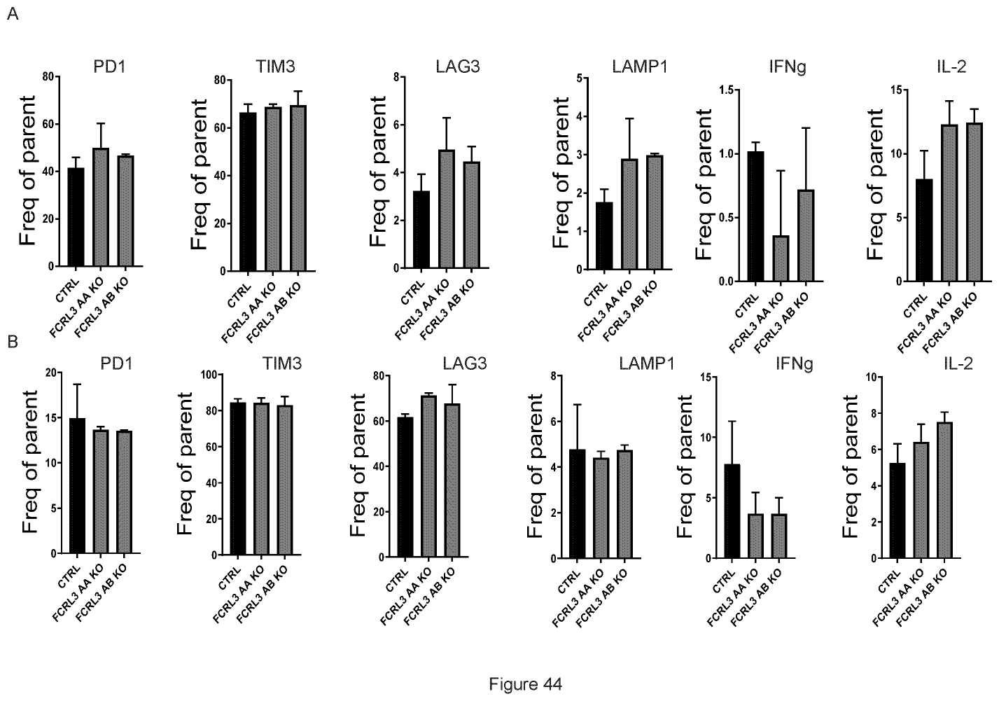

Human NSCLC TILs were modified by knocking out targets of interest, then the modified TILs were co-cultured with anti-CD3 expressing lung tumour cells. A series of markers of T cell differentiation and functionality were measured after 72 hours of coculture using flow cytometry. In particular, PD1, TIM3, and LAG3 are inhibitory receptors which are upregulated following T cell activation. As T cells get activated they upregulated these molecules to avoid Activation Induced Cell Death (AICD). Thus, if these genes are more expressed following KO of a target, this indicates that the target was relevant to T cell activation. LAMP1 is a degranulation marker. Its upregulation indicates that T cells produced and released cytokines. IFNg is an effector cytokine used by T cells to kill tumour cells. IL-2 is a cytokine produced by T cells following activation. IL-2 promotes T cell growth and survival. The results of this are shown on FIGS. 35 to 44 for the genes FURIN (FIG. 35), AXL (FIG. 36), IL1RAP (FIG. 37), STOM (FIG. 38), E2F1A (FIG. 39), SAMSN1 (FIG. 40), SIRPG (FIG. 41), CD7 (FIG. 42), CD82 (FIG. 43) and FCRL3 (FIG. 44). For each condition, the data shows the ° of cells positive for a certain marker (marked “Freq of parent”). The Mean fluorescence intensity (MFI) was also measured (data not shown). The Freq of parent is believed to be the most reliable measure in this assay. MFI is an indirect measure of expression and is believed to be affected by the machine behaviour so that it may be less reliable.

Note that the data for SIT1 (not shown) was consistent with the data in Example 3.5. No effect of the KO was seen on TILs in the presence of the anti-CD3 stimulation on tumour cells as opposed to the combined anti-CD3 anti-CD28 co-stimulation on beads which resulted in increased activation in the SITKO derived from PBMCs in Examples 3.3. and 3.4. Thus, the data confirms that modulation of SIT1, in particular negative modulation, is likely to enhance an immune response in a therapeutic context that does not rely directly on TILs, for example in the context of CART cell therapy or TCR T cell therapies that rely on the use of PBMCs.

FIG. 35A shows that the loss of FURIN on NSCLC TILs increased the PD1+, LAG3+, LAMP1+, IFNg+ and IL-2+CD4 T cell populations when the TILs were co-cultured with H2228-OKT3 expressing cells. A similar trend is observed on the KO CD8 T cell population (see FIG. 35B). This phenotype is indicative of a higher level of activation of T cells suggesting that FURIN has a role in both CD4 and CD8 T cell activation in the context of NSCLC TILs. Thus, this data indicates that modulation of FURIN, in particular negative modulation, is likely to enhance an immune response in a therapeutic context.

FIG. 36A shows that the loss of AXL on NSCLC TILs increased the PD1+, LAG3+, LAMP1+, IFNg+ and IL-2+CD4 T cell populations when TILs were co-cultured with H2228-OKT3 expressing cells. This phenotype is indicative of a higher level of activation of T cells which suggests that AXL has a role in CD4 T cell activation in the context of NSCLC TILs. Loss of AXL in the CD8 compartment did not result in the same change on the measured parameters, although changes in LAG3, LAMP1 and IL-2 were observed (FIG. 36B). Thus, this data indicates that modulation of AXL, in particular negative modulation at least in CD4 T cells, is likely to enhance an immune response in a therapeutic context.

FIG. 37A shows that the loss of IL1RaP on NSCLC TILs increased the PD1+, LAG3+, LAMP1+, IFNg+ and IL-2+CD4 T cell populations when TILs were co-cultured with H2228-OKT3 expressing cells. A similar trend is observed on the KO CD8 T cell populations (FIG. 37B). This phenotype is indicative of a higher level of activation of T cells which suggests that IL1RaP has an important role in both CD4 and CD8 T cell activation in the context of NSCLC TILs. Thus, this data indicates that modulation of AXL, in particular negative modulation, is likely to enhance an immune response in a therapeutic context.

FIG. 38A shows that the loss of STOMATIN on NSCLC TILs increased the PD1+, LAG3+, LAMP1+, IFNg+ and IL-2+CD4 T cell populations when TILs were co-cultured with H2228-OKT3 expressing cells. This phenotype is indicative of a higher level of activation of T cells which suggests that STOMATIN has a role in T cell activation in the context of NSCLC CD4 TILs. Loss of STOMATIN in the CD8 compartment did not result in such a clear picture primarily due to noise in the data, although increases in PD1, LAG3, LAMP1 and possibly IFNg may be present (FIG. 38B). Thus, this data indicates that modulation of STOMATIN, in particular negative modulation at least in CD4 T cells, is likely to enhance an immune response in a therapeutic context.

FIG. 39A shows that the loss of E2F1a on NSCLC TILs increased the PD1+, LAG3+, LAMP1+, IFNg+ and IL-2+CD4 T cell populations when TILs were co-cultured with H2228-OKT3 expressing cells. This phenotype is indicative of a higher level of activation of T cells which suggests that E2F1a has an important role in CD4 T cell activation in the context of NSCLC TILs. Loss of E2F1a in the CD8 compartment did not change the markers of T cell activation that were measured in this assay to the same extent when compared with the control KO CD8 NSCLC TILs (FIG. 39B). However, increases in TIM3, LAMP1 and IL-2 appear to also occur in the CD8 compartment. Thus, this data indicates that modulation of E2F1A, in particular negative modulation at least in CD4 T cells, is likely to enhance an immune response in a therapeutic context.

FIG. 40A shows that the loss of SAMSN1 on NSCLC TILs increased the PD1+, LAG3+, LAMP1+ and IL-2+CD4 T cell populations when TILs were co-cultured with H2228-OKT3 expressing cells. This phenotype is indicative of a higher level of activation of T cells which suggests that SAMSN1 has an important role in CD4 T cell activation in the context of NSCLC TILs. Loss of SAMSN1 in the CD8 compartment did not change the markers of T cell activation that were measured in this assay to the same extent when compared with the control KO CD8 NSCLC TILs (FIG. 40B). However, increases in LAG3 and IL-2 appear to also occur in the CD8 compartment. Thus, this data indicates that modulation of SAMSN1, in particular negative modulation at least in CD4 T cells, is likely to enhance an immune response in a therapeutic context.

FIG. 41A shows that the loss of SIRPG on NSCLC TILs increased the PD1+, TIM3, LAG3+, LAMP1+ and IL-2+CD4 T cell populations when TILs were co-cultured with H2228-OKT3 expressing cells. This phenotype is indicative of a higher level of activation of T cells which suggests that SIRPG has an important role in CD4 T cell activation in the context of NSCLC TILs. Loss of SIRPG in the CD8 compartment did not change the markers of T cell activation that were measured in this assay to the same extent when compared with the control KO CD8 NSCLC TILs (FIG. 41B). However, increases in LAG3 and IL-2 appear to also occur in the CD8 T cells compartment. Thus, this data indicates that modulation of SIRPG, in particular negative modulation at least in CD4 T cells, is likely to enhance an immune response in a TIL therapeutic context. We note that the data in Example 3.5 indicated a decrease of the PD1+LAMP1+ population of CD4 and CD8 T cells in PBMC derived cells. This may indicate that the effect of this gene is not the same in TILs and in PBMC-derived cells. Different isoforms of SIRPG exist, which have been shown to the expressed differently between different subsets of T cells (Li, Zhang & Ren, 2020). Thus, the isoform expressed on PBMCs nay be different from the isoform expressed on TILs, which may result in different function, explaining the difference in effect of SIRPGKO in this data and that of Example 3.5. Thus, although all of the data available suggests that modulation of SIRPG is likely to enhance an immune response in a therapeutic context, the data indicates that negative modulation may be effective in the context of TIL in particular, while in other contexts positive modulation may be effective.

FIG. 42A shows that the loss of CD7 on NSCLC TILs decreased the PD1+, LAG3+, LAMP1+, IFNg+ and IL-2+CD4 T cell populations when TILs were co-cultured with H2228-OKT3 expressing cells. This phenotype is indicative of a lower level of activation of T cells which suggests that CD7 has an important role in CD4 T cell activation in the context of NSCLC TILs. A similar trend is observed in CD8 T cells (FIG. 42B). Thus, this data indicates that modulation of CD7, in particular positive modulation, is likely to enhance an immune response in a TIL therapeutic context. We note that the data in Example 3.5 indicated an increase of the PD1+LAMP1+ population of CD4 and CD8 T cells in PBMC derived cells. This may indicate that the effect of this gene is not the same in TILs and in PBMC-derived cells. Thus, although all of the data available suggests that modulation of CD7 is likely to enhance an immune response in a therapeutic context, the data indicates that positive modulation may be effective in the context of TIL in particular, while in other contexts negative modulation may be effective. This is consistent with the findings by Lee et al. (1998) that 3 months mice with a knock-out of CD7 had higher numbers of developing T cells (thymocytes) suggesting a role in proliferation or T cell activation.

FIG. 43A shows that the loss of CD82 on NSCLC TILs increased the PD1+, LAG3+, LAMP1+, IFNg+ and IL-2+CD4 T cell populations when TILs were co-cultured with H2228-OKT3 expressing cells. This phenotype is indicative of a higher level of activation of T cells which suggests that CD82 has an important role in CD4 T cell activation in the context of NSCLC TILs. Loss of CD82 in the CD8 compartment did not change the markers of T cell activation that were measured in this assay to the same extent when compared with the control KO CD8 NSCLC TILs (FIG. 43B). However, increases in LAMP1 and IL-2 appear to also occur in the CD8 compartment. Thus, this data indicates that modulation of CD82, in particular negative modulation at least in CD4 T cells, is likely to enhance an immune response in a therapeutic context.

FIG. 44A shows that the loss of FCRL3 on NSCLC TILs increased the PD1+, LAG3+, LAMP1+, and IL-2+CD4 T cell populations when TILs were co-cultured with H2228-OKT3 expressing cells. This phenotype is indicative of a higher level of activation of T cells which suggests that CD82 has an important role in CD4 T cell activation in the context of NSCLC TILs. Loss of CD82 in CD8 T cells did not change the markers of T cell activation that were measured in this assay to the same extent when compared with the control KO CD8 NSCLC TILs (FIG. 44B). However, increases in LAG3 and IL-2 appear to also occur in the CD8 T cells. Thus, this data indicates that modulation of FCRL3, in particular negative modulation at least in CD4 T cells, is likely to enhance an immune response in a therapeutic context.

### Example 3—Conclusion

The data in this example indicates that all of the targets tested (SIT1, CD7, SIRPg, FURIN, STOM, IL1RAP, AXL, CD82, E2F1A, SAMSN1 and FCRL3) out of the set of targets identified (SIT1, SAMSN1, SIRPG, CD7, CD82, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3) showed some evidence of being involved in T cell activation. In other words, every single one of the targets identified that was tested was validated as a potential target for modulation to enhance an immune response in a therapeutic context. Thus, the data indicates that all of the targets identified (including those that have been validated and those that have not yet been validated due to time constraints) are likely to be useful targets for modulation to enhance an immune response in a therapeutic context.

In particular, the data in Example 3.3. indicates that negative modulation of SIT1 in PBMC derived T cells results in an increase in production of IFNg upon in vitro stimulation with aCD3/aCD28. The data in Example 3.4 indicates that negative modulation of SIT1 in TILs results in an increased proliferative capacity following restimulation with antiCD3/antiCD28. The data in Examples 3.5 and 3.7 indicates that negative modulation of SIT1 in PBMCs and TILs may not result in increased T cell activation upon CD3 stimulation alone in the context of a tumour cancer cell line that in addition to the CD3 stimulation provides a suppressive microenvironment characterised by the expression of PDL1 amongst other proteins that inhibit T cell function. This data indicates that modulation of SIT1, in particular negative modulation, is likely to enhance an immune response in a therapeutic context at least in contexts where such a suppressive microenvironment is not present or can be mitigated. For example, negative modulation of SIT1 is likely to enhance an immune response in a therapeutic context that does not rely directly on TILs, for example in the context of CART cell therapy or TCR T cell therapies that rely on the use of PBMCs (e.g. TCR transduced T cells, see e.g. D'Angelo et al., 2018).

The data in Example 3.5 indicates that negative modulation of CD7 in PBMCs results in increased T cell activation. The data in Example 3.7 indicates that negative modulation of CD7 in TILs results in decreased T cell activation. Thus, this data indicates that modulation of CD7 is likely to enhance an immune response in a therapeutic context, particularly when negative modulation is used except in the context of TILs where positive modulation may be preferable. Positive modulation may be obtained by cell engineering or by stimulation with an agonist.

The data in Examples 3.6 and 3.7 indicates that negative modulation of STOM, FURIN and IL1RaP in TILs results in increased T cell activation in both CD4 and CD8 T cells. This data indicates that modulation of STOM, FURIN and IL1RAP, in particular negative modulation, is likely to enhance an immune response in a therapeutic context. The data in Examples 3.6 and 3.7 indicates that negative modulation of AXL, E2F1A, CD82, SAMSN1, and FCRL3 in TILs results in increased T cell activation at least in CD4 T cells and possibly also in CD8 T cells.

The data in Example 3.5 indicates that negative modulation of SIRPG in PBMCs results in decreased T cell activation in both CD4 and CD8 T cells. The data in Example 3.7 indicates that negative modulation of SIRPG in TILs results in increased T cell activation in at least CD4 T cells and possibly also CD8 T cells. Thus, this data indicates that modulation of SIRPg is likely to enhance an immune response in a therapeutic context, particularly when positive modulation is used except in the context of TILS where negative modulation may be preferable. Positive modulation may be obtained by cell engineering or by stimulation with an agonist.

Positive modulation of any target gene described herein may be achieved by modifying target cells to increase expression of the target (e.g. using engineered immune cells). Instead or in addition to this, positive modulation of any target gene described herein may be achieved using an agonist antibody. Agonist antibodies having been shown to be promising for cancer immunotherapy (see e.g. Sakellariou-Thompson et al., 2017). Instead or in addition to this, positive modulation of any target gene described herein and that is a receptor may be achieved using an agonist of the receptor. For example, SIRPbeta2 has been shown to be expressed on T cells and activated NK cells, and to bind CD47 on antigen-presenting cells, resulting in enhanced T cell proliferation (Piccio et al, 2005). Thus, for example, an agonist of SIRPg may similarly be used to positively modulate SIRPg, thereby enhancing an immune response.

Negative modulation of any target gene described herein may be achieved by modifying target cells to decrease (e.g. knock down or knock out) expression of the target (e.g. using engineered immune cells). Instead or in addition to this, negative modulation of any target gene described herein may be achieved using a blocking antibody or small molecule inhibitor. For example, inhibition of kinases such as AXL with small molecule inhibitors has been shown to be possible. Further, inhibition of other kinases such as p38 and MAP Kinase have been shown to promote increased T cell immunity (Ebert et al., 2016; Gurusamy et al., 2020).

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

### REFERENCES

Thommen, D. S. & Schumacher, T. N. T Cell Dysfunction in Cancer. Cancer Cell 33, 547-562 (2018).
Borst, J., Ahrends, T., Babała, N., Melief, C. J. M. & Kastenmüller, W. CD4+ T cell help in cancer immunology and immunotherapy. Nat. Rev. Immunol. 18, 635-647 (2018).
Schietinger, A., Philip, M., Liu, R. B., Schreiber, K. & Schreiber, H. Bystander killing of cancer requires the cooperation of CD4(+) and CD8(+) T cells during the effector phase. J. Exp. Med. 207, 2469-77 (2010).
Philip, M. et al. Chromatin states define tumour-specific T cell dysfunction and reprogramming. Nature 545, 452-456 (2017).
Schietinger, A. & Greenberg, P. D. Tolerance and exhaustion: Defining mechanisms of T cell dysfunction. Trends Immunol. 35, 51-60 (2014).
Hombrink, P. et al. Programs for the persistence, vigilance and control of human CD8⁺ lung-resident memory T cells. Nat. Immunol. (2016). doi:10.1038/ni.3589
MacKay, L. K. et al. The developmental pathway for CD103+CD8+ tissue-resident memory T cells of skin. Nat. Immunol. 14, 1294-1301 (2013).
Appay, V., Van Lier, R. A. W., Sallusto, F. & Roederer, M. Phenotype and function of human T lymphocyte subsets: Consensus and issues. Cytometry Part A (2008). doi:10.1002/cyto.a.20643
Hock, H., Dorsch, M., Diamantstein, T. & Blankenstein, T. Interleukin 7 induces CD4+ T cell-dependent tumour rejection. J. Exp. Med. 174, 1291-8 (1991).
Linnemann, C. et al. High-throughput epitope discovery reveals frequent recognition of neo-antigens by CD4+ T cells in human melanoma. Nat. Med. 21, 81-5 (2015).
Sahin, U. et al. Personalized RNA mutanome vaccines mobilize poly-specific therapeutic immunity against cancer. Nature 547, 222-226 (2017).
Tran, E. et al. Cancer Immunotherapy Based on Mutation-Specific CD4+ T Cells in a Patient with Epithelial Cancer. Science (80-.). 9, 641-645 (2014).
Zinkernagel, R. M. et al. Antigen localisation regulates immune responses in a dose- and time-dependent fashion: a geographical view of immune reactivity. Immunol. Rev. 156, 199-209 (1997).
Neefjes, J. & Ovaa, H. A peptide's perspective on antigen presentation to the immune system. Nat. Chem. Biol. 9, 769-775 (2013).
Zhu, J., Yamane, H. & Paul, W. E. Differentiation of Effector CD4 T Cell Populations. Annu. Rev. Immunol. 28, 445-489 (2010).
Van Allen, E. M. et al. Genomic correlates of response to CTLA-4 blockade in metastatic melanoma. Science (80-.). 350, 207-211 (2015).
Rizvi, N. A. et al. Mutational landscape determines sensitivity to PD-1 blockade in non-small cell lung cancer. Science (80-.). 348, 124-8 (2015).
van, A. et al. Genetic Basis for Clinical Response to CTLA-4 Blockade in Melanoma. N. Engl. J. Med. 371, 2189-2199 (2014).
Goodman, A. M. et al. Tumour Mutational Burden as an Independent Predictor of Response to Immunotherapy in Diverse Cancers. Mol. Cancer Ther. 16, 2598-2608 (2017).
McGranahan, N. et al. Clonal neoantigens elicit T cell immunoreactivity and sensitivity to immune checkpoint blockade. Science (80-.). 351, 1463-1469 (2016).
Ganesan, A. P. et al. Tissue-resident memory features are linked to the magnitude of cytotoxic T cell responses in human lung cancer. Nat. Immunol. (2017).
Rosenthal, R. et al. Neoantigen-directed immune escape in lung cancer evolution. Nature 567, 479-485 (2019).
Ciurea, A., Hunziker, L., Klenerman, P., Hengartner, H. & Zinkernagel, R. M. Impairment of CD4(+) T cell responses during chronic virus infection prevents neutralizing antibody responses against virus escape mutants. J. Exp. Med. 193, 297-305 (2001).
Harari, A., Petitpierre, S., Vallelian, F. & Pantaleo, G. Skewed representation of functionally distinct populations of virus-specific CD4 T cells in HIV-1-infected subjects with progressive disease: Changes after antiretroviral therapy. Blood 103, 966-972 (2004).
Day, C. L. et al. PD-1 expression on HIV-specific T cells is associated with T-cell exhaustion and disease progression. Nature 443, 350-4 (2006).
Kaufmann, D. E. et al. Upregulation of CTLA-4 by HIV-specific CD4+ T cells correlates with disease progression and defines a reversible immune dysfunction. Nat. Immunol. 8, 1246-1254 (2007).
Frenz, T. et al. CD4+ T cells in patients with chronic inflammatory rheumatic disorders show distinct levels of exhaustion. J. Allergy Clin. Immunol. 138, 586-589.e10 (2016).
Han, S., Asoyan, A., Rabenstein, H., Nakano, N. & Obst, R. Role of antigen persistence and dose for CD4+ T-cell exhaustion and recovery. Proc. Natl. Acad. Sci. 107, 20453-20458 (2010).
Jelley-Gibbs, D. M. et al. Repeated stimulation of CD4 effector T cells can limit their protective function. J. Exp. Med. 201, 1101-12 (2005).
Palmer, B. E., Boritz, E. & Wilson, C. C. Effects of sustained HIV-1 plasma viremia on HIV-1 Gag-specific CD4+ T cell maturation and function. J. Immunol. 172, 3337-47 (2004).
Crawford, A. et al. Molecular and transcriptional basis of CD4+ T cell dysfunction during chronic infection. Immunity 40, 289-302 (2014).
Fletcher, J. M. et al. Cytomegalovirus-specific CD4+ T cells in healthy carriers are continuously driven to replicative exhaustion. J. Immunol. 175, 8218-25 (2005).
Palmer, B. E., Blyveis, N., Fontenot, A. P. & Wilson, C. C. Functional and phenotypic characterization of CD57+CD4+ T cells and their association with HIV-1-induced T cell dysfunction. J. Immunol. 175, 8415-23 (2005).
Patil, V. S. et al. Precursors of human CD4+ cytotoxic T lymphocytes identified by single-cell transcriptome analysis. Sci. Immunol. 3, eaan8664 (2018).
Di Mitri, D. et al. Reversible senescence in human CD4+CD45RA+CD27− memory T cells. J. Immunol. 187, 2093-100 (2011).
Okoye, A. et al. Progressive CD4+ central-memory T cell decline results in CD4+ effector-memory insufficiency and overt disease in chronic SIV infection. J. Exp. Med. 204, 2171-2185 (2007).
Wu, T. et al. The TCF1-Bcl6 axis counteracts type I interferon to repress exhaustion and maintain T cell stemness. Sci. Immunol. 1, (2016).
Paroni, M. et al. Recognition of viral and self-antigens by TH1 and TH1/TH17 central memory cells in patients with multiple sclerosis reveals distinct roles in immune surveillance and relapses. J. Allergy Clin. Immunol. 140, 797-808 (2017).
Orban, T. et al. Reduction in CD4 central memory T-cell subset in costimulation modulator abatacept-treated patients with recent-onset type 1 diabetes is associated with slower C-peptide decline. Diabetes 63, 3449-57 (2014).
Shin, B. et al. Effector CD4 T cells with progenitor potential mediate chronic intestinal inflammation. J. Exp. Med. 215, jem.20172335 (2018).
Sallusto, F., Lenig, D., Forster, R., Lipp, M. & Lanzavecchia, A. Two subsets of memory T lymphocytes with distinct homing potentials and effector functions. Nature 401, 708-12 (1999).
Jamal-Hanjani, M. et al. Tracking the Evolution of Non-Small-Cell Lung Cancer. N. Engl. J. Med. 376, 2109-2121 (2017).
Jamal-Hanjani, M. et al. Tracking Genomic Cancer Evolution for
Precision Medicine: The Lung TRACERx Study. PLoS Biol. 12, 1-7 (2014).
Ahmadzadeh, M. et al. Tumour-infiltrating human CD4+ regulatory T cells display a distinct TCR repertoire and exhibit tumour and neoantigen reactivity. Sci. Immunol. 4, eaao4310 (2019).
Becht, E. et al. Dimensionality reduction for visualizing single-cell data using UMAP. Nat. Biotechnol. 37, 38-44 (2018).
Mueller, S. N. & Mackay, L. K. Tissue-resident memory T cells: Local specialists in immune defence. Nat. Rev. Immunol. 16, 79-89 (2016).
Thommen, D. S. et al. A transcriptionally and functionally distinct PD-1+CD8+ T cell pool with predictive potential in non-small-cell lung cancer treated with PD-1 blockade. Nat. Med. 1-11 (2018). doi:10.1038/s41591-018-0057-z
Soares, L. R., Tsavaler, L., Rivas, a & Engleman, E. G. V7 (CD101) ligation inhibits TCR/CD3-induced IL-2 production by blocking Ca2+ flux and nuclear factor of activated T cell nuclear translocation. J. Immunol. (1998).
Mahnke, Y. D., Brodie, T. M., Sallusto, F., Roederer, M. & Lugli, E. The who's who of T-cell differentiation: Human memory T-cell subsets. Eur. J. Immunol. 43, 2797-2809 (2013).
Warrington, K. J., Vallejo, A. N., Weyand, C. M. & Goronzy, J. J. CD28 loss in senescent CD4+ T cells: reversal by interleukin-12 stimulation. Blood 101, 3543-3549 (2003).
Salazar-Fontana, L.-I. et al. Cell Surface CD28 Levels Define Four CD4+ T Cell Subsets: Abnormal Expression in Rheumatoid Arthritis. Clin. Immunol. 99, 253-265 (2001).
Guo, X. et al. Global characterization of T cells in non-small-cell lung cancer by single-cell sequencing. Nat. Med. 24, 978-985 (2018).
Tilstra, J. S. et al. Kidney-infiltrating T cells in murine lupus nephritis are metabolically and functionally exhausted. J. Clin. Invest. 128, 4884-4897 (2018).
Nanki, T. et al. Migration of CX3CR1-positive T cells producing type 1 cytokines and cytotoxic molecules into the synovium of patients with rheumatoid arthritis. Arthritis Rheum. 46, 2878-2883 (2002).
Cheuk, S. et al. CD49a Expression Defines Tissue-Resident CD8+ T Cells Poised for Cytotoxic Function in Human Skin. Immunity 46, 287-300 (2017).
Quezada, S. A., Jarvinen, L. Z., Lind, E. F. & Noelle, R. J. CD40/CD154 Interactions at the Interface of Tolerance and Immunity. Annu. Rev. Immunol. 22, 307-328 (2004).
Hirschhorn-Cymerman, D. et al. Induction of tumouricidal function in CD4+ T cells is associated with concomitant memory and terminally differentiated phenotype. J. Exp. Med. 209, 2113-26 (2012).
Charoentong, P. et al. Pan-cancer Immunogenomic Analyses Reveal Genotype-Immunophenotype Relationships and Predictors of Response to Checkpoint Blockade. Cell Rep. 18, 248-262 (2017).
Gattinoni, L. et al. Wnt signaling arrests effector T cell differentiation and generates CD8+ memory stem cells. Nat. Med. 15, 808-813 (2009).
Sun, L. et al. Interferon Regulator Factor 8 (IRF8) Limits Ocular Pathology during HSV-1 Infection by Restraining the Activation and Expansion of CD8+ T Cells. PLoS One 11, e0155420 (2016).
Chen, J. et al. NR4A transcription factors limit CAR T cell function in solid tumours. Nature (2019). doi:10.1038/s41586-019-0985-x
Doering, T. A. et al. Network Analysis Reveals Centrally Connected Genes and Pathways Involved in CD8+ T Cell Exhaustion versus Memory. Immunity 37, 1130-1144 (2012).
Aran, D., Hu, Z. & Butte, A. J. xCell: digitally portraying the tissue cellular heterogeneity landscape. Genome Biol. 18, 220 (2017).
Bindea, G. et al. Spatiotemporal Dynamics of Intratumoural Immune Cells Reveal the Immune Landscape in Human Cancer. Immunity 39, 782-795 (2013).
Hebenstreit, D. et al. LEF-1 negatively controls interleukin-4 expression through a proximal promoter regulatory element. J. Biol. Chem. 283, 22490-7 (2008).
Xing, S. et al. Tcf1 and Lef1 transcription factors establish CD8(+) T cell identity through intrinsic HDAC activity. Nat. Immunol. 17, 695-703 (2016).
Magnuson, A. M. et al. Identification and validation of a tumour-infiltrating Treg transcriptional signature conserved across species and tumour types. Proc. Natl. Acad. Sci. 115, E10672-E10681 (2018).
Im, S. J. et al. Defining CD8+ T cells that provide the proliferative burst after PD-1 therapy. Nature 537, 417-421 (2016)
Miller, B. C. et al. Subsets of exhausted CD8+ T cells differentially mediate tumour control and respond to checkpoint blockade. Nat. Immunol. 20, 326-336 (2019).
Sade-Feldman, M. et al. Defining T Cell States Associated with Response to Checkpoint Immunotherapy in Melanoma. Cell 175, 998-1013.e20 (2018).
Kurtulus, S. et al. Checkpoint Blockade Immunotherapy Induces Dynamic Changes in PD-1-CD8+ Tumour-Infiltrating T Cells. Immunity 50, 181-194.e6 (2019).
Gautam, S. et al. The transcription factor c-Myb regulates CD8+ T cell sternness and antitumour immunity. Nat. Immunol. 20, 337-349 (2019).
Simoni, Y. et al. Bystander CD8+ T cells are abundant and phenotypically distinct in human tumour infiltrates. Nature 557, 575-579 (2018).
Speiser, D. E. et al. T cell differentiation in chronic infection and cancer: functional adaptation or exhaustion? Nat. Rev. Immunol. 14, 768-774 (2014).
Liu, X. et al. Regulatory T cells trigger effector T cell DNA damage and senescence caused by metabolic competition. Nat. Commun. 9, 249 (2018).
Sawant, D. V. et al. Adaptive plasticity of IL-10+ and IL-35+ Treg cells cooperatively promotes tumour T cell exhaustion. Nat. Immunol. 1 (2019). doi:10.1038/s41590-019-0346-9
He, Y. et al. MHC class II expression in lung cancer. Lung Cancer 112, 75-80 (2017).
Plitas, G. et al. Regulatory T Cells Exhibit Distinct Features in Human Breast Cancer. Immunity 45, 1122-1134 (2016).
De Simone, M. et al. Transcriptional Landscape of Human Tissue Lymphocytes Unveils Uniqueness of Tumour-Infiltrating T Regulatory Cells. Immunity 45, 1135-1147 (2016).
Roth, A. et al. PyClone: statistical inference of clonal population structure in cancer. Nat. Methods 11, 396-398 (2014).
Dobin, A. et al. STAR: ultrafast universal RNA-seq aligner. Bioinformatics 29, 15-21 (2013).
Li, B. & Dewey, C. N. RSEM: accurate transcript quantification from RNA-Seq data with or without a reference genome. BMC Bioinformatics 12, 323 (2011).
Hendry, S. et al. Assessing Tumour-Infiltrating Lymphocytes in Solid Tumours. Adv. Anat. Pathol. 24, 311-335 (2017).
Hadrup, S. R. et al. Parallel detection of antigen-specific T-cell responses by multidimensional encoding of MHC multimers. Nat. Methods (2009). doi:10.1038/nmeth.1345
Kolde, R. Package ‘pheatmap’. Bioconductor (2012).
Denkert, C. et al. Standardized evaluation of tumour-infiltrating lymphocytes in breast cancer: results of the ring studies of the international immuno-oncology biomarker working group. Mod. Pathol. 29, 1155-1164 (2016).
Thorsson, V. et al. The Immune Landscape of Cancer. Immunity 48, 812-830.e14 (2018).
Liu, J. et al. An Integrated TCGA Pan-Cancer Clinical Data Resource to Drive High-Quality Survival Outcome Analytics. Cell 173, 400-416.e11 (2018).
Ellrott, K. et al. Scalable Open Science Approach for Mutation Calling of Tumour Exomes Using Multiple Genomic Pipelines. Cell Syst. 6, 271-281.e7 (2018).
Nowicka, M. et al. CyTOF workflow: differential discovery in high-throughput high-dimensional cytometry datasets. F1000Research 6, 748 (2017).
Hahne, F. et al. flowCore: a Bioconductor package for high throughput flow cytometry. BMC Bioinformatics 10, 106 (2009).
Levine, J. H. et al. Data-Driven Phenotypic Dissection of AML Reveals Progenitor-like Cells that Correlate with Prognosis. Cell 162, 184-97 (2015).
Van Gassen, S. et al. FlowSOM: Using self-organizing maps for visualization and interpretation of cytometry data. Cytom. Part A 87, 636-645 (2015).
Wilkerson, M. D. & Hayes, D. N. ConsensusClusterPlus: a class discovery tool with confidence assessments and item tracking. Bioinformatics 26, 1572-1573 (2010).
Robinson, M. D., McCarthy, D. J. & Smyth, G. K. edgeR: a Bioconductor package for differential expression analysis of digital gene expression data. Bioinformatics 26, 139-140 (2010).
Lun, A. T. L., Richard, A. C. & Marioni, J. C. Testing for differential abundance in mass cytometry data. Nat. Methods 14, 707-709 (2017).
Li, W. V. & Li, J. J. An accurate and robust imputation method scImpute for single-cell RNA-seq data. Nat. Commun. 9, 997 (2018).
Oetjen, K. A. et al. Human bone marrow assessment by single-cell RNA sequencing, mass cytometry, and flow cytometry. JCI Insight 3, (2018).
Soneson, C. & Robinson, M. D. Bias, robustness and scalability in single-cell differential expression analysis. Nat. Methods 15, 255-261 (2018).
Gu, Z., Eils, R. & Schlesner, M. Complex heatmaps reveal patterns and correlations in multidimensional genomic data. Bioinformatics 32, 2847-2849 (2016).
Sergushichev, A. A. An algorithm for fast preranked gene set enrichment analysis using cumulative statistic calculation. bioRxiv 060012 (2016). doi:10.1101/060012
Subramanian, A. et al. Gene set enrichment analysis: a knowledge-based approach for interpreting genome-wide expression profiles. Proc. Natl. Acad. Sci. U.S.A 102, 15545-50 (2005).
Godec, J. et al. Compendium of Immune Signatures Identifies
Conserved and Species-Specific Biology in Response to Inflammation. Immunity 44, 194-206 (2016).
Abbas, A. R., Wolslegel, K., Seshasayee, D., Modrusan, Z. & Clark, H. F. Deconvolution of blood microarray data identifies cellular activation patterns in systemic lupus erythematosus. PLoS One 4, e6098 (2009).
Chevalier, N. et al. CXCR5 expressing human central memory CD4 T cells and their relevance for humoral immune responses. J. Immunol. 186, 5556-68 (2011).
Jeffrey, K. L. et al. Positive regulation of immune cell function and inflammatory responses by phosphatase PAC-1. Nat. Immunol. 7, 274-283 (2006).
Danaher, P. et al. Gene expression markers of Tumour Infiltrating Leukocytes. J. Immunother. Cancer 5, 18 (2017).
Li, H. et al. Dysfunctional CD8 T Cells Form a Proliferative, Dynamically Regulated Compartment within Human Melanoma. Cell 176, 775-789.e18 (2019).
Reading, J. L. et al. The function and dysfunction of memory CD8+ T cells in tumor immunity. Immunol. Rev. 283, 194-212 (2018).
McGranahan, N., Furness, A. J. S., Rosenthal, R., Ramskov, S., Lyngaa, R., Saini, S. K., . . . Swanton, C. (2016). Clonal neoantigens elicit T cell immunoreactivity and sensitivity to immune checkpoint blockade. Science. Retriev. Science (80-.). 490, 1-11 (2016).
Djenidi, F. et al. CD8+CD103+ Tumor-Infiltrating Lymphocytes Are Tumor-Specific Tissue-Resident Memory T Cells and a Prognostic Factor for Survival in Lung Cancer Patients. J. Immunol. 194, 3475-3486 (2015).
Lee, L. N. et al. Chemokine gene expression in lung CD8 T cells correlates with protective immunity in mice immunized intra-nasally with Adenovirus-85A. BMC Med. Genomics 3, (2010).
Schietinger, A. et al. Tumor-Specific T Cell Dysfunction Is a Dynamic Antigen-Driven Differentiation Program Initiated Early during Tumorigenesis. Immunity 45, 389-401 (2016).
Oakes, T. et al. Quantitative characterization of the T cell receptor repertoire of naïve and memory subsets using an integrated experimental and computational pipeline which is robust, economical, and versatile. Front. Immunol. (2017). doi:10.3389/fimmu.2017.01267
Boldajipour, B., Nelson, A. & Krummel, M. F. Tumor-infiltrating lymphocytes are dynamically desensitized to antigen but are maintained by homeostatic cytokine. JCI Insight (2016). doi:10.1172/jci.insight.89289
Wilson, E. B. et al. Blockade of chronic type I interferon signaling to control persistent LCMV infection. Science (80-.). (2013). doi:10.1126/science.1235208
Chihara, N. et al. Induction and transcriptional regulation of the co-inhibitory gene module in T cells. Nature 558, 454-459 (2018).
Olesin, E., Nayar, R., Saikumar-Lakshmi, P. & Berg, L. J. The Transcription Factor Runx2 Is Required for Long-Term Persistence of Antiviral CD8+ Memory T Cells. ImmunoHorizons (2018). doi:10.4049/immunohorizons.1800046
Quintana, F. J. et al. Aiolos promotes T H17 differentiation by directly silencing 112 expression. Nat. Immunol. (2012). doi:10.1038/ni.2363
Smith, K. N. et al. Evolution of Neoantigen Landscape during Immune Checkpoint Blockade in Non-Small Cell Lung Cancer. Cancer Discov. 7, 264-276 (2016).
Riaz, N. et al. Tumor and Microenvironment Evolution during Immunotherapy with Nivolumab. Cell 171, 934-949.e15 (2017).
McGranahan, N. et al. Allele-Specific HLA Loss and Immune Escape in Lung Cancer Evolution. Cell 171, 1259-1271.e11 (2017).
Arrieta, V. A. et al. The possibility of cancer immune editing in gliomas. A critical review. OncoImmunology (2018). doi:10.1080/2162402X.2018.1445458
Marty, R. et al. MHC-I Genotype Restricts the Oncogenic Mutational Landscape. Cell (2017). doi:10.1016/j.cell.2017.09.050
Luksza, M. et al. A neoantigen fitness model predicts tumour response to checkpoint blockade immunotherapy. Nature 551, 517-520 (2017).
Havel, J. J., Chowell, D. & Chan, T. A. The evolving landscape of biomarkers for checkpoint inhibitor immunotherapy. Nat. Rev. Cancer 19, 133-150 (2019).
Chen, H. et al. Cytofkit: A Bioconductor Package for an Integrated Mass Cytometry Data Analysis Pipeline. PLoS Comput. Biol. (2016). doi:10.1371/journal.pcbi.1005112
Spitzer, M. H. et al. An interactive reference framework for modeling a dynamic immune system. Science (80-.). 349, (2015).
Bruggner, R. V., Bodenmiller, B., Dill, D. L., Tibshirani, R. J. & Nolan, G. P. Automated identification of stratifying signatures in cellular subpopulations. Proc. Natl. Acad. Sci. (2014). doi:10.1073/pnas.1408792111
Weber, L. M. & Robinson, M. D. Comparison of clustering methods for high-dimensional single-cell flow and mass cytometry data. Cytom. Part A (2016). doi:10.1002/cyto.a.23030
Jesse M. Zaretsky, B. S., Angel Garcia-Diaz, Ph.D., Daniel S. Shin, M. D., Helena Escuin-Ordinas, Ph.D., Willy Hugo, Ph.D., Siwen Hu-Lieskovan, M. D., Ph.D., Davis Y. Torrejon, M. D., Gabriel Abril-Rodriguez, M. Sc., Salemiz Sandoval, Ph.D., Lucas Barthly, M. Sc., Justin Saco, B. S., Blanca Homet Moreno, M. D., et al. Mutations Associated with Acquired Resistance to PD-1 Blockade in Melanoma. N Engl J Med 2016; 375:819-829 DOI: 10.1056/NEJMoa1604958
Gao J et al. Loss of IFN-γ Pathway Genes in Tumor Cells as a Mechanism of Resistance to Anti-CTLA-4 Therapy. Cell. 2016 Oct. 6; 167(2):397-404.e9. doi: 10.1016/j.cell.2016.08.069. Epub 2016 Sep. 22.
Kaplan D H, Shankaran V, Dighe A S, Stockert E, Aguet M, Old L J, Schreiber R D. Demonstration of an interferon gamma-dependent tumor surveillance system in immunocompetent mice. Proc Natl Acad Sci USA. 1998 Jun. 23; 95(13):7556-61.
Balkhi M Y, Wittmann G, Xiong F, Junghans R P. YY1 Upregulates Checkpoint Receptors and Downregulates Type I Cytokines in Exhausted, Chronically Stimulated Human T Cells. iScience. 2018; 2:105-122. doi:10.1016/j.isci.2018.03.009
David M. Lee, Herman F. Staats, John S. Sundy, Dhavalkumar D. Patel, Gregory D. Sempowski, Richard M. Scearce, Dawn M. Jones, Barton F. Haynes. Immunologic Characterization of CD7-Deficient Mice. The Journal of Immunology Jun. 15, 1998, 160 (12) 5749-5756.
Sakellariou-Thompson D, Forget M A, Creasy C, Bernard V, Zhao L, Kim Y U, Hurd M W, Uraoka N, Parra E R, Kang Y, Bristow C A, Rodriguez-Canales J, Fleming J B, Varadhachary G, Javle M, Overman M J, Alvarez H A, Heffernan T P, Zhang J, Hwu P, Maitra A, Haymaker C, Bernatchez C. 4-1BB Agonist Focuses CD8+ Tumor-Infiltrating T-Cell Growth into a Distinct Repertoire Capable of Tumor Recognition in Pancreatic Cancer. Clin Cancer Res. 2017 Dec. 1; 23(23):7263-7275. doi: 10.1158/1078-0432.CCR-17-0831.
Scott, A. C., Dündar, F., Zumbo, P. et al. TOX is a critical regulator of tumour-specific T cell differentiation. Nature 571, 270-274 (2019).
Jolanda Brummelman, Emilia M. C. Mazza, Giorgia Alvisi, Federico S. Colombo, Andrea Grilli, Joanna Mikulak, Domenico Mavilio, Marco Alloisio, Francesco Ferrari, Egesta Lopci, Pierluigi Novellis, Giulia Veronesi, Enrico Lugli; High-dimensional single cell analysis identifies stem-like cytotoxic CD8⁺ T cells infiltrating human tumors. J Exp Med 1 Oct. 2018; 215 (10): 2520-2535. doi: https://doi.org/10.1084/jem.20180684
D'Angelo S P, Melchiori L, Merchant M S, Bernstein D, Glod J, Kaplan R, Grupp S, Tap W D, Chagin K, Binder G K, Basu S, Lowther D E, Wang R, Bath N, Tipping A, Betts G, Ramachandran I, Navenot J M, Zhang H, Wells D K, Van Winkle E, Kari G, Trivedi T, Holdich T, Pandite L, Amado R, Mackall C L. Antitumor Activity Associated with Prolonged Persistence of Adoptively Transferred NY-ESO-1 ^{c259}T Cells in Synovial Sarcoma. Cancer Discov. 2018 August; 8(8):944-957.
Jiesheng Li, Zemin Zhang, Xianwen Ren. Landscape of transcript isoforms in single T cells infiltrating in non-small cell lung cancer. Journal of Genetics and Genomics. Volume 47, Issue 7, 20 Jul. 2020, Pages 373-388.
Dudley, M. E., Wunderlich, J. R., Shelton, T. E., Even, J., & Rosenberg, S. A. (2003). Generation of tumor-infiltrating lymphocyte cultures for use in adoptive transfer therapy for melanoma patients. Journal of immunotherapy (Hagerstown, Md.: 1997), 26(4), 332-342.
Peter J. R. Ebert, Jeanne Cheung, Yagai Yang, Erin McNamara, Rebecca Hong, Marina Moskalenko, Stephen E. Gould, Heather Maecker, Bryan A. Irving, Jeong M. Kim, Marcia Belvin, Ira Mellman, MAP Kinase Inhibition Promotes T Cell and Anti-tumor Activity in Combination with PD-L1 Checkpoint Blockade, Immunity, Volume 44, Issue 3, 2016, Pages 609-621.
Devikala Gurusamy, Amanda N. Henning, Tori N. Yamamoto, Zhiya Yu, Nikolaos Zacharakis, Sri Krishna, Rigel J. Kishton, Suman K. Vodnala, Arash Eidizadeh, Li Jia, Christine M. Kariya, Mary A. Black, Robert Eil, Douglas C. Palmer, Jenny H. Pan, Madhusudhanan Sukumar, Shashank J. Patel, Nicholas P. Restifo, Multi-phenotype CRISPR-Cas9 Screen Identifies p38 Kinase as a Target for Adoptive Immunotherapies, Cancer Cell, Volume 37, Issue 6, 2020, Pages 818-833.e9
Piccio L, Vermi W, Boles K S, Fuchs A, Strader C A, Facchetti F, Cella M, Colonna M. Adhesion of human T cells to antigen-presenting cells through SIRPbeta2-CD47 interaction costimulates T-cell proliferation. Blood. 2005 Mar. 15; 105(6):2421-7.

1. An engineered T cell for use in a method of treatment of a proliferative disorder in a mammalian subject, wherein the T cell has been engineered to have modulated expression of one or more genes selected from STOM, FURIN, SIT1, CD7, SAMSN1, SIRPG, CD82, FCRL3, IL1RAP, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3.

2. The engineered T cell for use of claim 1, wherein the one or more genes are selected from STOM, FURIN, SIT1, CD7, SAMSN1, SIRPG, CD82, FCRL3, IL1RAP, AXL, E2F1.

3. The engineered T cell for use according to any one of the preceding claims, wherein the one or more genes are selected from STOM, FURIN, SIT1 and CD7.

4. The engineered T cell for use of claim 1, wherein the engineered T cell has reduced expression of one or more genes selected from SIT1, SAMSN1, SIRPG, CD7, CD82, FCRL3, IL1RAP, FURIN, STOM, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3, and/or increased expression of CD7 and/or SIRPG or increased activity of CD7 and/or SIRPG.

5. The engineered T cell for use according to any one of the preceding claims, wherein the one or more genes are selected from CD7, CD82, COTL1, DUSP4, FABP5, ITM2A, PARK7, PHLDA1, RAB27A, RBPJ, RGS1, RGS2, SAMSN1, SIT1, SIRPG and TNIP3, preferably wherein the engineered T cell is a CD8⁺ T cell.

6. The engineered T cell for use according to any one of the preceding claims, wherein the engineered T cell is a CD8⁺ T cell and wherein the one or more genes are selected from SIT1, CD7, STOM, FURIN, IL1RAP, SIRPG, AXL, E2F1A, CD82, SAMSN1, and FCRL3, preferably wherein the one or more genes are selected from SIT1, CD7, STOM, FURIN, IL1RAP and SIRPG.

7. The engineered T cell for use of any of claims 1 to 4, wherein the one or more genes are selected from EPHA1, FCRL3, PECAM1, STOM, AXL, FURIN, IL1RAP, E2F1, C5ORF30, CLDND1, GFI1, RNASEH2A, SIRPG and SUV39H1, optionally wherein the one or more genes are selected from EPHA1, FCRL3, PECAM1, STOM, AXL, FURIN, SIRPG and IL1RAP, preferably wherein the engineered T cell is a CD4⁺ T cell, such as an effector CD4⁺ T cell.

8. The engineered T cell for use according to any one of claims 1 to 4, wherein the engineered T cell is a CD4⁺ T cell and wherein the one or more genes are selected from SIT1, CD7, STOM, FURIN, IL1RAP, SIRPG, AXL, E2F1A, CD82, SAMSN1, and FCRL3.

9. The engineered T cell for use of any one of the preceding claims, wherein the T cell comprises a chimeric antigen receptor T cell (CAR-T), an engineered T cell receptor (TCR) T cell, an engineered T cell derived from PBMCs or a Neoantigen-reactive T cell (NAR-T), optionally wherein the engineered T cell receptor T cell expresses a transgenic T cell receptor and/or wherein the one or more genes comprise SIT1 and the engineered T cell comprises a CAR-T cell or an engineered T cell derived from PBMCs.

10. The engineered T cell for use of any one of the preceding claims, wherein the T cell is autologous to said subject.

11. The engineered T cell for use according to any one of the preceding claims, wherein the proliferative disorder comprises a solid tumour, optionally wherein the tumour is selected from bladder cancer, gastric cancer, oesophageal cancer, breast cancer, colorectal cancer, cervical cancer, ovarian cancer, endometrial cancer, kidney cancer, lung cancer, brain cancer, melanoma, lymphoma, small bowel cancers, leukaemia, pancreatic cancer, hepatobiliary tumours, germ cell cancers, prostate cancer, head and neck cancers, thyroid cancer and sarcomas, and/or wherein the proliferative disorder is selected from lung adenocarcinoma, renal clear cell carcinoma, pancreatic adenocarcinoma, renal papillary carcinoma, hepatocellular carcinoma, adrenocortical carcinoma and mesothelioma.

12. The engineered T cell for use according to any one of the preceding claims, wherein the proliferative disorder comprises a tumour predicted to have high neoantigen load, optionally wherein the proliferative disorder is selected from melanoma, Lung squamous cell carcinoma, lung adenocarcinoma, bladder cancer, small cell lung cancer, oesophagus cancer, colorectal cancer, cervical cancer, head and neck cancer, stomach cancer, endometrial cancer, and liver cancer.

13. The engineered T cell for use according to any one of the preceding claims, wherein the proliferative disorder comprises a tumour predicted to have developed or be at risk of developing immune escape.

14. The engineered T cell for use according to claim 13, wherein the tumour is selected from: (i) a tumour in a patient that has already undergone immunotherapy and has failed to respond, or no longer responds to the immunotherapy, (ii) a tumour in a patient that is predicted to be unlikely to respond to immunotherapy, where the patient may be (immunotherapy) treatment naïve, (iii) a tumour that is determined to have no or low T-cell infiltration, and (iv) a tumour that has a high proportion of dysfunctional T cells in the tumour-infiltrating T cell population.

15. The engineered T cell for use according to any one of the preceding claims, wherein the engineered T cell has been engineered to knock-out or downregulate expression of the one or more genes.

16. The engineered T cell for use according to any one of the preceding claims, wherein the engineered T cell has been engineered to: (i) overexpress CD7 and/or SIRPG, optionally wherein the engineered T cell is a tumour-infiltrating lymphocyte engineered to overexpress CD7 or wherein the engineered T cell is not a tumour-infiltrating lymphocyte and the engineered T cell has been engineered to overexpress SIRPG; or
(ii) have reduced expression of CD7 and/or SIRPG, optionally wherein the engineered T cell is a tumour-infiltrating lymphocyte engineered to have reduced expression of SIRPG or wherein the engineered T cell is not a tumour-infiltrating lymphocyte and the engineered T cell has been engineered to have reduced expression of CD7.

17. The engineered T cell for use according to claim 15 or claim 16, wherein said knock-out or downregulation and/or said overexpression has been engineered by CRISPR-mediated gene editing, transcription activator-like effector nucleases (TALENs) transient downregulation using short hairpin RNA (shRNA), small interfering RNA (siRNA), microRNA (miRNA) or RNA constructs for overexpression.

18. The engineered T cell for use according to any one of the preceding claims, wherein said method of treatment further comprises simultaneous, sequential or separate administration of an immune checkpoint inhibitor therapy.

19. The engineered T cell for use according to any one of the preceding claims, wherein the engineered T cell is a CD4⁺ T cell having cytotoxic activity and/or a CD8⁺ T cell having cytotoxic activity.

20. A method of treatment of a proliferative disorder in a mammalian subject, comprising administering a therapeutically effective amount of an engineered T cell to the subject in need thereof, wherein the T cell has been engineered to have modulated expression of one or more genes selected from STOM, FURIN, SIT1, CD7, SAMSN1, SIRPG, CD82, FCRL3, IL1RAP, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3.

21. The method of claim 20, wherein the T cell has the features of any of claims 2 to 10 or 15 to 18, and/or wherein the proliferative disorder has the features of any of claims 11 to 14.

22. The method according to any one of claims 19 to 21, wherein the T cell is engineered to knock-out or downregulate expression of the one or more genes prior to being administered to the subject.

23. The method according to any one of claims 19 to 22, wherein the T cell is engineered to overexpress CD7 and/or SIRPG prior to being administered to the subject.

24. The method according to any one of claims 19 to 23, wherein said method of treatment further comprises simultaneous, sequential or separate administration of an immune checkpoint inhibitor therapy to the subject.

25. An activity modulator of a protein encoded by a gene selected from STOM, FURIN, SIT1, CD7, SAMSN1, SIRPG, CD82, FCRL3, IL1RAP, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3, for use in a method of enhancing immunotherapy in a subject having a proliferative disorder.

26. The activity modulator for use according to claim 25, wherein the gene is selected from: STOM, FURIN, CD7, SIT1, IL1RAP, SAMSN1, SIRPG, CD82, FCRL3, E2F1, and AXL, preferably wherein the activity modulator is an inhibitor, optionally wherein the inhibitor is a small molecule inhibitor or a blocking antibody.

27. The activity modulator for use according to claim 25, wherein the activity modulator is an activator, such as an agonist antibody or an agonist ligand, of CD7 and/or SIRPG.

28. The activity modulator for use according to claim 26, wherein the activity modulator is a small molecule inhibitor of AXL, CLDND1, E2F1, FABP5, FURIN, IL1RAP, SAMSN1, SUV39H1 or TNIP3.

29. The activity modulator for use according to claim 26, wherein the activity modulator is a (poly)peptide, such an antibody or fragment thereof, that binds to and inhibits AXL, CD7, FCRL3, EPHA1, IL1RAP, ITM2A, PARK7, PECAM1, TNIP3 or SIRPG.

30. The activity modulator for use according to any one of claims 25 to 29, wherein said immunotherapy comprises immune checkpoint inhibition, an anti-tumour vaccine or a T cell therapy, and/or wherein the amount of activity modulator administered to the subject is sufficient to enhance cytotoxic activity of CD4⁺ T cells and/or CD8+ T cells in the subject.

31. The activity modulator for use according to any one of claims 25 to 30, wherein the proliferative disorder has the features of any of claims 11 to 14.

32. An activity modulator of a protein encoded by a gene selected from STOM, FURIN, CD7, SIT1, IL1RAP, SAMSN1, SIRPG, CD82, FCRL3, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3, for use in a method of enhancing the immune response of a subject having a proliferative disorder, optionally wherein the activity modulator for use has the features of any of claim 25 to 29 or 31-32, and/or wherein the method further comprises administration of an immunotherapy.

33. The activity modulator for use according to any one of claims 25 to 32, wherein the method further comprises administration of an immunotherapy using an engineered T cell according to any one of claims 1 to 19.

34. The method according to any one of claims 19 to 24, wherein said method of treatment further comprises simultaneous, sequential or separate administration of an activity modulator according to any one of claims 25 to 33 to the subject.

35. A method of treatment of a proliferative disorder in a mammalian subject, comprising administering a therapeutically effective amount of an activity modulator of one or more proteins encoded by genes selected from STOM, FURIN, CD7, SIT1, IL1RAP, SAMSN1, SIRPG, CD82, FCRL3, AXL, E2F1, C5ORF30, CLDND1, COTL1, DUSP4, EPHA1, FABP5, GFI1, IL1RAP, ITM2A, PARK7, PECAM1, PHLDA1, RAB27A, RBPJ, RGS1, RGS2, RNASEH2A, SUV39H1, and TNIP3 to the subject, wherein the activity modulator enhances cytotoxic activity of one or more T cells in the subject and thereby treats the proliferative disorder.

36. The method of treatment according to claim 35, wherein the activity modulator has the features of any of claims 25 to 31, and/or wherein the method of treatment further comprises administering an engineered T cell according to any one of claims 1 to 19.

37. A method for producing an engineered T cell, comprising genetically engineering a T cell to modulate expression of one or more genes selected from STOM, FURIN, CD7, SIT1, IL1RAP, SAMSN1, SIRPG, FCRL3, AXL, E2F1, C5ORF30, CLDND1, COTL1, DUSP4, EPHA1, FABP5, GFI1, ITM2A, PARK7, PECAM1, PHLDA1, RAB27A, RBPJ, RGS1, RGS2, RNASEH2A, SUV39H1, and TNIP3, optionally wherein:
the modulation comprises knocking-out or downregulating expression of the one or more genes and/or the modulation comprises enhancing expression of CD7 and/or SIRPG; and/or
the engineered T cell is a tumour-infiltrating lymphocyte and the modulation comprises knocking-out or downregulating expression of one or more genes selected from STOM, FURIN, SIT1, IL1RAP, SAMSN1, SIRPG, FCRL3, AXL, and E2F1, and/or enhancing the expression of CD7; and/or
the engineered T cell is not a tumour-infiltrating lymphocyte and the modulation comprises enhancing the expression of SIRPG.

38. The method of claim 37, further comprising culturing the T cell under conditions suitable for expansion to provide an expanded cell population.

39. The method of claim 37 or claim 38, wherein the method is performed in vitro, and/or wherein the engineered T cell has the features of any one of claims 1 to 19.

40. The method of any one of claims 37 to 39, wherein genetically engineering a T cell is performed by CRISPR/Cas9-mediated gene editing, transcription activator-like effector nucleases (TALENs) transient downregulation using short hairpin RNA (shRNA), small interfering RNA (siRNA), microRNA (miRNA) or RNA constructs for overexpression or by introducing a nucleic acid or vector into the cell.
